(19) United States
(12) Patent Application Publication
(10) Pub. No.: US 2011/0034434 A1
(43) Pub. Date: Feb. 10, 2011

(54) PRODRUGS OF FUSED HETEROCYCLIC INHIBITORS OF D-AMINO ACID OXIDASE

(75) Inventors: Michele L. R. Heffernan, Worcester, MA (US); Richard Dennis, Garner, NC (US); James M. Dorsey, Durham, NC (US); Robert J. Foglesong, Durham, NC (US); Michael L. Jones, Chapel Hill, NC (US); Cyprian O. Ogbu, Durham, NC (US); Mustapha Soukri, Raleigh, NC (US); Kerry L. Spear, Concord, MA (US); Michael A. Orsini, Leominster, MA (US)

Correspondence Address: MORGAN, LEWIS & BOCKIUS LLP (SF), One Market, Spear Street Tower, Suite 2800, San Francisco, CA, 94105, US

(73) Assignee: SEPRACOR INC., Marlborough, MA (US)

(21) Appl. No.: 12/852,215

(22) Filed: Aug. 6, 2010

(30) Foreign Application Priority Data
Aug. 7, 2009 (AR) P090103040
Aug. 10, 2009 (TW) 098126779

(51) Int. Cl. A61K 31/397; C07D 491/04; A61K 31/407; C07D 401/14; A61K 31/4439; C07D 413/14; A61K 31/5377; A61K 31/454; C07D 403/14; A61K 31/496; A61K 31/4178; A61P 29/00; A61P 25/28

(52) U.S. Cl. 514/210.18; 548/453; 514/412; 514/414; 546/276.7; 514/338; 544/143; 514/233.8; 546/198; 514/321; 544/373; 514/254.08; 548/311.7; 514/397

(57) ABSTRACT

The invention relates to prodrugs of fused heterocyclic inhibitors of D-amino oxidase (DAAO) and methods of treating diseases and conditions, wherein modulation of D-amino acid oxidase activity, D-serine levels, D-serine oxidative products and NMDA receptor activity in the nervous system of a mammalian subject is effective.

### CROSS-REFERENCES TO RELATED APPLICATIONS

This application claims priority under 35 U.S.C. §119(a) to Argentine Patent Application No. P 09 01 03040 filed on Aug. 7, 2009, and R.O.C. (Taiwan) Patent Application No. 098126779 filed Aug. 10, 2009, each of which is incorporated herein by reference in its entirety for all purposes.

### FIELD OF THE INVENTION

This invention relates to prodrugs of enzyme inhibitors, particularly D amino acid oxidase (DAAO), and methods of treating diseases and conditions, wherein modulation of D-amino acid oxidase activity, D-serine levels, D-serine oxidative products and NMDA receptor activity in the nervous system of a mammalian subject is effective.

### BACKGROUND OF THE INVENTION

The enzyme D-amino acid oxidase (DAAO) metabolizes D-amino acids, and in particular, metabolizes D-serine in vitro at physiological pH. DAAO is expressed in the mammalian brain and periphery. D-Serine's role as a neurotransmitter is important in the activation of the N-methyl-D-aspartate (NMDA) selective subtype of the glutamate receptor, an ion channel expressed in neurons, here denoted as NMDA receptor.

NMDA receptors mediate many physiological functions. NMDA receptors are complex ion channels containing multiple protein subunits that act either as binding sites for transmitter amino acids and/or as allosteric regulatory binding sites to regulate ion channel activity. D-serine, released by glial cells, has a distribution similar to NMDA receptors in the brain and acts as an endogenous ligand of the allosteric “glycine” site of these receptors (Mothet et al., PNAS, 97:4926 (2000)), the occupation of which is required for NMDA receptor operation. D-serine is synthesized in the brain through serine racemase and degraded by D-amino oxidase (DAAO) after release.

Small organic molecules, which inhibit the enzymatic cycle of DAAO, may control the levels of D-serine, and thus influence the activity of the NMDA receptor in the brain. NMDA receptor activity is important in a variety of disease states, such as schizophrenia, psychosis, ataxias, ischemia, several forms of pain including neuropathic pain, and deficits in memory and cognition.

DAAO inhibitors may also control production of toxic metabolites of D-serine oxidation, such as hydrogen peroxide and ammonia. Thus, these molecules may influence the progression of cell loss in neurodegenerative disorders. Neurodegenerative diseases are diseases in which CNS neurons and/or peripheral neurons undergo a progressive loss of function, usually accompanied by (and perhaps caused by) a physical deterioration of the structure of either the neuron itself or its interface with other neurons. Such conditions include Parkinson's disease, Alzheimer's disease, Huntington's disease and neuropathic pain. N-methyl-D-aspartate (NMDA)-glutamate receptors are expressed at excitatory synapses throughout the central nervous system (CNS). These receptors mediate a wide range of brain processes, including synaptic plasticity, that are associated with certain types of memory formation and learning. NMDA-glutamate receptors require binding of two agonists to induce neurotransmission. One of these agonists is the excitatory amino acid L-glutamate, while the second agonist, at the so-called “strychnine-insensitive glycine site”, is now thought to be D-serine. In animals, D-serine is synthesized from L-serine by serine racemase and degraded to its corresponding ketoacid by DAAO. Together, serine racemase and DAAO are thought to play a crucial role in modulating NMDA neurotransmission by regulating CNS concentrations of D-serine.

Known inhibitors of DAAO include benzoic acid, pyrrole-2-carboxylic acids, and indole-2-carboxylic acids, as described by Frisell, et al., J. Biol. Chem., 223:75-83 (1956) and Parikh et al., JACS, 80:953 (1958). Indole derivatives and particularly certain indole-2-carboxylates have been described in the literature for treatment of neurodegenerative disease and neurotoxic injury. EP Publication No. 396124 discloses indole-2-carboxylates and derivatives for treatment or management of neurotoxic injury resulting from a CNS disorder or traumatic event or in treatment or management of a neurodegenerative disease. Several examples of traumatic events that may result in neurotoxic injury are given, including hypoxia, anoxia, and ischemia, associated with perinatal asphyxia, cardiac arrest or stroke. Neurodegeneration is associated with CNS disorders such as convulsions and epilepsy. U.S. Pat. Nos. 5,373,018; 5,374,649; 5,686,461; 5,962,496 and 6,100,289, to Cugola et al., disclose treatment of neurotoxic injury and neurodegenerative disease using indole derivatives. None of the above references mention improvement or enhancement of learning, memory or cognition.

WO/2003/039540 to Heefner et al. and U.S. Patent Application Publication Nos. 2005/0143443 to Fang et al. and 2005/0143434 to Fang et al. disclose DAAO inhibitors, including indole-2-carboxylic acids, and methods of enhancing learning, memory and cognition as well as methods for treating neurodegenerative disorders. Publication No. WO/2005/089753 discloses benzisoxazole analogs and methods of treating mental disorders, such as schizophrenia. However, a need for additional drug molecules that are effective in treating memory defects, impaired learning, loss of cognition, and other symptoms related to NMDA receptor activity, remains. Various embodiments of the present inventions address this and other needs.

### SUMMARY OF THE INVENTION

In various aspects, the present inventions provide novel prodrugs of inhibitors of D-amino acid oxidase that yield active drug molecule (e.g., upon metabolization of the prodrug) that are useful for enhancing learning, memory and/or cognition, and in the prevention and treatment of a variety of diseases and/or conditions including neurological disorders, pain, ataxia, and convulsion.

In various aspects, the present inventions provide a compound having a structure according to Formula A:

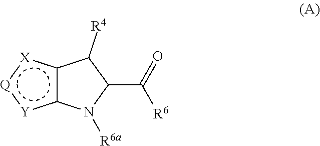

in which Q is a member selected from O, S, N and CR¹; X is a member selected from O, S, N, NR³ and CR^{2a}; and Y is a member selected from O, S, N, NR³ and CR^{2b}. R¹ is a member selected from H, F, substituted or unsubstituted arylalkyl, substituted or unsubstituted heteroarylalkyl, substituted or unsubstituted C₄-C₁₀ cycloalkyl, and substituted or unsubstituted C₄-C₁₀ heterocycloalkyl. R^{2a} is a member selected from H, F, Cl, Br, CN, substituted or unsubstituted C₃-C₆ alkyl, substituted or unsubstituted arylalkyl, substituted or unsubstituted heteroarylalkyl, substituted or unsubstituted C₄-C₁₀ cycloalkyl, substituted or unsubstituted C₄-C₁₀ heterocycloalkyl and alkenyl. R^{2b} is a member selected from H, F, substituted or unsubstituted C₃-C₆ alkyl, substituted or unsubstituted arylalkyl, substituted or unsubstituted heteroarylalkyl, substituted or unsubstituted C₄-C₁₀ cycloalkyl, and substituted or unsubstituted C₄-C₁₀ heterocycloalkyl and alkenyl. R³ is a member selected from H, substituted or unsubstituted C₁-C₆ alkyl, substituted or unsubstituted arylalkyl, substituted or unsubstituted heteroarylalkyl, substituted or unsubstituted C₄-C₁₀ cycloalkyl, and substituted or unsubstituted C₄-C₁₀ heterocycloalkyl.

R⁴ is a member selected from H, F, Cl, Br, CN, unsubstituted C₁-C₆ alkyl, substituted or unsubstituted arylalkyl, substituted or unsubstituted heteroarylalkyl, substituted or unsubstituted C₄-C₁₀ cycloalkyl and alkenyl.

R⁶ is a member selected from OR²⁶, O⁻M⁺, SR²⁷, OPO₃R^{27a}, and NR²⁸R²⁹, and R⁶ and R^{6a} are optionally joined to form a ring. R²⁶ is a member selected from H, acyl, substituted or unsubstituted alkyl, substituted or unsubstituted heteroalkyl, substituted or unsubstituted aryl, substituted or unsubstituted heteroaryl, substituted or unsubstituted heterocycloalkyl, and alkenyl. Exemplary moieties include substituted or unsubstituted arylalkyl, substituted or unsubstituted heteroarylalkyl, and substituted or unsubstituted cycloalkyl. M⁺ is a member selected from inorganic positive ions and organic positive ions. R²⁷ is substituted or unsubstituted alkyl. R^{27a} is substituted or unsubstituted alkyl or a positive organic or inorganic ion. R²⁸ and R²⁹ are members independently selected from H, OR³⁰, acyl, substituted or unsubstituted alkyl, substituted or unsubstituted heteroalkyl, substituted or unsubstituted aryl, substituted or unsubstituted heteroaryl, and substituted or unsubstituted heterocycloalkyl, and R²⁸ and R²⁹ together with the nitrogen to which they are bound optionally form a ring. R³⁰ is a member selected from H, acyl substituted or unsubstituted alkyl and substituted or unsubstituted heteroalkyl.

R^{6a} is a member selected from:

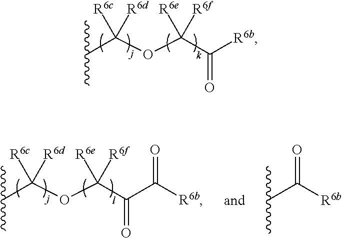

wherein R^{6c}, R^{6d}, R^{6e} and R^{6f} are members independently selected from H, substituted or unsubstituted alkyl, substituted or unsubstituted heteroalkyl, substituted or unsubstituted arylalkyl, substituted or unsubstituted heteroarylalkyl, substituted or unsubstituted cycloalkyl, substituted or unsubstituted heterocycloalkyl and alkenyl. R^{6c} and R^{6d} are optionally joined to form a ring. R^{6e} and R^{6f} are optionally joined to form a ring. R^{6c} and R^{6e} are optionally joined to form a ring. The indices j and l are integers independently selected from 1 to 5. The index k is an integer from 0 to 5. R^{6b} is a member selected from substituted or unsubstituted aryl, substituted or unsubstituted alkyl, substituted or unsubstituted heteroalkyl, substituted or unsubstituted arylalkyl, substituted or unsubstituted heteroarylalkyl, substituted or unsubstituted cycloalkyl, substituted or unsubstituted heterocycloalkyl and alkenyl.

In various aspects, the present inventions provide a compound having a structure according to Formula B:

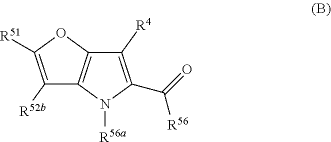

In Formula B, R⁵¹ and R^{52b} are members independently selected from H and F; R⁴ is as described for Formula A, above. In various preferred embodiments, R⁴ is selected from H and F. R^{56a} is a member selected from H, substituted or unsubstituted alkyl; substituted or unsubstituted heteroalkyl and R^{6a}; wherein only one of R^{56a} and R^{26b} can be H.

R⁵⁶ is a member selected from OR^{26b}, SR²⁷ and NR²⁸R²⁹. R⁵⁶ and R^{56a} are optionally joined to form a ring. In various embodiments, R^{26b} is a member selected from H, acyl, substituted or unsubstituted C₄ or larger alkyl, substituted or unsubstituted heteroalkyl, substituted or unsubstituted aryl, substituted or unsubstituted heteroaryl, substituted or unsubstituted arylalkyl, substituted or unsubstituted heteroarylalkyl, substituted or unsubstituted cycloalkyl, substituted or unsubstituted heterocycloalkyl and alkenyl, wherein only one of R^{56a} and R^{26b} can be H. Exemplary moieties for R²⁷ include substituted or unsubstituted alkyl; and R²⁸ and R²⁹ are members independently selected from H, OR³⁰, acyl, substituted or unsubstituted alkyl, substituted or unsubstituted heteroalkyl, substituted or unsubstituted aryl, substituted or unsubstituted heteroaryl, and substituted or unsubstituted heterocycloalkyl. R²⁸ and R²⁹ together with the nitrogen to which they are bound optionally form a ring. In various embodiments, R³⁰ is a member selected from H, acyl substituted or unsubstituted alkyl and substituted or unsubstituted heteroalkyl.

In various aspects, the present inventions provide a compound having a structure according to Formula C:

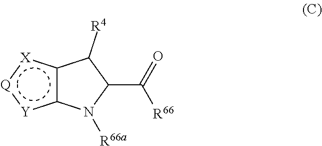

In Formula C, the radicals X, Q, Y and R⁴, are as described for Formula A above. R^{66a} a member selected from H, substituted or unsubstituted alkyl and substituted or unsubstituted heteroalkyl. R⁶⁶ is OR^{26c}. R^{66a} and R⁶⁶ are optionally joined to form a ring. R^{26c} is a member selected from the following:

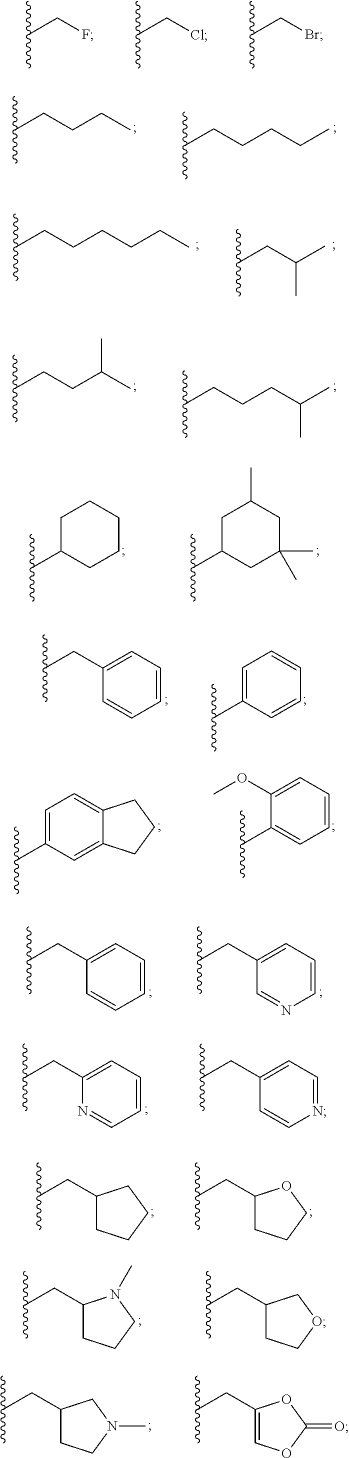

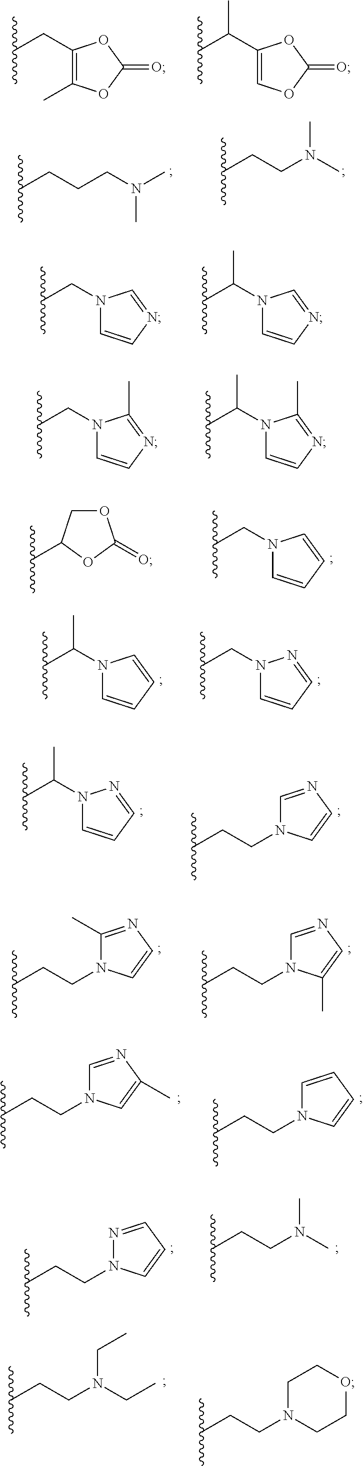

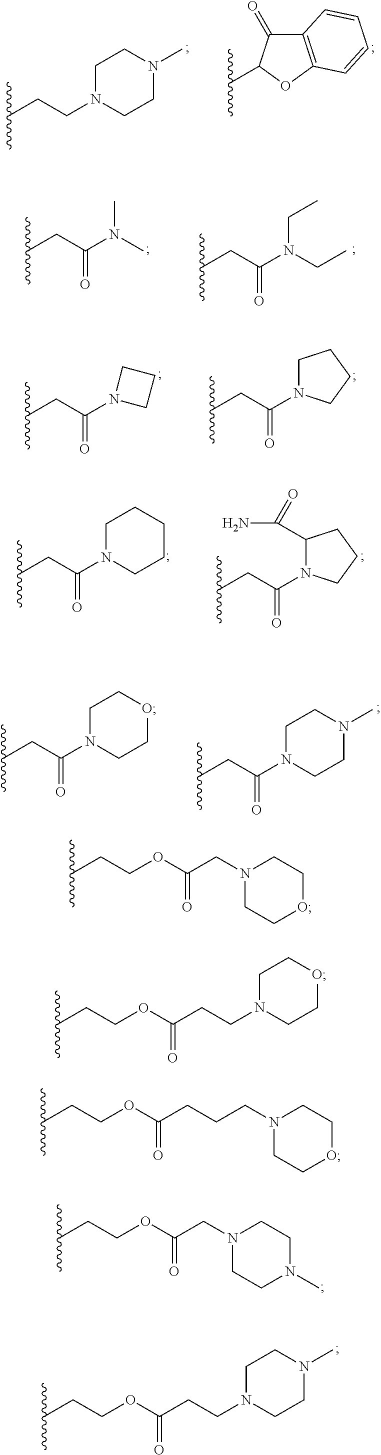

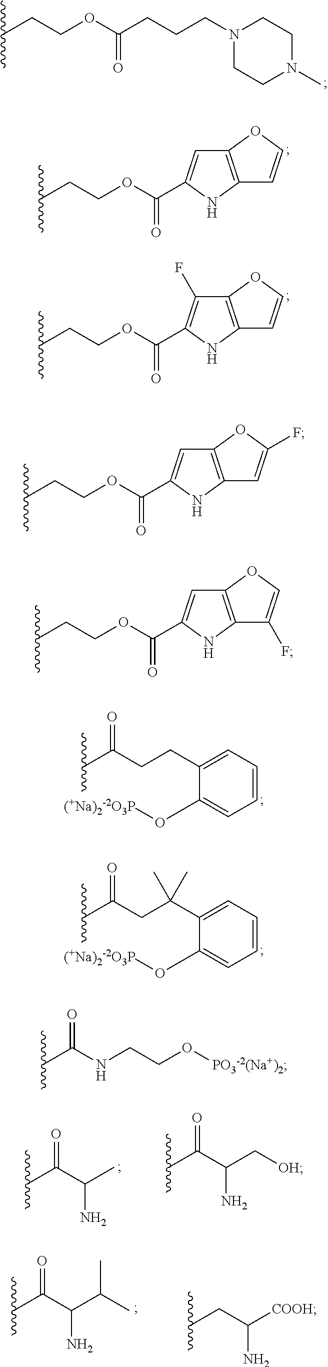

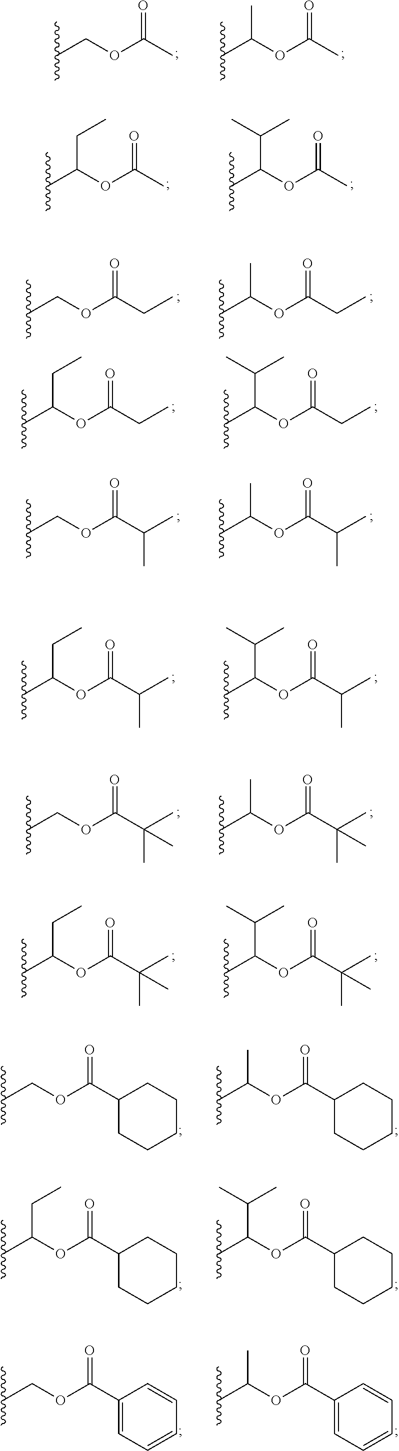

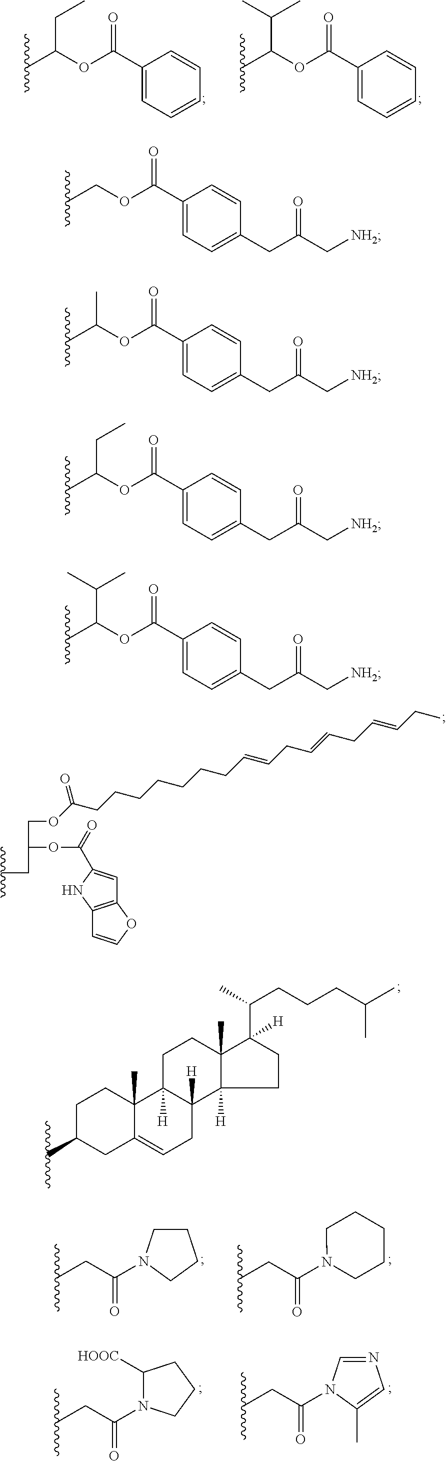

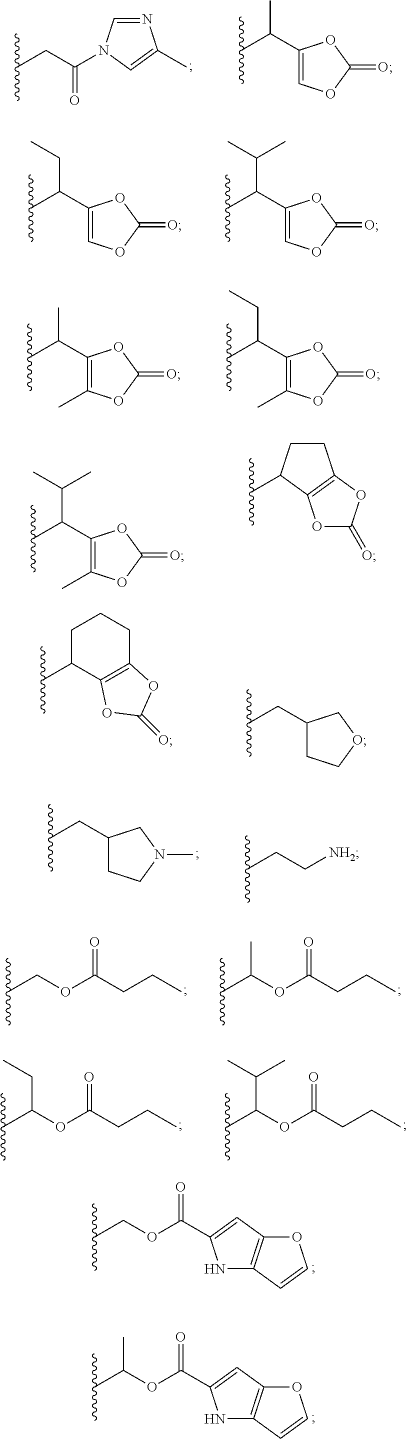

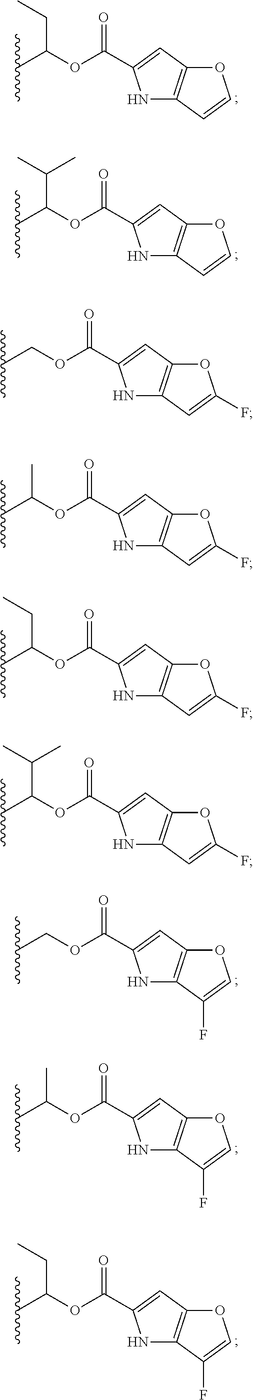

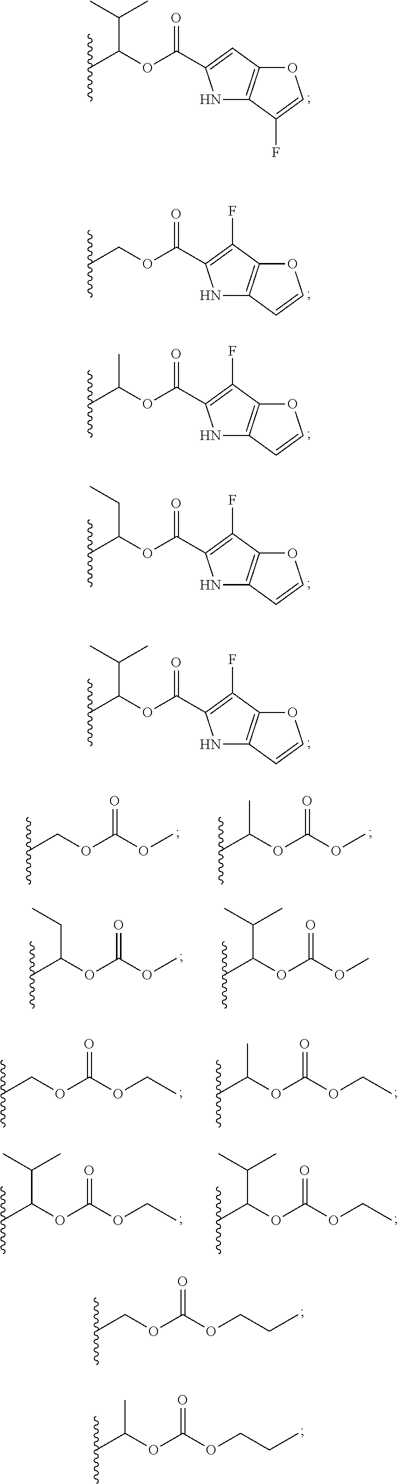

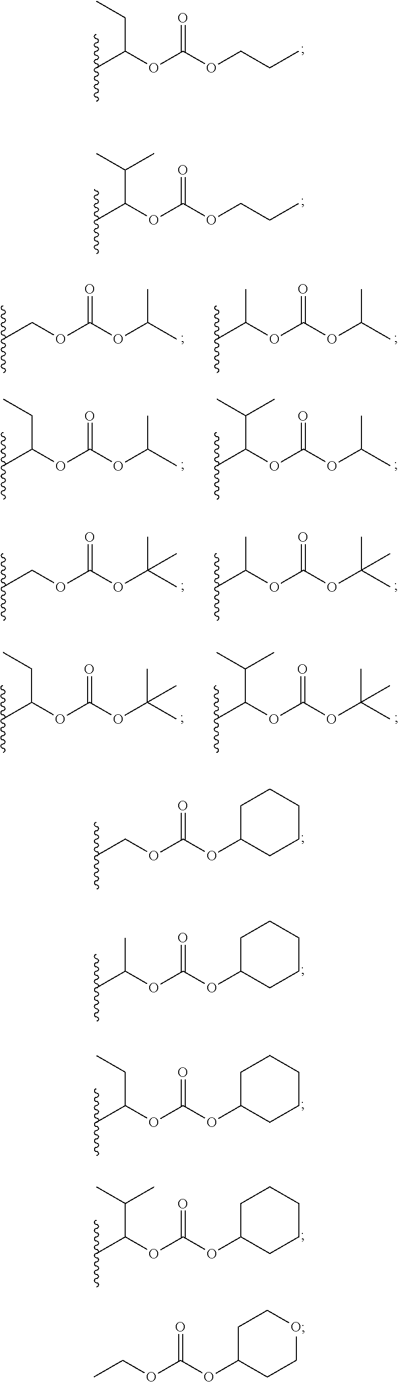

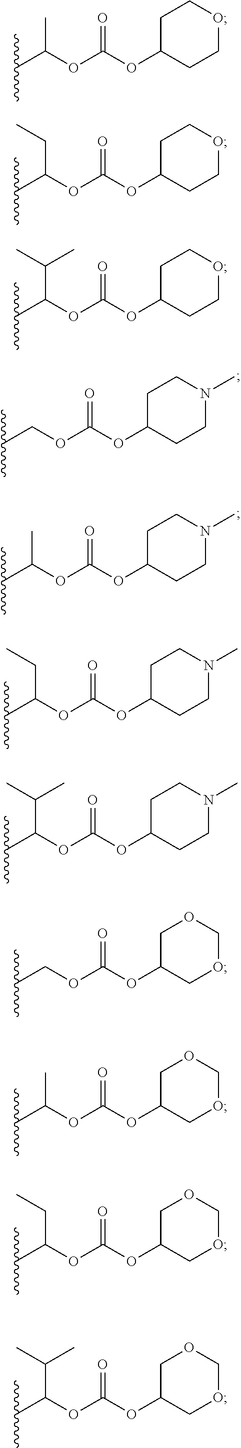

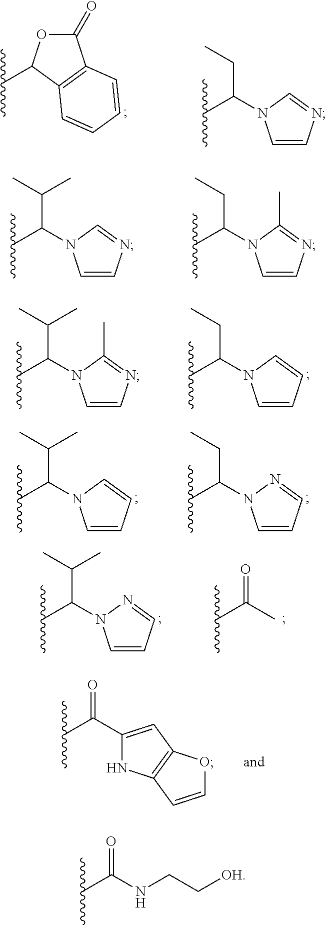

In various aspects, the present inventions provide a compound having a structure according to Formula Q

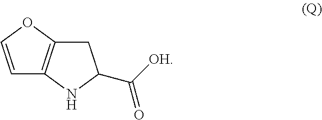

In various aspects, the inventions provide compounds comprising a first cyclic structure and a second cyclic structure linked by a linker moiety. The first and second cyclic structures are generally fused heterocycles provided by the present inventions, and the linker is as provided herein.

In various aspects, the present inventions provide methods for inhibition of DAAO, and/or influencing the activity of the NMDA receptor in the brain (e.g., by controlling the levels of D-serine at the NMDA receptor, in plasma, and/or cerebellum) by administration of a therapeutically effective amount of a compound of the present inventions. For example, in various embodiments, the administration of a compound of general formulae A, A^{x}, B, C and/or Q can be effective in treating conditions and disorders, especially CNS-related disorders, modulated by DAAO, D-serine and/or NMDA receptor activity. These conditions and disorders include, but are not limited to, neuropsychiatric disorders, such as schizophrenia, autism, attention deficit disorder (ADD and ADHD) and childhood learning disorders, and neurodegenerative diseases and disorders, such as MLS (cerebellar ataxia), Alzheimer's disease, Parkinson's disease, Huntington's disease, amyotrophic lateral sclerosis, Down syndrome, neuropathic pain, multi-infarct dementia, status epilepticus, contusive injuries (e.g., spinal cord injury and head injury), viral infection induced neurodegeneration, (e.g., AIDS, encephalopathies), epilepsy, benign forgetfulness, and closed head injury. In various embodiments, administration of a compound of general formulae A, A^{x}, B, C and/or Q can be useful for the treatment of neurotoxic injury which follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest.

In various aspects, the administration of a compound of general formulae A, A^{x}, B, C and/or Q for treating schizophrenia, for treating or preventing loss of memory and /or cognition associated with Alzheimer's disease, for treating ataxia, or for preventing loss of neuronal function characteristic of neurodegenerative diseases.

In various aspects, the administration of a compound of general formulae A, A^{x}, B, C and/or Q is useful for enhancing learning, memory and/or cognition, even in a subject not suffering from a disease or condition that causes loss of memory, cognition associated and/or loss of neuronal function.

In various aspects, the present inventions provide compounds of Formulae A, A^{x}, B, C and/or Q that provide for a composition with a PK profile of the released active compound that is different from that of the active compound when administered in its non-prodrug form. For example, in various embodiments, a compound of Formulae A, A^{x}, B, C and/or Q could provide more desirable absorption, distribution, metabolism, and/or elimination of the active compound allowing for different dosage regimes (e.g., once a day), dosage amounts (e.g., lesser dosages, greater dosages), etc.

In various aspects, the present inventions provide compounds of Formulae A, A^{x}, B, C and/or Q that provide for a composition with increased stability compared to one or more non-prodrug forms of the active compound. For example, in various embodiments, a compound of Formulae A, A^{x}, B, C and/or Q has increased stability in a solution with a pH of about 7.4. In various embodiments, a compound of Formulae A, A^{x}, B, C and/or Q has increased stability in a solution with a pH of about 2.0. In various embodiments, a compound of Formulae A, A^{x}, B, C and/or Q has increased stability in a solution with a pH of about 7.4 and in a solution with a pH of about 2.0.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a graph showing the conversion of compound 1 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 2 is a graph showing the conversion of compound 2 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 3 is a graph showing the conversion of compound 4 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 4 is a graph showing the conversion of compound 5 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 5 is a graph showing the conversion of compound 6 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 6 is a graph showing the conversion of compound 7 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 7 is a graph showing the conversion of compound 8 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 8 is a graph showing the conversion of compound 9 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 9 is a graph showing the conversion of compound 10 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 10 is a graph showing the conversion of compound 18 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 11 is a graph showing the conversion of compound 19 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 12 is a graph showing the conversion of compound 20 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 13 is a graph showing the conversion of compound 21 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 14 is a graph showing the conversion of compound 22 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 15 is a graph showing the conversion of compound 27 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 16 is a graph showing the conversion of compound 28 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 17 is a graph showing the conversion of compound 30 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 18 is a graph showing the conversion of compound 33 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 19 is a graph showing the conversion of compound 34 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 20 is a graph showing the conversion of compound 35 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 21 is a graph showing the conversion of compound 36 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 22 is a graph showing the conversion of compound 37 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 23 is a graph showing the conversion of compound 38 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 24 is a graph showing the conversion of compound 39 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 25 is a graph showing the conversion of compound 40 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 26 is a graph showing the conversion of compound 51 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 27 is a graph showing the conversion of compound 56 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 28 is a graph showing the conversion of compound 57 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 29 is a graph showing the conversion of compound 58 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 30 is a graph showing the conversion of compound 72 to 4H-furo[3,2-b]pyrrole-5-carboxylic acid in rat, dog and human plasma, wherein “[acid]” refers to the concentration of 4H-furo[3,2-b]pyrrole-5-carboxylic acid.

FIG. 31 is a graph showing the conversion of compound 73 to 4H-furo[3,2-b]pyrrole-5-carboxylic acid in rat, dog and human plasma, wherein “[acid]” refers to the concentration of 4H-furo[3,2-b]pyrrole-5-carboxylic acid.

### DETAILED DESCRIPTION OF THE INVENTION

### I. Definitions

Where substituent groups are specified by their conventional chemical formulae, written from left to right, they equally encompass the chemically identical substituents, which would result from writing the structure from right to left, e.g., —CH₂O— is intended to also recite —OCH₂—.

The term “alkyl,” by itself or as part of another substituent, means, unless otherwise stated, a straight or branched chain hydrocarbon radical, which is fully saturated and can include mono-, di- and multivalent radicals, having the number of carbon atoms designated (e.g. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

The term “alkenyl” by itself or as part of another substituent is used in its conventional sense, and refers to a radical derived from an alkene, as exemplified, but not limited, by substituted or unsubstituted vinyl and substituted or unsubstituted propenyl. Typically, an alkenyl group will have from 1 to 24 carbon atoms, with those groups having from 1 to 10 carbon atoms being useful exemplars.

The term “alkylene” by itself or as part of another substituent means a divalent radical derived from an alkane, as exemplified, but not limited, by —CH₂CH₂CH₂CH₂—. Typically, an alkyl (or alkylene) group will have from 1 to 24 carbon atoms, with those groups having 10 or fewer carbon atoms being useful exemplars in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain hydrocarbon radical consisting of the stated number of carbon atoms and at least one heteroatom selected from the group consisting of O, N, Si, S, B and P and wherein the nitrogen and sulfur atoms may optionally be oxidized and the nitrogen heteroatom may optionally be quaternized. The heteroatom(s) may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂,—S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —CO₂R′— represents both —C(O)OR′ and —OC(O)R′.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (preferably from 1 to 3 rings), which are fused together or linked covalently. The term “heteroaryl” refers to aryl groups (or rings) that contain from one to four heteroatoms selected from N, O, S, Si and B, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Exemplary substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: substituted or unsubstituted aryl, substituted or unsubstituted heteroaryl, substituted or unsubstituted heterocycloalkyl, —OR′, ═O, ═NR′, ═N—OR′, —NR′R″, —SR′, —halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the present inventions includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: substituted or unsubstituted alkyl, substituted or unsubstituted aryl, substituted or unsubstituted heteroaryl, substituted or unsubstituted heterocycloalkyl, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR′″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the present inventions includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula -T-C(O)—(CRR′)_{q}-U-, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “acyl” describes a substituent containing a carbonyl residue, C(O)R. Exemplary species for R include H, halogen, substituted or unsubstituted alkyl, substituted or unsubstituted aryl, substituted or unsubstituted heteroaryl, and substituted or unsubstituted heterocycloalkyl.

As used herein, the term “fused ring system” means at least two rings, wherein each ring has at least 2 atoms in common with another ring. “Fused ring systems” may include aromatic as well as non aromatic rings. Examples of “fused ring systems” are naphthalenes, indoles, quinolines, chromenes and the like.

As used herein, the term “heteroatom” includes oxygen (O), nitrogen (N), sulfur (S), silicon (Si) and boron (B).

The symbol “R” is a general abbreviation that represents a substituent group. Exemplary substituent groups include substituted or unsubstituted alkyl, substituted or unsubstituted heteroalkyl, substituted or unsubstituted aryl, substituted or unsubstituted heteroaryl, and substituted or unsubstituted heterocycloalkyl groups.

The phrase “therapeutically effective amount” as used herein means that amount of a compound, material, or composition comprising a compound of the present inventions which is effective for producing a desired therapeutic effect, at a reasonable benefit/risk ratio applicable to any medical treatment.

The term “pharmaceutically acceptable salts” includes salts of the compounds which are prepared with relatively nontoxic acids or bases, depending on the particular substituents found on the compounds described herein. When compounds of the present inventions contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present inventions contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, Berge et al., Journal of Pharmaceutical Science, 66: 1-19 (1977)). Certain specific compounds of the present inventions contain both basic and acidic functionalities that allow the compounds to be converted into either base or acid addition salts.

When a residue is defined as “O⁻” then the formula is meant to optionally include an organic or inorganic cationic counterion. Preferably, the resulting salt form of the compound is pharmaceutically acceptable.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

A prodrug is typically defined as a biologically inactive, or biologically less active, derivative of an active drug molecule that exerts its pharmacological effect only after chemical and/or enzymatic conversion to its active form in vivo. Prodrugs include those designed to circumvent problems associated with delivery of the active drug. This may be due to poor physicochemical properties, such as poor chemical stability or low aqueous solubility, and may also be due to poor pharmacokinetic properties, such as poor bioavailability or poor half-life. Thus, certain advantages of prodrugs may include improved chemical stability, absorption, and/or PK properties of the parent carboxylic acids. Prodrugs may also be used to make drugs more “patient friendly,” by minimizing the frequency (e.g., once daily) or route of dosing (e.g., oral), or to improve the taste or odor if given orally, or to minimize pain if given parenterally.

In various embodiments, the prodrugs effect a “slow-release” of the active drug, thereby changing the time-course of the active compound and/or D-serine increase in a manner that improves the efficacy of the parent compound. For example, compounds of the present inventions that extend D-serine level increases demonstrate improved efficacy in animal models of cognition (e.g., Contextual Fear Conditioning or Novel Object Recognition).

In various embodiments, the prodrugs are chemically more stable than the active drug. In various embodiments, enhanced chemical stability of the prodrug results in improved formulation and delivery of the active drug, compared to direct administration of the active the drug in its active form.

Carboxylic acid compounds of the present inventions may include one or more of a variety of cleavable moieties, which, upon cleavage, liberate an active carboxylic acid compound. In various embodiments, the cleavable moiety is an ester. In various embodiments, the pharmaceutical compositions of the present inventions include a carboxylic acid ester. In various embodiments, the compounds of the present inventions (e.g., a compound of Formulae A, A^{x}, B, C and/or Q) is suitable for treatment/prevention of those diseases and conditions that require the drug molecule to cross the blood brain barrier. In various embodiments, a a compound of Formulae A, A^{x}, B, C and/or Q enters the brain, where it is converted into the active form of the drug molecule. In various embodiments, a compound of Formulae A, A^{x}, B, C and/or Q can be used to enable an active drug molecule to reach the inside of the eye after topical application of the prodrug to the eye. In various embodiments, a compound of Formulae A, A^{x}, B, C and/or Q can be converted to an active compound by chemical or biochemical methods in an ex vivo environment. For example, a compound of Formulae A, A^{x}, B, C and/or Q can be slowly converted to its parent compound when placed in a transdermal patch reservoir with one or more suitable enzymes or chemical reagents.

Certain compounds of the present inventions can exist in unsolvated forms as well as solvated forms, including hydrated forms. In general, the solvated forms are equivalent to unsolvated forms and are encompassed within the scope of the present invention. Certain compounds of the present inventions may exist in multiple crystalline or amorphous forms (“polymorphs”). In general, all physical forms are of use in the methods contemplated and are intended to be within the scope of the present inventions. “Compound or a pharmaceutically acceptable salt, hydrate, polymorph or solvate of a compound” intends the inclusive meaning of “or”, in that materials meeting more than one of the stated criteria are included, e.g., a material that is both a salt and a solvate is encompassed.

Certain compounds of the present inventions possess asymmetric carbon atoms (optical centers) or double bonds; the racemates, diastereomers, geometric isomers and individual isomers are encompassed within the scope of the present inventions. Optically active (R)- and (S)-isomers and d and may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are included.

In various embodiments, the compounds of the present inventions may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present inventions, whether radioactive or not, are intended to be encompassed within the scope of the present inventions.

In the context of the present inventions, compounds that are considered to possess activity as DAAO inhibitors are those compounds of Formulae A, A^{x}, B, C or Q that liberate an active (or “parent”) compound displaying 50% inhibition of the enzymatic activity of DAAO (IC₅₀) at a concentration of not higher than about 100 μM, preferably, not higher than about 10 μM, preferably not higher than about 1 μM, preferably not higher than about 100 nM, more preferably not higher than about 25 nM, and even more preferably not higher than about 10 nM. In various embodiments, compounds that are considered to possess activity as DAAO inhibitors are those compounds of Formulae A, A^{x}, B, C or Q that liberate an active (or “parent”) compound displaying 50% inhibition of the enzymatic activity of DAAO (IC₅₀) at a concentration between about 1 nM to about 25 nM, between about 5 nM to about 25 nM, and/or between about 4 nM to about 6 nM.

The term “neurological disorder” refers to any condition of the central or peripheral nervous system of a mammal. The term “neurological disorder” includes neurodegenerative diseases (e.g., Alzheimer's disease, Parkinson's disease and amyotrophic lateral sclerosis), neuropsychiatric diseases (e.g., schizophrenia and anxieties, such as general anxiety disorder). Exemplary neurological disorders include MLS (cerebellar ataxia), Huntington's disease, Down syndrome, multi-infarct dementia, status epilepticus, contusive injuries (e.g. spinal cord injury and head injury), viral infection induced neurodegeneration, (e.g., AIDS, encephalopathies), epilepsy, benign forgetfulness, closed head injury, sleep disorders, depression (e.g., bipolar disorder), dementias, movement disorders, psychoses, alcoholism, post-traumatic stress disorder and the like. “Neurological disorder” also includes any condition associated with the disorder. For instance, a method of treating a neurodegenerative disorder includes methods of treating loss of memory and/or loss of cognition associated with a neurodegenerative disorder. Such method would also include treating or preventing loss of neuronal function characteristic of neurodegenerative disorder.

“Pain” is an unpleasant sensory and emotional experience. Pain classifications have been based on duration, etiology or pathophysiology, mechanism, intensity, and symptoms. The term “pain” as used herein refers to all categories of pain, including pain that is described in terms of stimulus or nerve response, e.g., somatic pain (normal nerve response to a noxious stimulus) and neuropathic pain (abnormal response of a injured or altered sensory pathway, often without clear noxious input); pain that is categorized temporally, e.g., chronic pain and acute pain; pain that is categorized in terms of its severity, e.g., mild, moderate, or severe; and pain that is a symptom or a result of a disease state or syndrome, e.g., inflammatory pain, cancer pain, AIDS pain, arthropathy, migraine, trigeminal neuralgia, cardiac ischaemia, and diabetic peripheral neuropathic pain (see, e.g., Harrison's Principles of Internal Medicine, pp. 93-98 (Wilson et al., eds., 12th ed. 1991); Williams et al., J. of Med. Chem. 42: 1481-1485 (1999), herein each incorporated by reference in their entirety). “Pain” is also meant to include mixed etiology pain, dual mechanism pain, allodynia, causalgia, central pain, hyperesthesia, hyperpathia, dysesthesia, and hyperalgesia.

“Somatic” pain, as described above, refers to a normal nerve response to a noxious stimulus such as injury or illness, e.g., trauma, burn, infection, inflammation, or disease process such as cancer, and includes both cutaneous pain (e.g., skin, muscle or joint derived) and visceral pain (e.g., organ derived).

“Neuropathic pain” is a heterogeneous group of neurological conditions that result from damage to the nervous system. “Neuropathic” pain, as described above, refers to pain resulting from injury to or dysfunctions of peripheral and/or central sensory pathways, and from dysfunctions of the nervous system, where the pain often occurs or persists without an obvious noxious input. This includes pain related to peripheral neuropathies as well as central neuropathic pain. Common types of peripheral neuropathic pain include diabetic neuropathy (also called diabetic peripheral neuropathic pain, or DN, DPN, or DPNP), post-herpetic neuralgia (PHN), and trigeminal neuralgia (TGN). Central neuropathic pain, involving damage to the brain or spinal cord, can occur following stroke, spinal cord injury, and as a result of multiple sclerosis. Other types of pain that are meant to be included in the definition of neuropathic pain include pain from neuropathic cancer pain, HIV/AIDS induced pain, phantom limb pain, and complex regional pain syndrome. In various embodiments, the compounds of the present inventions are of use for treating neuropathic pain.

Common clinical features of neuropathic pain include sensory loss, allodynia (non-noxious stimuli produce pain), hyperalgesia and hyperpathia (delayed perception, summation, and painful aftersensation). Pain is often a combination of nociceptive and neuropathic types, for example, mechanical spinal pain and radiculopathy or myelopathy.

“Acute pain” is the normal, predicted physiological response to a noxious chemical, thermal or mechanical stimulus typically associated with invasive procedures, trauma and disease. It is generally time-limited and may be viewed as an appropriate response to a stimulus that threatens and/or produces tissue injury. “Acute pain”, as described above, refers to pain which is marked by short duration or sudden onset.

“Chronic pain” occurs in a wide range of disorders, for example, trauma, malignancies and chronic inflammatory diseases such as rheumatoid arthritis. Chronic pain usually lasts more than about six months. In addition, the intensity of chronic pain may be disproportionate to the intensity of the noxious stimulus or underlying process. “Chronic pain”, as described above, refers to pain associated with a chronic disorder, or pain that persists beyond resolution of an underlying disorder or healing of an injury and that is often more intense than the underlying process would predict. It may be subject to frequent recurrence.

“Inflammatory pain” is pain in response to tissue injury and the resulting inflammatory process. Inflammatory pain is adaptive in that it elicits physiologic responses that promote healing. However, inflammation may also affect neuronal function. Inflammatory mediators, including PGE₂ induced by the COX2 enzyme, bradykinins, and other substances, bind to receptors on pain-transmitting neurons and alter their function, increasing their excitability and thus increasing pain sensation. Much chronic pain has an inflammatory component. “Inflammatory pain”, as described above, refers to pain which is produced as a symptom or a result of inflammation or an immune system disorder.

“Visceral pain”, as described above, refers to pain which is located in an internal organ.

“Mixed etiology” pain, as described above, refers to pain that contains both inflammatory and neuropathic components.

“Dual mechanism” pain, as described above, refers to pain that is amplified and maintained by both peripheral and central sensitization.

“Causalgia”, as described above, refers to a syndrome of sustained burning, allodynia, and hyperpathia after a traumatic nerve lesion, often combined with vasomotor and sudomotor dysfunction and later trophic changes.

“Central” pain, as described above, refers to pain initiated by a primary lesion or dysfunction in the central nervous system.

“Hyperesthesia”, as described above, refers to increased sensitivity to stimulation, excluding the special senses.

“Hyperpathia”, as described above, refers to a painful syndrome characterized by an abnormally painful reaction to a stimulus, especially a repetitive stimulus, as well as an increased threshold. It may occur with allodynia, hyperesthesia, hyperalgesia, or dysesthesia.

“Dysesthesia”, as described above, refers to an unpleasant abnormal sensation, whether spontaneous or evoked. Special cases of dysesthesia include hyperalgesia and allodynia,

“Hyperalgesia”, as described above, refers to an increased response to a stimulus that is normally painful. It reflects increased pain on suprathreshold stimulation.

“Allodynia”, as described above, refers to pain due to a stimulus that does not normally provoke pain.

The term “pain” includes pain resulting from dysfunction of the nervous system: organic pain states that share clinical features of neuropathic pain and possible common pathophysiology mechanisms, but are not initiated by an identifiable lesion in any part of the nervous system.

The term “Diabetic Peripheral Neuropathic Pain” (DPNP, also called diabetic neuropathy, DN or diabetic peripheral neuropathy) refers to chronic pain caused by neuropathy associated with diabetes mellitus. The classic presentation of DPNP is pain or tingling in the feet that can be described not only as “burning” or “shooting” but also as severe aching pain. Less commonly, patients may describe the pain as itching, tearing, or like a toothache. The pain may be accompanied by allodynia and hyperalgesia and an absence of symptoms, such as numbness.

The term “Post-Herpetic Neuralgia”, also called “Postherpetic Neuralgia” (PHN), is a painful condition affecting nerve fibers and skin. It is a complication of shingles, a second outbreak of the varicella zoster virus (VZV), which initially causes chickenpox.

The term “neuropathic cancer pain” refers to peripheral neuropathic pain as a result of cancer, and can be caused directly by infiltration or compression of a nerve by a tumor, or indirectly by cancer treatments such as radiation therapy and chemotherapy (chemotherapy-induced neuropathy).

The term “HIV/AIDS peripheral neuropathy” or “HIV/AIDS related neuropathy” refers to peripheral neuropathy caused by HIV/AIDS, such as acute or chronic inflammatory demyelinating neuropathy (AIDP and CIDP, respectively), as well as peripheral neuropathy resulting as a side effect of drugs used to treat HIV/AIDS.

The term “Phantom Limb Pain” refers to pain appearing to come from where an amputated limb used to be. Phantom limb pain can also occur in limbs following paralysis (e.g., following spinal cord injury). “Phantom Limb Pain” is usually chronic in nature.

The term “Trigeminal Neuralgia” (TN) refers to a disorder of the fifth cranial (trigeminal) nerve that causes episodes of intense, stabbing, electric-shock-like pain in the areas of the face where the branches of the nerve are distributed (lips, eyes, nose, scalp, forehead, upper jaw, and lower jaw). It is also known as the “suicide disease”.

The term “Complex Regional Pain Syndrome (CRPS),” formerly known as Reflex Sympathetic Dystrophy (RSD), is a chronic pain condition. The key symptom of CRPS is continuous, intense pain out of proportion to the severity of the injury, which gets worse rather than better over time. CRPS is divided into type 1, which includes conditions caused by tissue injury other than peripheral nerve, and type 2, in which the syndrome is provoked by major nerve injury, and is sometimes called causalgia.

The term “Fibromyalgia” refers to a chronic condition characterized by diffuse or specific muscle, joint, or bone pain, along with fatigue and a range of other symptoms. Previously, fibromyalgia was known by other names such as fibrositis, chronic muscle pain syndrome, psychogenic rheumatism and tension myalgias.

The term “convulsion” refers to a CNS disorder and is used interchangeably with “seizure,” although there are many types of seizure, some of which have subtle or mild symptoms instead of convulsions. Seizures of all types may be caused by disorganized and sudden electrical activity in the brain. Convulsions are a rapid and uncontrollable shaking During convulsions, the muscles contract and relax repeatedly.

### II. Compositions

### A. Prodrugs of Fused Heterocycles

In various aspects, the present inventions provide novel prodrugs of inhibitors of D-amino acid oxidase. On cleavage of one or more labile groups, the prodrugs release an active inhibitor of D-amino acid oxidase. The active drug compounds that inhibit D-amino acid oxidase include, but are not limited to, various fused aromatic acids described herein and in Patent Publications WO/2008/005456, US/2008/0058395, and US/2008/0004327 and U.S. patent application Ser. Nos. 11/883,903 and 12/016,954, all incorporated by reference in their entireties.

In various embodiments, the inventions provide prodrugs of biologically active fused heterocycles, the prodrugs having a structure according to Formula A:

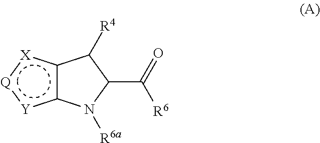

where Q, X, Y, R⁴, R⁶ and R^{6a} are as discussed previously with respect to Formula A.

In various embodiments, R⁶ is a member selected from OR²⁶, and R²⁶ is selected from substituted or unsubstituted C₄ or larger alkyl, substituted or unsubstituted heteroalkyl, substituted or unsubstituted aryl, substituted or unsubstituted heteroaryl, and substituted or unsubstituted heterocycloalkyl.

In various embodiments, R⁶ is a member selected from OH, O⁻M⁺ and OMe and OEt, and R^{6a} is:

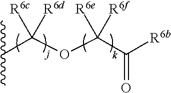

where of M⁺ and each of the radicals and the indices j and k are as discussed previously with respect to Formula A.

In various embodiments, R⁴ is selected from H, F, Cl, Br and unsubstituted C₁-C₆. In various embodiments, R⁴ is selected from unsubstituted C₁-C₄ alkyl, unsubstituted C₁-C₃ alkyl, and /or preferably unsubstituted C₁-C₂ alkyl.

In various embodiments, the inventions provide prodrugs of biologically active fused heterocycles, the prodrugs having a structure according to Formula B:

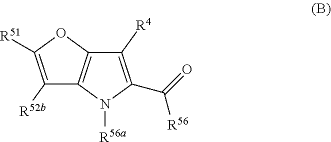

where R⁵¹, R^{52b}, R⁴, R⁵⁶ and R^{56a} are as described previously with respect to Formula B. In various preferred embodiments, R⁴ is selected from H and F.

As discussed previously with respect to Formula B, in various embodiments R⁵⁶ is OR^{26b}. In various preferred embodiments, R^{56a} is H or an alkyl, and R^{26b} is a member selected from the following:

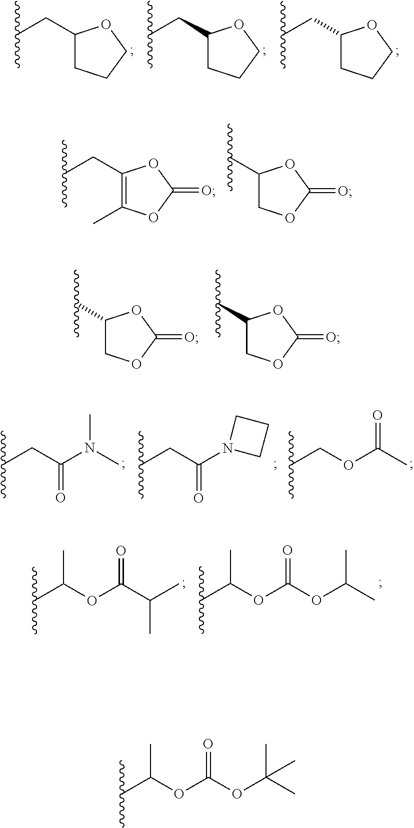

In various embodiments, the inventions provide prodrugs of biologically active fused heterocycles, the prodrugs having a structure according to Formula C:

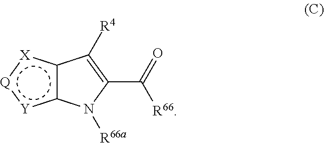

where X, Q, Y, R⁴, R⁶⁶ and R^{66a} are as discussed previously with respect to Formula C.

In various embodiments, the compounds of Formula C have the formula:

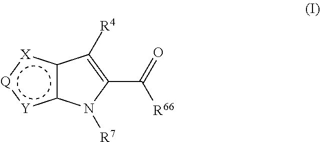

wherein X, Y, Q, R⁴ and R⁶⁶ are as discussed in the context of Formula C. In Formula I, R⁷ is H or substituted or unsubstituted alkyl. In various embodiments, R⁴ is selected from H, F, Cl, Br and unsubstituted C₁-C₆ (preferably unsubstituted C₁-C₄ alkyl, more preferably unsubstituted C₁-C₃ alkyl, and most preferably unsubstituted C₁-C₂ alkyl).

In various embodiments, the compounds of Formula C have a structure, which is a member selected from Formula (IIa) and Formula (IIb):

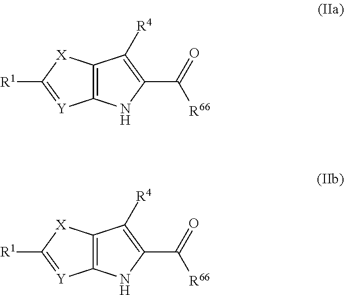

where X, Y, R¹ and R⁶⁶ are as discussed in the context of Formula C.

As discussed previously with respect to Formula C, R⁶⁶ is OR^{26c}. In various preferred embodiments, R^{26c} is a member selected from the following:

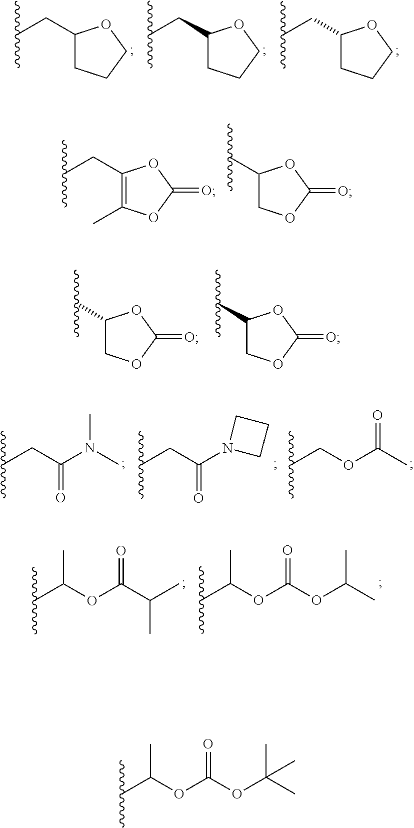

In various embodiments according to one or more of the general formulae A, B and C set forth herein, R⁶ and R^{6a}, R⁵⁶ and R^{56a}, and/or R⁶⁶ and R^{66a}, respectively, are joined to form a ring structure. For example, in various embodiments of the compounds of formulae A and C, the compounds have a structure having a formula which is a member selected from:

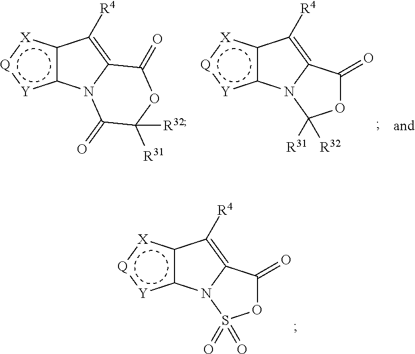

and in various embodiments the compounds of Formula B have a structure having a formula which is a member selected from:

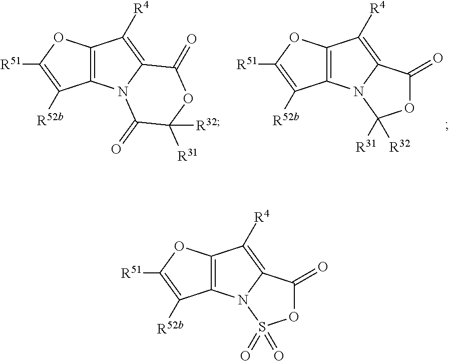

wherein R³¹ and R³² are members independently selected from H, acyl, substituted or unsubstituted alkyl, substituted or unsubstituted heteroalkyl, substituted or unsubstituted aryl, substituted or unsubstituted heteroaryl and substituted or unsubstituted heterocycloalkyl. R³¹ and R³² together with the carbon to which they are attached are optionally joined to form a ring. Optionally, together R³¹ and R³² and the carbon to which they are attached form C═O.

In various embodiments according to the general formulae A and B herein, R²⁶ and R^{26b}, respectively, is a member selected acyl, substituted or unsubstituted C₄-C₁₀ alkyl, 3- to 8-membered heterocycloalkyl, substituted or unsubstituted phenyl, and a steroid. In various embodiments, R²⁶ and R^{26b}, is selected from acyl, substituted or unsubstituted C₃-C₈ cycloalkyl and substituted or unsubstituted 3- to 8-membered heterocycloalkyl. In various embodiments, in formulae A and B R²⁶ and R^{26b}, respectively, comprises an amino acid residue. In various embodiments, in formulae A and B R²⁶ and R^{26b}, respectively, comprise P(O)(OR³³)(O⁻)M⁺ and R³³ is a member selected from substituted or unsubstituted alkyl and substituted or unsubstituted heteroalkyl; and M⁺ is an organic or inorganic cation

In various embodiments of the compounds according to formulae A or B R²⁶ and R^{26b}, respectively, comprises a moiety selected from:

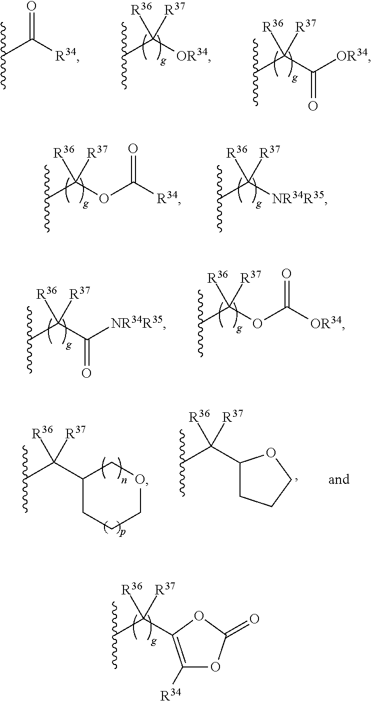

wherein R³⁴ and R³⁵ are members selected from H, acyl, substituted or unsubstituted alkyl, substituted or unsubstituted heteroalkyl, substituted or unsubstituted aryl, substituted or unsubstituted heteroaryl and substituted or unsubstituted heterocycloalkyl. When R³⁴ and R³⁵ are bound to nitrogen, R³⁴ and R³⁵, together with the nitrogen to which they are bound, are optionally joined to form a ring (e.g., a 3-, 4-, 5-, 6- or 7-membered ring which is a member selected from substituted or unsubstituted cycloalkyl, substituted or unsubstituted heterocycloalkyl, substituted or unsubstituted heteroaryl and is optionally fused to an aryl moiety). R³⁶ and R³⁷ each is a member independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, or R³⁶ and R³⁷ taken together represent C═O. R³⁶ and R³⁷, together with the carbon to which they are bound, are optionally joined to form a ring. The index g is an integer selected from 1 to 10, and in various embodiments from 1 to 3. The indices n and p are independently integers from 0 to 2. R³⁴ and R³⁶ are optionally joined to form a ring.

In various embodiments, R³⁴ is

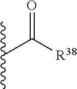

wherein R³⁸ is a member selected from OR³⁹, NR³⁹R⁴⁰, substituted or unsubstituted alkyl, and substituted or unsubstituted heterocycloalkyl; and R³⁹ and R⁴⁰ are members independently selected from H, substituted or unsubstituted alkyl, substituted or unsubstituted heteroalkyl, substituted or unsubstituted aryl, substituted or unsubstituted heteroaryl, and substituted or unsubstituted heterocycloalkyl. In various embodiments, R³⁸ is a member selected from substituted or unsubstituted arylalkyl, and substituted or unsubstituted heteroarylalkyl. In various embodiments, a member selected from R³⁴, R³⁶, R³⁷ and combinations thereof is substituted with one or more halogen atoms.

Other embodiments include any combination of the embodiments and examples set forth herein for compounds according to the formulae A, B, or C set forth herein.

In various embodiments according to the formulae set forth herein, R²⁶ and R^{26b} is a member selected from the following:

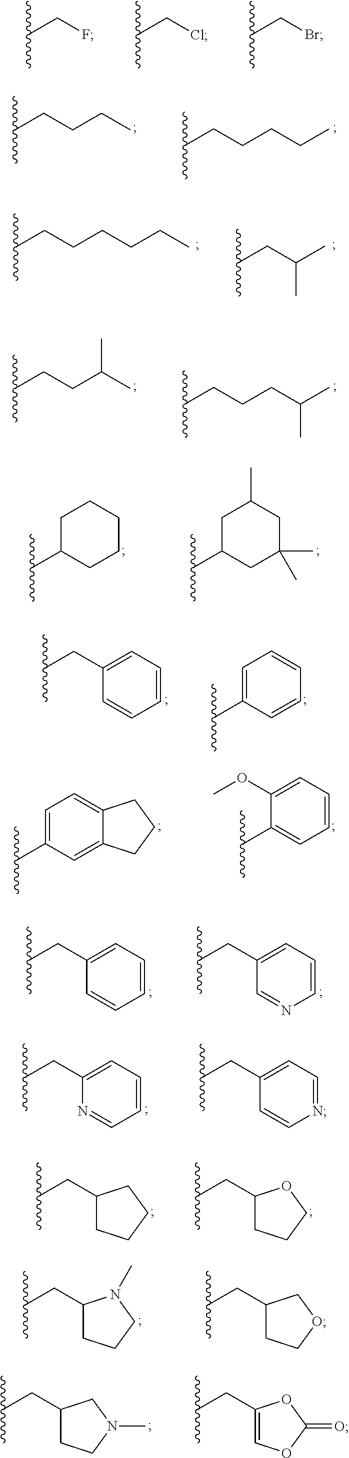

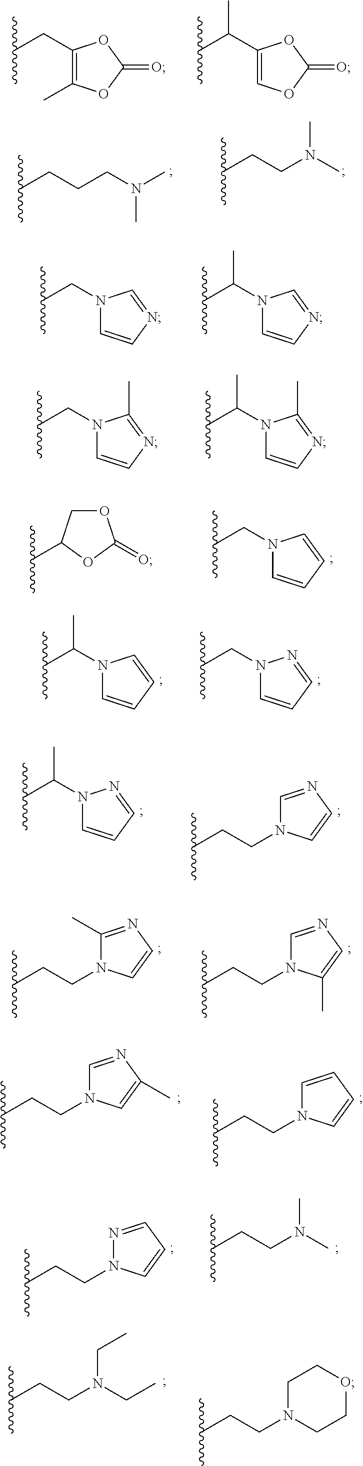

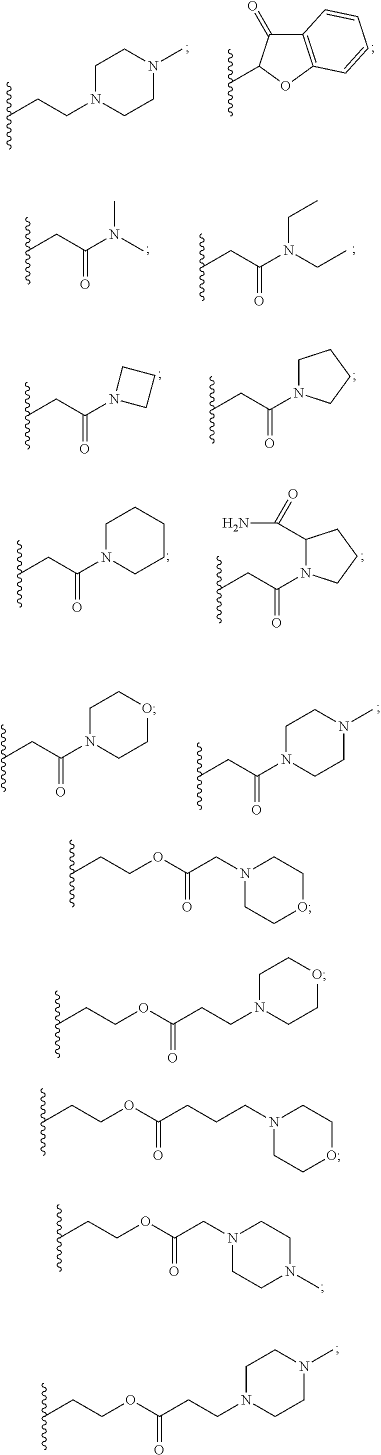

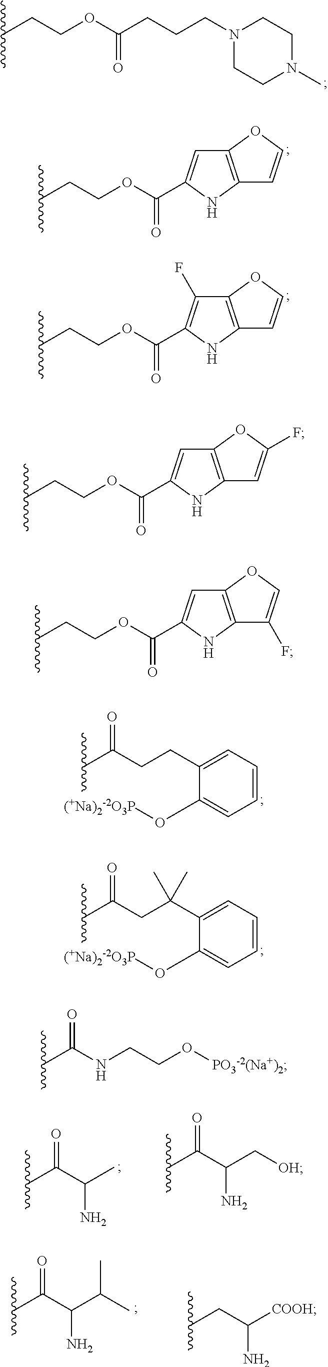

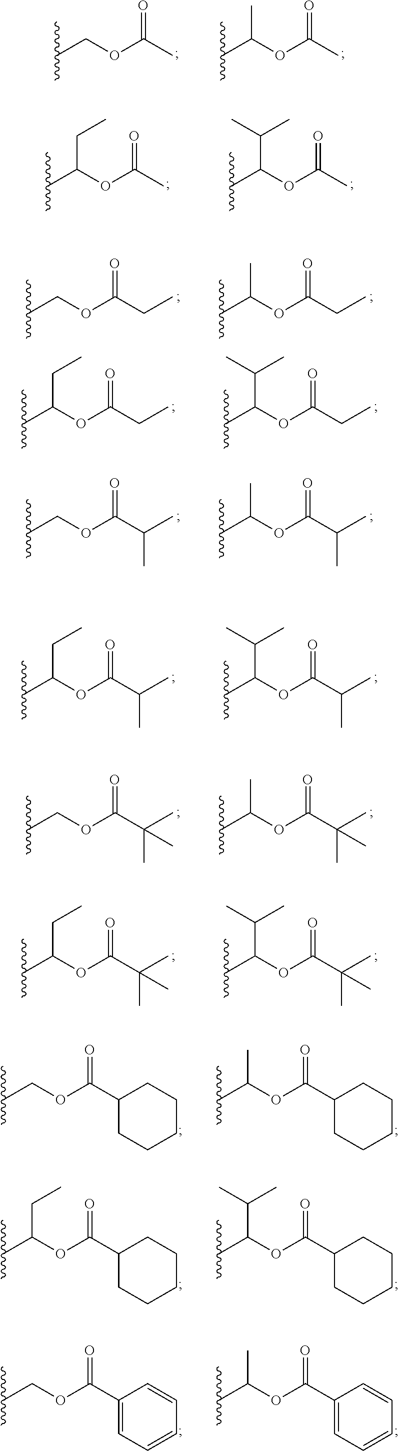

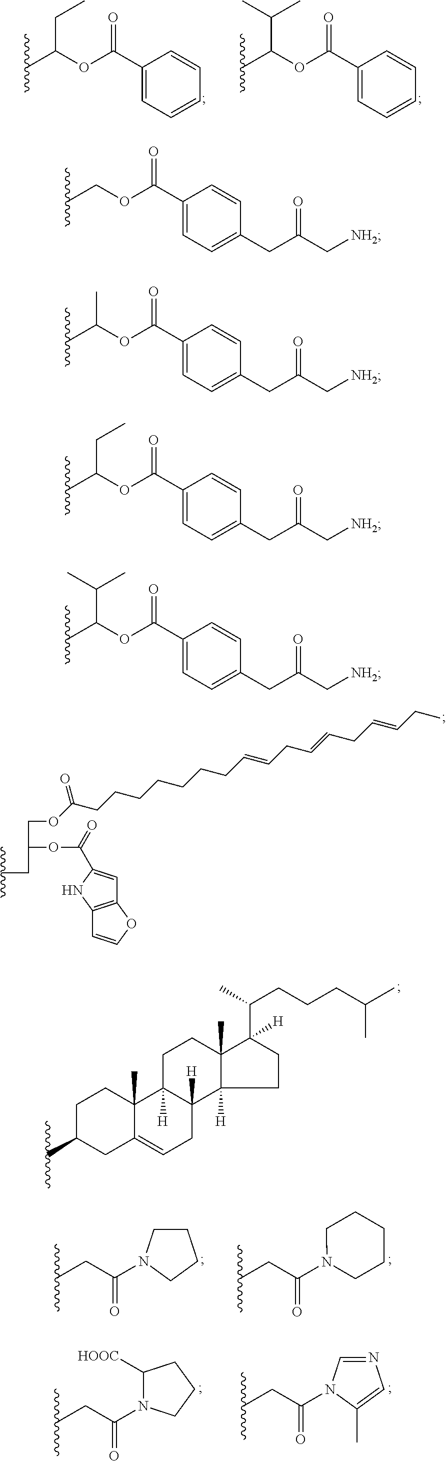

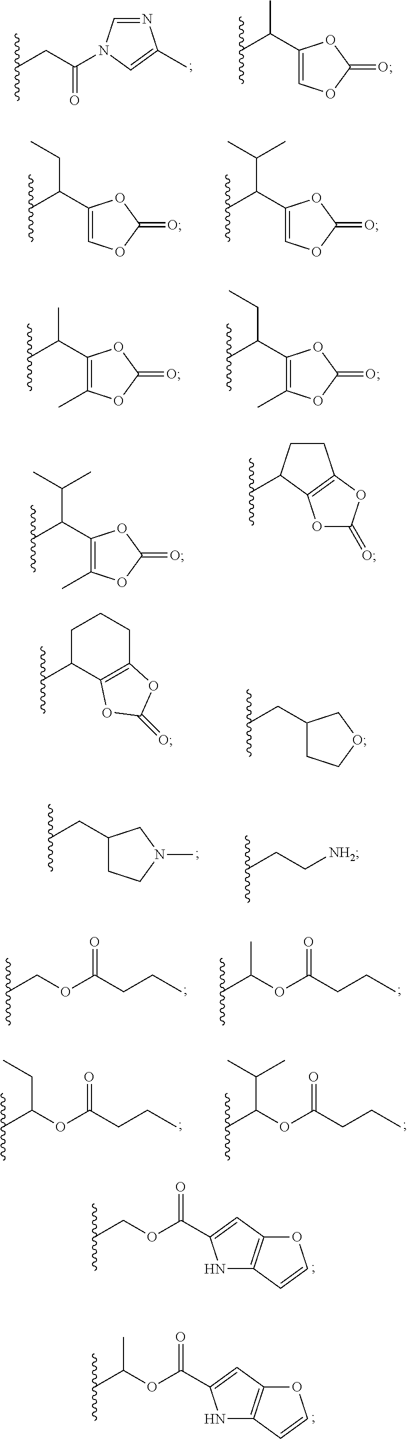

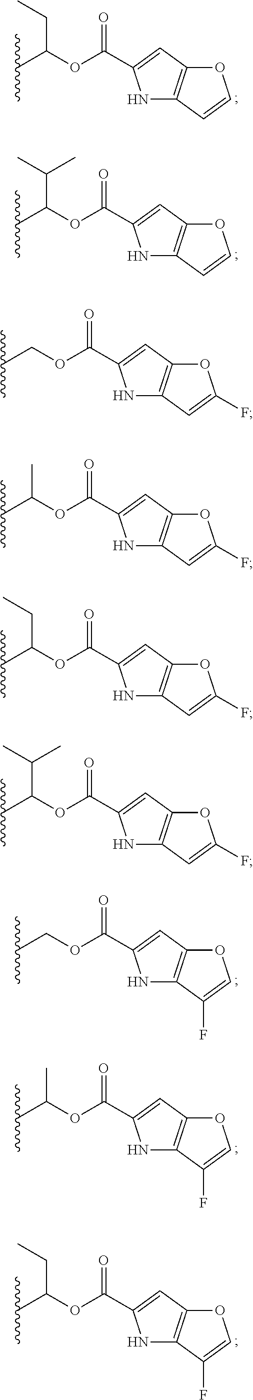

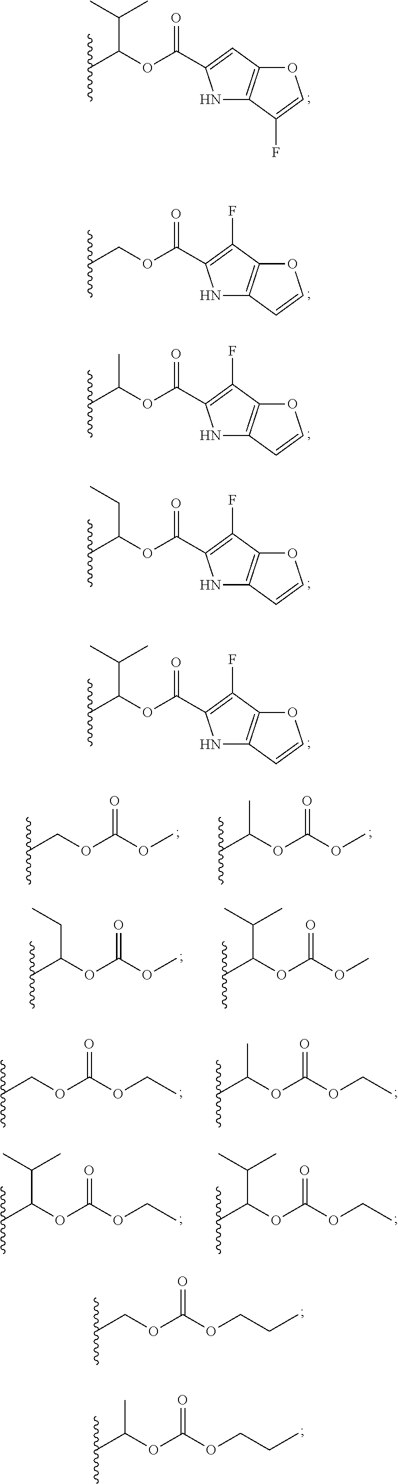

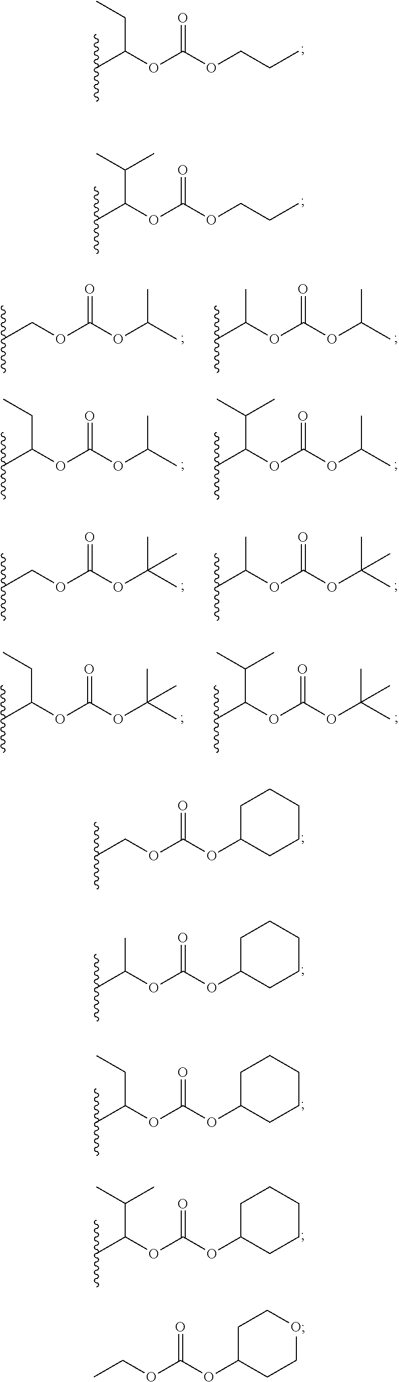

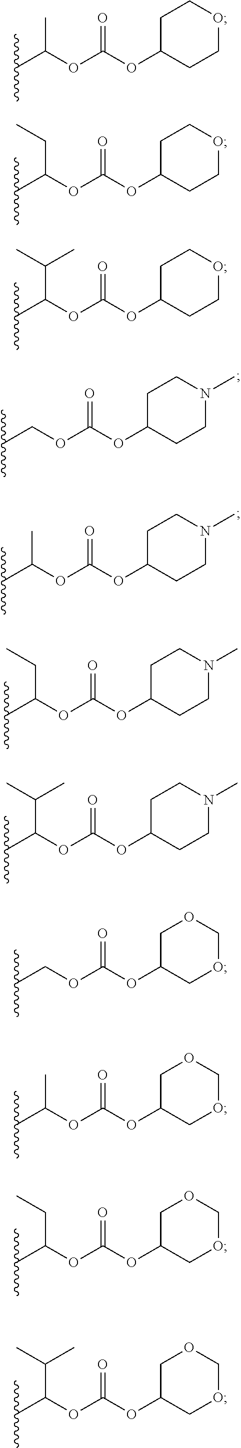

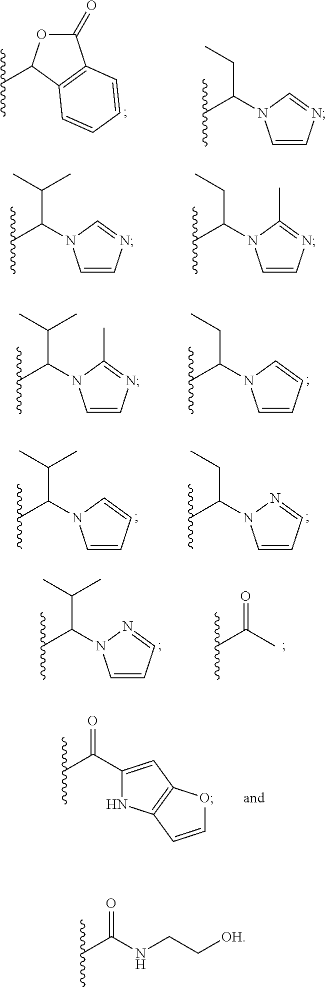

In various aspects, the present inventions provide a compound having a structure according to Formula A^{x}:

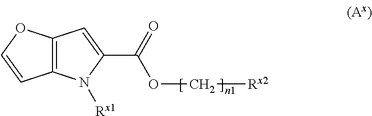

wherein R^{x1} is a member selected from H and

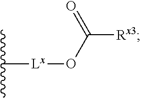

n1 is selected from the integers from 0 to 10; and R^{x2} is a member selected from H, unsubstituted alkyl, substituted or unsubstituted heteroaryl, substituted or unsubstituted aryl, substituted or unsubstituted cycloalkyl,

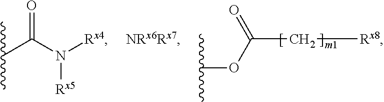

halogen and

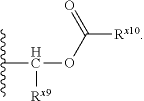

L^{x} is a member selected from substituted or unsubstituted alkyl and substituted or unsubstituted heteroalkyl. R^{x3} is a member selected from substituted or unsubstituted alkyl and substituted or unsubstituted aryl. R^{x4} and R^{x5} are independently selected substituted or unsubstituted C₁-C₁₀ alkyl. R^{x4} and R^{x5}, together with the nitrogen atom to which they are attached, are optionally joined to form a substituted or unsubstituted 3- to 8-membered heterocycloalkyl ring. R^{x6} and R^{x7} are independently selected substituted or unsubstituted C₁-C₁₀ alkyl. m1 is selected from the integers from 0 to 3. R^{x8} is a member selected from substituted or unsubstituted piperazinyl and 4H-furo[3,2-b]pyrrol-5-yl. R^{x9} is a member selected from H, methyl, ethyl, and iso-propyl. R^{x10} is a member selected from unsubstituted C₁-C₄ alkyl and 4H-furo[3,2-b]pyrrol-5-yl.

In various embodiments, n1 is 0, 1, 2, 3, 4, 5, 6, 7, 8, 9 or 10.

In various embodiments, R^{x4} and R^{x5} are independently selected from optionally substituted C₁, C₂, C₃, C₄, C₅, C₆, C₇, C₈, C₉, and C₁₀ alkyl. In various embodiments, R^{x4} and R^{x5} are independently selected from optionally substituted C₂-C₁₀ alkyl. In various embodiments, R^{x4} and R^{x5} are independently selected from optionally substituted C₃-C₁₀ alkyl.

In various embodiments, R^{x4} and R^{x5}, together with the nitrogen atom to which they are attached, are joined to form an optionally substituted 3-, 4-, 5-, 6-, 7- or 8-membered heterocycloalkyl ring. In various embodiments, when R^{x4} and R^{x5}, together with the nitrogen atom to which they are attached, are joined to form an optionally substituted 3-, 4-, 5-, 6-, 7- or 8-membered heterocycloalkyl ring, the heterocycloalkyl ring comprises up to one additional non-oxygen heteroatom. In various embodiments, when R^{x4} and R^{x5}, together with the nitrogen atom to which they are attached, are joined to form an optionally substituted 3-, 4-, 5-, 6-, 7- or 8-membered heterocycloalkyl ring, the heterocycloalkyl ring is other than unsubstituted azetidine or substituted pyrrolidine.

In various embodiments, R^{x6} and R^{x7} are independently selected from optionally substituted C₁, C₂, C₃, C₄, C₅, C₆, C₇, C₈, C₉, and C₁₀ alkyl.

In various embodiments, ml is 0, 1, 2 or 3.

In various embodiments, R^{x10} is a member selected from unsubstituted C₁, C₂, C₃, and C₄ alkyl. In various embodiments, when R^{x1} and R^{x9} are H, R^{x10} is other than methyl. In various embodiments, when R^{x1} is H and R^{x9} is methyl, R^{x10} is other than methyl, ethyl or iso-propyl. In various embodiments, when R^{x1} is H and R^{x9} is iso-propyl, R^{x10} is other than methyl.

In various embodiments, when R^{x1} is H, R^{x2} is other than H or unsubstituted alkyl. In various embodiments, when R^{x1} is H and n1 is 1, R^{x2} is other than phenyl or imidazol-1-yl. In various embodiments, when R^{x1} is H and n1 is 2, R^{x2} is other than imidazol-1-yl. In various embodiments, when n1 is other than 0, R² is other than

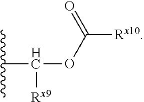

In various embodiments, when R^{x2} is H, n1 is 0 and R^{x1} is

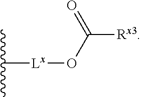

Any of the combinations of R^{x1}, R^{x2}, R^{x3}, R^{x4}, R^{x5}, R^{x6}, R^{x7}, R^{x8}, R^{x9}, R^{x10}, L^{x}, n1, and m1 are encompassed by this disclosure and specifically provided by the invention.

In various embodiments, R^{x1} is

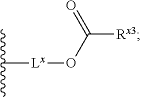

and R^{x2} is a member selected from H, methyl, ethyl and —CH₂O(CO)CH₃.

In various aspects, the present inventions provide a compound having a structure according to a formula selected from:

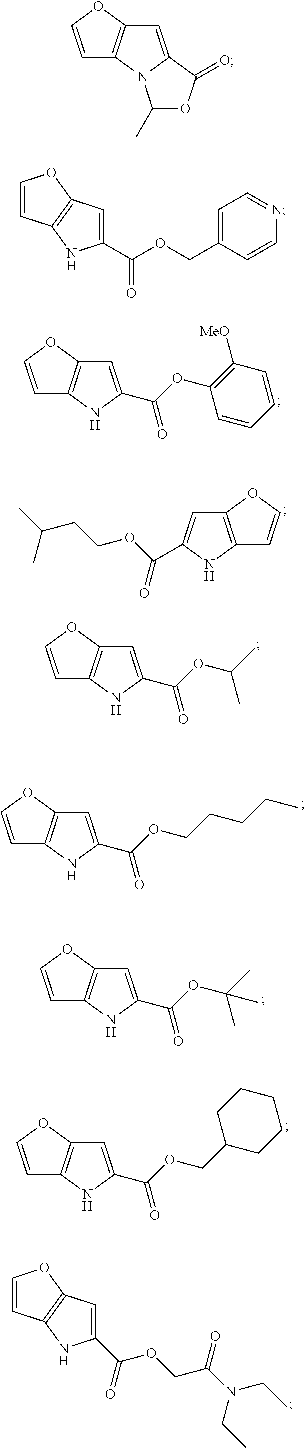

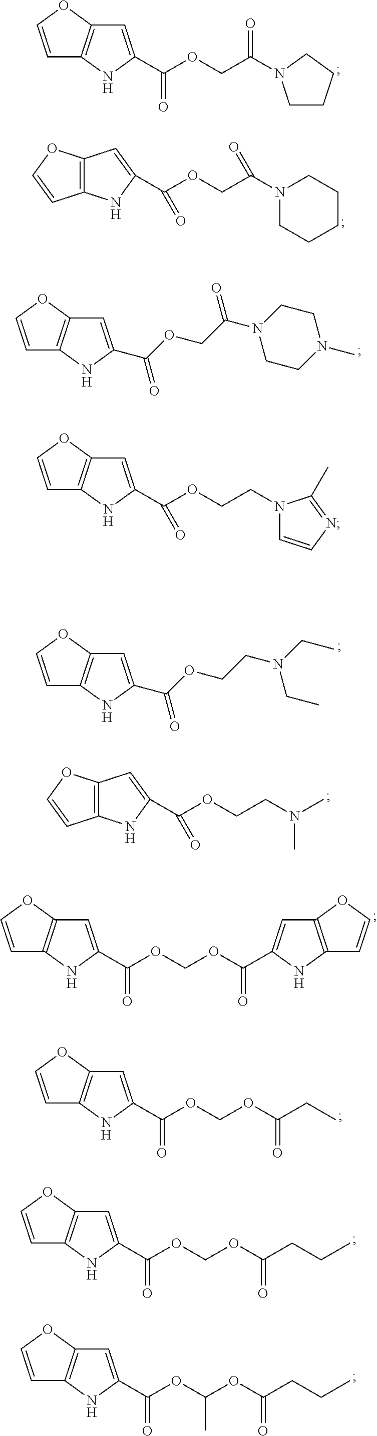

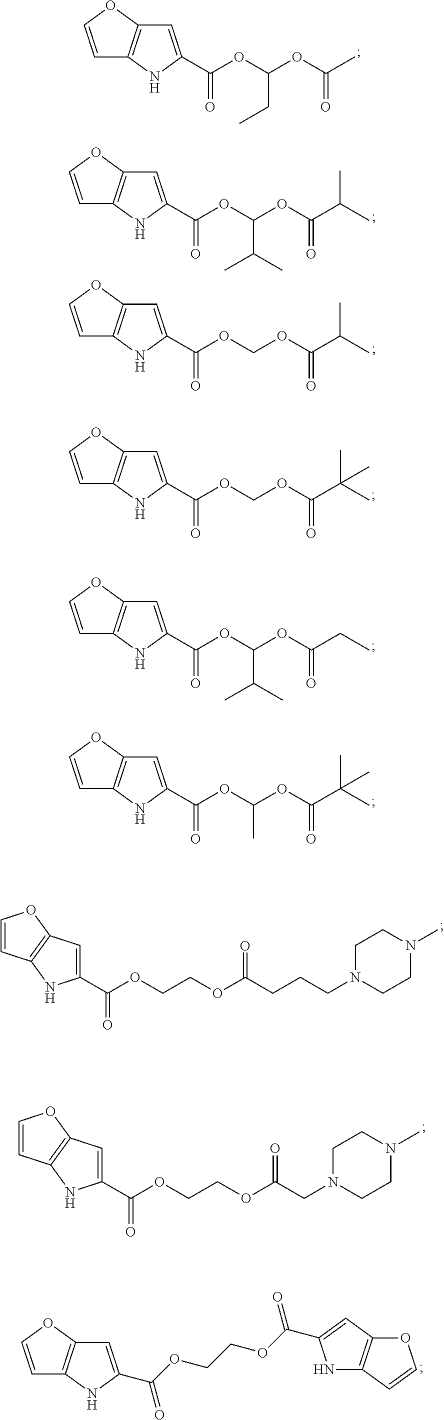

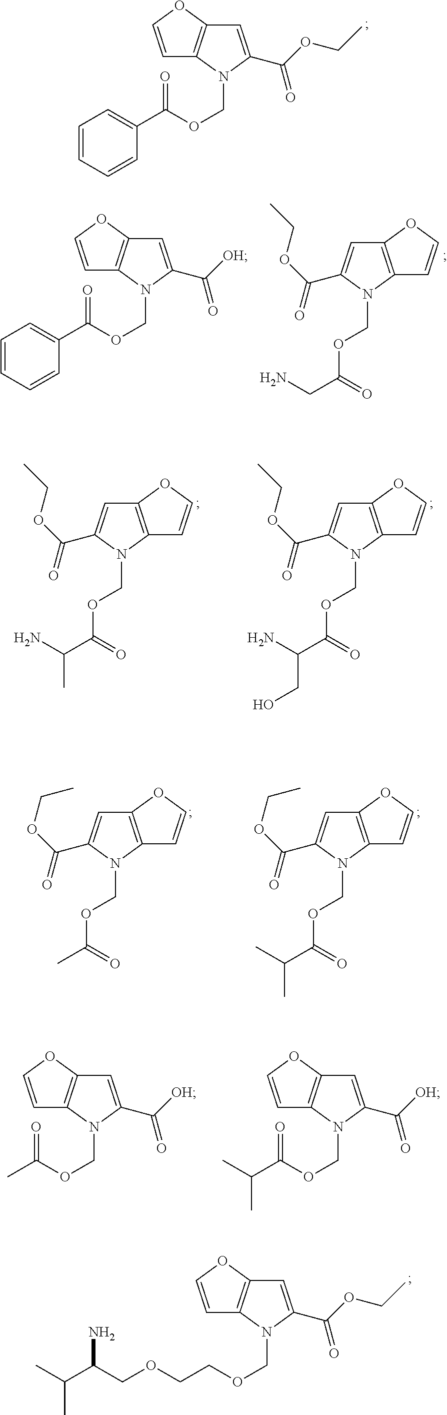

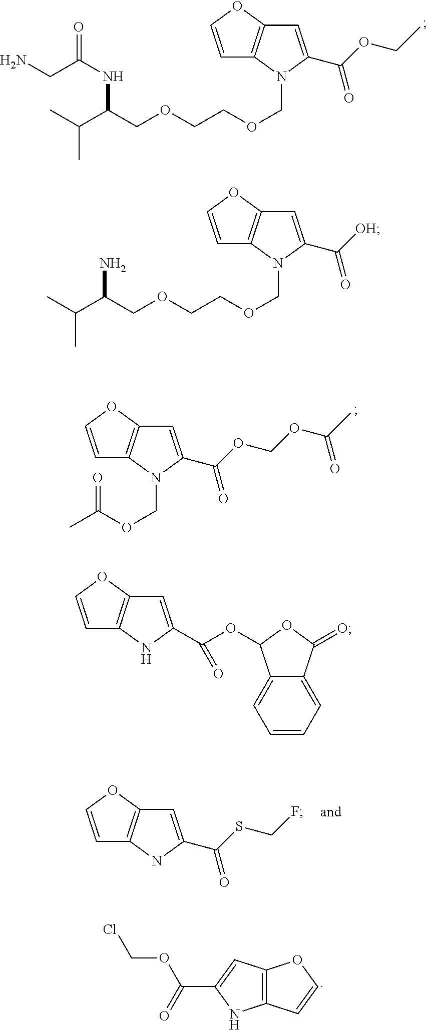

In one embodiment, the present inventions provide a compound having a structure according to the formula:

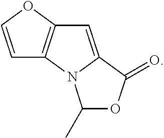

In another embodiment, the present inventions provide a compound having a structure according to the formula:

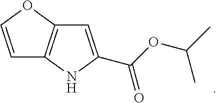

In another embodiment, the present inventions provide a compound having a structure according to the formula:

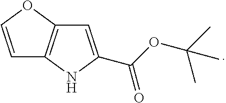

In another embodiment, the present inventions provide a compound having a structure according to the formula:

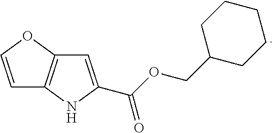

In another embodiment, the present inventions provide a compound having a structure according to the formula:

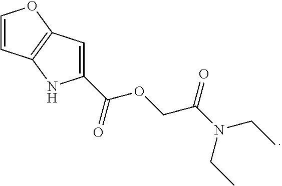

In another embodiment, the present inventions provide a compound having a structure according to the formula:

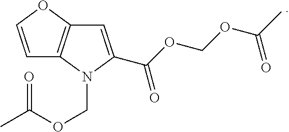

### B. Compounds Released by Prodrugs of Fused Heterocycles

Compounds released by prodrugs (e.g., active compounds) of the present inventions can be exemplified by reference to certain fused pyrrole analogs. Those of skill in the art will understand that the applicable scope of compounds released by compounds of Formulae A, A^{x}, B, C and/or Q, including pyrrole analogs, is of greater breadth than is exemplified herein. In various embodiments, the invention provides prodrugs of thiophenes and furans. In the following sections, the structures of various embodiments of compounds released by the prodrugs are set forth. It will be apparent to those of skill in the art that the structures of the released compounds set forth hereinbelow implicate the structure of corresponding prodrugs according to the various prodrug motifs set forth herein. Thus, and of the various substitution patterns and substitutions of the prodrug formula discussed herein are readily combinable with the structures of the released compounds discussed below to unambiguously provide various embodiments and compounds of the present inventions. Furthermore, it will be apparent to one of skill that the various compounds released from compounds of Formulae A, A^{x}, B, C and/or Q, are also appropriate precursors for compounds of Formulae A, A^{x}, B, C and/or Q.

### Pyrrole Analogs

In various embodiments, of compounds according to the general formulae set forth herein, A is NR⁷, for example, NH, and the compound released from the prodrug is a fused pyrrole.

In various embodiments of the compounds of the present inventions (e.g., Formulae A and C), at least one of X and Y is N. In various embodiments, these prodrugs release active compounds that have a structure selected from:

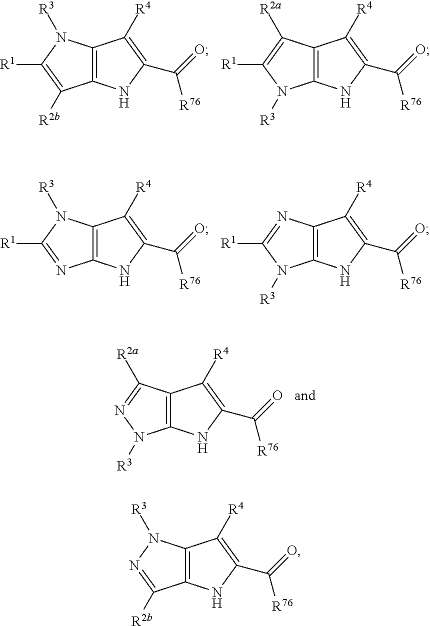

wherein R¹, R^{2a}, R^{2b}, R³, and R⁴ are as discussed previously; and R⁷⁶ is a member selected from OH and O⁻M⁺, and where M⁻ is a positive ion, which is a member selected from inorganic positive ions and organic positive ions.

In various embodiments of the compounds of Formulae A and B, either X or Y is S. In various embodiments, these prodrugs release active compounds that have a structure selected from:

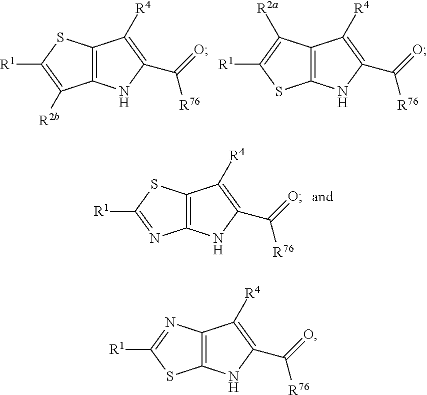

wherein R¹, R^{2a}, R^{2b}, R³, and R⁴ are as discussed previously; and R⁷⁶ is a member selected from OH and O⁻M⁺, and where M⁻ is a positive ion, which is a member selected from inorganic positive ions and organic positive ions.

In various embodiments of the compounds of Formulae A and B, either X or Y is O. In various embodiments, these prodrugs release active compounds that have a structure selected from:

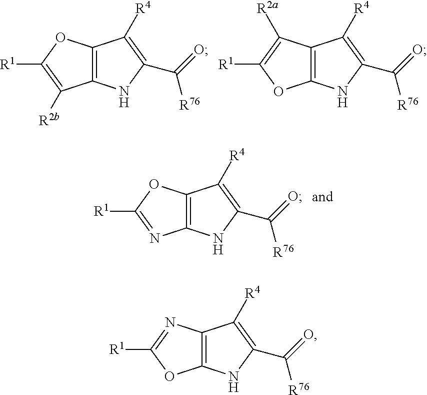

wherein R¹, R^{2a}, R^{2b}, R³, and R⁴ are as discussed previously; and R⁷⁶ is a member selected from OH and O⁻M⁺, and where M⁻ is a positive ion, which is a member selected from inorganic positive ions and organic positive ions.

In various embodiments of the compounds of Formulae A and C, Q is O or S. In various embodiments, these prodrugs release active compounds that have a structure selected from:

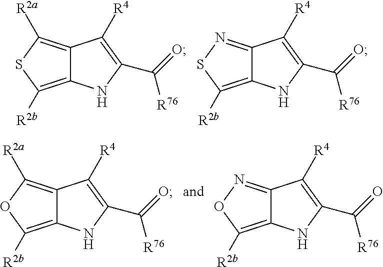

wherein R¹, R^{2a}, R^{2b}, R³, and R⁴ are as discussed previously; and R⁷⁶ is a member selected from OH and O⁻M⁺, and where M⁻ is a positive ion, which is a member selected from inorganic positive ions and organic positive ions.

### C. Linked Cyclic Structures

In various aspects, the present inventions provides for prodrugs comprising a first cyclic structure and a second cyclic structure that are linked by a linker moiety. The first and second cyclic structures that are linked include any of the compounds of formulae A, B, C, and/or Q, and the number of linked cyclic structures can be more than 2.

The term “linker moiety” refers to a group that covalently joins a cyclic structure to any other cyclic structure. A linker moiety can comprise alkyl, heteroalkyl, cycloalkyl, heterocycloalkyl, aryl, heteroaryl and their substituted counterparts. A linker moiety can also comprise any combination of any of these groups.

In various embodiments, the linker moiety L^{y} joins groups Z and Z^{a} of, respectively, a first and second cyclic structure according to the structure:

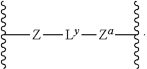

In various embodiments, Z and Z^{a} are members independently selected from NR⁴⁵, O, S and CR⁴⁶R⁴⁷ wherein each R⁴⁵, R⁴⁶ and R⁴⁷ is a member independently selected from H, OR⁴⁸, acyl, substituted or unsubstituted alkyl and substituted or unsubstituted heteroalkyl, wherein R⁴⁸ is a member selected from H, substituted or unsubstituted alkyl and substituted or unsubstituted heteroalkyl.

In various embodiments, —Z-L^{y}-Z^{a}— has the structure

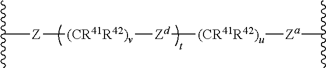

wherein each R⁴¹ and R⁴² are independently selected from H, OR⁴³, NR⁴³R⁴⁴, CN, halogen, acyl, substituted or unsubstituted alkyl, substituted or unsubstituted heteroalkyl, substituted or unsubstituted aryl, substituted or unsubstituted heteroaryl, substituted or unsubstituted heterocycloalkyl. R⁴³ and R⁴⁴ are members selected from H, acyl, substituted or unsubstituted alkyl and substituted or unsubstituted heteroalkyl. Z, Z^{a} and Z^{d} are members independently selected from NR⁴⁵, O, S and CR⁴⁶R⁴⁷. Each R⁴⁵, R⁴⁶ and R⁴⁷ is a member independently selected from H, OR⁴⁸, acyl, substituted or unsubstituted alkyl and substituted or unsubstituted heteroalkyl. R⁴⁸ is a member selected from H, substituted or unsubstituted alkyl and substituted or unsubstituted heteroalkyl. U and v are integers independently selected from 0 to 10.

In various embodiments, the linker L^{y} has the structure

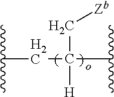

wherein o is an integer selected from 0 to 1,000, Z^{b} is a member selected from H, OR⁴⁹, substituted or unsubstituted alkyl, and substituted or unsubstituted heteroalkyl. R⁴⁹ is a member selected from H, acyl, substituted or unsubstituted alkyl and substituted or unsubstituted heteroalkyl.

In various embodiments, the linker L^{y} has the structure

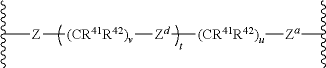

wherein each R⁴¹ and R⁴² are independently selected from H, OR⁴³, NR⁴³R⁴⁴, CN, halogen, acyl, substituted or unsubstituted alkyl, substituted or unsubstituted heteroalkyl, substituted or unsubstituted aryl, substituted or unsubstituted heteroaryl, substituted or unsubstituted heterocycloalkyl. R⁴³ and R⁴⁴ are members selected from H, acyl, substituted or unsubstituted alkyl and substituted or unsubstituted heteroalkyl. Z, Z^{a} and Z^{d} are members independently selected from NR⁴⁵, O, S and CR⁴⁶R⁴⁷ wherein each R⁴⁵, R⁴⁶ and R⁴⁷ is a member independently selected from H, OR⁴⁸, acyl, substituted or unsubstituted alkyl and substituted or unsubstituted heteroalkyl. R⁴⁸ is a member selected from H, substituted or unsubstituted alkyl and substituted or unsubstituted heteroalkyl. u and v are integers independently selected from 0 to 10, and t is an integer selected from 0 to 20,000.

### D. Deuterated Analogs

In various embodiments, the invention provides prodrugs and compounds released from prodrugs in which at least one of R¹, R^{2a}, R^{2b}, R⁵¹, R^{52b} and R⁴ in any of Formulas A, B, C and/or Q is deuterium. Examples of such compounds include:

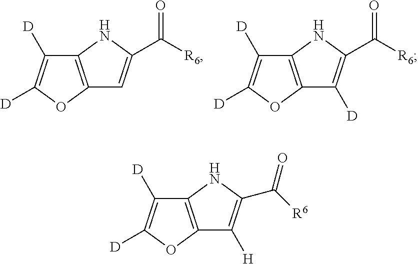

and mixtures thereof, wherein R⁶ can include deuterium. The compounds can optionally be labeled with another isotope, such as ¹³C. For example, in various embodiments, the carbon atom of the carbonyl group is ¹³C.

### E. Fluoro-Substituted Analogs

In various embodiments, the compounds of the present inventions (e.g., a compound of Formulae A, B, C and/or Q) are fluoro-substituted. For example, with reference to Formulae A and C, the inventions provide prodrugs and compounds released from prodrugs in which at least one member selected from R¹, R^{2a}, R^{2b} and R⁴ is F.

In various embodiments, the fluoro-substituted compound of the inventions has a structure according to Formula B and at least one of R⁵¹, R^{52b} and R⁴ is F.

Exemplary compounds according to these various embodiments include:

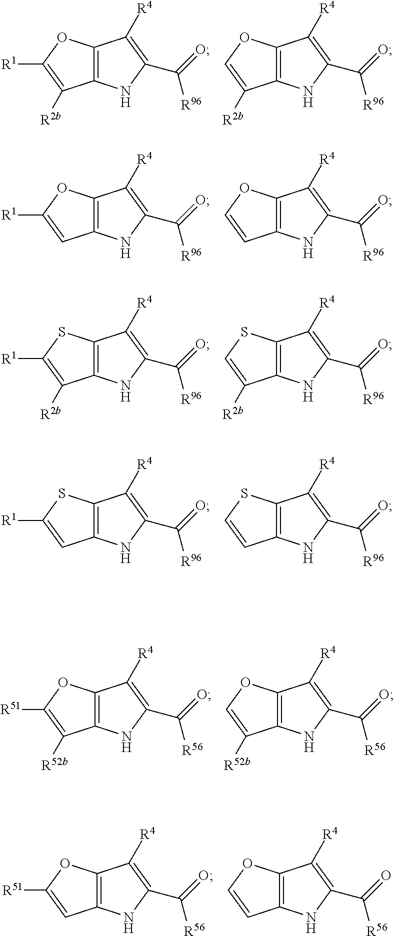

wherein R¹, R^{2b} and R⁴ are selected from H and F with the proviso that at least one of these radicals is F, wherein R⁹⁶ is R⁶ or R⁶⁶; wherein R⁵¹, R^{52b} and R⁴ are selected from H and F with the proviso that at least one of these radicals is F, and wherein R⁵⁶ is as discussed previously with respect to Formula B.

Exemplary compounds according to these various embodiments include:

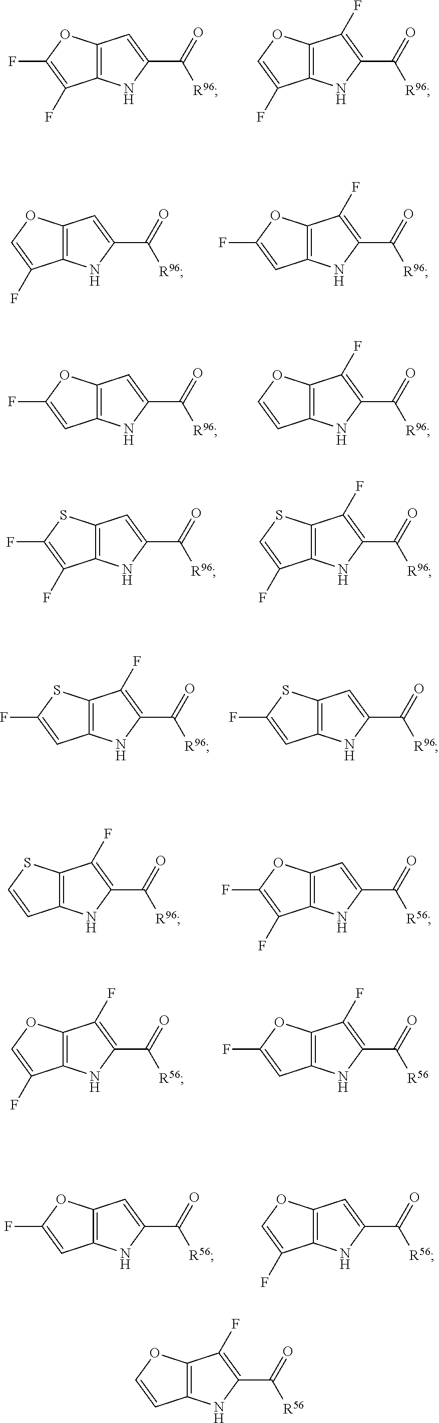

wherein R⁹⁶ is R⁶ or R⁶⁶; and wherein R⁵⁶ is as discussed previously with respect to Formula B.

In various embodiments, the inventions provide prodrugs that release F-substituted compounds. In various embodiments the released F-substituted compound has an IC₅₀ (DAAO inhibition) below about 1 μM, preferably below about 100 nM and more preferably below about 50 nM. In various embodiments, the prodrug releases a F-substituted compound having an IC₅₀ below about 25 nM, below about 10 nM, and/or below about 6 nM. In various embodiments, the prodrug (having Formulae A, B, C and/or Q) releases a F-substituted compound having an IC₅₀ that is at least about one order of magnitude lower than the IC₅₀ measured for at least one of the corresponding Br- or Cl-substituted analogs. In one example, the IC₅₀ is measured using an in vitro DAAO enzyme inhibition assay described herein.

In various embodiments, the inventions provide a F-substituted prodrug that releases a compound that increases D-serine levels in the cerebellum of a test animal. D-Serine levels may be determined following the experimental procedures known in the art. In various embodiments, the F-substituted active compound released from the prodrug (at 50 mg/kg) increases D-serine levels in the cerebellum of mice (measured 2 hours after i.p. dosing) between about 1.5 fold and 2 fold and preferably more than 2 fold when compared to vehicle.

In various embodiments, the inventions provide prodrugs releasing F-substituted compounds capable of maintaining an elevated D-serine level for at least 6 hours. For example, those F-substituted compounds released from such prodrugs, at e.g., 50 mg/kg, increase D-serine levels between about 1.5 fold and 2-fold and preferably more than 2-fold even when measured 6 hours after dosing, are generally preferred.

In various embodiments, the inventions provide prodrugs that release F-substituted compounds that increase D-serine levels at a lower dose of 10 mg/kg between about 1.5 fold and 2 fold and preferably more than 2 fold when measured 2 hours after dosing. For example, certain such prodrugs release F-substituted compounds that increase D-serine levels (at a lower dose of 10 mg/kg) between about 1.5 fold and about 2 fold and preferably more than 2 fold even when measured 6 hours after dosing.

In various embodiments, compounds of the present inventions can be useful in the treatment of pain and/or the improvement of cognition. Examples include those compounds of the present inventions that show activity in a pain model, such as the Chung model or Bennett model, as well as a model of cognition, such as a contextual fear conditioning or novel object recognition model.

In one example, the activity of a compound released from a compound of Formulae A, A^{x}, B, C and/or Q is measured using an in vitro DAAO enzyme inhibition assay. Such assays are known in the art. In various embodiments, compounds released from a compound of Formulae A, A^{x}, B, C and/or Q are judged to be sufficiently potent if they have an IC₅₀ below about 25 nM. In various embodiments, compounds released from a compound of

Formulae A, A^{x}, B, C and/or Q are judged to be sufficiently potent if they have an IC₅₀ below about 10 nM. This level of activity, for example, can be of use for pain treatments, e.g., treatment of neuropathic pain and other types of pain described herein. In various embodiments, compounds released from a compound of Formulae A, A^{x}, B, C and/or Q are judged to be sufficiently potent if they have an IC₅₀ below about 6 nM.

In one example, the activity of a compound released from a compound of Formulae A, A^{x}, B, C and/or Q is determined by measuring D-serine levels in vivo. Elevation of the D-serine level in a certain brain area (e.g., the cerebellum) of a test animal (e.g., mouse, rat, pig and the like) is indicative of DAAO inhibition in vivo. An exemplary assay format is one that measures D-serine levels (LC/MS/MS) in the cerebellum of mice two hours and six hours after intraperitoneal (i.p.) dosing. Increases in D-serine levels were determined through comparison with vehicle. Useful variations of this assay will be apparent to those of skill in the art. Compounds released from a compound of Formulae A, A^{x}, B, C and/or Q are judged to be sufficiently active in this assay when at least one, at least two, at least three or all four of the following criteria are met:

1) At a dose of 50 mg/kg, a compound released from a compound of Formulae A, A^{x}, B, C and/or Q causes an elevation of D-serine level (measured about 2 hours after dosing) of greater than about 2 fold when compared to vehicle.

2) At a dose of 50 mg/kg, a compound released from a compound of Formulae A, A^{x}, B, C and/or Q causes an elevated D-serine level (measured about 6 hours after dosing) of greater than about 2 fold when compared to vehicle.

3) At a dose of 10 mg/kg, a compound released from a compound of Formulae A, A^{x}, B, C and/or Q causes an elevation of D-serine level (measured 2 hours after dosing) of greater than about 2 fold when compared to vehicle.

4) At a dose of 10 mg/kg, a compound released from a compound of Formulae A, A^{x}, B, C and/or Q causes an elevation of D-serine level (measured 6 hours after dosing) of greater than about 2 fold when compared to vehicle.

In various embodiments, compounds of the present inventions (a compound of Formulae A, A^{x}, B, C and/or Q) are judged appropriate for pharmaceutical development upon demonstration of release of an active compound in a manner that provides sufficient activity against the enzyme DAAO both in vitro (e.g., DAAO enzyme inhibition assay) and in vivo (e.g., elevation of D-serine levels in the cerebellum of mice).

### F. Synthesis

Compounds of the present inventions, including compounds of Formulae A, A^{x}, B,

C and Q, may be prepared by methods known in the art. One of ordinary skill in the art will know how to modify procedures to obtain the analogs of the present inventions. Suitable procedures are described e.g., in WO/2004/031194 to Murray, P. et al.; Yarovenko, V. N., Russian Chemical Bulletin, International Edition (2003), 52(2): 451-456; Krayushkin M. M. et al., Organic Letters (2002), 4(22): 3879-3881; Eras J. et al., Heterocyclic Chem. (1984), 21: 215-217, each of which is incorporated herein by reference in its entirety. Further, procedures that may be useful to one of ordinary skill in the art in synthesizing various embodiments of the present inventions can be found in Reference items 1 to 28 listed below, each of which is incorporated herein by reference in its entirety. In addition, compounds may be prepared using the methods described below and in Examples A, and 1-10 or modified versions thereof.

Active drug compounds for which prodrugs are of interest include fused aromatic acids, such those found in United States Patent Application Publications WO/2008/005456, US/2008/0058395, and US/2008/0004327 and U.S. patent application Ser. Nos. 11/883,903 and 12/016,954, for example.

In various embodiments, compounds described herein as compounds released from a compound of the present inventions (e.g. a compound of general formulae A, A^{x}, B, C and/or Q) are appropriate precursors for synthesis of the compounds of the present inventions. In various embodiments, a precursor for a prodrug is chosen from the following list:

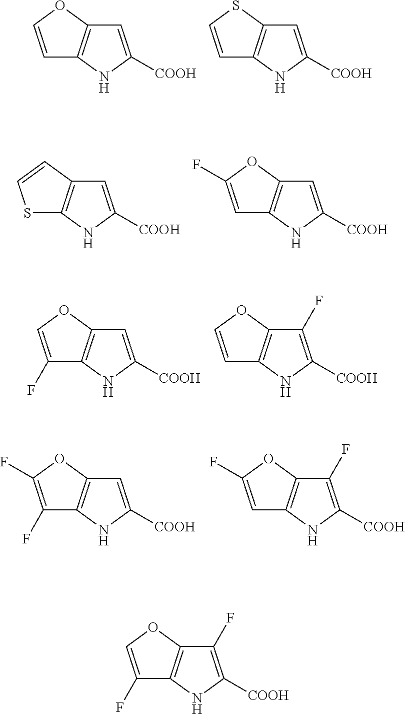

In the following description and elsewhere in the present description, the use of 4H-furo[3,2-b]pyrrole-5-carboxylic acid or any other acid as a precursor is for the sake of convenience only, and it should be understood that other fused heterocycles as disclosed herein are equally suitable precursors as well.

In various embodiments, the compound to be synthesized has the structure of

Formula D

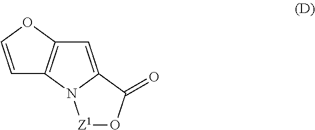

Z¹ is a member selected from SO₂, C(O)CR³¹R³² and CR³¹R³², wherein R³¹ and R³² are members independently selected from H, acyl, substituted or unsubstituted alkyl, substituted or unsubstituted heteroalkyl, substituted or unsubstituted aryl, substituted or unsubstituted heteroaryl and substituted or unsubstituted heterocycloalkyl. R³¹ and R³² together with the carbon to which they are attached are optionally joined to form a ring. Optionally, together R³¹ and R³² and the carbon to which they are attached form C═O.

Lactone containing compounds such as a compound of structure D, with Z¹ equal to C(O)CR³¹R³² may be synthesized according to the scheme below as well as similar by similar chemistry that couples a fused pyrrole acid with a substituted or unsubstituted 2-hydroxyacetic acid equivalent followed by cyclization to the desired lactone.

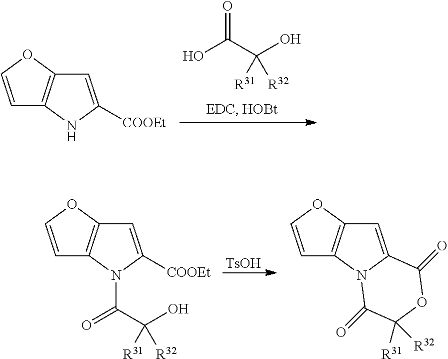

Oxazolidinedione-containing prodrugs such as a compound of structure D, with Z¹ equal to C(O) can be synthesized according to the scheme below.

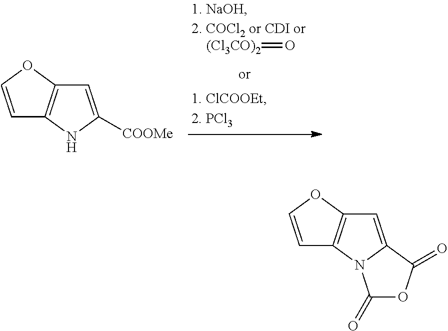

Various ethyloxazolidinone-containing prodrugs such as a compound of structure D, with Z¹ equal to CHR³¹ can be synthesized according to the scheme below.

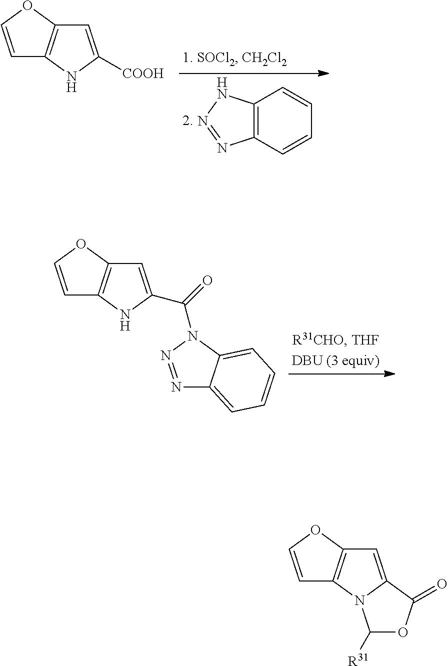

Various oxazolidinone-containing prodrugs such as a compound of structure D, with Z¹ equal to CR³¹R³² can be synthesized according to the scheme below.

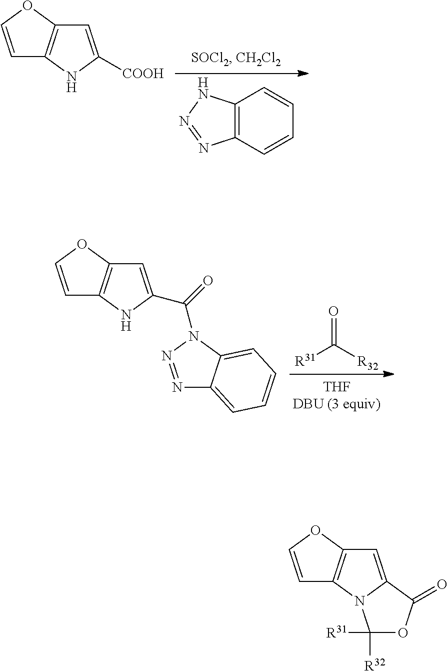

In various embodiments, the compound to be synthesized has the structure

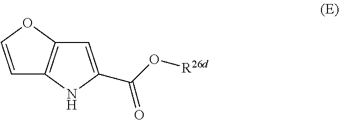

wherein R^{26d} is R²⁶, R^{26b} or R^{26c} as discussed hereinabove.

Examples of compounds according to Formula E include:

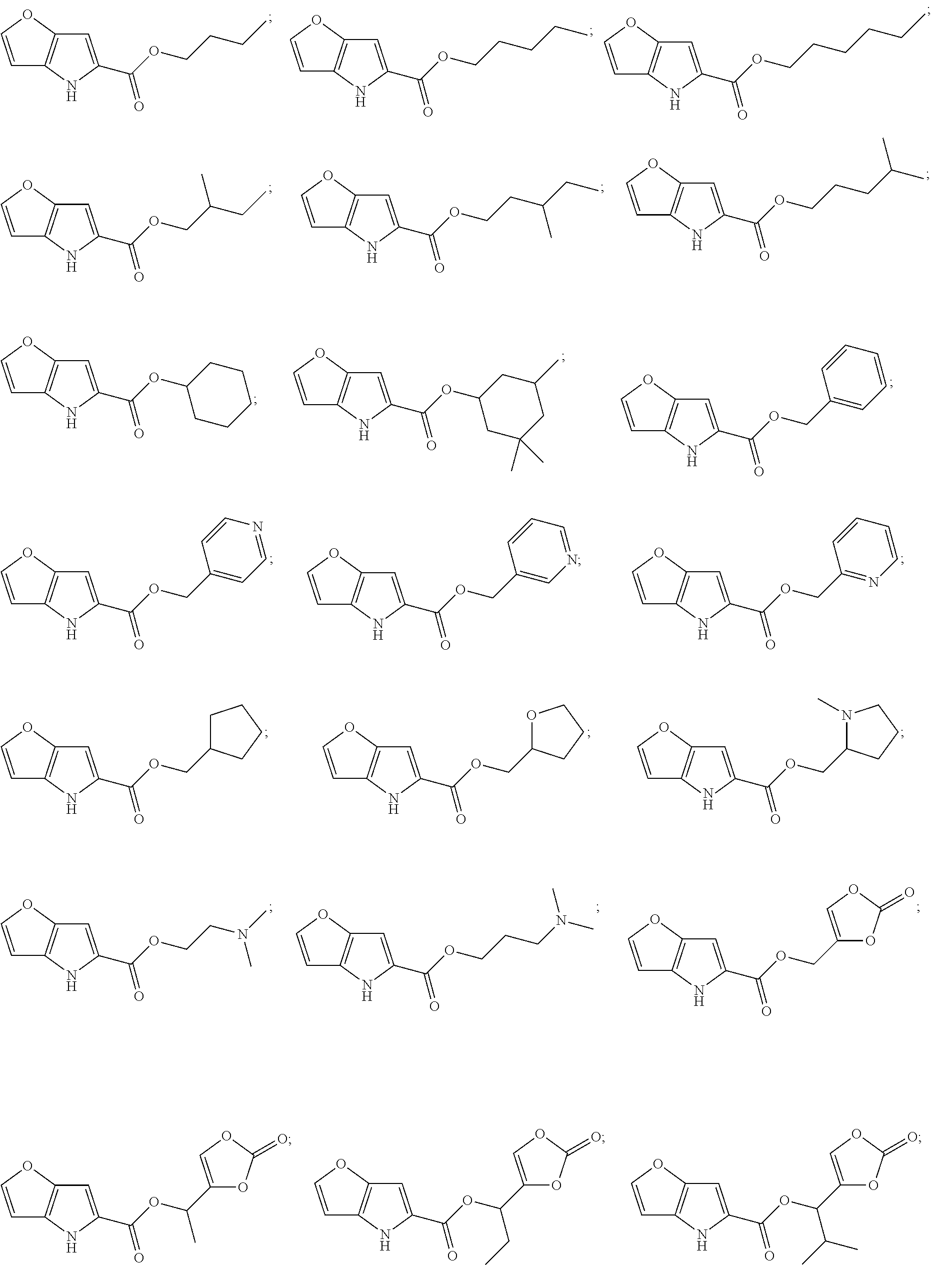

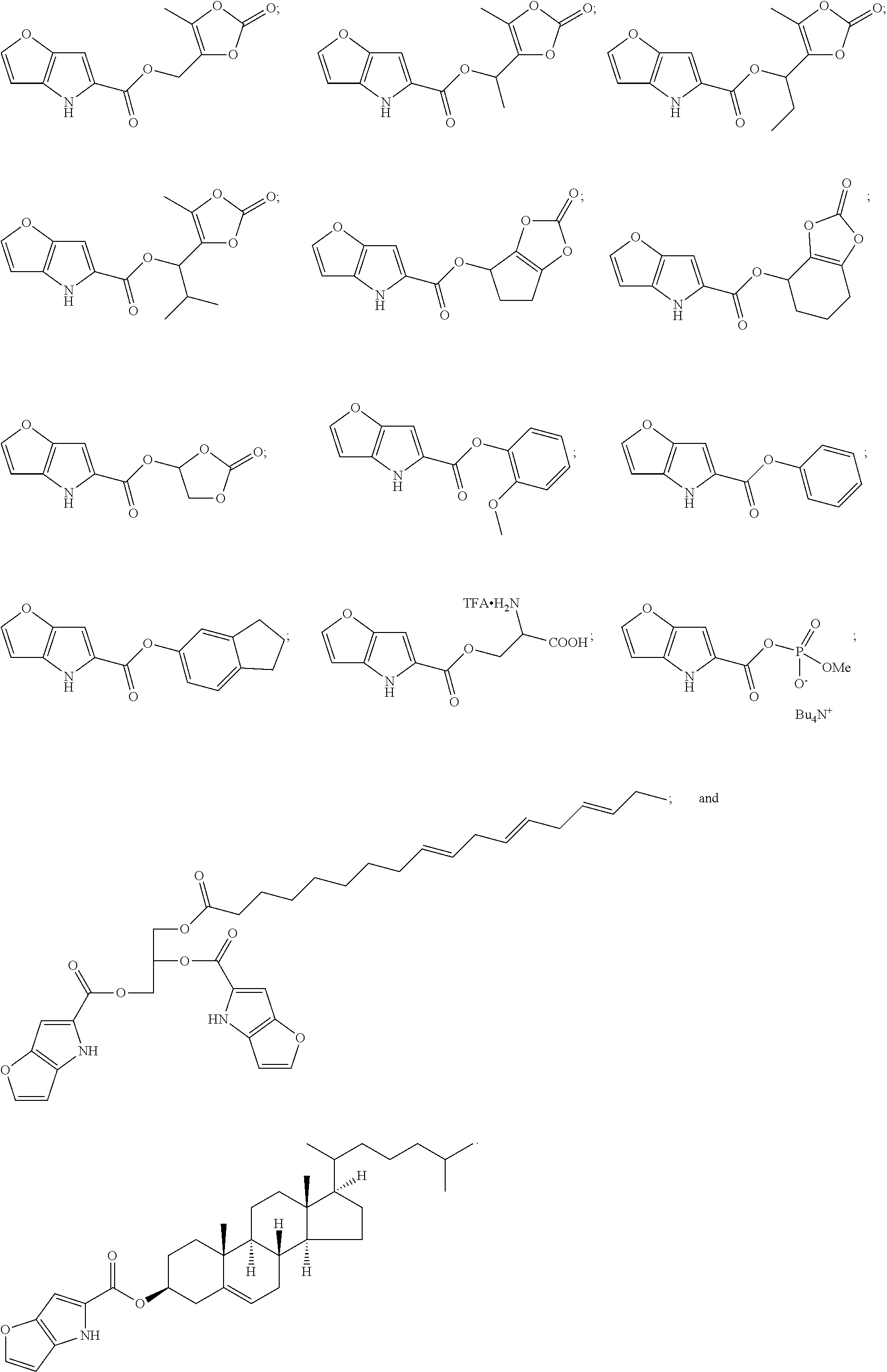

Various ester-containing prodrugs such as compounds according to Formula E can be synthesized according to the scheme below.

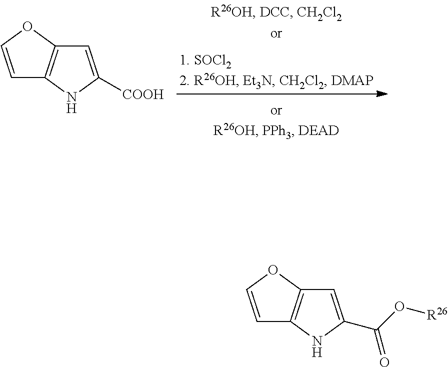

In various embodiments, ester-containing prodrugs such as compounds according to Formula E can be synthesized according to the schemes below:

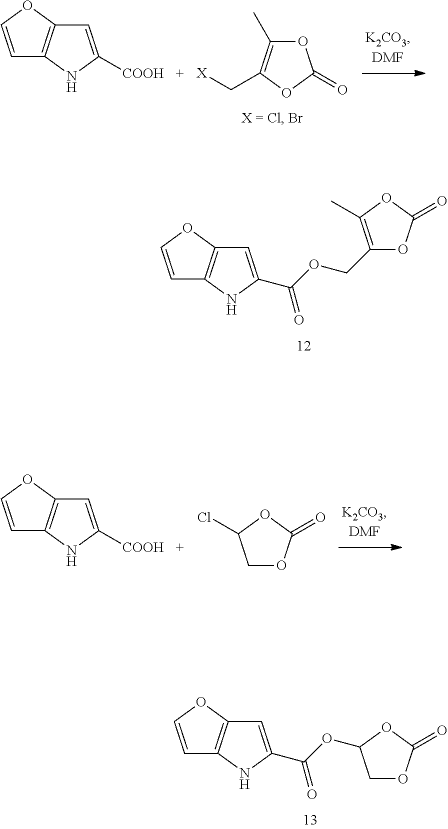

Amino acid-derived ester prodrugs such as 16 can be synthesized according to the scheme below.

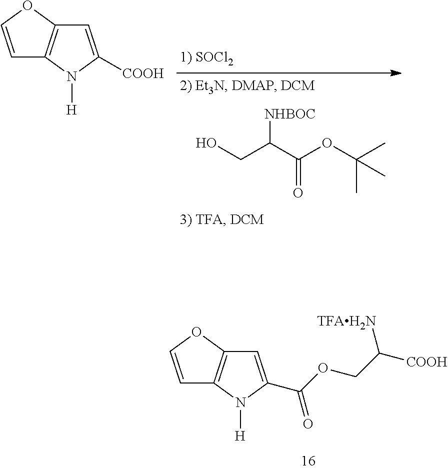

Phosphate-containing prodrugs such as 17 can be synthesized according to the scheme below.

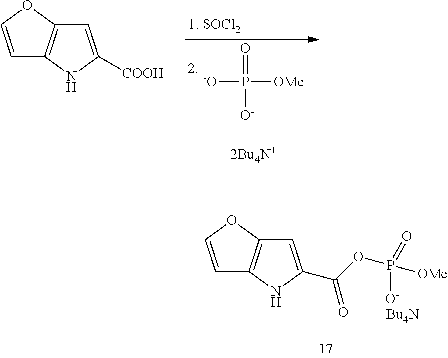

Ester-containing prodrugs such as 18 can be synthesized according to the scheme below.

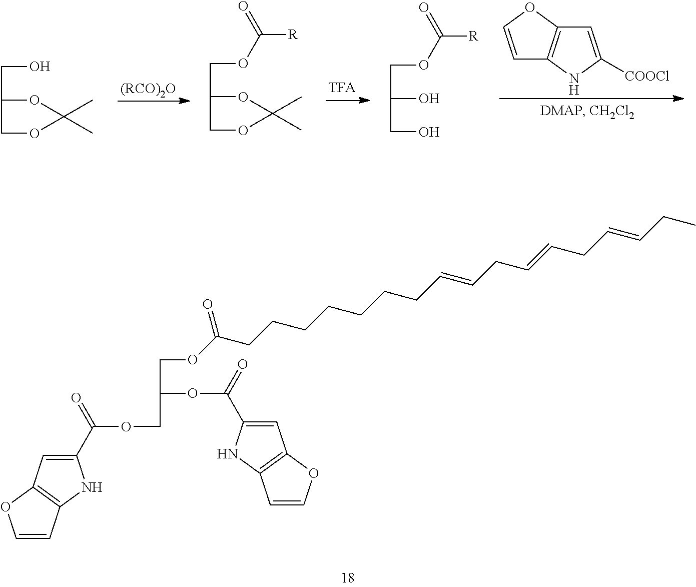

In various embodiments, the compound has the structure of Formula F

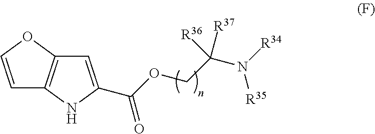

wherein R³⁴ and R³⁵ are as described above. R³⁶ and R³⁷ are as described above; and n=0 or 1.

Exemplary compounds according to Formula F are shown below:

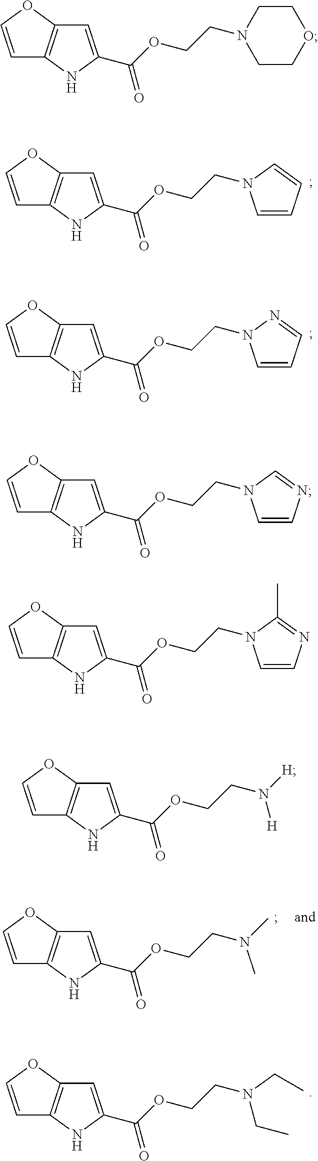

Various aminoethyl ester compounds of the present inventions can be synthesized according to the schemes below.

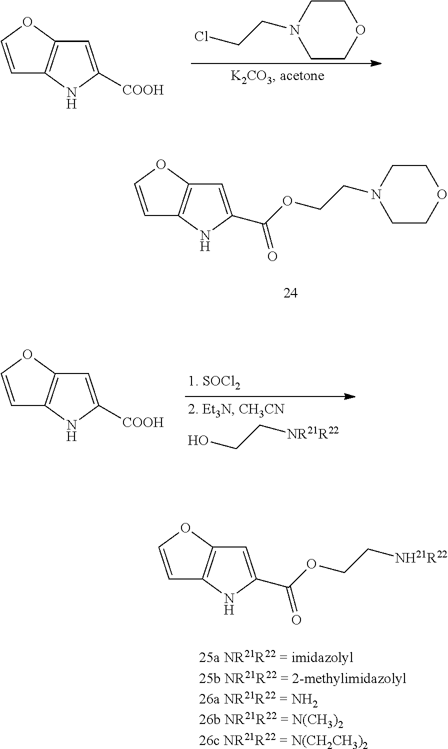

In various embodiments, the compound has the structure of Formula G

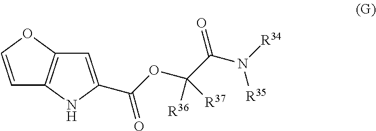

wherein R³⁴, R³⁵, R³⁶ and R³⁷ are as described above.

Exemplary compounds according to Formula G are shown below:

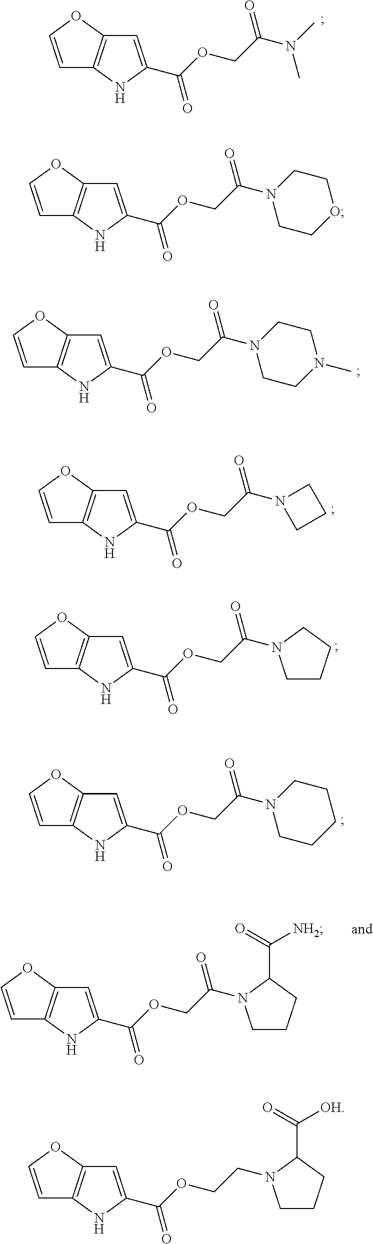

Glycolamide ester prodrugs such as 20-23 can be synthesized according to the scheme below.

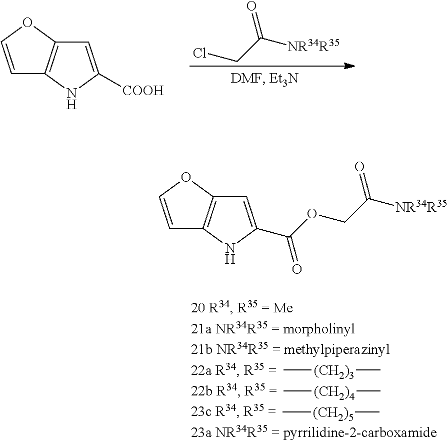

In various embodiments, the compound has the structure of Formula H

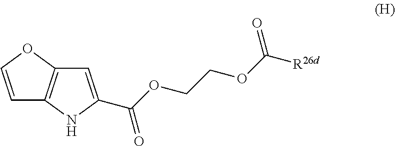

wherein R^{26d} is as discussed above.

Exemplary compounds according to Formula H are shown below:

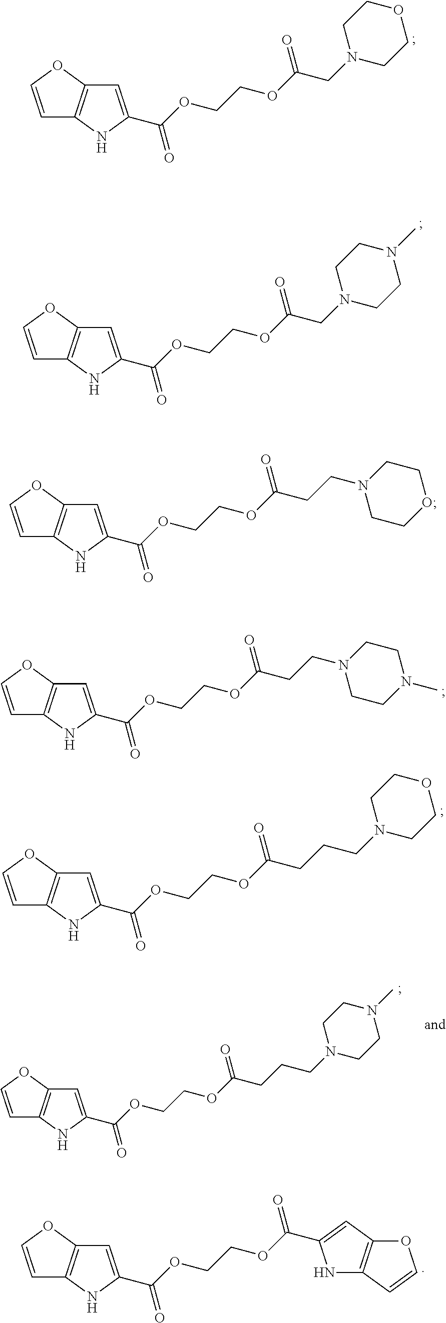

Exemplary ester prodrugs such as 27 and 28 can be synthesized according to the schemes below.

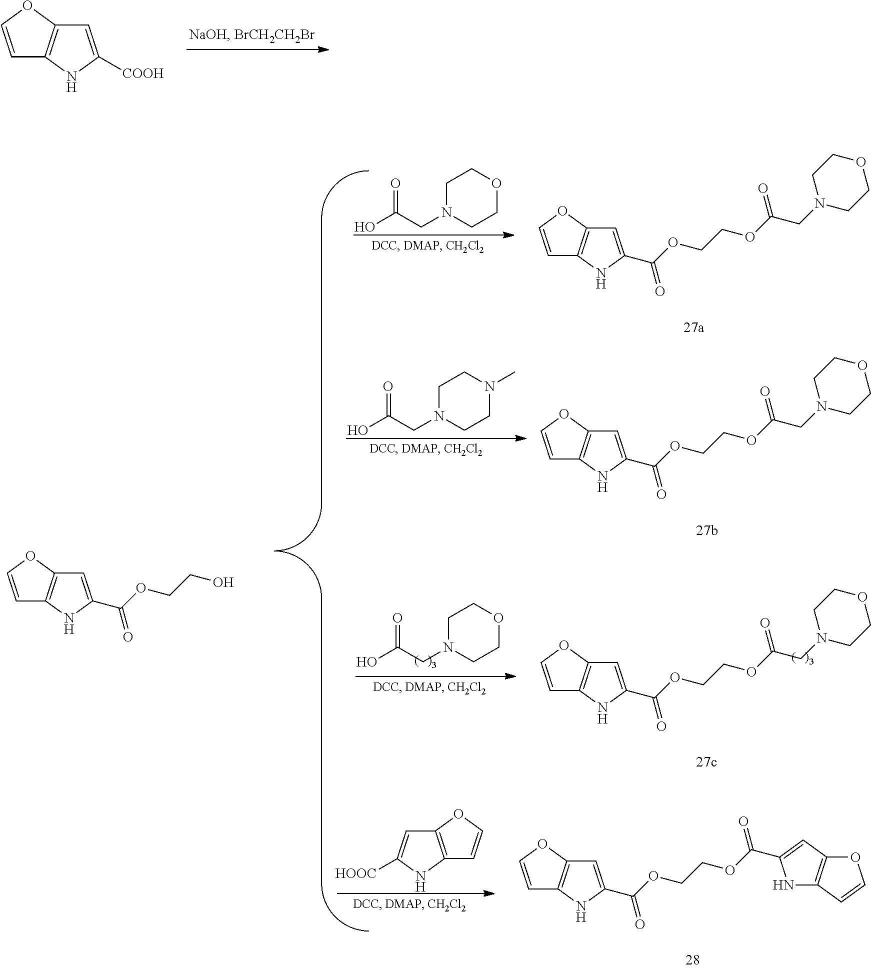

In various embodiments, the compound has the structure of Formula J

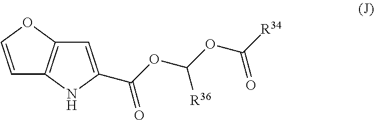

wherein R³⁴ and R³⁶ are as described above; and R³⁴ and R³⁶ are optionally joined to form a ring.

Exemplary compounds according to Formula J are shown below:

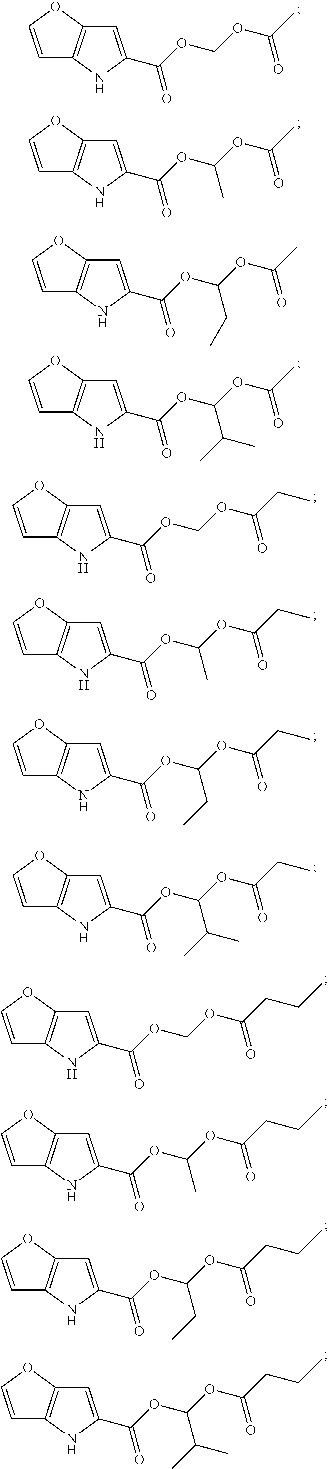

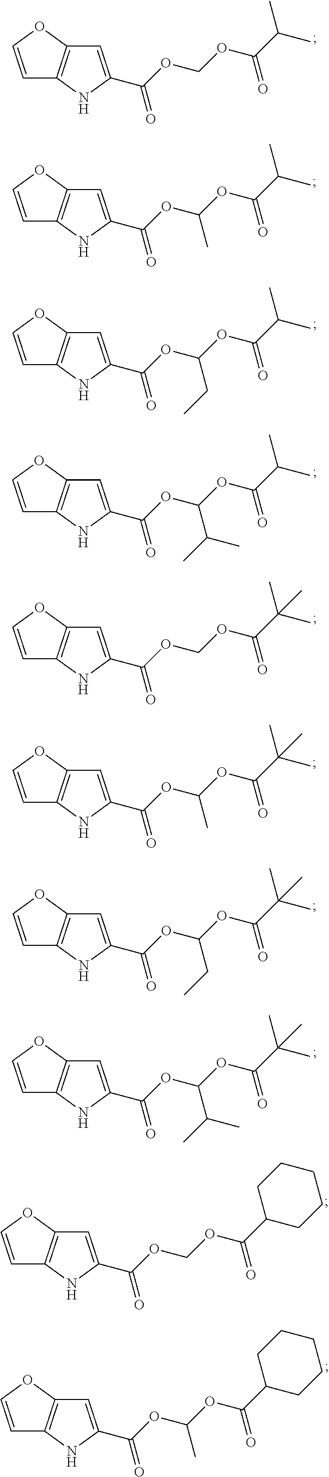

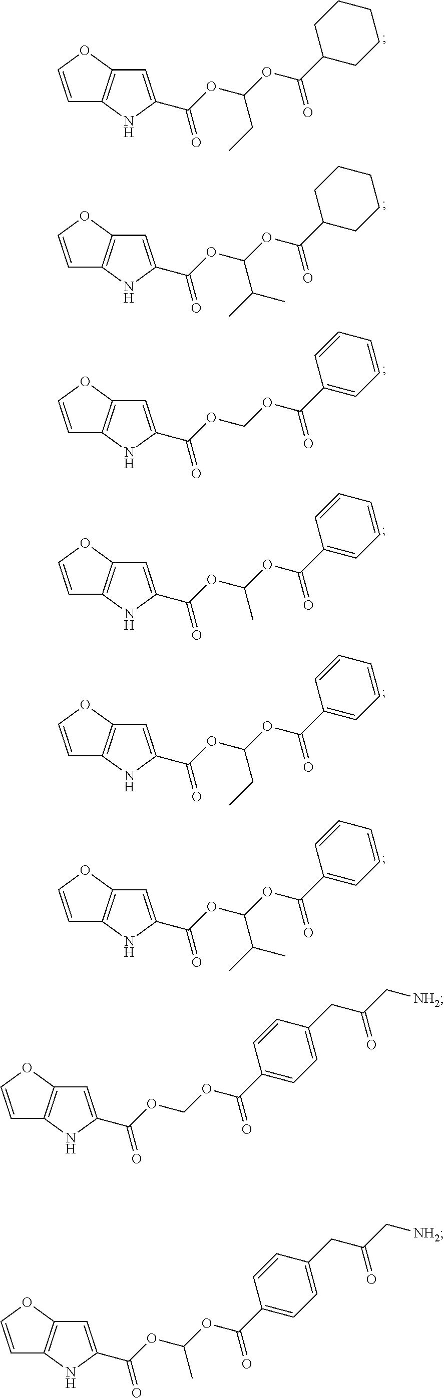

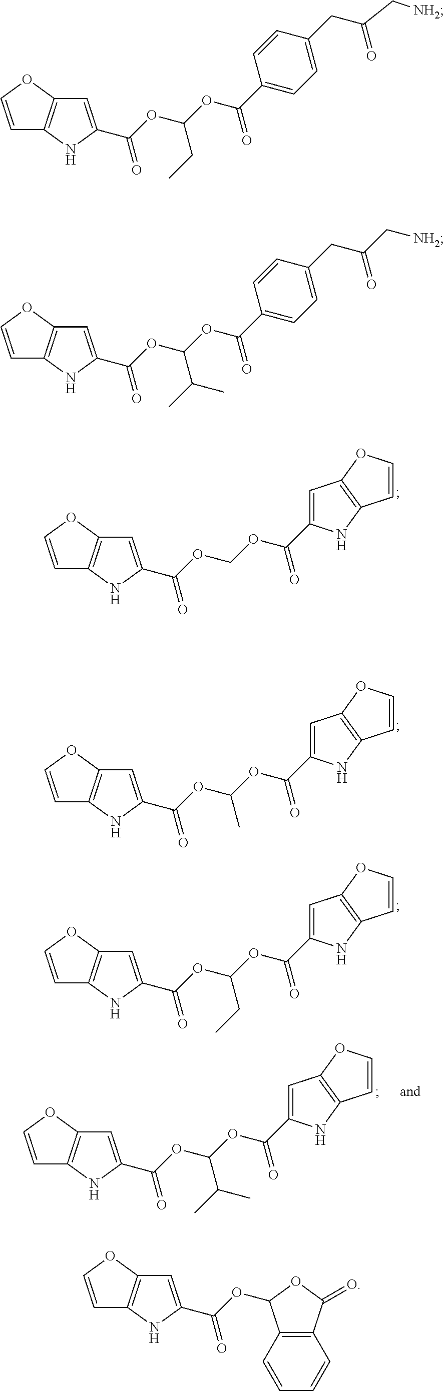

Acyloxymethylester prodrugs such as 29-33 can be synthesized according to the scheme below.

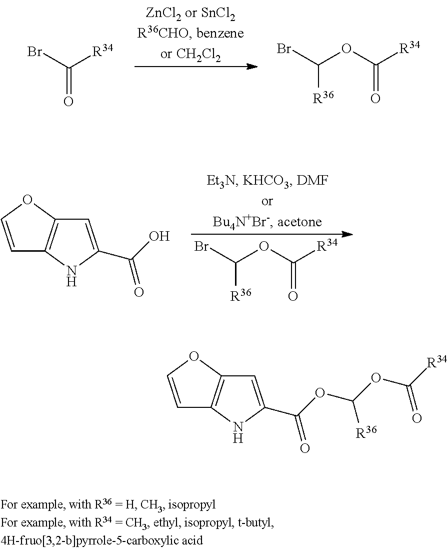

Ester prodrugs such as 39 can be synthesized according to the scheme below:

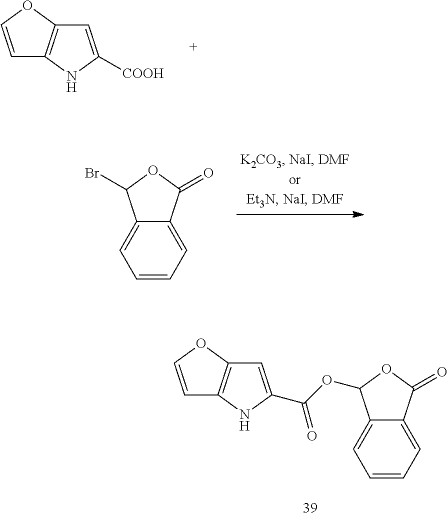

In various embodiments, the compound has the structure Formula K

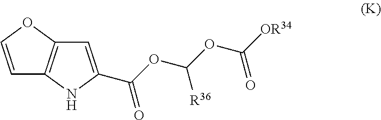

wherein R³⁴ and R³⁶ are as described above; and R³⁴ and R³⁶ are optionally joined to form a ring.

Exemplary compounds according to Formula K are shown below:

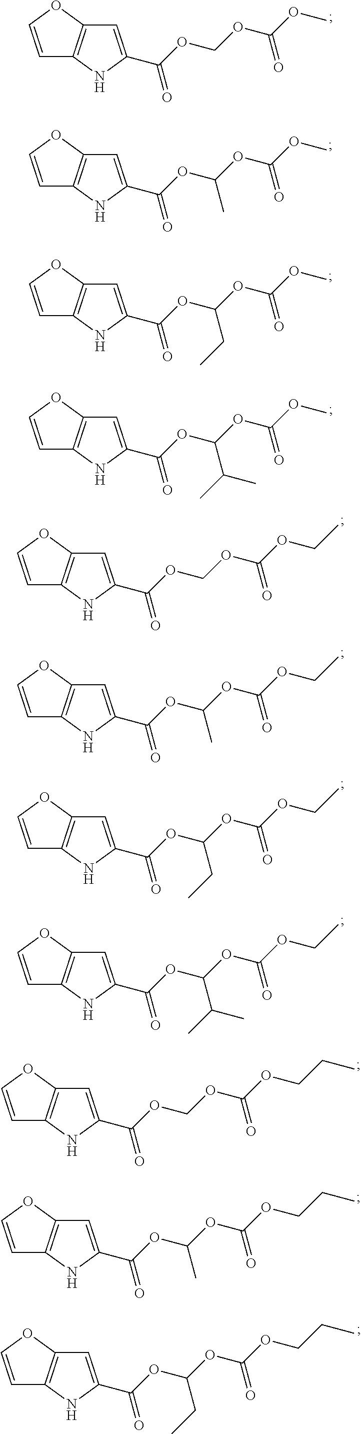

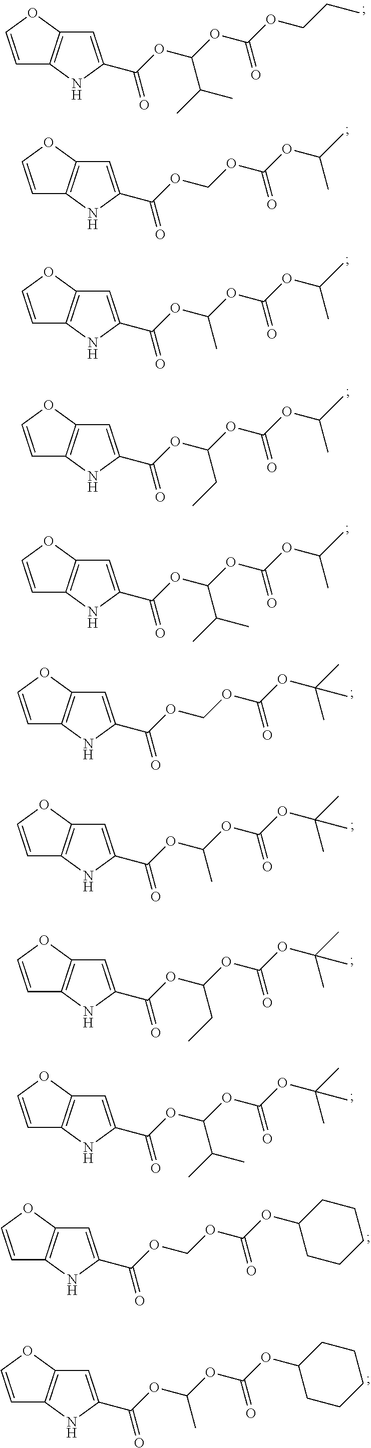

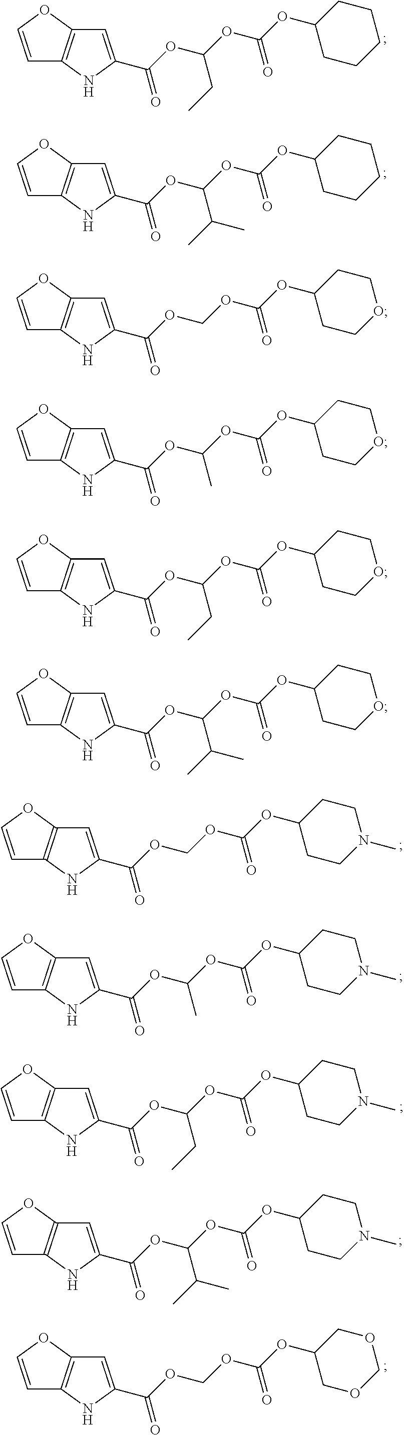

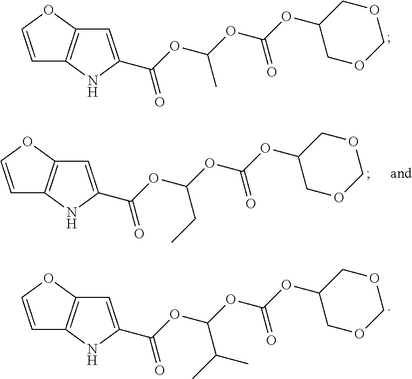

Acyloxymethylester prodrugs such as 21-23, and 43, 34-38 and 42 can be synthesized according to the scheme below.

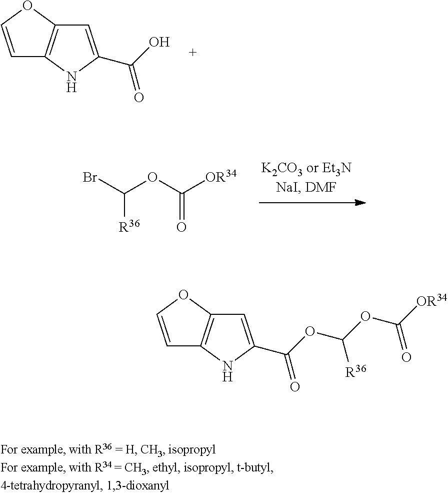

Ester prodrugs such as 39 can be synthesized according to the scheme below:

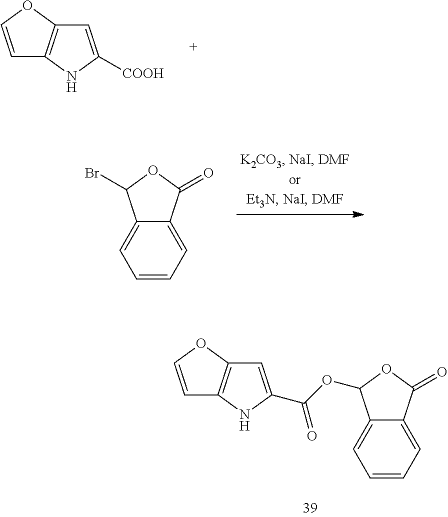

In various embodiments, the compound has the structure of Formula L

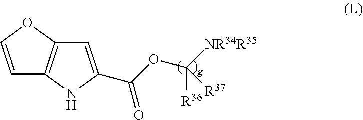

wherein R³⁴, R³⁵, R³⁶, R³⁷ and g are as described above.

Exemplary compounds according to Formula L are shown below:

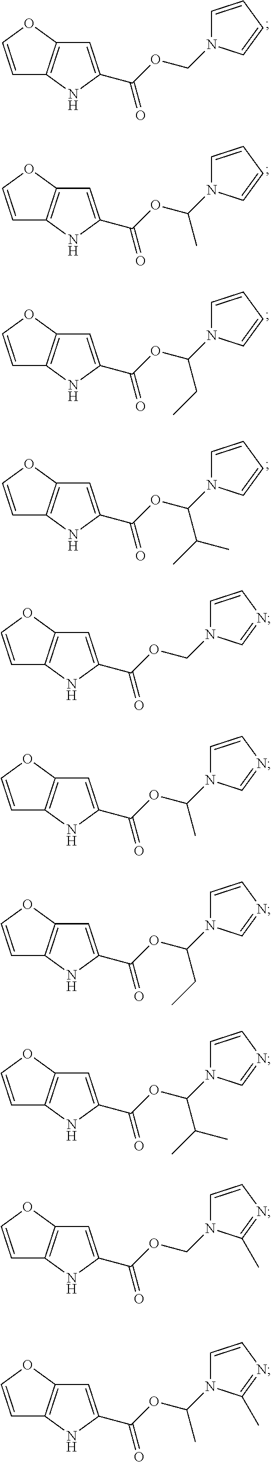

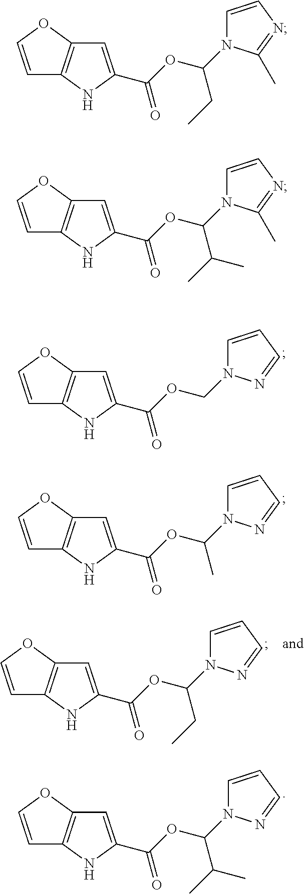

Compounds such as α-(1H-imidazol-1-yl)alkyl (IMIDA) carboxylic acid ester-containing prodrugs such as 40a and 40b can be synthesized according to the scheme below.

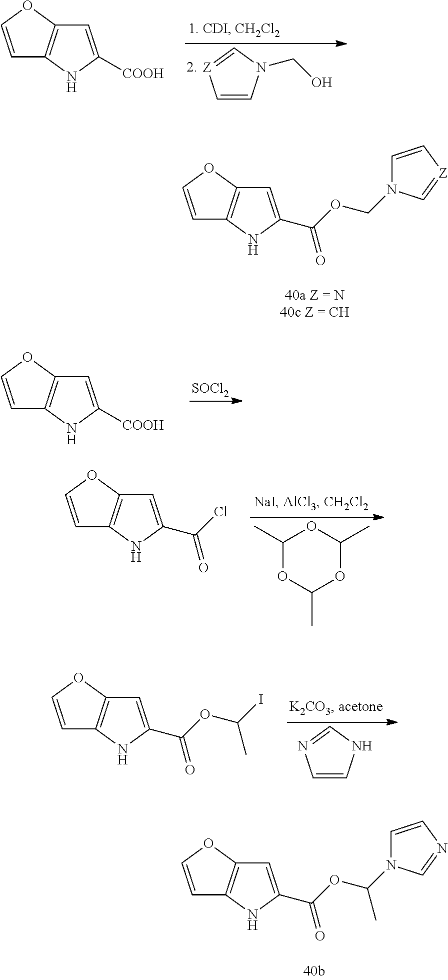

In various embodiments, a compound of the present inventions has the structure of Formula M

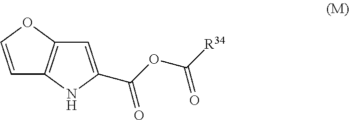

wherein R³⁴ is as described above.

Exemplary compounds according to Formula M are shown below:

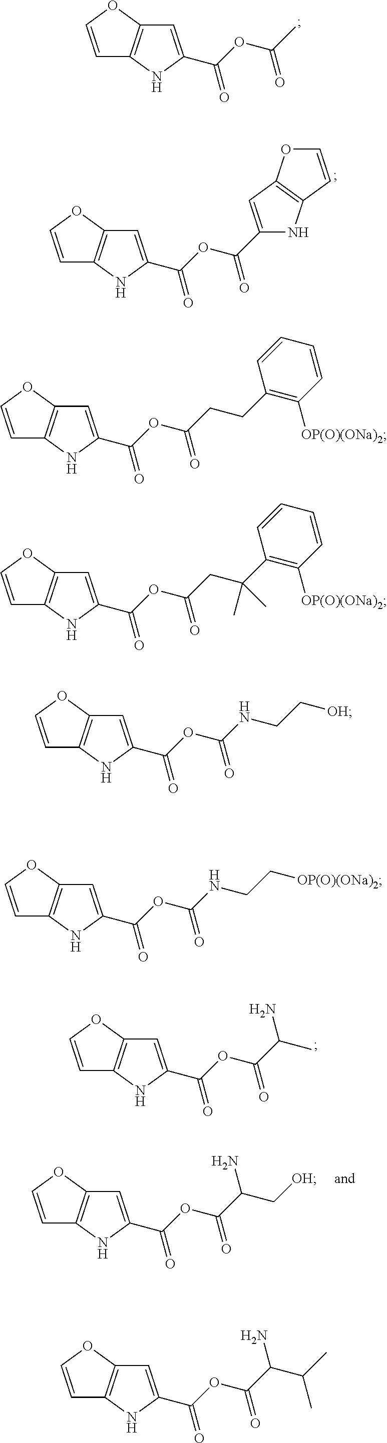

Anhydrides such as 41 to 45 can be synthesized using standard conditions. Preparation of the requisite linkers for 43 is reported in the literature. Representative syntheses are shown in the following schemes:

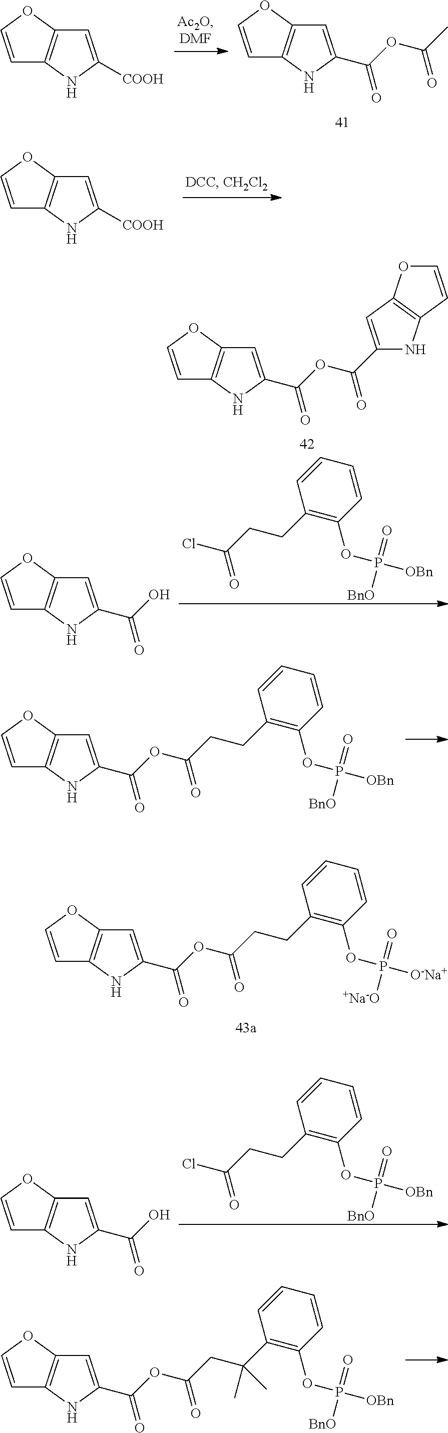

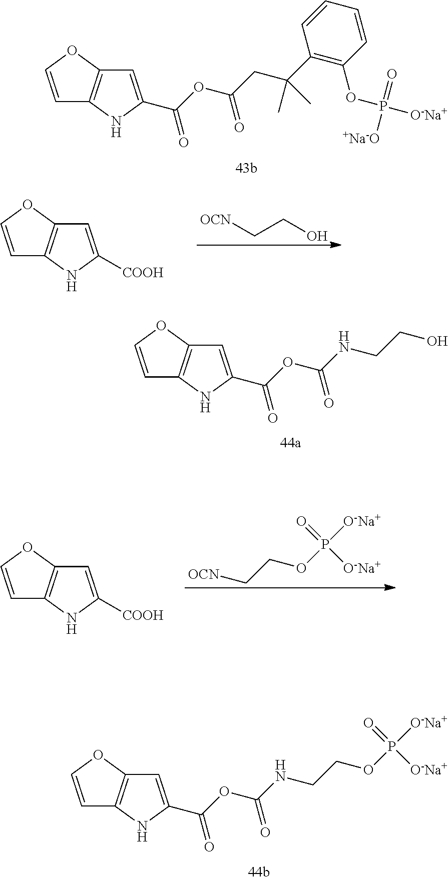

In various embodiments, the compound has the structure of Formula N

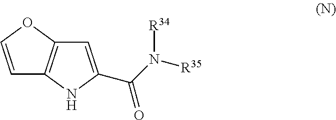

wherein R³⁴ and R³⁵ are as described above.

Exemplary compounds according to Formula N are shown below:

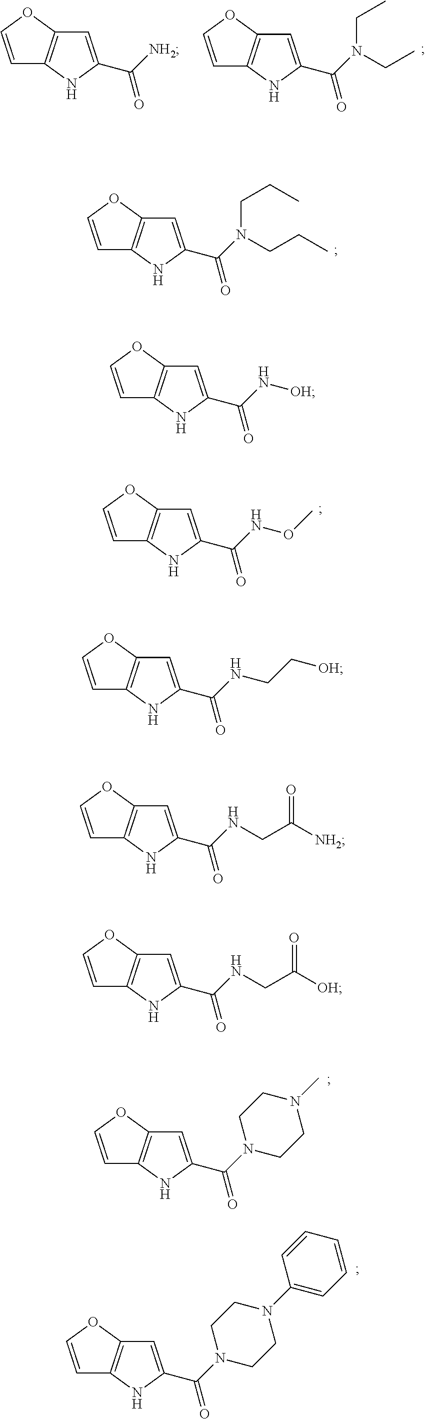

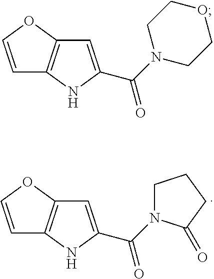

Various amide-containing prodrugs such as 46 can be synthesized according to the scheme below.

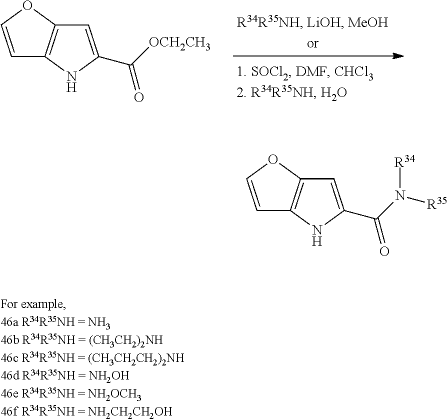

Amide-containing prodrugs such as 47 can be synthesized according to the scheme below:

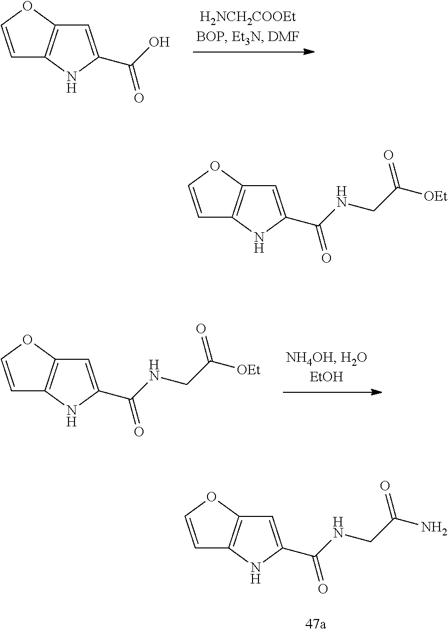

Amide-containing prodrugs such as 48 can be synthesized according to the scheme below:

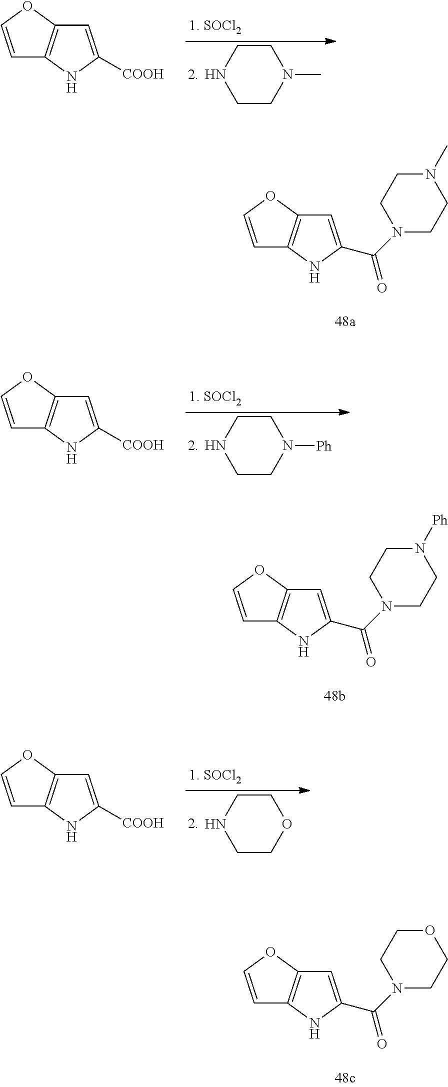

Amide-containing prodrugs such as 49 can be synthesized according to the scheme below:

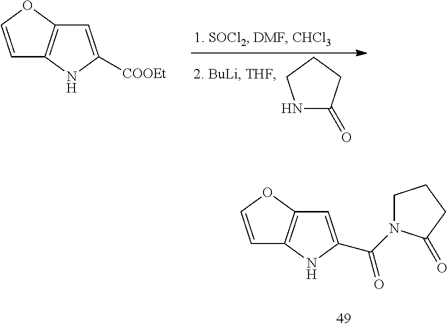

In various embodiments, the compound has the structure of Formula P

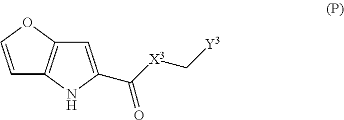

wherein X³ is a member selected from O and S; and Y³ is a member selected from Cl and F.

Exemplary compounds according to Formula P are shown below:

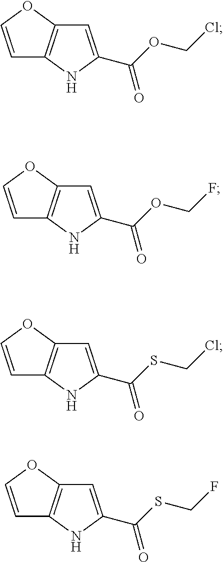

Chloromethyl-containing prodrugs such as 50a can be synthesized according to the scheme below.

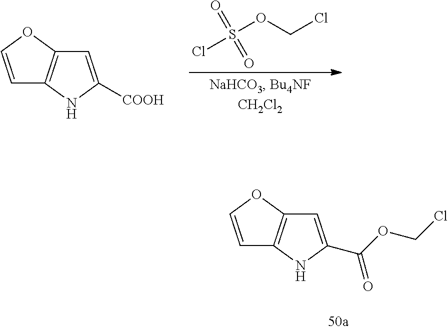

Fluoromethyl-containing prodrugs such as 50b can be synthesized according to the scheme below.

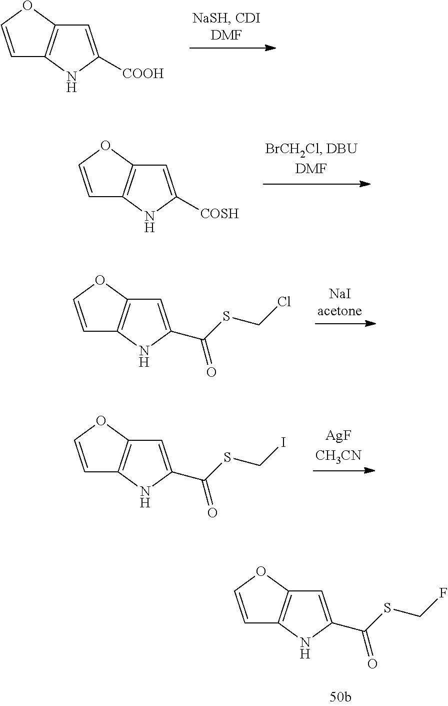

In various embodiments, the compound has the structure according to Formula Q.

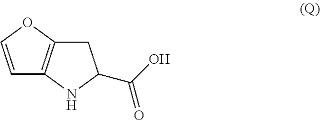

An example representing a stereoisomer of Formula Q is shown below:

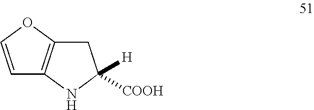

Prodrugs such as 51 can be synthesized as shown below. There are a variety of conditions to affect the desired cyclization.

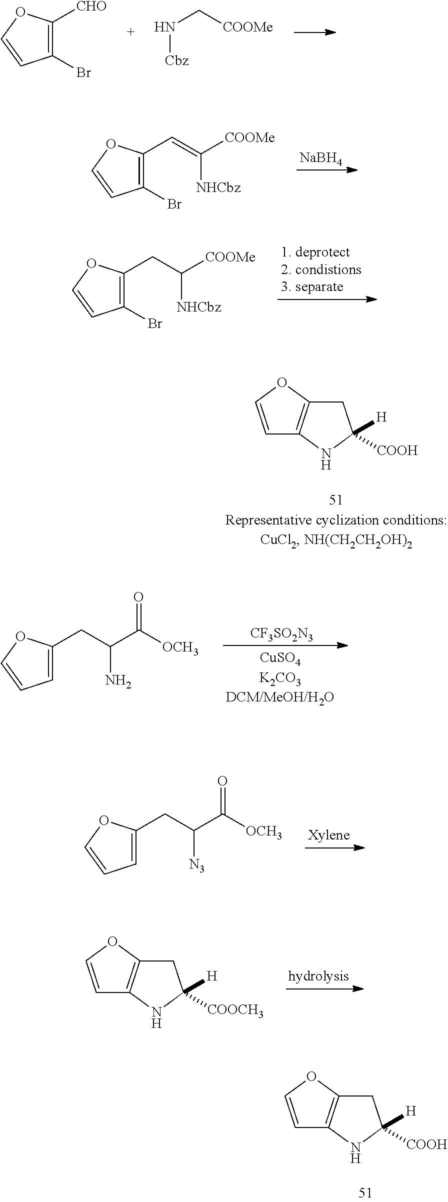

In various embodiments, R⁶ is a member selected from OH, OCH₂CH₃, and OCH₃, and R⁶a is

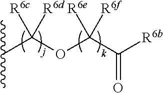

wherein R^{6a}, R^{6d}, R^{6e} and R^{6f} are members independently selected from H, substituted or unsubstituted heteroalkyl, substituted or unsubstituted arylalkyl, substituted or unsubstituted heteroarylalkyl, substituted or unsubstituted cycloalkyl, substituted or unsubstituted heterocycloalkyl and alkenyl, and where R^{6c} and R^{6d} are optionally joined to form a ring, and where R^{6e} and R^{6f} are optionally joined to form a ring, and where R^{6c} and R^{6e} are optionally joined to form a ring. The index j is an integer independently selected from 1 to 5. The index k is an integer selected from 0 to 5. R^{6b} is a member selected from substituted or unsubstituted aryl, substituted or unsubstituted alkyl, substituted or unsubstituted heteroalkyl, substituted or unsubstituted arylalkyl, substituted or unsubstituted heteroarylalkyl, substituted or unsubstituted cycloalkyl, substituted or unsubstituted heterocycloalkyl and alkenyl.

In various embodiments, the compounds have structures according to the following Table 1:

TABLE 1

| Commercially | | |
|---|---|---|
| available precursor | Prodrugs | |
| 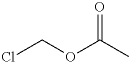 | 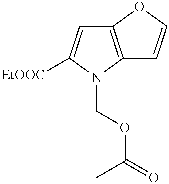 | 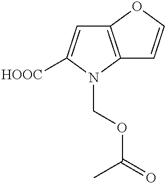 |
| 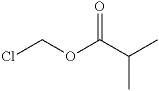 | 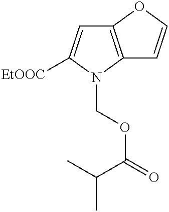 | 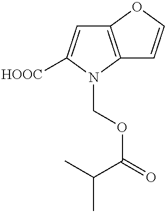 |
| 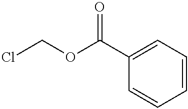 | 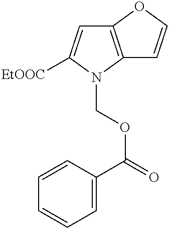 | 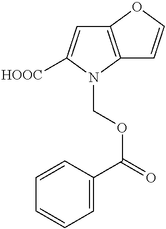 |
| 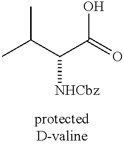 | 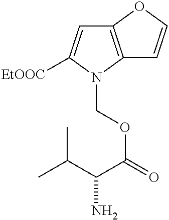 | 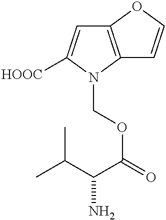 |
| 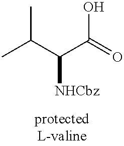 | 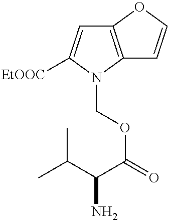 | 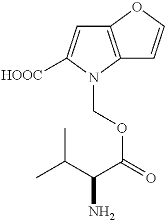 |
| 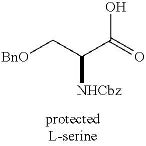 | 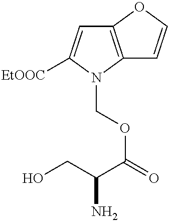 | 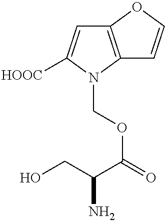 |
| 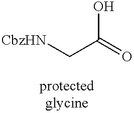 | 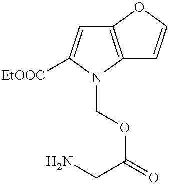 | 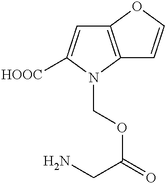 |
| 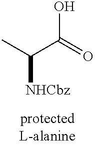 | 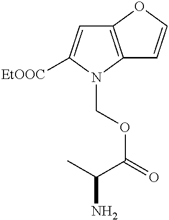 | 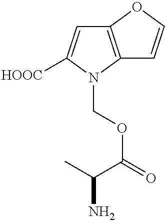 |

The above compounds can be synthesized according to the following scheme:

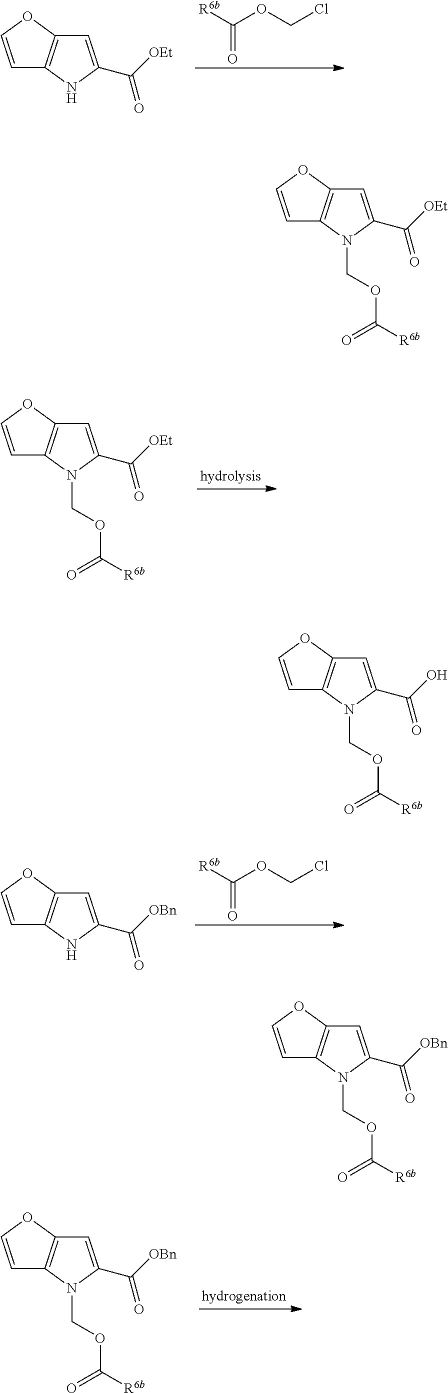

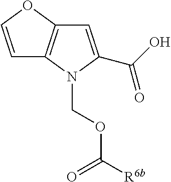

In an exemplary synthetic procedure, when the R-group of the chloromethyl ester is derived from an amino acid, a protecting group strategy is employed. For example, protection of the amino acid nitrogen with Cbz.

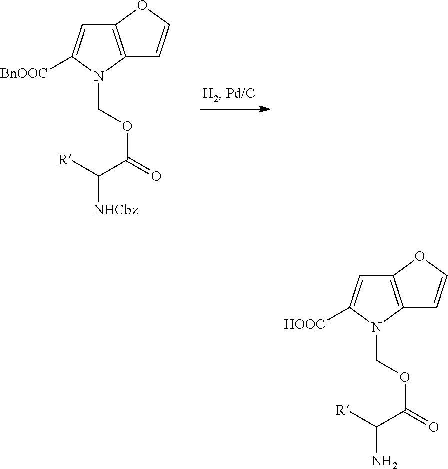

The following scheme outlines an alternate route to various compounds of the present inventions.

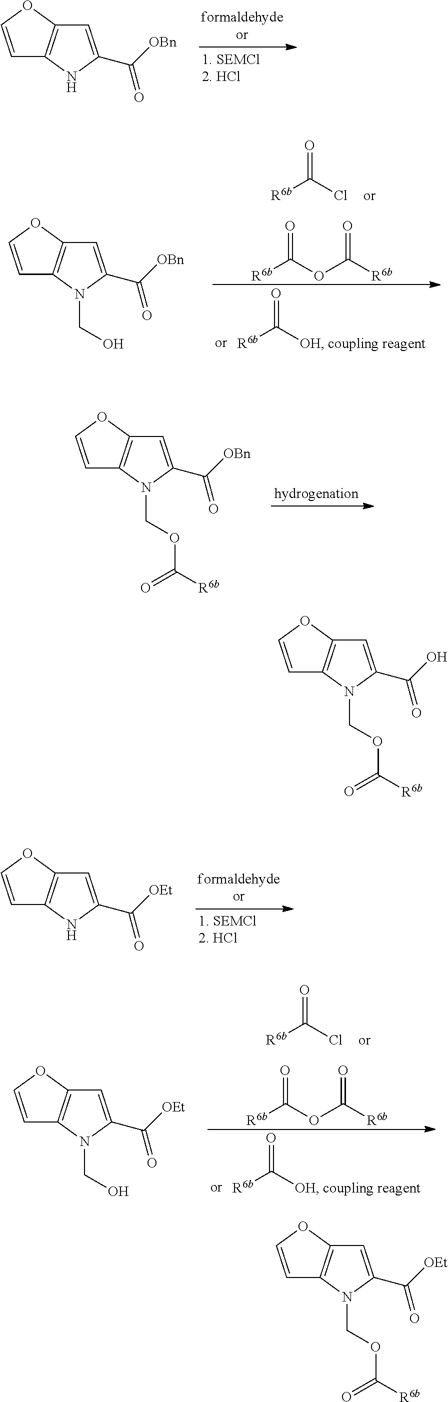

One of ordinary skill in the art using the teachings herein and synthetic routes know to the art can synthesize dipeptide prodrugs, such as those in the following Table 2:

TABLE 2
Prodrugs

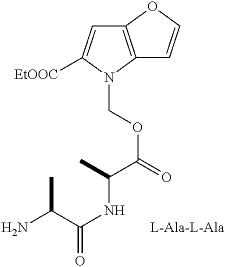

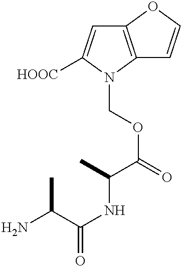

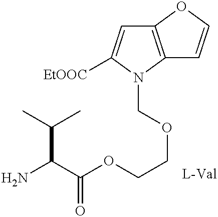

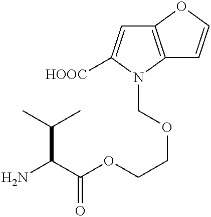

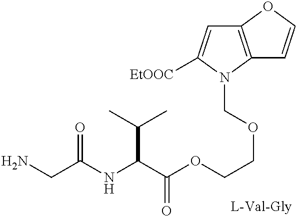

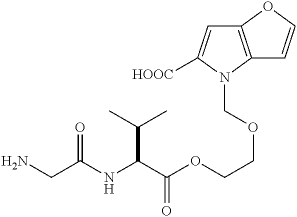

In various embodiments, the compounds have the structures of Table 3 below:

TABLE 3
Prodrugs

| | |
|---|---|
| 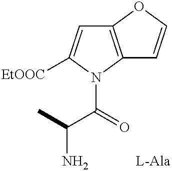 | 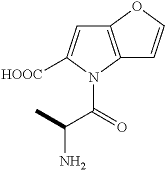 |
| 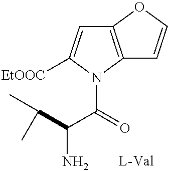 | 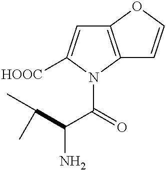 |

The above compounds may be synthesized according to the following scheme:

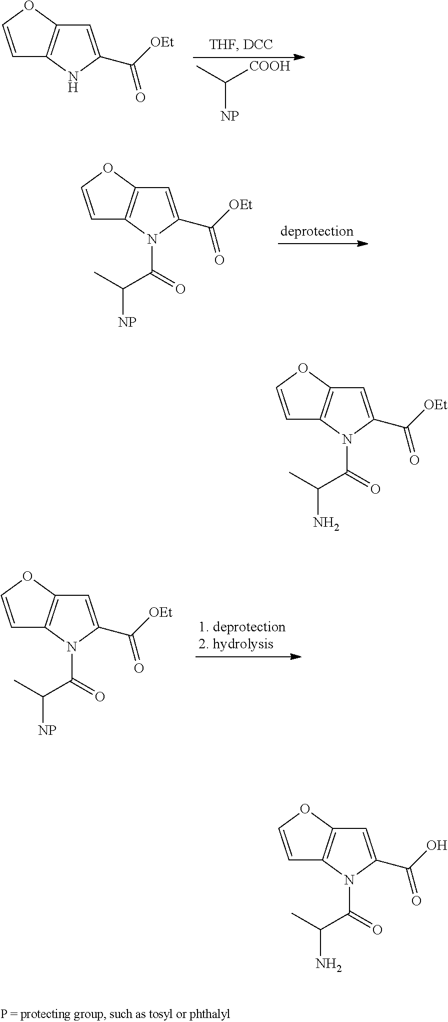

### G. Pharmaceutical Compositions

While it is possible for compounds of the present inventions to be administered neat, without formulation, it is preferable to provide them as a pharmaceutical composition. In various embodiments, the present inventions provide a pharmaceutical composition comprising a compound of Formulae A-Q (including Formula A^{x}) or a pharmaceutically acceptable salt, solvate, or hydrate thereof, together with one or more pharmaceutical carrier and optionally one or more other therapeutic ingredient. The carrier(s) must be “acceptable” in the sense of being compatible with the other ingredients of the formulation and not deleterious to the recipient thereof. The term “pharmaceutically acceptable carrier” includes vehicles and diluents.

The formulations include those suitable for oral, parenteral (including subcutaneous, intradermal, intramuscular, intravenous and intraarticular), rectal and topical (including dermal, buccal, sublingual and intraocular) administration, as well as those for administration by inhalation. The most suitable route may depend upon the condition and disorder of the recipient. The formulations may conveniently be presented in unit dosage form and may be prepared by any of the methods well known in the art of pharmacy. All methods include the step of bringing into association a compound or a pharmaceutically acceptable salt or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation. Oral formulations are well known to those skilled in the art, and general methods for preparing them are found in any standard pharmacy school textbook, for example, Remington: The Science and Practice of Pharmacy., A. R. Gennaro, ed. (1995), the entire disclosure of which is incorporated herein by reference.

Pharmaceutical compositions containing one or more compounds of Formulae A-Q (including Formula A^{x}) may be conveniently presented in unit dosage form and prepared by any of the methods well known in the art of pharmacy. Exemplary unit dosage formulations are those containing an effective dose, or an appropriate fraction thereof, of the active ingredient, or a pharmaceutically acceptable salt thereof. The magnitude of a prophylactic or therapeutic dose typically varies with the nature and severity of the condition to be treated and the route of administration. The dose, and perhaps the dose frequency, will also vary according to the age, body weight and response of the individual patient. In general, the total daily dose (in single or divided doses) ranges from about 1 mg per day to about 7000 mg per day, preferably about 1 mg per day to about 100 mg per day, and more preferably, from about 10 mg per day to about 100 mg per day, and even more preferably from about 20 mg to about 100 mg, to about 80 mg or to about 60 mg. In various embodiments, the total daily dose may range from about 50 mg to about 500 mg per day, and preferably, about 100 mg to about 500 mg per day. It is further recommended that children, patients over 65 years old, and those with impaired renal or hepatic function, initially receive low doses and that the dosage be titrated based on individual responses and/or blood levels. It may be necessary to use dosages outside these ranges in some cases, as will be apparent to those in the art. Further, it is noted that the clinician or treating physician knows how and when to interrupt, adjust or terminate therapy in conjunction with individual patient's response.

It should be understood that in addition to the ingredients particularly mentioned above, the formulations of this invention may include other agents conventional in the art having regard to the type of formulation in question, for example those suitable for oral administration may include flavoring agents.

Formulations of the present inventions suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally using one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein. Oral and parenteral sustained release drug delivery systems are well known to those skilled in the art, and general methods of achieving sustained release of orally or parenterally administered drugs are found, for example, in Remington: The Science and Practice of Pharmacy, pages 1660-1675 (1995).

Formulations for parenteral administration include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described. Formulations for rectal administration may be presented as a suppository with the usual carriers such as cocoa butter or polyethylene glycol. Formulations for topical administration in the mouth, for example, buccally or sublingually, include lozenges comprising the active ingredient in a flavored basis such as sucrose and acacia or tragacanth, and pastilles comprising the active ingredient in a basis such as gelatin and glycerin or sucrose and acacia.

The pharmaceutically acceptable carrier may take a wide variety of forms, depending on the route desired for administration, for example, oral or parenteral (including intravenous). In preparing the composition for oral dosage form, any of the usual pharmaceutical media may be employed, such as, water, glycols, oils, alcohols, flavoring agents, preservatives, and coloring agents in the case of oral liquid preparation, including suspension, elixirs and solutions. Carriers such as starches, sugars, microcrystalline cellulose, diluents, granulating agents, lubricants, binders and disintegrating agents may be used in the case of oral solid preparations such as powders, capsules and caplets. Exemplary solid oral preparations are tablets or capsules, because of their ease of administration. If desired, tablets may be coated by standard aqueous or nonaqueous techniques. Oral and parenteral sustained release dosage forms may also be used.

Exemplary formulations, are well known to those skilled in the art, and general methods for preparing them are found in any standard pharmacy school textbook, for example, Remington, THE SCIENCE AND PRACTICE OF PHARMACY, 21st Ed., Lippincott.

In various embodiments, the invention provides a pharmaceutical formulation that includes a combination of one or more of a compound of Formulae A, A^{x}, B, C and/or Q and one or more active compounds having a fused ring core motif common with at least one of compounds of Formulae A, A^{x}, B, C and Q. In various embodiments, the core motif is derivable by metabolic cleavage of a group from a compound of Formulae A, A^{x}, B, C and/or Q. In various embodiments, the active compound is a precursor for one or more compounds of Formulae A, A^{x}, B, C and Q.

### II. Methods

### A. Methods of Treatment or Prevention

Subjects for treatment according to methods of the present inventions include humans (patients) and other mammals. In one example, the subject is in need of therapy for the stated condition.

In various aspects, the inventions provide methods for treating or preventing a disease or condition which is a member selected from a neurological disorder, pain, ataxia and convulsion. In various embodiments, the methods include administering to a subject in need thereof a therapeutically effective amount of a compound of the present inventions or a pharmaceutically acceptable salt, solvate, hydrate thereof.

In various aspects, the inventions provide for the use of a compound of the present inventions (e.g., a compound of Formulae A, A^{x}, B, C and/or Q) in the manufacture of a medicament for the treatment of a disease or condition in a mammal (e.g., a human patient), wherein said disease or condition is a neurological disorder, pain, ataxia or convulsion.

In various aspects, the inventions provide for the use of a compound of the present inventions (e.g., a compound of Formulae A, A^{x}, B, C and/or Q) in the manufacture of a medicament for the enhancement of cognition in a mammal (e.g., a human).

In various aspects, the inventions provide a compound of the present inventions (e.g., a compound of Formulae A, A^{x}, B, C and/or Q) for use in treating a neurological disorder in a mammal (e.g., human). Exemplary neurological disorders are provided herein.

In various aspects, the inventions provide a compound of the present inventions (e.g., a compound of Formulae A, A^{x}, B, C and/or Q) for use in treating pain (e.g., neuropathic pain), ataxia or convulsion in a mammal (e.g., a human).

In various aspects, the inventions provide a compound of the present inventions (e.g., a compound of Formulae A, A^{x}, B, C and/or Q) for use in enhancing cognition in a mammal (e.g., a human).

In various embodiments, a compound released a compound of Formulae A, A^{x}, B, C and/or Q possess unique pharmacological characteristics with respect to inhibition of DAAO and influence the activity of the NMDA receptor in the brain, particularly by controlling the levels of D-serine. Therefore, these compounds can be effective in treating conditions and disorders (especially CNS-related disorders), which are modulated by DAAO, D-serine and/or NMDA receptor activity. In various embodiments, compounds of the present inventions (e.g., a compound of Formulae A, A^{x}, B, C and/or Q) are associated with diminished side effects compared to administration of the corresponding active compound released from a compound of Formulae A, A^{x}, B, C and/or Q.

In various embodiments, the present inventions relate to methods for increasing the concentration of D-serine and/or decreasing the concentration of toxic products of D-serine oxidation by DAAO in a mammal. In various embodiments the inventions provide a method for treating or preventing a disease or condition, such as those disclosed herein. In one example, the disease or condition is selected from a neurological disorder, pain, ataxia and convulsion. In various embodiments, the inventions provide a method of enhancing the cognitive capabilities of a human subject.

In various embodiments, the inventions provide a method of enhancing cognition in a mammalian subject (e.g., human). The method includes administering to the subject an effective amount of a compound of the present inventions (e.g., a compound of Formulae A, A^{x}, B, C and/or Q) or a pharmaceutically acceptable salt, solvate thereof. In one example, the subject has been diagnosed with a neurological disorder, such as a neurodegenerative disease disclosed herein (e.g., Alzheimer's disease), with brain injury or spinal cord injury. In another example, the subject benefits from enhanced cognitive capabilities with respect to increased quality of life, performance (e.g., test situations) or coping with stressful situations. For example, the subject is mentally disabled (e.g., due to brain injury). In another example, various embodiments of compounds of the present inventions (e.g., a compound of Formulae A, A^{x}, B, C and/or Q) are useful in relieving negative symptoms of stress, sleep deprivation (e.g., arising from emergency situations) and disruptions of the circadian rhythm (e.g., jet-lag, night-shifts, time adjustments, such as those to daylight savings time, and the like).

In various embodiments, a method of the present inventions includes administering to a mammalian subject (e.g., a human patient) in need thereof a therapeutically effective amount of a compound of the present inventions (e.g., a compound of Formulae A, A^{x}, B, C and/or Q) or a pharmaceutically acceptable salt, solvate, hydrate thereof

In various embodiments, compounds of the present inventions release selective DAAO inhibitors. In various embodiments, the compounds of Formulae A, A^{x}, B, C and/or Q provide medicaments that exhibit an advantageous profile of activity, including good bioavailability for example, of the released compound. Accordingly, in various embodiments, compounds of Formulae A, A^{x}, B, C and/or Q can offer advantages over art-known methods for treating disorders modulated by DAAO, D-serine or NMDA receptor activity. For example, unlike many conventional antipsychotic therapeutics, DAAO inhibitors can produce a desirable reduction in the cognitive symptoms of schizophrenia. Conventional antipsychotics often produce undesirable side effects, including tardive dyskinesia (irreversible involuntary movement disorder), extra pyramidal symptoms, and akathesia, and these can be reduced or eliminated by administering compounds of the present inventions (e.g., a compound of Formulae A, A^{x}, B, C and/or Q).

In various embodiments, compounds of the present inventions may be used in combination with one or more other drugs in the treatment, prevention, control, amelioration, or reduction of risk of diseases or conditions for which compounds of the present inventions or the other drugs may have utility, where the combination of the drugs together are safer or more effective than either drug alone. Such other drug(s) may be administered, by a route and in an amount commonly used therefore, contemporaneously or sequentially with a compound of the present inventions. When a compound of the present inventions is used contemporaneously with one or more other drugs, a pharmaceutical composition in unit dosage form containing such other drugs and one or more compounds of the present inventions can be utilized. However, the combination therapy may also include therapies in which a compound of the present inventions and one or more other drugs are administered on different overlapping schedules. It is also contemplated that when used in combination with one or more other active ingredients, the compounds of the present inventions and the other active ingredients may be used in lower doses than when each is used singly. Accordingly, the pharmaceutical compositions of the present inventions include those that contain one or more other active ingredients, in addition to a compound of the present inventions. The above combinations include combinations of a compound of the present inventions not only with one other active compound, but also with two or more other active compounds or prodrugs. Likewise, compounds of the present inventions may be used in combination with other drugs that are used in the prevention, treatment, control, amelioration, or reduction of risk of the diseases or conditions for which compounds of the present inventions are useful. Such other drugs may be administered, by a route and in an amount commonly used therefore, contemporaneously or sequentially with a compound of the present inventions. When a compound of the present inventions is used contemporaneously with one or more other drugs, a pharmaceutical composition containing such other drugs in addition to a compound of the present inventions can be used. Accordingly, the pharmaceutical compositions of the present inventions include those that also contain one or more other active ingredients, in addition to a compound of the present inventions. The weight ratio of a compound of the present inventions to the second active ingredient may be varied and will depend upon the effective dose of each ingredient. Generally, an effective dose of each will be used. Thus, for example, when a compound of the present inventions is combined with another agent, the weight ratio of a compound of the present inventions to the other agent will generally range from about 1000:1 to about 1:1000, preferably about 200:1 to about 1:200. Combinations of a compound of the present inventions and other active ingredients will generally also be within the aforementioned range, but in each case, an effective dose of each active ingredient should be used.

In such combinations a compound of the present inventions and other active agents may be administered separately or in conjunction. In addition, the administration of one element may be prior to, concurrent to, or subsequent to the administration of other agent(s). Accordingly, the subject compounds may be used alone or in combination with other agents which are known to be beneficial in the subject indications or other drugs that affect receptors or enzymes that either increase the efficacy, safety, convenience, or reduce unwanted side effects or toxicity of the compounds of the present inventions. The subject compound and the other agent may be co-administered, either in concomitant therapy or in a fixed combination.

In various embodiments, compounds of the present inventions can also be used in conjunction with therapy involving administration of D-serine or an analog thereof, such as a salt of D-serine, an ester of D-serine, alkylated D-serine, D-cycloserine or a precursor of D-serine.

In various embodiments, compounds of the present inventions can also be used in conjunction with therapy for neuropathic pain. Agents for this purpose include tricyclic antidepressants, such as imipramine (Tofranil), amitriptyline (Elavil), and nortriptyline (Pamelor, Aventyl); selective serotonin reuptake inhibitors (SSRIs), such as citalopram (Celexa), escitalopram (Lexpro), fluoxetine (Prozac), paroxetine (Paxil) and sertraline (Zoloft); serotonin and norepinephrine reuptake inhibitors (SNRIs), such as Cymbalta (duloxetine); anticonvulsants, such as gabapentin (Neurontin) and pregabalin (Lyrica); opioids such as morphine, oxycodone (OxyContin, Percoset), and fentanyl; and carbamazepine, lidocaine and lamotrigine.

In various embodiments, compounds of the present inventions can also be used in conjunction with cognition enhancing agents, e.g., MAO inhibitors, such as selegiline (Eldepryl); cholinesterase inhibitors, such as galantamine (Razadyne), rivastigmine (Exelon), donepezil (Aricept) and Memantine (NMDA antagonist).

In various embodiments, compounds of the present inventions can also be used in conjunction with antipsychotics for schizophrenia, which include risperidone (Risperidal), Olanzapine (Zyprexa), Clozapine (Clozaril), Paliperidone (Invega), Quetiapine (Seroquel), Ziprasidone (Geodon), Aripiprazole (Abilify), Asenapine and Lloperidone.

In various embodiments, compounds of the present inventions can also be used in conjunction with therapy involving administration of antipsychotics (for treating schizophrenia and other psychotic conditions, such as risperidone, olanzapine, clozapine, paliperidone, quetiapine, ziprasidone, aripiprazole, asenapine, loperidone), psychostimulants (for treating attention deficit disorder, depression, or learning disorders), antidepressants, nootropics (for example, piracetam, oxiracetam or aniracetam), acetylcholinesterase inhibitors (for example, galantamine, rivastigmine, the physostigmine related compounds, tacrine or donepezil), GABA analogs (e.g., gabapentin) or GABA receptor modulators, Alzheimer's disease therapeutics (e.g., memantine hydrochloride, and selegiline) and/or analgesics (for treating of persistent or chronic pain, e.g., neuropathic pain). Such methods for conjoint therapies are included within various embodiments of the present inventions.

In various embodiments, compounds of the present inventions can be employed in combination with anti-Alzheimer's agents, beta-secretase inhibitors, gamma-secretase inhibitors, HMG-CoA reductase inhibitors, NSAID's including ibuprofen, vitamin E, and anti-amyloid antibodies. In various embodiments, the subject compound may be employed in combination with sedatives, hypnotics, anxiolytics, antipsychotics, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amisulpride, amitriptyline, amobarbital, amoxapine, aripiprazole, bentazepam, benzoctamine, brotizolam, bupropion, busprione, butabarbital, butalbital, capuride, carbocloral, chloral betaine, chloral hydrate, clomipramine, clonazepam, cloperidone, clorazepate, chiordiazepoxide, clorethate, chiorpromazine, clozapine, cyprazepam, desipramine, dexclamol, diazepam, dichloralphenazone, divalproex, diphenhydramine, doxepin, estazolam, ethchlorvynol, etomidate, fenobam, flunitrazepam, flupentixol, fluphenazine, flurazepam, fluvoxamine, fluoxetine, fosazepam, glutethimide, halazepam, haloperidol, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, olanzapine, oxazepam, paraldehyde, paroxetine, pentobarbital, perlapine, perphenazine, phenelzine, phenobarbital, prazepam, promethazine, propofol, protriptyline, quazepam, quetiapine, reclazepam, risperidone, roletamide, secobarbital, sertraline, suproclone, temazepam, thioridazine, thiothixene, tracazolate, tranylcypromaine, trazodone, triazolam, trepipam, Iricetamide, triclofos, trifluoperazine, trimetozine, trimipramine, uldazepam, venlafaxine, zaleplon, ziprasidone, zola.zepam, zolpidem, and salts thereof, and combinations thereof, and the like, or the subject compound may be administered in conjunction with the use of physical methods such as with light therapy or electrical stimulation. In various embodiments, the subject compound may be employed in combination with levodopa (with or without a selective extracerebral decarboxylase inhibitor such as carbidopa or benserazide), anticholinergics such as biperiden (optionally as its hydrochloride or lactate salt) and trihexyphenidyl (benzhexol) hydrochloride, COMT inhibitors such as entacapone, MAO-B inhibitors, antioxidants, A₂ₐ adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form. In various embodiments, the subject compound may be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the subject compound may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form. Thus, the subject compound may be employed in combination with acetophenazine, alentemol, aripiprazole, amisulpride, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, quetiapine, risperidone, sulpiride, tetrabenazine, trihexyphenidyl, thioridazine, thiothixene, trifluoperazine or ziprasidone.

In various embodiments, compounds of the present inventions can be employed in combination with an anti-depressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT_{1A} agonists or antagonists, especially 5-HT_{1A} partial agonists, and corticotropin releasing factor (CRF) antagonists. Specific agents include: amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

In various embodiments, compounds of the present inventions can be employed in combination with a compound useful in the treatment of pain, for example carbamazepine, lidocaine, and lamotrigine, an NSAID such as ibuprofen, an antinociceptive agent such as an NR2B antagonist, a COX-2 inhibitor such as ARCOXIA, a Selective Serotonin Reuptake Inhibitor (SSRI) such as citalopram, escitalopram, fluoxetine, paroxetine, and sertraline, a Serotonin and Norepinephrine Reuptake Inhibitor (SNRI) such as Cymbalta, an anticonvulsants such as gabapentin (Neurontin) and pregabalin (Lyrica), an opioids such as morphine, oxycodone, and fentanyl, a tricyclic antidepressants such as imipramine, amitriptyline, and nortriptyline, or a sodium channel blocker.

In various embodiments, compounds of the present inventions can also be used in conjunction (coadministration) with one or more other therapeutic compound. For example, in various embodiments various compounds of the present inventions can be used in conjunction with therapy involving administration of antipsychotics (e.g., for treating schizophrenia and other psychotic conditions), psychostimulants (e.g., for treating attention deficit disorder, depression, or learning disorders), antidepressants, nootropics (for example, piracetam, oxiracetam or aniracetam), acetylcholinesterase inhibitors (for example, physostigmine related compounds, tacrine or donepezil), GABA analogs (e.g., gabapentin or pregabalin) or GABA receptor modulators, Alzheimer's disease therapeutics (e.g., memantine hydrochloride) and/or analgesics (e.g., for treating persistant or chronic pain, e.g., neuropathic pain). Such methods for conjoint therapies are included within various embodiments of the present inventions.

In another example, in various embodiments the present inventions provide a methods of inhibiting D-amino acid oxidase (DAAO) enzyme activity, in various embodiments said methods comprising contacting said DAAO with an active compound released from a compound of Formulae A, A^{x}, B, C and/or Q. In various embodiments, the DAAO is located within a cell (e.g., a mammalian cell). In one example, the cell is located within a mammal. For example, the cell is located within the central (e.g., brain) or peripheral nervous system of a mammal. In various embodiments, the inventions also provide a composition comprising a compound of the inventions and a mammalian cell. In various embodiments, the present inventions provide a composition comprising a compound of the inventions and a DAAO enzyme.

### Conditions and Disorders

In various aspects, the present inventions provide methods for inhibition of DAAO, and/or influencing the activity of the NMDA receptor in the brain (e.g., by controlling the levels of D-serine at the NMDA receptor, in plasma, and/or cerebellum) by administration of a therapeutically effective amount of a compound of the present inventions. For example, in various embodiments, the administration of a compound of general formulae A, A^{x}, B, C and/or Q can be effective in treating conditions and disorders, especially CNS-related disorders, modulated by DAAO, D-serine and/or NMDA receptor activity. These conditions and disorders include, but are not limited to, neuropsychiatric disorders, such as schizophrenia, autism, attention deficit disorder (ADD and ADHD) and childhood learning disorders, and neurodegenerative diseases and disorders, such as MLS (cerebellar ataxia), Alzheimer's disease, Parkinson's disease, Huntington's disease, amyotrophic lateral sclerosis, Down syndrome, neuropathic pain, multi-infarct dementia, status epilepticus, contusive injuries (e.g., spinal cord injury and head injury), viral infection induced neurodegeneration, (e.g., AIDS, encephalopathies), epilepsy, benign forgetfulness, and closed head injury. In various embodiments, administration of a compound of general formulae A, A^{x}, B, C and/or Q can be useful for the treatment of neurotoxic injury which follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest.

In various aspects, the administration of a compound of general formulae A, A^{x}, B, C and/or Q of the present inventions is useful for enhancing learning, memory and/or cognition, even in a subject not suffering from a disease or condition that causes loss of memory, cognition associated and/or loss of neuronal function.

In various embodiments, the compounds of the present inventions are useful for the treatment of neurological disorders, pain (e.g., neuropathic pain), ataxia and convulsion. Neurological disorders include neurodegenerative diseases (e.g., Alzheimer's disease) and neuropsychiatric disorders (e.g., schizophrenia).

In various embodiments, compounds of the present inventions are useful for the treatment of neurological disorders, pain (e.g., neuropathic pain), ataxia and convulsion, including the treatment of schizoaffective disorder, delusional disorder, brief psychotic disorder, shared psychotic disorder, psychotic disorder due to a general medical condition and substance-induced or drug-induced (phencyclidine, ketamine, and other dissociative anaesthetics, amphetamine and other psychostimulants and cocaine) psychosispsychotic disorder, psychosis associated with affective disorders, brief reactive psychosis, schizoaffective psychosis, “schizophrenia-spectrum” disorders such as schizoid or schizotypal personality disorders, or illnesses associated with psychosis (such as major depression, manic depressive (bipolar) disorder, Alzheimer's disease and post-traumatic stress syndrome), including both the positive and negative symptoms of schizophrenia and other psychoses; cognitive disorders including dementia (associated with Alzheimer's disease, ischemia, multi-infarct dementia, trauma, vascular problems or stroke, HIV disease, Parkinson's disease, Huntington's disease, Pick's disease, Creutzfeldt-Jacob disease, perinatal hypoxia, other general medical conditions or substance abuse); delirium, amnestic disorders or age-related cognitive decline; anxiety disorders including acute stress disorder, agoraphobia, generalized anxiety disorder, obsessive-compulsive disorder, panic attack, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); obesity, bulimia nervosa and compulsive eating disorders; bipolar disorders, mood disorders including depressive disorders; depression including unipolar depression, seasonal depression and post-partum depression, premenstrual syndrome (PMS) and premenstrual dysphoric disorder (PDD), mood disorders due to a general condition, and substance-induced mood-disorders; learning disorders, pervasive development disorder including autistic disorder, attention disorders including attention-deficit hyperactivity disorder (ADHD) and conduct disorder; NMDA-related disorders such as autism, depression, benign forgetfulness, childhood learning disorders and closed head injury; movement disorders, including akinesias and akinetic-rigid syndromes (including Parkinson's disease, drug-induced parkinsonism, postencephalitic parkinsonism, progressively supranuclear palsy, multiple system atrophy, corticobasal degeneration, parkinsonism-ALS dementia complex and basal gangli calcification), medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, muscular spasms and disorders associated with muscular spasticity or weakness including tremors; dyskinesias [including tremor (such as rest tremor, postural tremor, and intention tremor), chorea (such as Sydenham's chorea, Huntington's disease, benign hereditary chorea, neuroacanthocytosis, symptomatic chorea, drug-induced chorea and hemiballism), myoclonus (including generalized myoclonus and focal cyloclonus), tics (including simple tics, complex tics, and symptomatic tics), and dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia)]; urinary incontinence; neuronal damage including ocular damage, retinopathy or macular degeneration of the eye, tinnitus, hearing impairment and loss, and brain edema; emesis; and sleep disorders including insomnia and narcolepsy.

### Neuropsychiatric Disorders

In various embodiments, compounds of Formulae A, A^{x}, B, C and/or Q can be used treat neuropsychiatric disorders. Neuropsychiatric disorders include schizophrenia, autism, and attention deficit disorder. Clinicians recognize a distinction among such disorders, and there are many schemes for categorizing them. The Diagnostic and Statistical Manual of Mental Disorders, Revised, Fourth Ed., (DSM-IV-R), published by the American Psychiatric Association, provides a standard diagnostic system upon which persons of skill rely, and is incorporated herein by reference. According to the framework of the DSM-IV, the mental disorders of Axis I include: disorders diagnosed in childhood (such as Attention Deficit Disorder (ADD) and Attention Deficit-Hyperactivity Disorder (ADHD)) and disorders diagnosed in adulthood. The disorders diagnosed in adulthood include (1) schizophrenia and psychotic disorders; (2) cognitive disorders; (3) mood disorders; (4) anxiety related disorders; (5) eating disorders; (6) substance related disorders; (7) personality disorders; and (8) “disorders not yet included” in the scheme.

ADD and ADHD are disorders that are most prevalent in children and are associated with increased motor activity and a decreased attention span. These disorders are commonly treated by administration of psychostimulants such as methylphenidate and dextroamphetamine sulfate. In various embodiments, the compounds of the present inventions are also effective for treating disruptive behavior disorders, such as attention deficit disorder (ADD) and attention deficit disorder/hyperactivity (ADHD), which is in accordance with its accepted meaning in the art, as provided in the DSM-IV-TR™. These disorders are defined as affecting one's behavior resulting in inappropriate actions in learning and social situations. Although most commonly occurring during childhood, disruptive behavior disorders can also occur in adulthood.

Schizophrenia represents a group of neuropsychiatric disorders characterized by dysfunctions of the thinking process, such as delusions, hallucinations, and extensive withdrawal of the patient's interests from other people. Approximately one percent of the worldwide population is afflicted with schizophrenia, and this disorder is accompanied by high morbidity and mortality rates. So-called negative symptoms of schizophrenia include affect blunting, anergia, alogia and social withdrawal, which can be measured using SANS (Andreasen, 1983, Scales for the Assessment of Negative Symptoms (SANS), Iowa City, Iowa). Positive symptoms of schizophrenia include delusion and hallucination, which can be measured using PANSS (Positive and Negative Syndrome Scale) (Kay et al., 1987, Schizophrenia Bulletin 13:261-276). Cognitive symptoms of schizophrenia include impairment in obtaining, organizing, and using intellectual knowledge which can be measured by the Positive and Negative Syndrome Scale-cognitive subscale (PANSS-cognitive subscale) (Lindenmayer et al., 1994, J. Nerv. Ment. Dis. 182:631-638) or with cognitive tasks such as the Wisconsin Card Sorting Test. Conventional antipsychotic drugs, which act on the dopamine D₂ receptor, can be used to treat the positive symptoms of schizophrenia, such as delusion and hallucination. In general, conventional antipsychotic drugs and atypical antipsychotic drugs, which act on the dopamine D₂ and 5HT₂ serotonin receptor, are limited in their ability to treat cognitive deficits and negative symptoms such as affect blunting (e.g., lack of facial expressions), anergia, and social withdrawal.

Disorders treatable with the various compounds of the present inventions include, but are not limited to, depression, bipolar disorder, chronic fatigue disorder, seasonal affective disorder, agoraphobia, generalized anxiety disorder, phobic anxiety, obsessive compulsive disorder (OCD), panic disorder, acute stress disorder, social phobia, posttraumatic stress disorder, premenstrual syndrome, menopause, perimenopause and male menopause.

In various embodiments, compounds and compositions of the present inventions are also effective for treating substance-related disorders and addictive behaviors: Particular substance-related disorders and addictive behaviors are persisting dementia, persisting amnestic disorder, psychotic disorder or anxiety disorder induced by substance abuse; and tolerance of, dependence on or withdrawal from substances of abuse.

In various embodiments, compounds and compositions of the present inventions are also effective for treating eating disorders. Eating disorders are defined as a disorder of one's appetite or eating habits or of inappropriate somatotype visualization. Eating disorders include, but are not limited to, anorexia nervosa; bulimia nervosa, obesity and cachexia.

In addition to their beneficial therapeutic effects, various compounds of the present inventions provide the additional benefit of avoiding one or more of the adverse effects associated with conventional mood disorder treatments. Such side effects include, for example, insomnia, breast pain, weight gain, extrapyramidal symptoms, elevated serum prolactin levels and sexual dysfunction (including decreased libido, ejaculatory dysfunction and anorgasmia).

### Learning, Memory and Cognition

Various embodiments of the compounds of the present inventions have utility in treating or improving mammalian brain function, especially human cognition. For example, in various embodiments the compounds have utility improving brain function in human disease conditions such as Alzheimer's, schizophrenia, autism, dyslexia, obsessive-compulsive disorder, depression, anxiety, insomnia, sleep deprivation, and in brain injuries.

In various embodiments, compounds of the present inventions can be used for improving or enhancing learning and memory in subjects with or without cognitive deficits. Patients, who can benefit from such treatment, include those exhibiting symptoms of dementia or learning and memory loss. Individuals with an amnesic disorder are impaired in their ability to learn new information or are unable to recall previously learned information or past events. The memory deficit is most apparent on tasks to require spontaneous recall and can also be evident when the examiner provides stimuli for the person to recall at a later time. The memory disturbance must be sufficiently severe to cause marked impairment in social or occupational functioning and must represent a significant decline from a previous level of functioning. The memory deficit can be age-related or the result of disease or other cause. Dementia is characterized by multiple clinically significant deficits in cognition that represent a significant change from a previous level of functioning, including memory impairment involving inability to learn new material or forgetting of previously learned material. Memory can be formally tested by measuring the ability to register, retain, recall and recognize information. A diagnosis of dementia also requires at least one of the following cognitive disturbances: aphasia, apraxia, agnosia or a disturbance in executive functioning. These deficits in language, motor performance, object recognition and abstract thinking, respectively, must be sufficiently severe in conjunction with the memory deficit to cause impairment in occupational or social functioning and must represent a decline from a previously higher level of functioning.

In various embodiments, compounds of the present inventions are useful for preventing loss of neuronal function, which is characteristic of neurodegenerative diseases. For example, therapeutic treatment improves and/or enhances memory, learning and cognition. In various embodiments, compounds of the present inventions can be used to treat a neurodegenerative disease such as Alzheimer's, Huntington's disease, Parkinson's disease and amyotrophic lateral sclerosis, as well as MLS (cerebellar ataxia), Down syndrome, multi-infarct dementia, status epilepticus, contusive injuries (e.g., spinal cord injury and head injury), viral infection induced neurodegeneration, (e.g., AIDS, encephalopathies), epilepsy, benign forgetfulness, and closed head injury. In various embodiments, compounds of the present inventions are useful for treating or preventing loss of memory and/or cognition associated with a neurodegenerative disease. In various embodiments, the compounds can ameliorate cognitive dysfunctions associated with aging and improve catatonic schizophrenia.

Alzheimer's disease is manifested as a form of dementia that typically involves mental deterioration, reflected in memory loss, confusion, and disorientation. In the context of the present invention, dementia is defined as a syndrome of progressive decline in multiple domains of cognitive function, eventually leading to an inability to maintain normal social and/or occupational performance. Early symptoms include memory lapses and mild but progressive deterioration of specific cognitive functions, such as language (aphasia), motor skills (apraxia) and perception (agnosia). The earliest manifestation of Alzheimer's disease is often memory impairment, which is required for a diagnosis of dementia in both the National Institute of Neurological and Communicative Disorders and Stroke-Alzheimer's Disease-and the Alzheimer's Disease and Related Disorders Association (NINCDS-ADRDA) criteria (McKhann et al., 1984, Neurology 34:939-944), which are specific for Alzheimer's disease, and the American Psychiatric Association's Diagnostic and Statistical Manual of Mental Disorders, Fourth Edition (DSM-IV) criteria, which are applicable for all forms of dementia. The cognitive function of a patient can also be assessed by the Alzheimer's disease Assessment Scale-cognitive subscale (ADAS-cog; Rosen et al., 1984, Am. J. Psychiatry 141:1356-1364). Alzheimer's disease is typically treated by acetylcholine esterase inhibitors such as tacrine hydrochloride or donepezil. Unfortunately, the few forms of treatment for memory loss and impaired learning available at present are not considered effective enough to make any significant difference to a patient, and there is currently a lack of a standard nootropic drug for use in such treatment.

Other conditions that are manifested as deficits in memory and learning include benign forgetfulness and closed head injury. Benign forgetfulness refers to a mild tendency to be unable to retrieve or recall information that was once registered, learned, and stored in memory (e.g., an inability to remember where one placed one's keys or parked one's car). Benign forgetfulness typically affects individuals after 40 years of age and can be recognized by standard assessment instruments such as the Wechsler Memory Scale. Closed head injury refers to a clinical condition after head injury or trauma. Such a condition, which is characterized by cognitive and memory impairment, can be diagnosed as “amnestic disorder due to a general medical condition” according to DSM-IV.

In various embodiments, compounds and compositions of the present inventions are also effective for treating cerebral function disorders. The term cerebral function disorder, as used herein, includes cerebral function disorders involving intellectual deficits, and can be exemplified by senile dementia, Alzheimer's type dementia, memory loss, amnesia/amnestic syndrome, epilepsy, disturbances of consciousness, coma, lowering of attention, speech disorders, Parkinson's disease and autism.

In various embodiments the present inventions provide methods for improving mammalian (e.g., human) brain function related to associative learning, executive function, attention, rehearsal, retrieval, early consolidation, late consolidation, declarative memory, implicit memory, explicit memory, episodic memory, semantic memory, rote learning, informal learning, formal learning, multimedia learning, electronic learning, play, imprinting, social cognition including theory of mind, learning, empathy, cooperativity, altruism, language, non-verbal and verbal communicative skills, telepathy, and sensory integration of environmental cues including temperature, odor, sounds, touch, and taste. The skilled artisan will recognize that there are various methods of measuring improvements in brain function and are practices in behavioral and psychological testing that detect improvements in brain function.

Particular tests of associative learning where various embodiments of the compounds of the present inventions have utility are classical or respondant conditioning including forward conditioning, simultaneous conditioning, backward conditioning, temporal conditioning, unpaired conditioning, CS-alone conditioning, discrimination reversal conditioning, interstimulus interval conditioning, latent inhibition conditioning, conditioned inhibition conditioning, blocking, aversion therapy, systematic desensitization, or any other form of conditioning known in the psychological and behavioral literature to those skilled in the art of measuring brain function.

Particular tests of the brain function, e.g., cognitive improvement, of various embodiments of the compounds of the present inventions include tests classified as operant conditioning including reinforcement, punishment, and extinction, operant variability, avoidance learning, verbal behavior, four term contingency, operant hoarding, or other tests of modified behaviors.

In various embodiments, compounds of Formulae A, A^{x}, B, C and/or Q can have utility for improving brain function in conditions that are not characterized as diseased impairments, e.g., such as normal aging, low IQ, mental retardation, or any other mental capacity characterized by low brain function. In various embodiments, compounds of Formulae A, A^{x}, B, C and/or Q can have utility in improving brain function of humans with normal mental status, for example in performing defined tasks such as during extended time periods, in which concentration, attention, problem-solving skills and/or learning is required. For example, in various embodiments, compounds of the present inventions can be used by people operating machinery for extended time periods or people working in emergency or combat situations.

### Pain

In various embodiments, compounds of the present inventions are useful to treat any kind of acute or chronic pain. In various preferred embodiments, compounds of the present inventions are useful to treat chronic pain. In various preferred embodiments, compounds of the present inventions are useful to treat neuropathic pain. The term “pain” includes central neuropathic pain, involving damage to the brain or spinal cord, such as can occur following stroke, spinal cord injury, and as a result of multiple sclerosis. It also includes peripheral neuropathic pain, which includes diabetic neuropathy (DN or DPN), post-herpetic neuralgia (PHN), and trigeminal neuralgia (TGN). It also includes dysfunctions of the nervous system such as Complex Regional Pain Syndrome (CRPS), formerly known as Reflex Sympathetic Dystrophy (RSD), and causalgia, and neuropathic pain symptoms such as sensory loss, allodynia, hyperalgesia and hyperpathia. It further includes mixed nociceptive and neuropathic pain types, for example, mechanical spinal pain and radiculopathy or myelopathy, and the treatment of chronic pain conditions such as fibromyalgia, low back pain and neck pain due to spinal nerve root compression, and reflex sympathetic dystrophy.

Various embodiments of the compounds of the present inventions are of use in the prevention or treatment of diseases and conditions in which pain and/or inflammation predominates, including chronic and acute pain conditions. In addition to those stated elsewhere, various embodiments of the compounds of the present inventions are of use in the treatment and prevention of pain associated with the conditions which include rheumatoid arthritis; osteoarthritis; post-surgical pain; musculo-skeletal pain, particularly after trauma; spinal pain; myofascial pain syndromes; headache, including migraine, acute or chronic tension headache, cluster headache, temporomandibular pain, and maxillary sinus pain; ear pain; episiotomy pain; burns, and especially primary hyperalgesia associated therewith; deep and visceral pain, such as heart pain, muscle pain, eye pain, orofacial pain, for example, odontalgia, abdominal pain, gynaecological pain, for example, dysmenorrhoea, pain associated with cystitis and labor pain; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; itching conditions including pruritis, itch due to hemodialysis, and contact dermatitis; pain (as well as broncho-constriction and inflammation) due to exposure (e.g., via ingestion, inhalation, or eye contact) of mucous membranes to capsaicin and related irritants such as tear gas, hot peppers or pepper spray; chemotherapy-induced neuropathy and “non-painful” neuropathies; pain associated with carcinoma, often referred to as cancer pain; sciatica and ankylosing spondylitis; gout; scar pain; irritable bowel syndrome; bone and joint pain; repetitive motion pain; dental pain; inflammatory bowel disease; urinary incontinence including bladder detrusor hyper-reflexia and bladder hypersensitivity; respiratory diseases including chronic obstructive pulmonary disease (COPD), chronic bronchitis, cystic fibrosis and asthma; autoimmune diseases; and immunodeficiency disorders.

In various embodiments, compounds of the present inventions are useful to treat other conditions and disorders including autism, childhood learning disorders, depressions, anxieties and sleep disorders. In various embodiments, compounds of the present inventions are also useful for the treatment of neurotoxic injury that follows cerebral stroke, thromboembolic stroke, hemorrhagic stroke, cerebral ischemia, cerebral vasospasm, hypoglycemia, amnesia, hypoxia (including e.g., sleep/breathing disorders, such as sleep apnea), anoxia, perinatal asphyxia and cardiac arrest.

In various embodiments, the term “treating” when used in connection with the foregoing disorders means amelioration, prevention or relief from the symptoms and/or effects associated with these disorders and includes the prophylactic administration of a compound of the present inventions, a mixture thereof, a solvate (e.g., hydrate), prodrug (e.g., ethyl or methyl esters of the current carboxylic acid inhibitors) or a pharmaceutically acceptable salt of either, to substantially diminish the likelihood or seriousness of the condition.

### B. Models of Disease

Several established animal models of learning and memory are available to examine beneficial, cognitive enhancing effects as well as potential side effects associated with administration of the compounds of the present inventions. Exemplary methods that can be employed to assess changes in cognition in non-human species are described in the following references, which are incorporated by reference into this application in their entirety: Sarter M, Intern. J. Neuroscience 1987, 32:765-774; Methods and Findings in Experimental and Clinical Pharmacology 1998, 20(3): 249-277; Indian Journal of Pharmacology 1997, 29(4): 208-221.

In one example, compounds of the present inventions are tested using the “Morris Water Maze” (see, e.g., Stewart and Morris, “Behavioral Neuroscience. A Practical Approach. Volume I”, 1993, R. Saghal, Ed., 107-122; Journal of Neuroscience Methods 1984, 11(1): 47-60). The Morris water maze is one of the best-validated models of learning and memory, and it is sensitive to the cognitive enhancing effects of a variety of pharmacological agents. The task performed in the maze is particularly sensitive to manipulations of the hippocampus in the brain, an area of the brain important for spatial learning in animals and memory consolidation in humans. Moreover, improvement in Morris water maze performance is predictive of clinical efficacy of a compound as a cognitive enhancer. For example, treatment with cholinesterase inhibitors or selective muscarinic cholinergic agonists reverse learning deficits in the Morris maze animal model of learning and memory, as well as in clinical populations with dementia. In addition, this animal paradigm accurately models the increasing degree of impairment with advancing age and the increased vulnerability of the memory trace to pre-test delay or interference which is characteristic of amnesiac patients.

In another example, compounds of the present inventions are tested using “Contextual Fear Conditioning” (see, e.g., Barad, Metal., Proc Natl Acad Sci USA 1998, 95(25): 15020-5 and Bourtchouladze, R et al., Cell, 1994, 79: 59-68). Contextual fear conditioning is a form of associative learning in which animals learn to fear a new environment (or an emotionally neutral conditioned stimulus) because of its temporal association with an aversive unconditioned stimulus (US), such as a foot shock. When exposed to the same context or conditioned stimulus at a later time, conditioned animals show a variety of conditioned fear responses, including freezing behavior. Because robust learning can be triggered with a single training trial, contextual fear conditioning has been used to study temporally distinct processes of short-term and long-term memory. Contextual fear conditioning is believed to be dependent on both the hippocampus and amygdala function.

In another example, compounds of the present inventions are tested using “Conditioned Fear Extinction” (see, e.g., Walker, D L et al., J Neurosci. 2002, 22(6): 2343-51 and Davis, M et al., Biol. Psychiatry 2006, 60: 369-375). Fear extinction is an example of learning and is a process exhibited in both human and animals, including rodents. Extinction of fear refers to the reduction in the measured level of fear to a cue previously paired with an aversive event when that cue is presented repeatedly in the absence of the aversive event. Extinction of fear is not the erasure of the original fear memory, but instead results from a new form of learning that acts to inhibit or suppress the original fear memory (Bouton, M D and Bolles, R C; J. Exp. Psychol. Anim. Behav. Process. 1979, 5: 368-378; Konorski, J. Inegrative Activity of the Brain: An Interdiscipinary Approach, 1967, Chicago: The University of Chicago Press; Pavlov, I. P. Conditioned Reflexes. 1927, Oxford, United Kingdom: Oxford University Press.). The literature also suggests that glutamate acting at the NMDA receptor is critically involved in learning and memory (Bear, M. F. Proc. Nat. Acad. Sci. 1996, 93: 13453-13459; Castellano, C.; Cestari, V.; Ciamei, A. Curr. Drug Targets 2001, 2: 273-283; Morris, R. G.; Davis, S.; Butcher, S. P. Philos. Trans. R Soc. Lond. B Biol. Sci. 1990, 329: 187-204; Newcomer, J. W.; Krystal, J. H. Hippocampus 2001, 11: 529-542). There is also evidence that the NMDA receptor is involved with extinction of fear. For example, NMDA antagonists such as 2-amino-5-phosphopentanoic acid (APV) are known to block fear extinction (Davis, M. et al., Biol. Psychiatry 2006, 60: 369-375; Kehoe, E. J.; Macrae, M.; Hutchinson, C. L. Psychobiol. 1996, 24: 127-135; Lee, H.; Kim, J. J. J. Neurosci. 1998, 18: 8444-8454; Szapiro, G. et al., Hippocampus 2003, 13: 53-58). NMDA agonists (such as the partial agonsist D-cycloserine), are known to facilitate fear extinction (Davis, M et al., Biol. Psychiatry 2006, 60: 369-375; Ledgerwood, L.; Richardson, R.; Cranney, J. Behv. Neurosci. 2003, 117: 341-349; and Walker, D. L. et al., J. Neurosci. 2002, 22: 2343-2351). Additional experimental conditions for fear extinction tests can be found in the references incorporated herein by reference.

In human exposure therapy, a patient is repeatedly exposed for prolonged periods to a feared object or situation in the absence of aversive consequences. As a result, the patient is often able to face their feared cues or situations with less fear and avoidance (extinction retention) due to the learning that took place during exposure therapy (extinction training) It has been shown that agents, such as D-cycloserine, that improve extinction in animals also improve the effectiveness of exposure-based psychotherapy. Examples of exposure based cognitive-behavioral therapy (CBT) improved by agents that improve extinction include exposure to phobic objects as therapy for phobia disorders (see, e.g., Davis, M et al., Biol. Psychiatry 2006, 60: 369-375; Ressler, K. J. et al., Archives Gen. Psychiatry 2004, 61: 1136-1144), exposure to phobic situations as therapy for panic disorders (for social anxiety disorder, see e.g., Hoffmann, S. G. et al., Arch. Gen. Psychiatry 2006, 63: 298-304; Hofmann, S. G.; Pollack, M. H.; Otto, M. W. CNS Drug Reviews 2006, 12: 208-217), recollection of traumatic memories as therapy for post-traumatic stress disorder, exposure to cues associated with drug cravings as therapy for drug addiction, and exposure to cues associated with smoking as therapy for smoking cessation. Because of the cognitive, learning aspects associated with psychotherapy based treatment for disorders such as phobias, anxiety, post-traumatic stress disorder and addiction, in various embodiments, compounds of the present inventions are useful as an adjunct with psychotherapy for the treatment of these conditions. For example, as an adjunct to shorten the number of therapy sessions required or to improve the therapeutic outcome of therapy.

In another example, compounds of the present inventions are tested using “Delayed Non-Match to Sample” (see e.g., Bontempi, B. et al., Journal of Pharmacology and Experimental Therapeutics 2001, 299(1): 297-306; Alvarez, P. et al., Proc Natl Acad Sci USA 1994, 7;91(12), 5637-41); “Delayed Alternation” (also called delayed non-matching to position) (see, e.g., Roux, S. et al., Pharmacol Biochem Behav. 1994, 49(3): 83-88; Ohta, H. et al., Jpn J Pharmacol. 1991, 56(3): 303-9); “Social Discrimination Models” (see, e.g., Engelmann, M. et al., Physiol Behav. 1995, 58(2): 315-21); “Social Recognition Test” (also called delay-induced forgetting) (see e.g., Lemaire, M. et al., Psychopharmacology (Berl). 1994, 115(4):435-40).

In humans, improved learning and memory can be measured by such tests as the Wechsler Memory Scale and the Minimental test. A standard clinical test for determining if a patient has impaired learning and memory is the Minimental Test for Learning and Memory (see e.g., Folstein et al., J. Psychiatric Res. 1975, 12:185), especially for those suffering from head trauma, Korsakoffs disease or stroke. The test result serves as an index of short-term, working memory of the kind that deteriorates rapidly in the early stages of dementing or amnesiac disorders. Ten pairs of unrelated words (e.g., army-table) are read to the subject. Subjects are then asked to recall the second word when given the first word of each pair. The measure of memory impairment is a reduced number of paired-associate words recalled relative to a matched control group. Improvement in learning and memory constitutes either (a) a statistically significant difference between the performance of treated patients as compared to members of a placebo group; or (b) a statistically significant change in performance in the direction of normality on measures pertinent to the disease model.

Animal models or clinical instances of disease exhibit symptoms which are by definition distinguishable from normal controls. Thus, the measure of effective pharmacotherapy will be a significant, but not necessarily complete, reversal of symptoms. Improvement can be facilitated in both animal and human models of memory pathology by clinically effective “cognitive enhancing” drugs which serve to improve performance of a memory task. For example, cognitive enhancers which function as cholinomimetic replacement therapies in patients suffering from dementia and memory loss of the Alzheimer's type significantly improve short-term working memory in such paradigms as the paired-associate task. Another potential application for therapeutic interventions against memory impairment is suggested by age-related deficits in performance which are effectively modeled by the longitudinal study of recent memory in aging mice.

The Wechsler Memory Scale is a widely used pencil-and-paper test of cognitive function and memory capacity. In the normal population, the standardized test yields a mean of 100 and a standard deviation of 15, so that a mild amnesia can be detected with a 10-15 point reduction in the score, a more severe amnesia with a 20-30 point reduction, and so forth. During the clinical interview, a battery of tests, including, but not limited to, the Minimental test, the Wechsler memory scale, or paired-associate learning are applied to diagnose symptomatic memory loss. These tests provide general sensitivity to both general cognitive impairment and specific loss of learning/memory capacity (Squire, 1987). Apart from the specific diagnosis of dementia or amnestic disorders, these clinical instruments also identify age-related cognitive decline which reflects an objective diminution in mental function consequent to the aging process that is within normal limits given the person's age (DSM IV, 1994). As noted above, “improvement” in learning and memory within the context of the present invention occurs when there is a statistically significant difference in the direction of normality in the paired-associate test, for example, between the performance of therapeutic agent treated patients as compared to members of the placebo group or between subsequent tests given to the same patient.

In animals, many established models of schizophrenia are available to examine the beneficial effects of treatment; many of which are described in the following references, as well as references cited therein: Saibo Kogaku 2007, 26(1): 22-27; Cartmell, J. et al., J. Pharm. Exp. Ther. 1999, 291(1): 161-170; Rowley, M; Bristow, L. J.; Hutson, P. H. J. Med. Chem. 2001, 1544(4): 477-501; Geyer, M. A.; Ellenbroek, B; Prog Neuropsychopharmacol Biol Psychiatry 2003, 27(7):1071-1079; Geyer, M. A. et al., Psychopharmacology (Berl). 2001, 156(2-3):117-54; Jentsch, J. D.; Roth, R. H. Neuropsychopharmacology 1999, 20(3):201-25. The tests include “Prepulse Inhibition” (see e.g., Dulawa, S. C.; Geyer, M. A. Chin J Physiol. 1996, 39(3):139-46); “PCP Stereotypy Test” (see e.g., Meltzer et al., (“PCP (Phencyclidine): Historical and Current Perspectives”, ed. E. F. Domino, NPP Books, Ann Arbor, 1981: 207-242); “Amphetamine Stereotypy Test” (see e.g., Simon and Chermat, J. Pharmacol. (Paris) 1972, 3: 235-238); “PCP Hyperactivity” (se e.g., Gleason, S. D.; Shannon, H. E. Psychopharmacology (Berl). 1997, 129(1):79-84); and “MK-801 Hyperactivity” (see e.g., Corbett, R. et al., Psychopharmacology (Berl). 1995, 120(1):67-74), the disclosures of which are each incorporated herein by reference.

The prepulse inhibition test can be used to identify compounds that are effective in treating schizophrenia. The test is based upon the observations that animals or humans that are exposed to a loud sound will display a startle reflex and the observation that animals or humans exposed to a series of lower intensity sounds prior to the higher intensity test sound will no longer display as intense of a startle reflex. This is termed prepulse inhibition. Patients diagnosed with schizophrenia display defects in prepulse inhibition, that is, the lower intensity prepulses no longer inhibit the startle reflex to the intense test sound. Similar defects in prepulse inhibition can be induced in animals via drug treatments (scopolamine, ketamine, PCP or MK-801) or by rearing offspring in isolation. These defects in prepulse inhibition in animals can be partially reversed by drugs known to be efficacious in schizophrenia patients. It is felt that animal prepulse inhibition models have face value for predicting efficacy of compounds in treating schizophrenia patients.

In animals, many established models of pain are available to examine the beneficial effects of treatment; many of which are reviewed in Methods in Pain Research, CRC Press, 2001, Kruger, L. (Editor). Tests of acute pain include the tail flick (see e.g., d'Amour and Smith, J. Pharmacol. Exp. Ther. 1941, 72: 74-79), hot plate (see e.g., Eddy, N. B.; Leimbach, D. J Pharmacol Exp Ther. 1953, 107(3):385-93), and paw withdrawal tests. The phenylbenzoquinone writhing assay is a measure of peritoneovisceral or visceral pain. Persistent pain tests, which use an irritant or foreign chemical agent as the nociceptive stimulus, include the formalin test (see e.g., Wheeler-Aceto, H; Cowan, A Psychopharmacology (Berl). 1991, 104(1):35-44), Freund's adjuvant (see e.g., Basile, A. S. et al., Journal of Pharmacology and Experimental Therapeutics 2007, 321(3): 1208-1225; Ackerman, N. R. et al; Arthritis & Rheumatism 1979, 22(12): 1365-74), capsaicin (see e.g., Barrett, A. C. et al., Journal of Pharmacology and Experimental Therapeutics 2003, 307(1): 237-245), and carrageenin models. These models have an initial, acute phase, followed by a second, inflammatory phase.

Neuropathic pain models are reviewed in Wang and Wang, Advanced Drug Delivery Reviews 2003, and include the “Spinal Nerve Ligation (SNL) model” (also called the “Chung Model”) (see e.g., Kim, S. H.; Chung, J. M. Pain 1992, 50(3):355-63; Chaplan et al., Journal of Neuroscience Methods 1994, 53(1):55-63); “Chronic Constriction Injury (CCI) model” (also called the “Bennett Model”) (see e.g., Bennett, G. J; Xie, Y. K Pain 1988, 33(1):87-107); “Progressive Tactile Hypersensitivity (PTH) model) (see e.g., Decosterd, I. Pain 2002, 100(1): 155-162; Anesth. Analg. 2004, 99: 457-463); “Spared Nerve Injury (SNI) model” (see e.g., Decosterd, I., Pain 2002, 100(1): 155-162; Anesth. Analg. 2004, 99: 457-463); “lumbar nerve ligation model” (see e.g., Ringkamp, M. et al., Pain 1999, 79(2-3): 143-153); and “streptozocin—or chemotherapy induced diabetic neuropathy” (see e.g., Courteix, C.; Eschalier, A.; Lavarenne, J. Pain 1993, 53(1): 81-88; Aubel, B. et al Pain 2004, 110(1-2): 22-32.).

Opioids, such as morphine, display robust efficacy in models of acute pain, such as the tail flick and hot plate tests, as well as in both the initial, acute phase and the second, inflammatory phase of persistent pain tests, such as the formalin test. Opioids also display efficacy in neuropathic pain models, such as the Spinal Nerve Ligation (SNL) model. The general analgesic effects of opiate compounds such as morphine in neuropathic pain models, however, are suggested by the increase in paw withdrawal threshold (PWT) in both the injured and the contralateral (uninjured) paw. Compounds that are useful specifically for the treatment of persistent or chronic pain states (e.g., neuropathic pain), such as gabapentin, tend to display efficacy in models of persistent inflammatory and neuropathic pain, such as the formalin (second phase) and SNL models. Compounds of this type, however, tend to increase PWT in the SNL model in only the injured paw. In addition, these compounds fail to display efficacy in acute tests such as the tail flick test and the hot plate test, and also fail to display efficacy in the initial, acute phase of the formalin test. The lack of effect of compounds in the acute pain tests supports the notion that the antinociceptive action of these compounds is related to specific mechanisms associated with a central sensitized state following injury. As a result, compounds that are efficacious in neuropathic pain model(s), such as the SNL (Chung) model, and the second phase of the formalin test, but are not efficacious in acute pain models, such as hot plate and tail flick, or in the first phase of the formalin test suggest that these compounds are more likely to be effective in persistent and chronic, rather than acute, pain states (see Table 4). In addition, their ability to increase PWT in the SNL model should be specific for the ipsilateral (injured) paw. Relevant references follow, and are included by reference. Singh, L. et al, Psychopharmacology 1996, 127: 1-9. Field, M. J. et al., Br. J. Pharmacol. 1997, 121: 1513-1522. Iyengar, S. et al, J. Pharmacology and Experimental Therapeutics 2004, 311: 576-584. Shimoyama, N. et al Neuroscience Letters 1997, 222: 65-67. Laughlin, T. M. et al., J. Pharmacology and Experimental therapeutics 2002, 302: 1168-1175. Hunter, J. C. et al., European J. Pharmacol. 1997, 324: 153-160. Jones, C. K. et al., J. Pharmacology and Experimental therapeutics 2005, 312: 726-732. Malmberg, A. B.; Yaksh, T. L. Anesthesiology 1993, 79: 270-281. Bannon, A. W. et al., Brain Res. 1998, 801: 158-63.

In various embodiments, the compounds of the present inventions are useful for the treatment of persistent or chronic pain states (e.g., neuropathic pain). As described above, such compounds can be profiled in vivo by evaluating their efficacy in models of both acute and neuropathic pain. Various compounds demonstrate efficacy in neuropathic pain models, but not in acute pain models.

TABLE 4
Profile of morphine and gabapentin in a variety of animal models

| Animal Model | Morphine | Gabapentin |
|---|---|---|
| Acute Pain | | |
| Hot plate | + | − |
| Tail flick | + | − |
| Formalin (early phase) | + | − |
| Tissue Injury/Inflammatory Pain | | |
| Formalin (second phase) | + | + |
| Carrageenan | + | + |
| NERVE INJURY/NEUROPATHIC PAIN | | |
| Spinal Nerve Ligation (SNL; Chung) | + | + |
| Chronic Constriction Injury (CCI; Bennet) | + | + |

There are various animal models with chronic brain dysfunctions thought to reflect the processes underlying human epilepsy and seizures/convulsions, such as those described in Epilepsy Res. 2002, 50(1-2):105-23. Such chronic models include the “kindling model of temporal lobe epilepsy” (TLE); “post-status models of TLE”, in which epilepsy develops after a sustained status epilepticus; and genetic models of different types of epilepsy. Currently, the kindling model and post-status models, such as the pilocarpine or kainate models, are the most widely used models for studies on epileptogenic processes and on drug targets by which epilepsy can be prevented or modified. Furthermore, the seizures in these models can be used for testing of antiepileptic drug effects. A comparison of the pharmacology of chronic models with models of acute (reactive or provoked) seizures in previously healthy (non-epileptic) animals, such as the maximal electroshock seizure test, demonstrates that drug testing in chronic models of epilepsy yields data which are more predictive of clinical efficacy and adverse effects.

Exemplary embodiments are summarized herein below.

In an exemplary embodiment, the invention provides a compound having a structure according to the formula

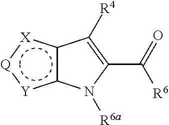

wherein Q is a member selected from O, S, N and CR¹; X is a member selected from O, S, N, NR³ and CR^{2a}; and Y is a member selected from O, S, N, NR³ and CR^{2b}. R¹ is a member selected from H, F, substituted or unsubstituted arylalkyl, substituted or unsubstituted heteroarylalkyl, substituted or unsubstituted C₄-C₁₀ cycloalkyl, and substituted or unsubstituted C₄-C₁₀ heterocycloalkyl. R^{2a} is a member selected from H, F, Cl, Br, CN, substituted or unsubstituted C₃-C₆ alkyl, substituted or unsubstituted arylalkyl, substituted or unsubstituted heteroarylalkyl, substituted or unsubstituted C₄-C₁₀ cycloalkyl, substituted or unsubstituted C₄-C₁₀ heterocycloalkyl and alkenyl. R^{2b} is a member selected from H, F, substituted or unsubstituted C₃-C₆ alkyl, substituted or unsubstituted arylalkyl, substituted or unsubstituted heteroarylalkyl, substituted or unsubstituted C₄-C₁₀ cycloalkyl, and substituted or unsubstituted C₄-C₁₀ heterocycloalkyl and alkenyl. R³ is a member selected from H, substituted or unsubstituted C₁-C₆ alkyl, substituted or unsubstituted arylalkyl, substituted or unsubstituted heteroarylalkyl, substituted or unsubstituted C₄-C₁₀ cycloalkyl, and substituted or unsubstituted C₄-C₁₀ heterocycloalkyl.

R⁴ is a member selected from H, F, Cl, Br, CN, unsubstituted C₁-C₆ alkyl, substituted or unsubstituted arylalkyl, substituted or unsubstituted heteroarylalkyl, substituted or unsubstituted C₄-C₁₀ cycloalkyl and alkenyl.

R⁶ is a member selected from OR²⁶, O⁻M⁺, SR²⁷, OPO₃R^{27a} and NR²⁸R²⁹; and R⁶ and R^{6a} are optionally joined to form a ring. R²⁶ is a member selected from H, acyl, substituted or unsubstituted alkyl, substituted or unsubstituted heteroalkyl, substituted or unsubstituted aryl, substituted or unsubstituted heteroaryl, substituted or unsubstituted heterocycloalkyl, and alkenyl. M⁺ is a member selected from inorganic positive ions and organic positive ions. R²⁷ is substituted or unsubstituted alkyl. R^{27a} is substituted or unsubstituted alkyl or a positive organic or inorganic ion. R²⁸ and R²⁹ are members independently selected from H, OR³⁰, acyl, substituted or unsubstituted alkyl, substituted or unsubstituted heteroalkyl, substituted or unsubstituted aryl, substituted or unsubstituted heteroaryl, and substituted or unsubstituted heterocycloalkyl, and R²⁸ and R²⁹ together with the nitrogen to which they are bound optionally form a ring. R³⁰ is a member selected from H, acyl, substituted or unsubstituted alkyl, and substituted or unsubstituted heteroalkyl.

R^{6a} is a member selected from:

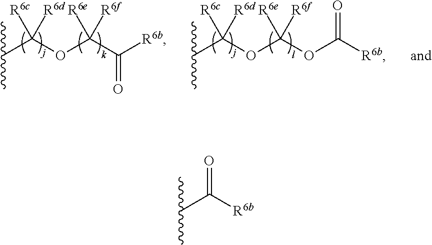

wherein R^{6c}, R^{6d}, R^{6e} and R^{6f} are members independently selected from H, substituted or unsubstituted alkyl, substituted or unsubstituted heteroalkyl, substituted or unsubstituted arylalkyl, substituted or unsubstituted heteroarylalkyl, substituted or unsubstituted cycloalkyl, substituted or unsubstituted heterocycloalkyl and alkenyl, and where R^{6c} and R^{6d} are optionally joined to form a ring, and where R^{6e} and R^{6f} are optionally joined to form a ring; and where R^{6c} and R^{6e} are optionally joined to form a ring. The indices j and 1 are integers independently selected from 1 to 5. The index k is an integer selected from 0 to 5. R^{6b} is a member selected from substituted or unsubstituted alkyl, substituted or unsubstituted heteroalkyl, substituted or unsubstituted arylalkyl, substituted or unsubstituted heteroarylalkyl, substituted or unsubstituted cycloalkyl, substituted or unsubstituted heterocycloalkyl and alkenyl.

In an exemplary embodiment, R⁴ is a member selected from H, F, Cl, Br, CN and unsubstituted C₁-C₄ alkyl.

In an exemplary embodiment, the compound has the formula

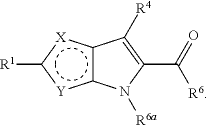

In an exemplary embodiment, R⁴ is a member selected from H, F, Cl, Br, CN and unsubstituted C₁-C₄ alkyl.

In an exemplary embodiment, one member selected from X and Y is O and the other member is CR², CR^{2a} or N.

In an exemplary embodiment, R¹, R², R^{2a} and R⁴ are members independently selected from H and F.

In an exemplary embodiment, R⁶ and R^{6a} are joined to form a ring structure, the compound having a formula which is a member selected from:

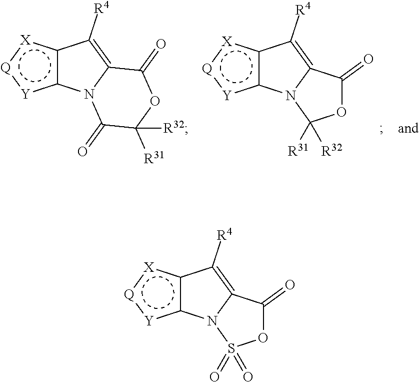

wherein R³¹ and R³² are members independently selected from H, acyl, substituted or unsubstituted alkyl, substituted or unsubstituted heteroalkyl, substituted or unsubstituted aryl, substituted or unsubstituted heteroaryl and substituted or unsubstituted heterocycloalkyl. R³¹ and R³² together with the carbon to which they are attached are optionally joined to form a ring, or together R³¹ and R³² and the carbon to which they are attached form C═O.

In an exemplary embodiment, R²⁶ is a member selected from substituted or unsubstituted C₄-C₁₀ alkyl, substituted or unsubstituted 3- to 8-membered heterocycloalkyl, acyl, substituted or unsubstituted phenyl, a steroid and P(O)(OR³³)(O⁻)M⁺. R³³ is a member selected from substituted or unsubstituted alkyl and substituted or unsubstituted heteroalkyl. M⁺ is an organic or inorganic cation.

In an exemplary embodiment, R²⁶ comprises an amino acid residue.

In an exemplary embodiment, R²⁶ is selected from acyl, substituted or unsubstituted C₃-C₈ cyclic alkyl and substituted or unsubstituted 3- to 8-membered heterocycloalkyl.

In an exemplary embodiment, R²⁶ is a member selected from the group of:

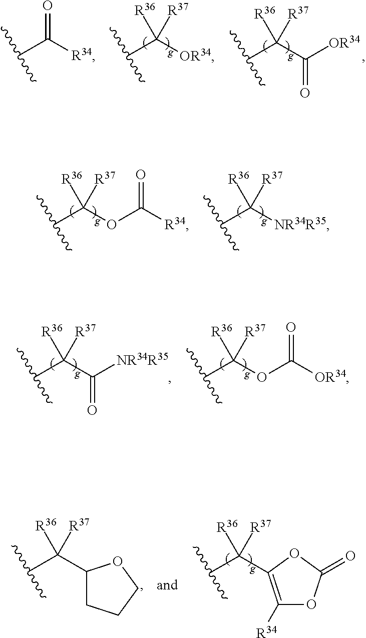

wherein R³⁴ is a member selected from H, acyl, substituted or unsubstituted alkyl, substituted or unsubstituted heteroalkyl, substituted or unsubstituted aryl, substituted or unsubstituted heteroaryl and substituted or unsubstituted heterocycloalkyl. R³⁶ and R³⁷ each is a member independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl. R³⁶ and R³⁷, together with the carbon to which they are bound, are optionally joined to form a ring. The index g is an integer selected from 1 to 10. R³⁴ and R³⁶ are optionally joined to form a ring.

In an exemplary embodiment, R²⁶ comprises

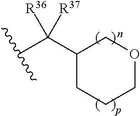

wherein R³⁶ and R³⁷ each is a member independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl. R³⁶ and R³⁷, together with the carbon to which they are bound, are optionally joined to form a ring. The index g is an integer selected from 1 to 10. The indices n and p are integers independently selected from 0 to 2.

In an exemplary embodiment, R³⁴ is:

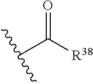

wherein R³⁸ is a member selected from OR³⁹, NR³⁹R⁴⁰, substituted or unsubstituted alkyl, and substituted or unsubstituted heterocycloalkyl. R³⁹ and R⁴⁰ are members independently selected from H, substituted or unsubstituted alkyl, substituted or unsubstituted heteroalkyl, substituted or unsubstituted aryl, substituted or unsubstituted heteroaryl, and substituted or unsubstituted heterocycloalkyl.

In an exemplary embodiment, R³⁸ is a member selected from substituted or unsubstituted arylalkyl, and substituted or unsubstituted heteroarylalkyl.

In an exemplary embodiment, a member selected from R³⁴, R³⁶, R³⁷ and combinations thereof is substituted with one or more halogen atoms.

In an exemplary embodiment, the invention provides a compound having the formula:

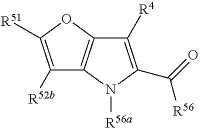

wherein R⁵¹ and R^{52b} are members independently selected from H and F. R⁴ is a member selected from H, F, Cl, Br, CN, unsubstituted C₁-C₆ alkyl, substituted or unsubstituted arylalkyl, substituted or unsubstituted heteroarylalkyl, substituted or unsubstituted C₄-C₁₀ cycloalkyl and alkenyl. R^{56a} a member selected from H, substituted or unsubstituted alkyl and substituted or unsubstituted heteroalkyl.

R⁵⁶ is a member selected from OR^{26b}, SR²⁷ and NR²⁸R²⁹. R⁵⁶ and R^{56a} are optionally joined to form a ring. R^{26b} is a member selected from H, acyl, substituted or unsubstituted C₄ or larger alkyl, substituted or unsubstituted heteroalkyl, substituted or unsubstituted aryl, substituted or unsubstituted heteroaryl, substituted or unsubstituted arylalkyl, substituted or unsubstituted heteroarylalkyl, substituted or unsubstituted cycloalkyl, substituted or unsubstituted heterocycloalkyl and alkenyl, wherein only one of R^{56a} and R^{26b} can be H. R²⁷ is substituted or unsubstituted alkyl. R²⁸ and R²⁹ are members independently selected from H, OR³⁰, acyl, substituted or unsubstituted alkyl, substituted or unsubstituted heteroalkyl, substituted or unsubstituted aryl, substituted or unsubstituted heteroaryl, and substituted or unsubstituted heterocycloalkyl, and R²⁸ and R²⁹ together with the nitrogen to which they are bound optionally form a ring. R³⁰ is a member selected from H, acyl substituted or unsubstituted alkyl and substituted or unsubstituted heteroalkyl.

In an exemplary embodiment, R^{56a} is H and R^{26b} is a member selected from acyl, substituted or unsubstituted C₄ or larger alkyl, substituted or unsubstituted heteroalkyl, substituted or unsubstituted aryl, substituted or unsubstituted heteroaryl, substituted or unsubstituted arylalkyl, substituted or unsubstituted heteroarylalkyl, substituted or unsubstituted cycloalkyl, substituted or unsubstituted heterocycloalkyl and alkenyl.

In an exemplary embodiment, R⁵⁶ is a member selected from OR^{26b} and SR²⁷; wherein R^{26b} is a member selected from acyl, substituted or unsubstituted C₄ or larger alkyl, substituted or unsubstituted heteroalkyl, substituted or unsubstituted aryl, substituted or unsubstituted heteroaryl, substituted or unsubstituted arylalkyl, substituted or unsubstituted heteroarylalkyl, substituted or unsubstituted cycloalkyl, substituted or unsubstituted heterocycloalkyl and alkenyl; and wherein R⁵⁶ and R^{56a}, are optionally joined to form a ring.

In an exemplary embodiment, R^{26b} is a member selected from

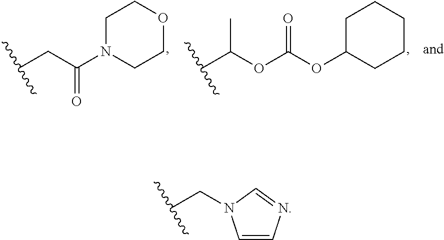

In an exemplary embodiment, R^{26b} is a member selected from

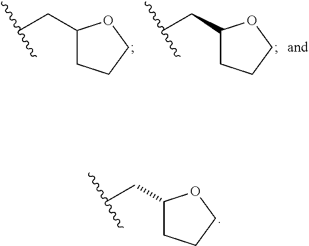

In an exemplary embodiment, R^{26b} is a member selected from

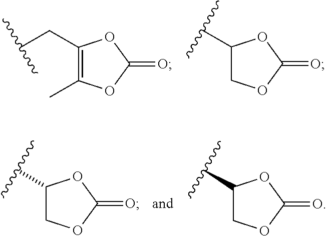

In an exemplary embodiment, R^{26b} is a member selected from

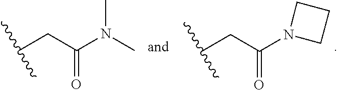

In an exemplary embodiment, R^{26b} is a member selected from

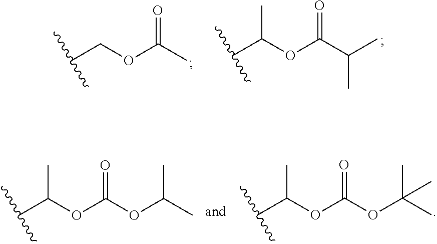

In an exemplary embodiment, the invention provides a compound having the formula:

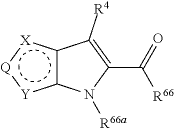

wherein Q is a member selected from O, S, N and CR¹; X is a member selected from O, S, N, NR³ and CR^{2a}; and Y is a member selected from O, S, N, NR³ and CR^{2b}. R¹ is a member selected from H, F, substituted or unsubstituted arylalkyl, substituted or unsubstituted heteroarylalkyl, substituted or unsubstituted C₄-C₁₀ cycloalkyl, and substituted or unsubstituted C₄-C₁₀ heterocycloalkyl. R^{2a} is a member selected from H, F, Cl, Br, CN, substituted or unsubstituted C₃-C₆ alkyl, substituted or unsubstituted arylalkyl, substituted or unsubstituted heteroarylalkyl, substituted or unsubstituted C₄-C₁₀ cycloalkyl, substituted or unsubstituted C₄-C₁₀ heterocycloalkyl and alkenyl. R^{2b} is a member selected from H, F, substituted or unsubstituted C₃-C₆ alkyl, substituted or unsubstituted arylalkyl, substituted or unsubstituted heteroarylalkyl, substituted or unsubstituted C₄-C₁₀ cycloalkyl, and substituted or unsubstituted C₄-C₁₀ heterocycloalkyl and alkenyl. R³ is a member selected from H, substituted or unsubstituted C₁-C₆ alkyl, substituted or unsubstituted arylalkyl, substituted or unsubstituted heteroarylalkyl, substituted or unsubstituted C₄-C₁₀ cycloalkyl, and substituted or unsubstituted C₄-C₁₀ heterocycloalkyl.

R⁴ is a member selected from H, F, Cl, Br, CN, unsubstituted C₁-C₆ alkyl, substituted or unsubstituted arylalkyl, substituted or unsubstituted heteroarylalkyl, substituted or unsubstituted C₄-C₁₀ cycloalkyl and alkenyl.

R^{66a} is a member selected from H, substituted or unsubstituted alkyl and substituted or unsubstituted heteroalkyl.

R⁶⁶ is OR^{26c}. R^{26c} is a member selected from:

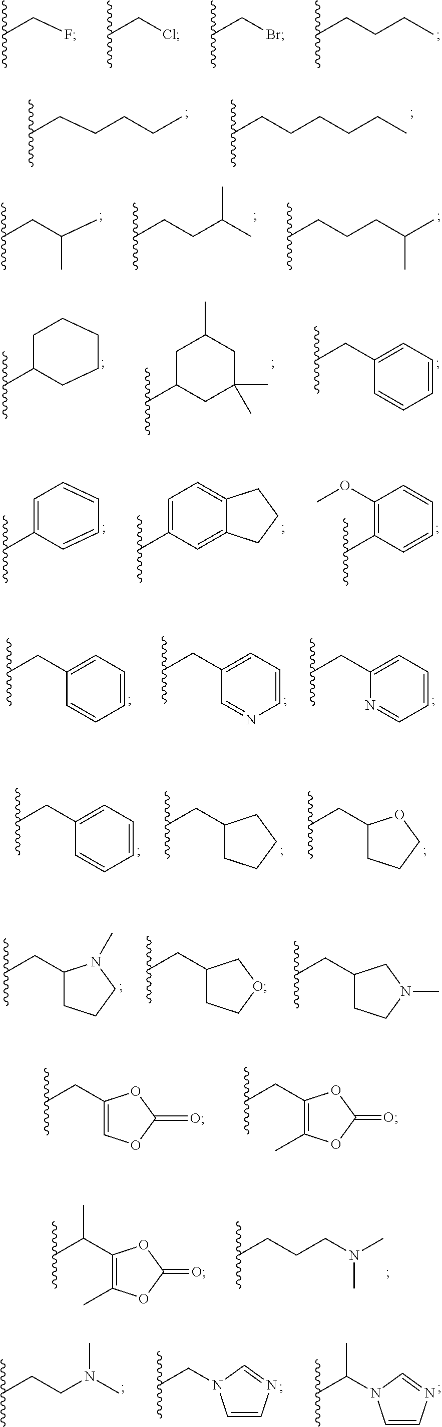

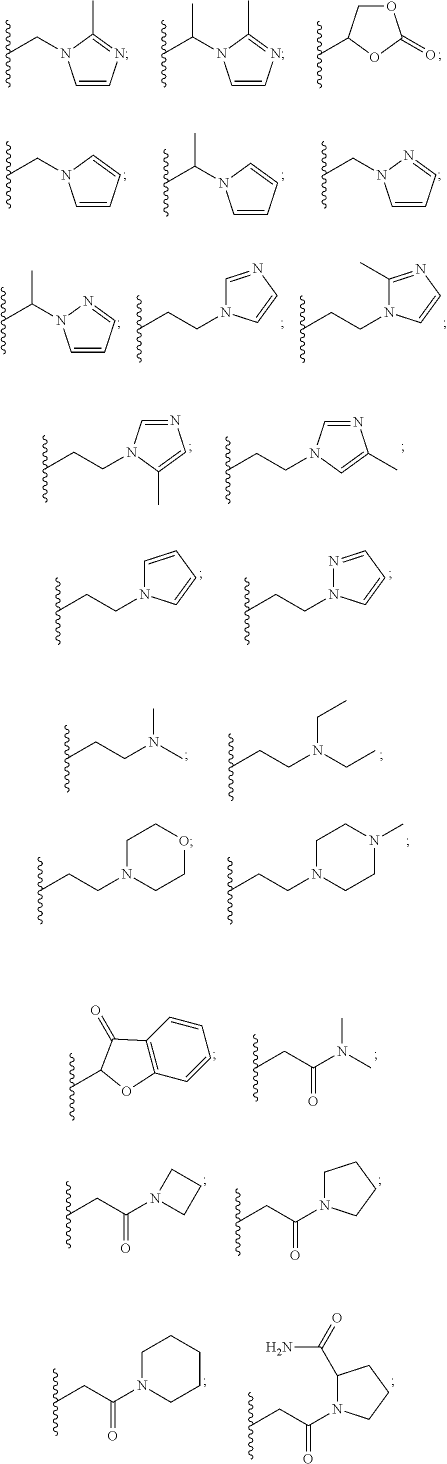

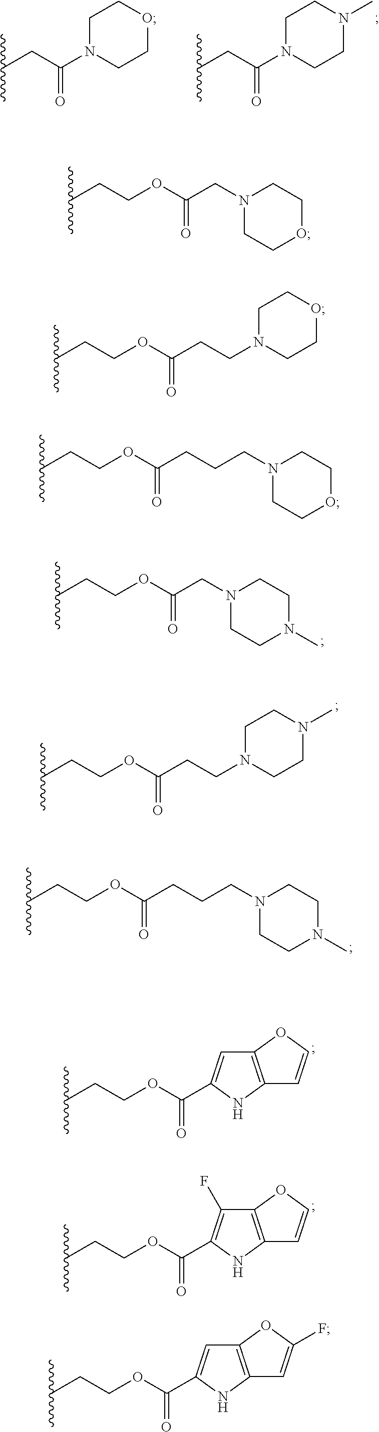

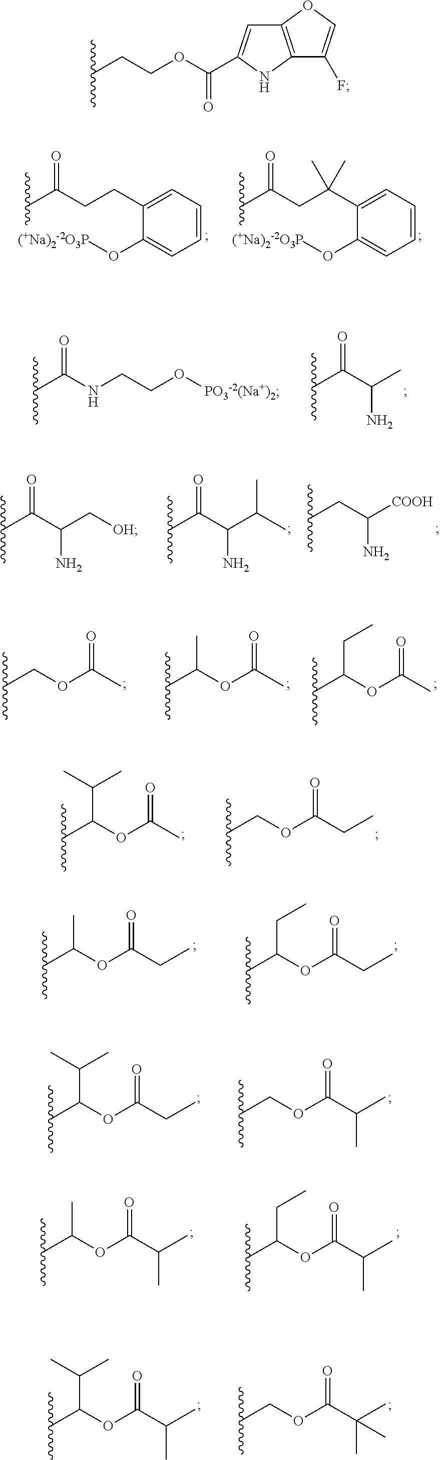

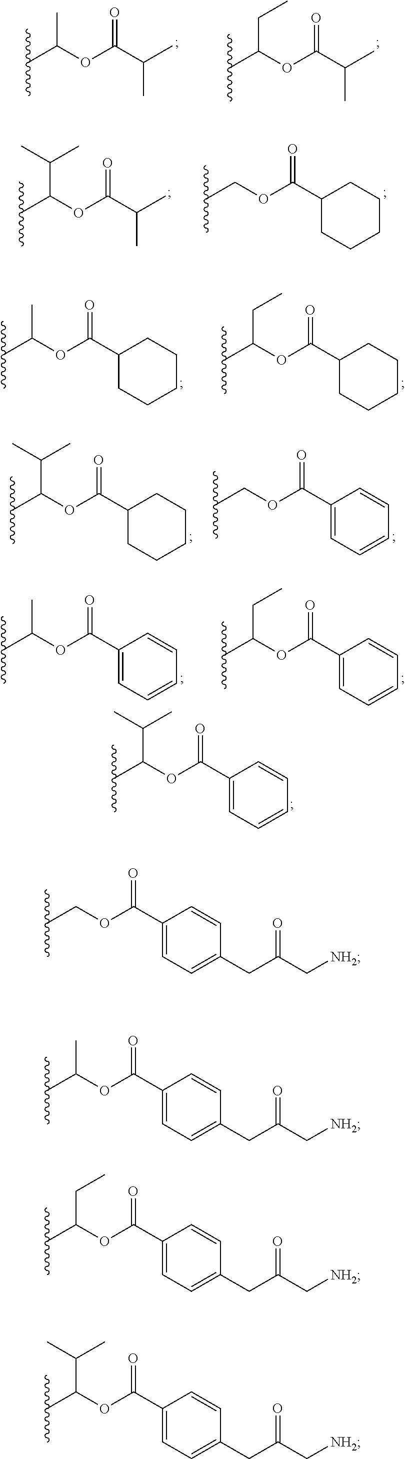

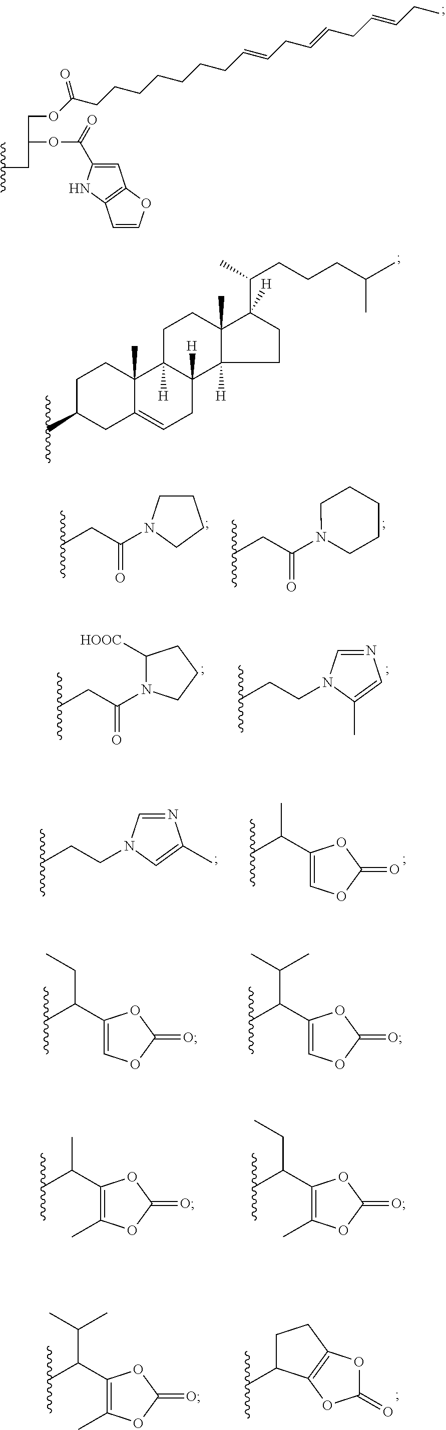

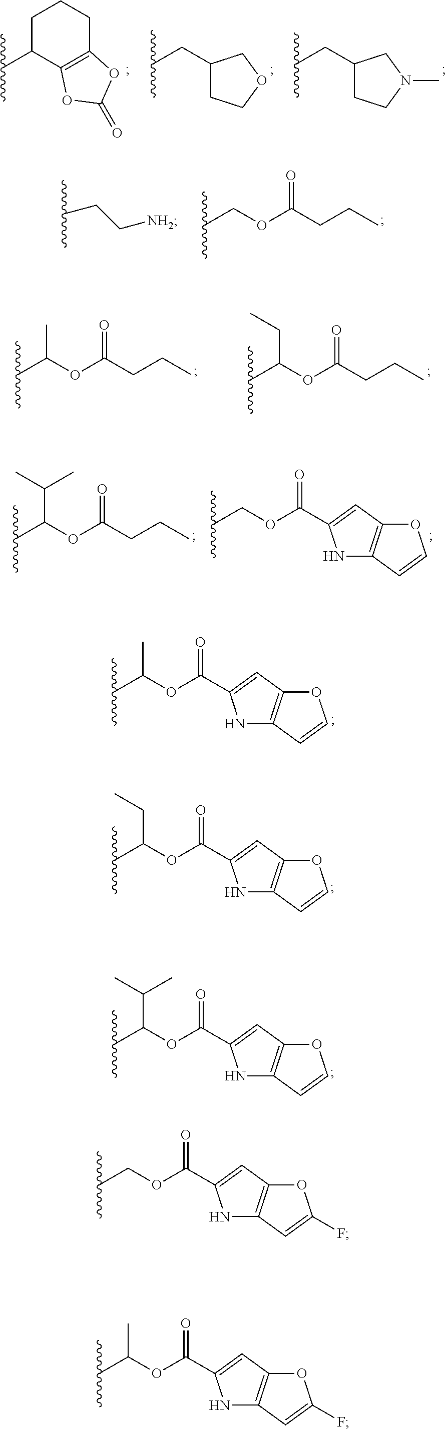

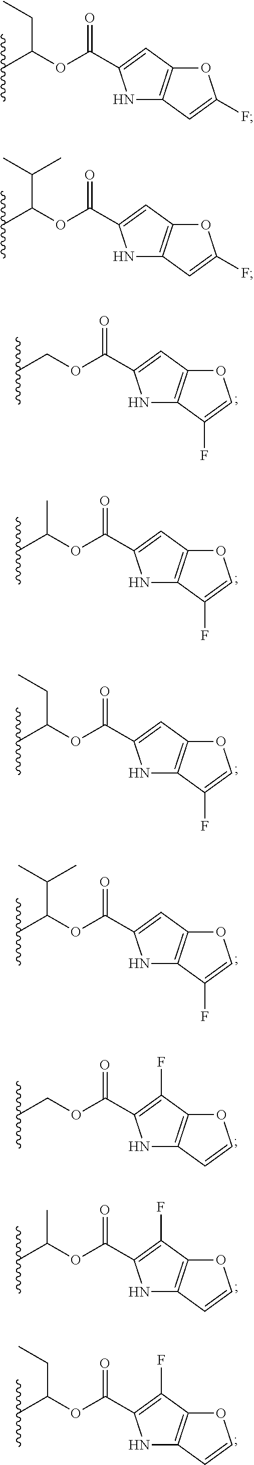

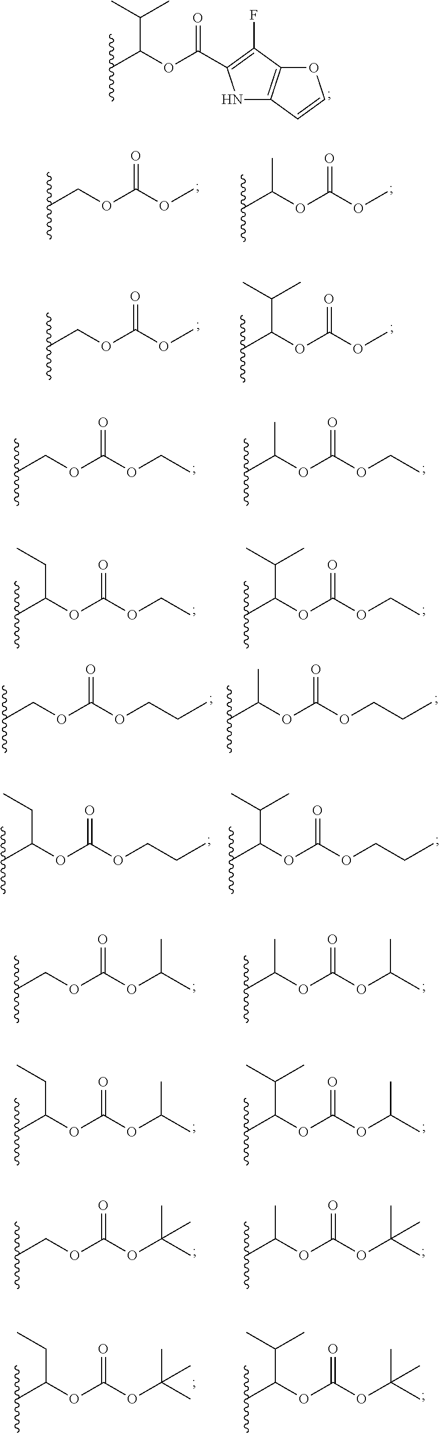

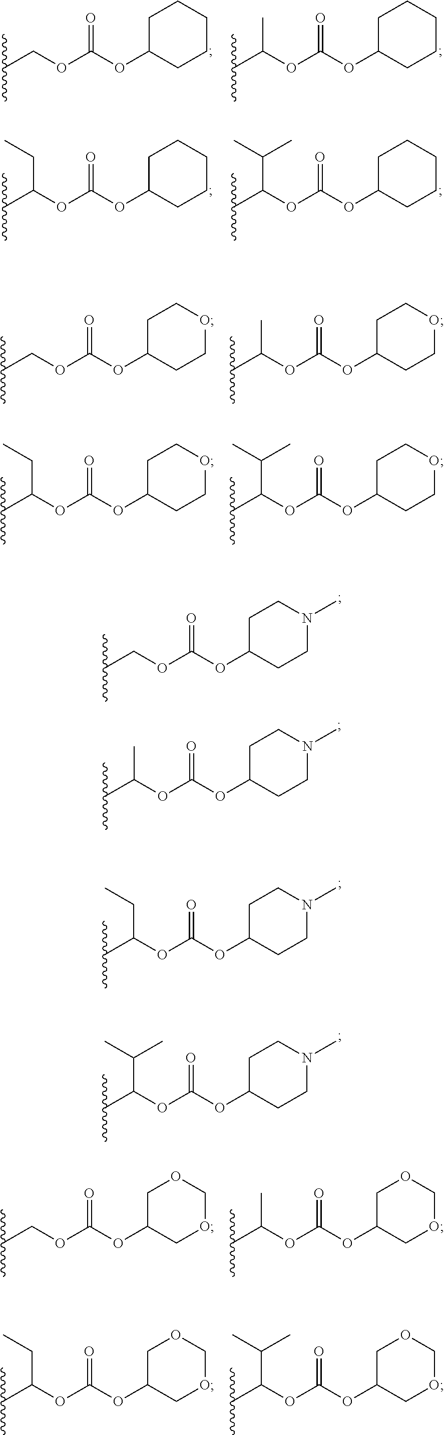

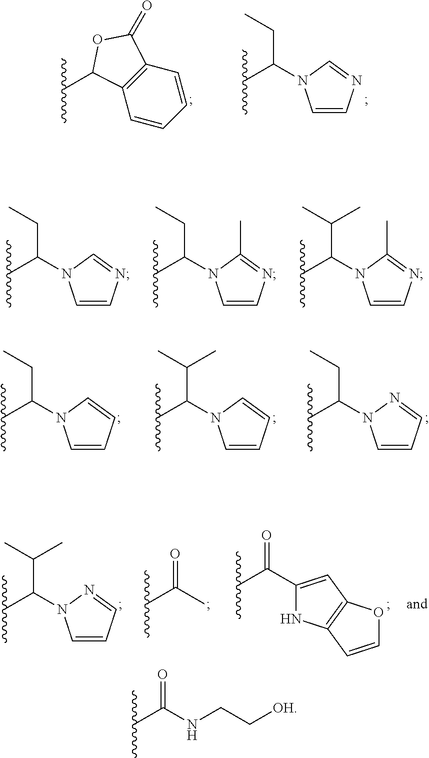

In an exemplary embodiment, R^{26c} is a member selected from

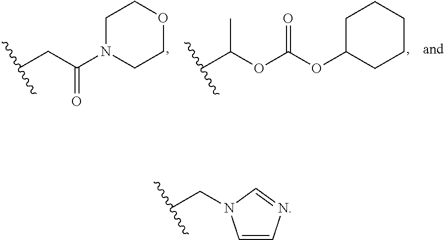

In an exemplary embodiment, R^{26c} is a member selected from

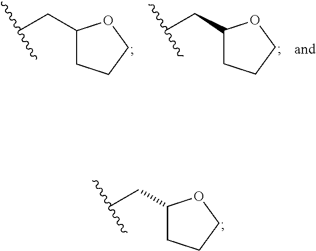

In an exemplary embodiment, R^{26c} is a member selected from

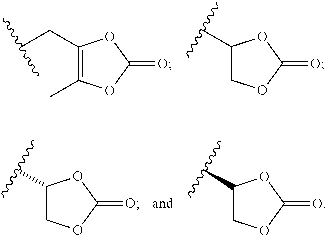

In an exemplary embodiment, R^{26c} is a member selected from

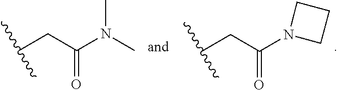

In an exemplary embodiment, R^{26c} is a member selected from

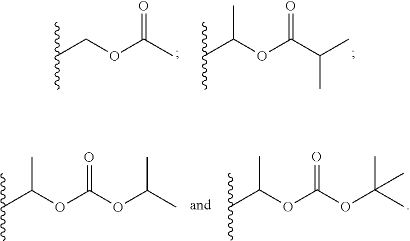

In an exemplary embodiment, the invention provides a pharmaceutical composition comprising (i) a compound set forth herein, or a pharmaceutically acceptable salt thereof and (ii) a pharmaceutically acceptable carrier.

In an exemplary embodiment, the invention provides a method for treating or preventing a condition which is a member selected from a neurological disorder, pain, ataxia and convulsion, said method comprising administering to a subject in need thereof a therapeutically effective amount of a compound set forth herein or a pharmaceutically acceptable salt or hydrate thereof.

In an exemplary embodiment, said neurological disorder is a neurodegenerative disease.

In an exemplary embodiment, said neurodegenerative disease is a member selected from Alzheimer's disease, Parkinson's disease and amyotrophic lateral sclerosis.

In an exemplary embodiment, said neurological disorder is a neuropsychiatric disease.

In an exemplary embodiment, said neuropsychiatric disease is schizophrenia.

In an exemplary embodiment, said pain is neuropathic pain.

In an exemplary embodiment, said pain is a member selected from diabetic neuropathy, post-herpetic neuralgia, spinal cord injury induced pain, neuropathic cancer pain, HIV/AIDS induced pain, phantom limb pain, trigeminal neuralgia, complex regional pain syndrome, chronic migraine, fibromyalgia and lower back pain.

In an exemplary embodiment, the method further comprises co-administering to said subject a modulator of NMDA neurotransmission.

In an exemplary embodiment, said modulator is a member selected from D-serine, cycloserine and analogs thereof.

In an exemplary embodiment, the invention provides a compound comprising a first cyclic structure and a second cyclic structure linked by a linker moiety, said compound having a structure selected from the group:

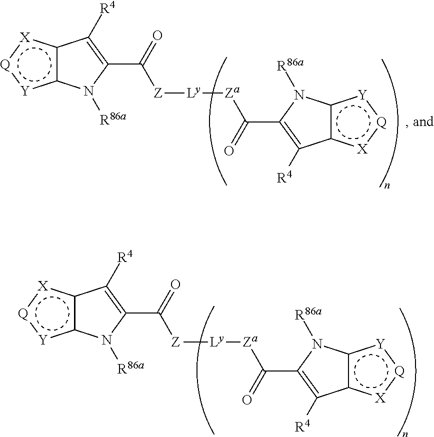

wherein L^{y} is a linker moiety joining said first cyclic structure covalently to said second cyclic structure. Z and each Z^{a} are members independently selected from NR⁴⁵, O, S and CR⁴⁶R⁴⁷. Each Q is a member independently selected from O, S, N and CR¹. Each X is a member independently selected from O, S, N, NR³ and CR^{2a}. Each Y is a member independently selected from O, S, N, NR³ and CR^{2b}. The index n is an integer selected from 1 to 2. Each R¹ is a member independently selected from H, F, substituted or unsubstituted arylalkyl, substituted or unsubstituted heteroarylalkyl, substituted or unsubstituted C₄-C₁₀ cycloalkyl, and substituted or unsubstituted C₄-C₁₀ heterocycloalkyl. Each R^{2a} is a member independently selected from H, F, Cl, Br, CN, substituted or unsubstituted C₃-C₆ alkyl, substituted or unsubstituted arylalkyl, substituted or unsubstituted heteroarylalkyl, substituted or unsubstituted C₄-C₁₀ cycloalkyl, substituted or unsubstituted C₄-C₁₀ heterocycloalkyl and alkenyl. Each R^{2b} is a member independently selected from H, F, substituted or unsubstituted C₃-C₆ alkyl, substituted or unsubstituted arylalkyl, substituted or unsubstituted heteroarylalkyl, substituted or unsubstituted C₄-C₁₀ cycloalkyl, and substituted or unsubstituted C₄-C₁₀ heterocycloalkyl and alkenyl. Each R³ is a member independently selected from H, substituted or unsubstituted C₁-C₆ alkyl, substituted or unsubstituted arylalkyl, substituted or unsubstituted heteroarylalkyl, substituted or unsubstituted C₄-C₁₀ cycloalkyl, and substituted or unsubstituted C₄-C₁₀ heterocycloalkyl. Each R⁴ is a member independently selected from H, F, Cl, Br, CN, unsubstituted C₁-C₆ alkyl, substituted or unsubstituted arylalkyl, substituted or unsubstituted heteroarylalkyl, substituted or unsubstituted C₄-C₁₀ cycloalkyl and alkenyl. Each R^{86a} is a member independently selected from H, substituted or unsubstituted alkyl and substituted or unsubstituted heteroalkyl. Each R^{86a} is optionally joined to Z or Z^{a} to form a ring. Each R⁴⁵, R⁴⁶ and R⁴⁷ is a member independently selected from H, OR⁴⁸, acyl, substituted or unsubstituted alkyl and substituted or unsubstituted heteroalkyl. R⁴⁸ is a member selected from H, substituted or unsubstituted alkyl and substituted or unsubstituted heteroalkyl.

In an exemplary embodiment, each X is O; each Q is CR¹, wherein each R¹ is a member independently selected from H, F, substituted or unsubstituted arylalkyl, substituted or unsubstituted heteroarylalkyl, substituted or unsubstituted C₄-C₁₀ cycloalkyl, and substituted or unsubstituted C₄-C₁₀ heterocycloalkyl; and each Y is CR^{2b}, wherein each R^{2a} is a member independently selected from H, F, Cl, Br, CN, substituted or unsubstituted C₃-C₆ alkyl, substituted or unsubstituted arylalkyl, substituted or unsubstituted heteroarylalkyl, substituted or unsubstituted C₄-C₁₀ cycloalkyl, substituted or unsubstituted C₄-C₁₀ heterocycloalkyl and alkenyl.

In an exemplary embodiment,

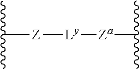

has the formula:

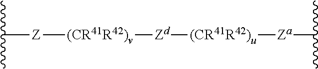

wherein each R⁴¹ and each R⁴² are independently selected from H, OR⁴³, NR⁴³R⁴⁴, CN, halogen, acyl, substituted or unsubstituted alkyl, substituted or unsubstituted heteroalkyl, substituted or unsubstituted aryl, substituted or unsubstituted heteroaryl, substituted or unsubstituted heterocycloalkyl. Each R⁴³ and each R⁴⁴ are members independently selected from H, acyl, substituted or unsubstituted alkyl and substituted or unsubstituted heteroalkyl. Z, each Z^{a} and each Z^{d} are members independently selected from NR⁴⁵, O, S and CR⁴⁶R⁴⁷. Each R⁴⁵, each R⁴⁶ and each R⁴⁷ are members independently selected from H, OR⁴⁸, acyl, substituted or unsubstituted alkyl and substituted or unsubstituted heteroalkyl. Each R⁴⁸ is a member independently selected from H, substituted or unsubstituted alkyl and substituted or unsubstituted heteroalkyl. The indices u and v are integers independently selected from 0 to 10.

In an exemplary embodiment,

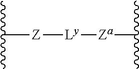

has the formula:

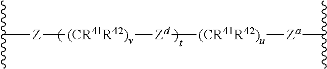

wherein each R⁴¹ and each R⁴² are members independently selected from H, OR⁴³, NR⁴³R⁴⁴, CN, halogen, acyl, substituted or unsubstituted alkyl, substituted or unsubstituted heteroalkyl, substituted or unsubstituted aryl, substituted or unsubstituted heteroaryl, substituted or unsubstituted heterocycloalkyl. Each R⁴³ and each R⁴⁴ are members independently selected from H, acyl, substituted or unsubstituted alkyl and substituted or unsubstituted heteroalkyl. Z, each Z^{a} and each Z^{d} are members independently selected from NR⁴⁵, O, S and CR⁴⁶R⁴⁷. Each R⁴⁵, each R⁴⁶ and each R⁴⁷ are members independently selected from H, OR⁴⁸, acyl, substituted or unsubstituted alkyl and substituted or unsubstituted heteroalkyl. Each R⁴⁸ is a member independently selected from H, substituted or unsubstituted alkyl and substituted or unsubstituted heteroalkyl. The indices u and v are independently selected integers from 0 to 10. The index t is an integer from 0 to 20,000.

In an exemplary embodiment, the invention provides a compound comprising a first cyclic structure and a second cyclic structure linked by a linker moiety, said compound having a structure:

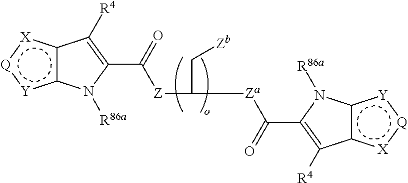

wherein o is an integer from 0 to 1,000; and each Z^{b} is a member independently selected from H, OR⁴⁹, substituted or unsubstituted alkyl, and substituted or unsubstituted heteroalkyl. Z and Z^{a} are members independently selected from NR⁴⁵, O, S and CR⁴⁶R⁴⁷. Each Q is a member independently selected from O, S, N and CR¹. Each X is a member independently selected from O, S, N, NR³ and CR^{2a}. Each Y is a member independently selected from O, S, N, NR³ and CR^{2b}. Each R¹ is a member independently selected from H, F, substituted or unsubstituted arylalkyl, substituted or unsubstituted heteroarylalkyl, substituted or unsubstituted C₄-C₁₀ cycloalkyl, and substituted or unsubstituted C₄-C₁₀ heterocycloalkyl. Each R^{2a} is a member independently selected from H, F, Cl, Br, CN, substituted or unsubstituted C₃-C₆ alkyl, substituted or unsubstituted arylalkyl, substituted or unsubstituted heteroarylalkyl, substituted or unsubstituted C₄-C₁₀ cycloalkyl, substituted or unsubstituted C₄-C₁₀ heterocycloalkyl and alkenyl. Each R^{2b} is a member independently selected from H, F, substituted or unsubstituted C₃-C₆ alkyl, substituted or unsubstituted arylalkyl, substituted or unsubstituted heteroarylalkyl, substituted or unsubstituted C₄-C₁₀ cycloalkyl, and substituted or unsubstituted C₄-C₁₀ heterocycloalkyl and alkenyl. Each R³ is a member independently selected from H, substituted or unsubstituted C₁-C₆ alkyl, substituted or unsubstituted arylalkyl, substituted or unsubstituted heteroarylalkyl, substituted or unsubstituted C₄-C₁₀ cycloalkyl, and substituted or unsubstituted C₄-C₁₀ heterocycloalkyl. Each R⁴ is a member independently selected from H, F, Cl, Br, CN, unsubstituted C₁-C₆ alkyl, substituted or unsubstituted arylalkyl, substituted or unsubstituted heteroarylalkyl, substituted or unsubstituted C₄-C₁₀ cycloalkyl and alkenyl. Each R^{86a} is a member independently selected from H, substituted or unsubstituted alkyl and substituted or unsubstituted heteroalkyl. Each R^{86a} is optionally joined to Z or Z^{a} to form a ring. Each R⁴⁵, R⁴⁶ and R⁴⁷ is a member independently selected from H, OR⁴⁸, acyl, substituted or unsubstituted alkyl and substituted or unsubstituted heteroalkyl. R⁴⁸ is a member selected from H, substituted or unsubstituted alkyl and substituted or unsubstituted heteroalkyl. Each R⁴⁹ is a member independently selected from H, acyl, substituted or unsubstituted alkyl and substituted or unsubstituted heteroalkyl.

In an exemplary embodiment, the invention provides a compound having a structure according to any of the prodrugs of Table 1.

In an exemplary embodiment, the invention provides a compound having a structure according to any of the prodrugs of Table 2.

In an exemplary embodiment, the invention provides a compound having a structure according to any of the prodrugs of Table 3.

In an exemplary embodiment, the invention provides a compound having a structure according to any of the prodrugs of Formulae D-P.

The following examples are provided to illustrate selected embodiments of the present inventions and are not to be construed as limiting its scope.

### Examples

### General Procedures for Synthesizing Prodrug Precursors

### General Procedure A: Synthesis of Fused Pyrrole Analogs

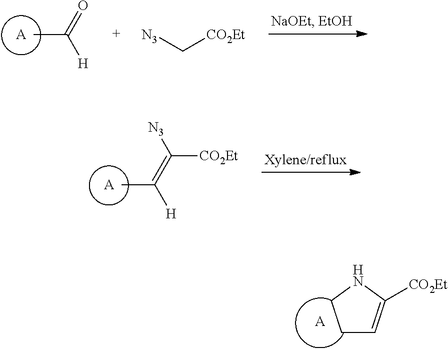

In the above Scheme, ring A represents any substituted or unsubstituted 5-membered, aromatic ring. Exemplary aromatic rings include thiophenes, furans, thiazoles and pyrroles.

### A) Condensation of an Aldehyde with Ethyl Azidoacetate

A solution of the aldehyde (e.g., 1.61 g, 8.41 mmol) and about 4 to about 7 equivalents of ethyl azidoacetate (e.g., 4.34 g, 33.7 mmol) in anhydrous EtOH (e.g., 10.5 mL) was added dropwise to a solution of sodium (e.g., 0.8 g) in anhydrous EtOH (e.g., 50.0 mL) at a temperature between about 0° C. and about −45° C. (typically between about −10 and about −5° C. (e.g., NaCl/ice)). The reaction mixture was stirred for about 1 hour (h) while the temperature was maintained below 0° C. and was then allowed to warm to ambient temperature (also called room temperature, rt) (e.g., overnight). The mixture was quenched with a cold solution of saturated aqueous NH₄Cl or was diluted with water (e.g., 0.5 L). The product was extracted with diethyl ether or ethyl acetate (EtOAc) (e.g., 3×0.2 L) and the combined organic phases were washed with saturated aqueous NaCl solution (2×0.1 L), dried (e.g., over Na₂SO₄) and filtered. The solvent was removed in vacuo to give the ethyl azidoacrylate. Alternatively, the solvent was reduced in vacuo (e.g., to about 50 mL) and the resulting solution was used in the next reaction step.

### B) Cyclization of the Ethyl Azidoacrylate

A solution of the above ethyl azidoacrylate in o- or m-xylene (e.g., 150 mL) was heated to reflux for a time period between about 15 minutes (min) and 14 h (typically about 1 h). The reaction mixture was then allowed to cool to ambient temperature. The solution was concentrated in vacuo and the crude product was purified (e.g., silica gel column chromatography) to give the fused pyrrole ethyl ester.

### General Procedure B: Saponification of Esters

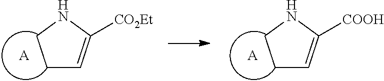

To a solution or suspension of the ester (e.g., 0.33 g, 1.2 mmol) in MeOH or EtOH (e.g., 16.5 mL) was added an aqueous base, such as 10M NaOH (e.g., 0.6 mL, 6 mmol), 5M KOH (e.g., 1.2 mL, 6 mmol) or 1M LiOH (e.g., 6 mL). The solution was heated to a temperature between about 80° C. and reflux for a time period between about 30 min and about 20 h (e.g., 5 h). The reaction mixture was cooled to room temperature (rt) and was then acidified. In one example, the mixture was poured into water (e.g., 200 mL) and the pH of the resulting mixture was adjusted to about pH 1-2 with HCl. In one example, excess solvent was removed in vacuo and the residue was dissolved in 5% aqueous citric acid (e.g., 15 mL). In other examples, the solvent was removed in vacuo and the residue was dissolved in a saturated solution of NH₄Cl (e.g., 15 mL). The acidified solution was then extracted (e.g., 3×100 mL EtOAc) and the combined organic layers were washed (e.g., with brine), dried (e.g., over Na₂SO₄), filtered and concentrated in vacuo to give the carboxylic acid.

### Synthesis of 4H-furo[3,2-b]pyrrole-5-carboxylic acid

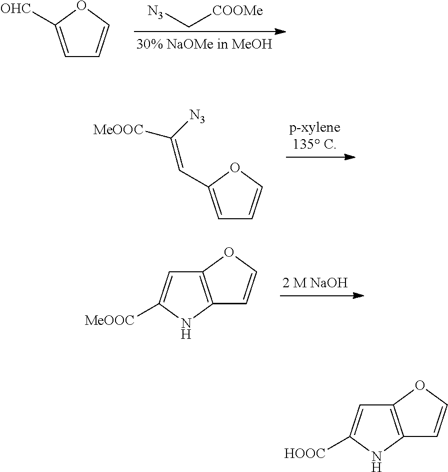

The synthesis was described in WO/2008/005456. ¹H NMR (400 MHz, DMSO-d₆) δ (ppm) 12.34 (s, 1H) 11.48 (s, 1H) 7.75 (s, 1H) 6.68 (s, 1H) 6.57 (s, 1H).

### General Procedures for Synthesizing Prodrugs

### General Procedure 1: Synthesis of Fused pyrrole[1,2-c]oxazol-1(3H)-one Derivatives

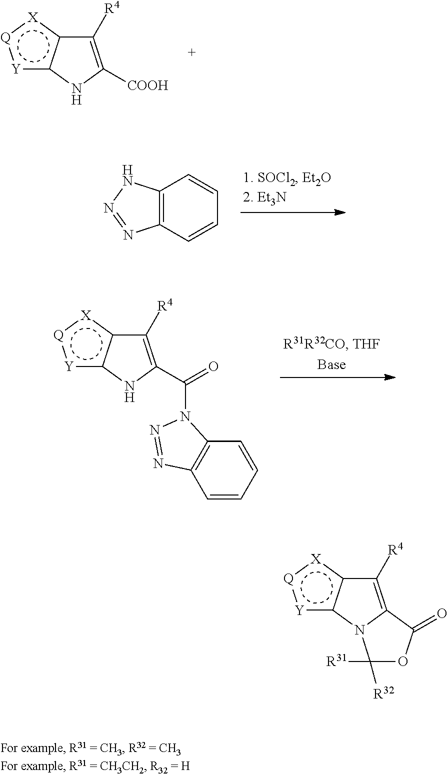

### 1.A) Synthesis of N-acylbenzotriazoles

To a solution of benzotriazole (1 equiv) in anhydrous ether at room temperature (rt) was added SOCl₂ (2 equiv). After stirring for 0.5 hours (h), a fused pyrrole carboxylic acid starting material (e.g., 4H-furo[3,2-b]pyrrole-5-carboxylic acid) (1 equiv) was added in one portion. After stirring for 2 h, triethylamine was added dropwise. After an additional 2 h, the mixture was filtered, and the filtrate was washed with 10% NaOH, dried over Na₂SO₄. After filtration and concentration in vacuo, the N-acylbenzotriazole intermediate (e.g., (1H-benzo[d][1,2,3]triazol-1-yl)(4H-furo[3,2-b]pyrrol-5-yl)methanone) was used directly for the next step without further purification. (Reference: Synthesis, 2003, 18, 2795-2798.)

### 1.B) Ring Closure to Form Fused pyrrole[1,2-c]oxazol-1(3H)-one Derivatives

A mixture of the N-acylbenzotriazole (e.g., (1H-benzo[d][1,2,3]triazol-1-yl)(4H-furo[3,2-b]pyrrol-5-yl)methanone), a corresponding aldehyde (e.g., acetaldehyde) or ketone (e.g., acetone) (1.2 equiv) and DBU (4 equiv) in THF was stirred under reflux for 3 h. The reaction mixture was then concentrated and purified by column chromatography to give the desired product (e.g.,
5,10-dioxa-1-azatricyclo[6.3.0.0^{2,6}]undeca-2(6),3,7-trien-9-one, or
11-methyl-5,10-dioxa-1-azatricyclo[6.3.0.0^{2,6}]undeca-2(6),3,7-trien-9-one, respectively). (Reference: J. Org. Chem., 2004, 69, 9313-9315.)

### General Procedure 2.1: Synthesis of Alkyl Ester and Aryl Ester Derivatives

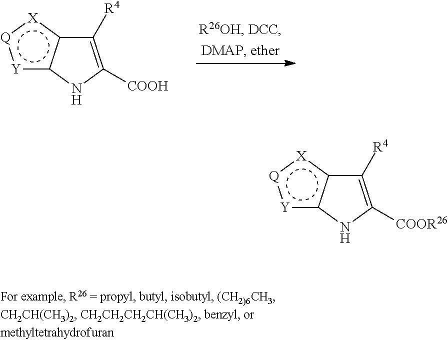

A solution of a fused pyrrole carboxylic acid starting material (e.g., 4H-furo[3,2-b]pyrrole-5-carboxylic acid) (1 equiv) in diethyl ether was added slowly to a mixture of N, N′-dicyclohexylcarbodiimide (DCC) (1.2 equiv) and a catalytic amount of dimethylaminopyridine (DMAP) in a corresponding alcohol (e.g., benzyl alcohol) (7-8 equiv). The mixture was stirred at rt for 6 h. The reaction mixture was filtered to remove the precipitated N, N′-dicyclohexylurea (DCU). The filtrate was concentrated in vacuo and the resulting crude product was purified by column chromatography to give an alkyl ester derivative (e.g., benzyl 4H-furo[3,2-b]pyrrole-5-carboxylate). (Reference: Tetrahedron, 1992, 48(16), 3437-3444.)

### General Procedure 2.2: Synthesis of Alkyl Ester Derivatives

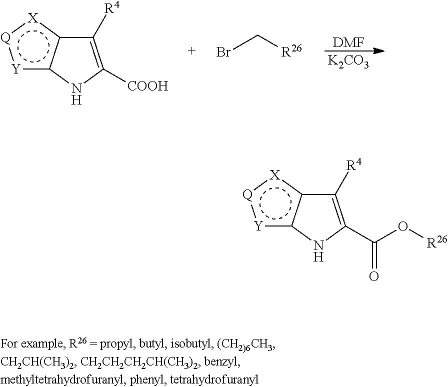

A solution of a fused pyrrole carboxylic acid starting material (e.g., 4H-furo[3,2-b]pyrrole-5-carboxylic acid) (1 equiv), the alkyl bromide (e.g., 2-(bromomethyl)-tetrahydrofuran) (1 equiv), and K₂CO₃ (0.60 equiv) in DMF (0.75 M in fused pyrrole carboxylic acid) was heated (approximately 115° C.) and stirred overnight. The reaction mixture was cooled to rt and treated with active carbon for 10 min. The slurry was filtered. The filtrate was diluted with H₂O and extracted with EtOAc (3 times). The combined organic layer was washed with 10% NaOH (2 times) and brine (2 times). The organic layer was dried over MgSO₄ and filtered. The crude product was crystallized (EtOAc:n-hexane=1:4) to produce the desired product.

### General Procedure 2.3: Synthesis of Alkyl Ester and Aryl Ester Derivatives

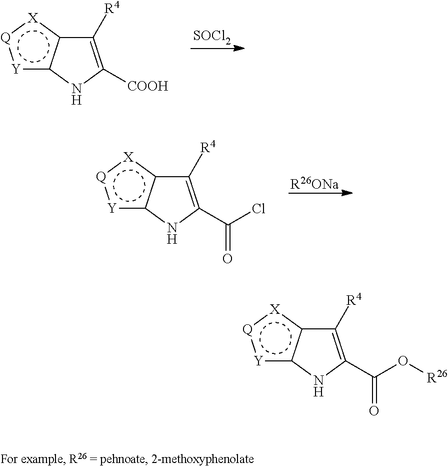

### 2.3.A) Synthesis of Sodium Salt of Alkyl or Aryl Alcohol

To a solution of the alkyl or aryl alcohol (e.g., 2-methoxyphenol) (4.03 mmol) in dichloromethane (DCM) (15 mL) was added NaH (150 mg, 60%) in several portions. After stirring at rt for 0.5 h, the reaction mixture was filtered. The crude product was used directly for the next step.

### 2.3.B) Synthesis of pyrrole-5-carbonyl chlorides

To a solution of a fused pyrrole carboxylic acid starting material (e.g., 4H-furo[3,2-b]pyrrole-5-carboxylic acid) (1 equiv) and a catalytic amount of DMF in DCM was added SOCl₂ (2-3 equiv) dropwise. The resulting mixture was stirred at 30° C. for 40 min, and then concentrated in vacuo to give the corresponding crude acyl chloride (e.g., 4H-furo[3,2-b]pyrrole-5-carbonyl chloride).

### 2.3.C) Synthesis of Alkyl Ester and Aryl Ester Derivatives

To a solution of the sodium salt of an alkyl or aryl alcohol (e.g., sodium 2-methoxyphenolate) (1 mmol) in DCM (10 mL) was added the acyl chloride (e.g., 4H-furo[3,2-b]pyrrole-5-carbonyl chloride) (1 mmol) in DCM (5 mL) dropwise. After addition, the mixture was diluted with H₂O and extracted with EtOAc (3 times). The combined organic layer was washed with brine and dried over Na₂SO₄. After filtration, the organic solution was concentrated and purified by column chromatography to give the desired product.

### General Procedure 2.4: Synthesis of Alkyl Ester and Aryl Ester Derivatives

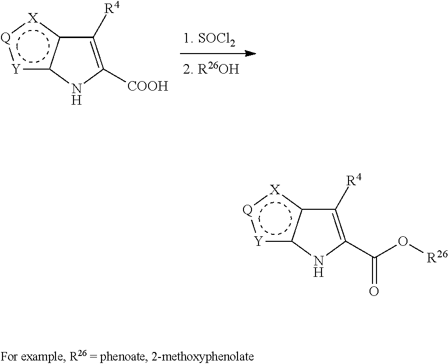

A fused pyrrole carboxylic acid starting material (e.g., 4H-furo[3,2-b]pyrrole-5-carboxylic acid) (1.3 mmol) was converted to the corresponding acyl chloride (e.g., 4H-furo[3,2-b]pyrrole-5-carbonyl chloride) according to General Procedure 2.3.B. The acyl chloride was filtered, and then the alcohol (e.g., 3-methylbutan-1-ol) (2.6 mmol) was added. The mixture was stirred at 40° C. for 2 h. After cooling to rt, the reaction mixture was diluted with H₂O and extracted with EtOAc (3 times). The combined organic layer was washed with brine and dried over Na₂SO₄. After concentration, the crude product was purified by column chromatography to give the desired product.

### General Procedure 3: Synthesis of Amino Acid Derived Ester Derivatives

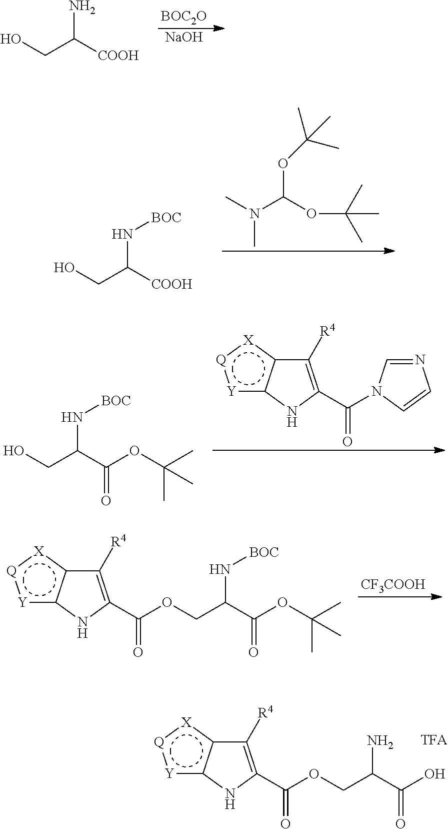

### 3.A) Synthesis of N-Boc-L-serine

A solution of di-tert-butyldicarbonate (Boc₂O) (2.6 g, 0.0119 mol) in 1,4-dioxane was added to a mixture of L-serine (1.05 g, 0.01 mol) and NaOH (20.5 mL, 0.0205 mol) at 0° C. The reaction mixture was stirred at 0° C. for 0.5 h and at rt for 3 h. The reaction mixture was then concentrated to half of its original volume and the pH of the solution was adjusted to around 3. The mixture was extracted with EtOAc (3 times) and dried over Na₂SO₄. After filtration and concentration, the N-Boc-L-serine intermediate was obtained and used for the next step without further purification. (Reference: J. Org. Chem., 2005, 70(7), 2430-2438.)

### 3.B) Synthesis of N-Boc-L-serine tent-butyl Ester Derivatives

A solution of above N-Boc-L-serine (1 equiv) and N, N-dimethylformamide di-tert-butylacetal (2 equiv) in dry benzene was refluxed under N₂ for 19 h. After cooling to rt, the mixture was treated with 5% aq. NaHCO₃ solution and was stirred for 30 min, followed by addition of an adequate amount of methanol to form a homogeneous solution. The solution was extracted with EtOAc. The organic layer was rinsed with H₂O (3 times), brine, and dried over Na₂SO₄. After filtration, the filtrate was concentrated in vacuo to give N-Boc-L-serine tent-Butyl ester as a yellow oil. (Reference: Chem. Pharm. Bull, 2000, 48(2), 278-280.)

### 3.C) Synthesis of N-acylimidazoles

A solution of a fused pyrrole carboxylic acid (e.g., 4H-furo[3,2-b]pyrrole-5-carboxylic acid) starting material (1 equiv) and CDI (1.05 equiv) in acetonitrile (CH₃CN) was stirred at rt overnight. The mixture was then filtered to give a solution of the desired N-acylimidazole intermediate, which was used for the next step without isolation.

### 3.D) Synthesis of the Intermediate of Amino Acid Derived Esters

A mixture of N-acylimidazoles (1.6 equiv), Et₃N (0.03 equiv), a catalytical amount of DMAP, and N-Boc-L-serine tert-Butyl ester (1 equiv) in CH₃CN was stirred under reflux for 2 days. After cooled to rt, the reaction mixture was concentrated and the residue was diluted with EtOAc. The EtOAc solution was washed by brine, dried over Na₂SO₄, filtered and concentrated. The resulting crude product was purified by column chromatography to give the desired product.

### 3.E) Synthesis of the Salt of Amino Acid Derived Esters

To a solution of protected amino acid derived ester intermediate in DCM was added trifluoroacetic acid (TFA) (80-85 equiv, large excess) dropwise. After stirring at rt for 2 h, the reaction mixture was concentrated to give a crude product. The crude product was dissolved in EtOAc and filtered. The filtrate was concentrated to give an oil which was triturated with a small amount of diethyl ether to form a solid product.

### General Procedure 4: Synthesis of glycolamide Ester Derivatives

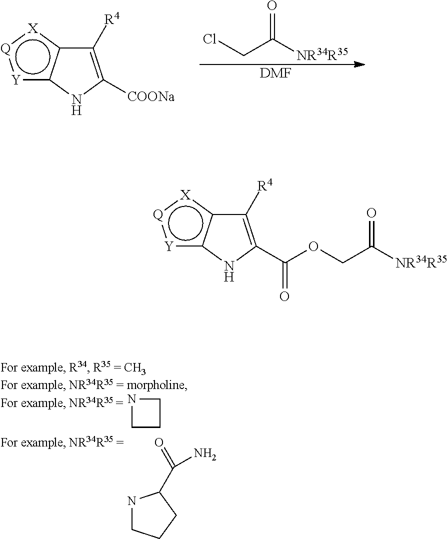

### 4.A) Synthesis of chloroacetylamides

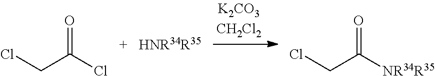

The amine (e.g., azetidine) (2.105 mmol) and anhydrous K₂CO₃ (1 g, 7.246 mmol) were dissolved in anhydrous DCM (15 g) and cooled to 0° C. Chloroacetyl chloride (250 mg, 2.212 mmol) was added dropwise. The resulting reaction mixture was stirred at rt for 42 h. The reaction mixture was then filtered, washed with 5 mL DCM. The combined filtrate was concentrated in vacuo to give the desired chloroacetylamides (e.g., 1-(azetidin-1-yl)-2-chloroethanone).

### 4.B) Synthesis of chloroacetylamides

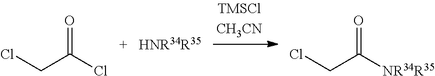

Trimethylchlorosilane (1.54 g, 14.3 mmol) was added to the amine (e.g., solution of L-proline) (1.15 g, 10 mmol) in dry CH₃CN (5 mL)). The reaction mixture was stirred at 50° C. for 1 h. 2-Chloroacetyl chloride (0.8 mL, 10 mmol) and a catalytic amount of DMF were added dropwise to the reaction solution. The resulting reaction mixture was stirred at rt overnight. After removal of solvents under reduced pressure, the residue was treated with water, and extracted with ethyl acetate. After separation of layers, the organic layer was dried over anhydrous magnesium sulfate. After filtration, the solvent was removed by distillation to give the desired crude product (e.g., 1-(2-chloroacetyl)pyrrolidine-2-carboxylic acid) as a yellow oil, which was used directly for the next step without further purification.

### 4.C) Synthesis of glycolamide Ester Derivatives

A solution of the sodium salt of a fused pyrrole carboxylic acid starting material (e.g., 4H-furo[3,2-b]pyrrole-5-carboxylic acid) (1 equiv) and the chloroacetylamide derivative (e.g., 2-chloro-N,N-dimethylacetamide) (1 equiv) in DMF was heated to 95° C. until the reaction was complete (approximately 1.5 to 2 h). After cooling to rt, the mixture was treated with H₂O and extracted with EtOAc (4 times). The combined organic layers were washed with 1% NaOH solution, brine, and dried over MgSO₄. The mixture was filtered and concentrated to give the desired glycolamide ester derivative (e.g., 2-(dimethylamino)-2-oxoethyl 4H-furo[3,2-b]pyrrole-5-carboxylate). (Reference: Angew. Chem. Intl. Ed. Eng., 1965, 4, 417-429.)

### General Procedure 5: Synthesis of Imidazole Derived Carboxylic Ester Derivatives

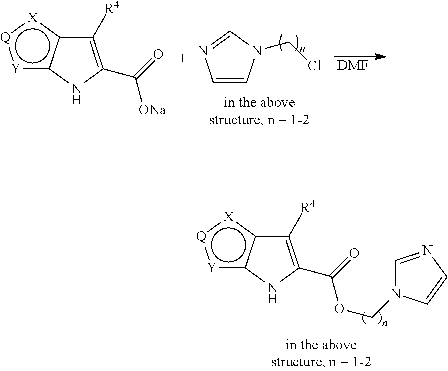

A mixture of the sodium salt of a fused pyrrole carboxylic acid (e.g., 4H-furo[3,2-b]pyrrole-5-carboxylic acid) starting material (1 equiv) and chloroalkylimidazole (e.g., 1-(chloromethyl)-1H-imidazole) (1-2 equiv) in DMF (0.1 M in fused pyrrole carboxylic acid) was heated (90-120° C.) and stirred overnight. After cooling to rt, the reaction mixture was treated with water and extracted with EtOAc. After separation of layers, the organic layer was washed with 10% NaOH solution, brine, and dried over anhydrous Na₂SO₄. After filtration, the filtrate was concentrated in vacuo to give an oil, which was triturated with a small amount of EtOAc to form a desired imidazole-derived carboxylic ester product (e.g., (1H-imidazol-1-yl)methyl 4H-furo[3,2-b]pyrrole-5-carboxylate). (Reference: Tetrahedron Lett., 2001, 42, 6097-6100; Tetrahedron Lett., 2007, 48, 4609-4611.)

### General Procedure 6: Synthesis of 2-aminoethyl pyrrole-5-carboxylate Ester Derivatives

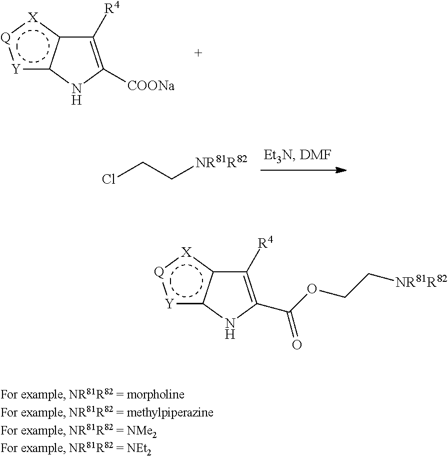

A solution of sodium salt of a fused pyrrole carboxylic acid starting material (e.g., 4H-furo[3,2-b]pyrrole-5-carboxylic acid) (1 equiv), the 2-chloroethylamine (e.g., N-(2-chloroethyl)morpholine hydrochloride) (1.05-1.5 equiv), and Et₃N (1.8 equiv) in DMF (0.12 M in fused pyrrole carboxylic acid) was heated (100-120° C.) and stirred until the reaction was complete (approximately 24-40 h). After cooling to rt, the reaction mixture was treated with H₂O and extracted with EtOAc. After separation of layers, the organic layer was washed with H₂O (3 times) and dried over MgSO₄. The mixture was filtered and concentrated to give the desired 2-aminoethyl pyrrole-5-carboxylate ester derivative (e.g., 2-morpholinoethyl 4,6a-dihydro-3aH-furo[3,2-b]pyrrole-5-carboxylate).

### General Procedure 7: Synthesis of Acyloxymethyl Ester Derivatives

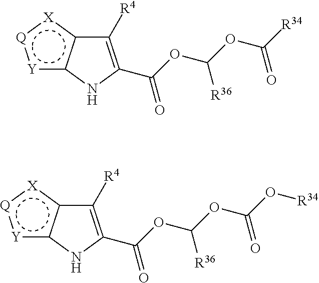

### 7.A) Synthesis of 1-chloromethyl Acyl Ester Derivatives

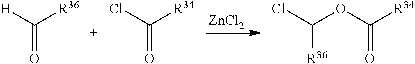

To a flask charged with a catalytic amount of ZnCl₂ under N₂ was added an acyl chloride (e.g., acetyl chloride) (1 equiv), and the mixture was cooled to −5 to −10° C. An aldehyde (e.g., acetaldehyde) (1.1 equiv) was then added dropwise and the resulting reaction mixture was stirred at rt for 1 h. The mixture was concentrated under the reduced pressure to afford the crude product. The crude product (e.g., 1-chloroethyl acetate) was used directly in next step without any further purification. (Reference: Synlett, 2006, 10, 1485-1490.)

### 7.B) Synthesis of 1-chloromethyl Acyl Ester Derivatives

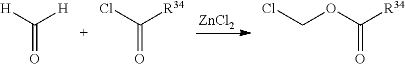

To a flask charged with a catalytic amount of ZnCl₂ under N₂ was added an acyl chloride (e.g., propionyl chloride) (1 equiv), and the mixture was cooled to −5 to −10° C. Paraformaldehyde (1.38 g, 46 mmol) was then added dropwise, and the resulting reaction mixture was stirred at rt for 1 h. The mixture was concentrated under the reduced pressure to afford the crude product. The crude product (e.g., chloromethyl propionate) was used directly in next step without any further purification.

### 7.C) Synthesis of 1-chloromethyl Acyloxy Ester Derivatives

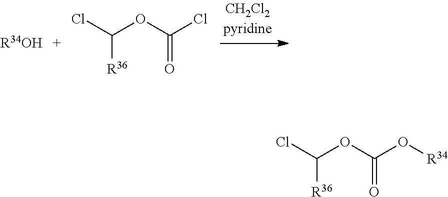

To a solution of the chloromethyl carbonochloridate (e.g., chloro(methyl)methyl carbonochloridate) (1.07 equiv) in DCM was added the alcohol (e.g., tert-butyl alcohol) (1 equiv) at 0-5° C., followed by the addition of pyridine (1.07 equiv) dropwise over 2 min. The resulting mixture was stirred at 0-5° C. for 30 min. The reaction mixture was then washed successively with water, brine, and 0.5M HCl solution. The organic layer was dried over anhydrous MgSO₄. After filtration, the solvent was removed by distillation to yield the crude product (e.g., tert-butyl 1-chloroethyl carbonate), which was used directly for the next step without further purification.

### 7.D) Synthesis of 1-acetoxyethyl Derived pyrrole-5-carboxylate Esters

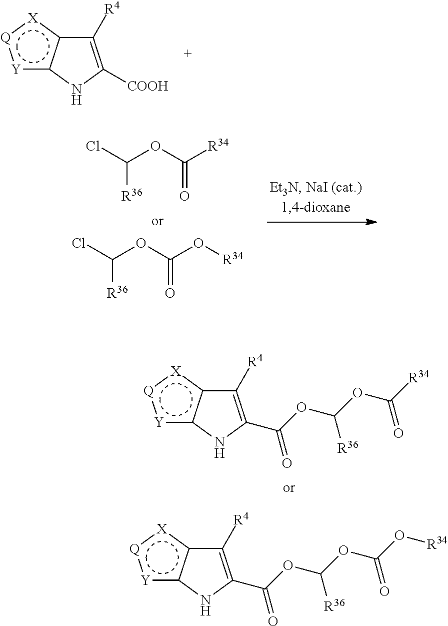

A catalytic amount of sodium iodide was added to a stirred solution of the 1-chloromethyl acyl ester (e.g., 1-chloroethyl acetate) or 1-chloromethyl acyloxy ester (e.g., tent-butyl 1-chloroethyl carbonate) (1-1.1 equiv) and a fused pyrrole carboxylic acid starting material (e.g., 4H-furo[3,2-b]pyrrole-5-carboxylic acid) (1 equiv) in dry dioxane at rt. The reaction mixture was heated (80-90° C.) and stirred until judged complete (approximately 8-24 h). The reaction mixture was concentrated in vacuo. The crude product (e.g., 1-acetoxyethyl 4H-furo[3,2-b]pyrrole-5-carboxylate or 1-(tert-butoxycarbonyloxy)ethyl 4H-furo[3,2-b]pyrrole-5-carboxylate, respectively) was isolated by silica gel column chromatographic purification.

### General Procedure 8: Synthesis of Ethylene Linked Diester Derivatives

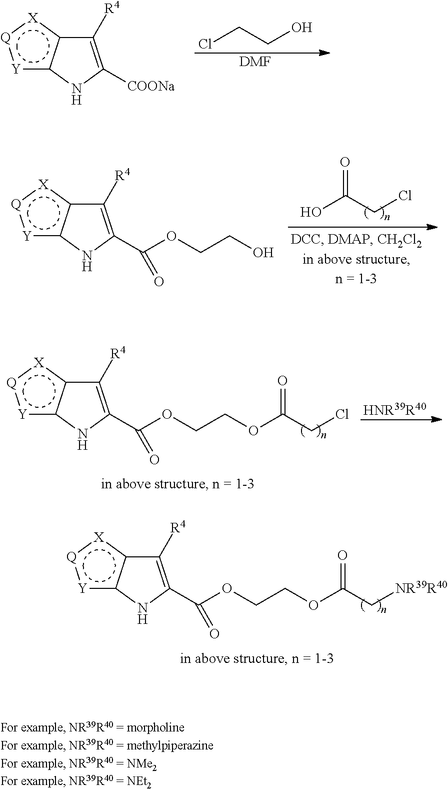

### 8.A) Synthesis of 2-hydroxyethyl pyrrole-5-carboxylate Derivatives

A solution of sodium salt of a fused pyrrole carboxylic acid starting material (e.g., 4H-furo[3,2-b]pyrrole-5-carboxylic acid) (1 equiv) and 2-chloroethanol (1.1-2 equiv) in DMF (0.3 M in fused pyrrole carboxylic acid) was heated (120-125° C.) and stirred until the reaction was complete (approximately 4-12 h). After cooling to rt, the reaction mixture was treated with H₂O and extracted with EtOAc (3 times). After separation of layers, the combined organic layer was washed with H₂O (3 times) and dried over MgSO₄. After filtration, the filtrate was concentrated in vacuo to give the crude product. The crude product was washed with a mixture of EtOAc and hexane (1/5 ratio) to give the desired 2-hydroxyethyl pyrrole-5-carboxylate derivative (e.g., 2-hydroxyethyl 4,6a-dihydro-3aH-furo[3,2-b]pyrrole-5-carboxylate).

### 8.B) Synthesis of 2-(4-chloroacyloxy)ethyl pyrrole-5-carboxylate Derivatives

A mixture of the 2-hydroxyethyl pyrrole-5-carboxylate derivative (e.g., 2-hydroxyethyl 4H-furo[3,2-b]pyrrole-5-carboxylate) (1 equiv), the chloroalkanoic acid (e.g., 4-chlorobutanoic acid) (1.3 equiv), DCC (1.3 equiv), and a catalytic amount of DMAP in DCM was stirred at rt for 4 h. The reaction mixture was filtered and concentrated in vacuo to give the crude product. The crude product was suspended in a mixture of Et₂O and hexane (1/1 ratio) and stirred for 4 h. The suspension was filtered and the filtrate was concentrated to give the desired product (e.g., 2-(4-chlorobutanoyloxy)ethyl 4H-furo[3,2-b]pyrrole-5-carboxylate).

### 8.C) Synthesis of Ethylene Linked pyrrole-5-carboxylate Diester Derivatives

A mixture of 2-(chloroacyloxy)ethyl pyrrole-5-carboxylate derivative (e.g., 2-(4-chlorobutanoyloxy)ethyl 4H-furo[3,2-b]pyrrole-5-carboxylate) (1 equiv) and the amine (e.g., morpholine) (5.25 equiv) in DMF was stirred at 100° C. for 8 h. After cooling to rt, the reaction mixture was treated with H₂O and extracted with EtOAc (3 times). After separation of layers, the combined organic layer was washed with H₂O, and dried over MgSO₄. After filtration, the solution was concentrated in vacuo to give the desired product (e.g., 2-(4-morpholinobutanoyloxy)ethyl 4H-furo[3,2-b]pyrrole-5-carboxylate).

### General Procedure 9.1: Synthesis of pyrrole-5-carboxamide Derivatives

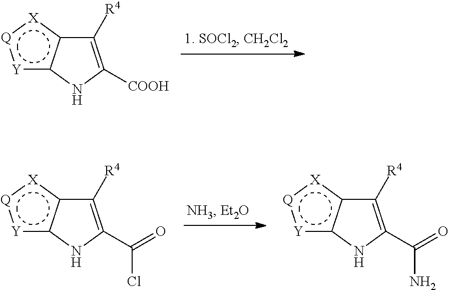

### 9.1.A) Synthesis of pyrrole-5-carbonyl chlorides

To a solution of a fused pyrrole carboxylic acid starting material (e.g., 4H-furo[3,2-b]pyrrole-5-carboxylic acid) (1 equiv) and a catalytic amount of DMF in DCM was added SOCl₂ (2-3 equiv) dropwise. The resulting mixture was stirred at 30° C. for 40 min, and then concentrated in vacuo to give the corresponding crude acid chloride (e.g., 4H-furo[3,2-b]pyrrole-5-carbonyl chloride).

### 9.1.B) Synthesis of pyrrole-5-carboxamide Derivatives

The crude acid chloride (e.g., 4H-furo[3,2-b]pyrrole-5-carbonyl chloride) was dissolved in diethyl ether, and NH₃ gas was bubbled into the diethyl ether solution. The progress of the reaction was monitored by LC-MS. After the completion of the reaction, the insoluble material was removed by filtration. The filter cake was rinsed with diethyl ether and ethyl acetate. The combined filtrate was concentrated to give the desired pyrrole-5-carboxamide derivative (e.g., 4H-furo[3,2-b]pyrrole-5-carboxamide).

### General Procedure 9.2: Synthesis of pyrrole-5-carboxamide Derivatives

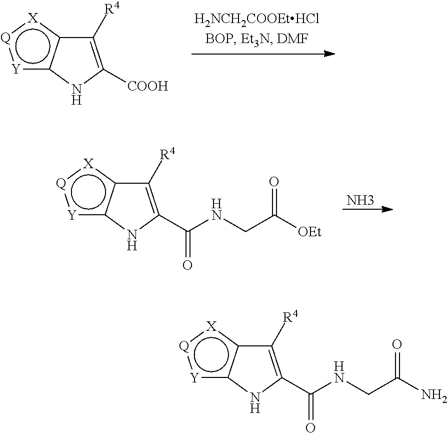

### 9.2.A) Synthesis of ethyl (pyrrole-5-carboxamido)acetate Derivatives

A solution of a fused pyrrole carboxylic acid starting material (e.g., 4H-furo[3,2-b]pyrrole-5-carboxylic acid) (1 equiv), ethyl 2-aminoacetate hydrochloride (2.4 equiv), BOP (1.17 equiv), and Et₃N (3.8 equiv) in DMF was stirred at 30-35° C. for 5 days. After cooling to rt, the reaction mixture was treated with H₂O and extracted with EtOAc (3 times). After separation of layers, the organic layer was washed with 5% Na₂CO₃ solution, H₂O (3 times), and dried over MgSO₄. After filtration, the solution was concentrated in vacuo to give the crude ethyl (pyrrole-5-carboxamido)acetate derivative (e.g., ethyl 2-(4H-furo[3,2-b]pyrrole-5-carboxamido)acetate). (Reference: J. Med. Chem., 2004, 47, 3892-3896.)

### 9.2.B) Synthesis of N-(2-amino-2-oxoethyl)-pyrrole-5-carboxamide derivatives

The crude ethyl (pyrrole-5-carboxamido)acetate derivative (e.g., ethyl 2-(4H-furo[3,2-b]pyrrole-5-carboxamido)acetate) was dissolved in anhydrous ethanol and heated to 60° C. Ammonia gas was bubbled into the reaction solution of alcohol for 3 h. After cooling to rt, the reaction mixture was stirred overnight and concentrated in vacuo to give the crude product. The crude product was dissolved in methanol. The resulting solution was poured into the mixture of Et₂O and hexane (3/1 ratio) and stirred for 2 h. After filtration, the filtrate was concentrated in vacuo to give the desired product (e.g., N-(2-amino-2-oxoethyl)-4H-furo[3,2-b]pyrrole-5-carboxamide).

### General Procedure 9.3: Synthesis of pyrrole-5-carboxamide derivatives

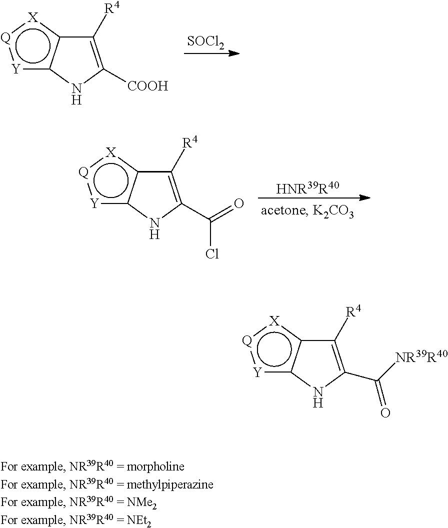

To a solution of crude acid chloride (e.g., 4H-furo[3,2-b]pyrrole-5-carbonyl chloride) (1 equiv) prepared as in General Procedure 9.1.A in acetone was added K₂CO₃ (1.1 equiv) and the amine (e.g., 1-methylpiperazine) (7.4 equiv). The resulting mixture was stirred at 30° C. for 21 h. The reaction mixture was filtered and the filtrate was concentrated in vacuo to give the crude product. The crude product was suspended in hexane and stirred for 3 h and then filtered. The solid product was collected by vacuum filtration and dried under vacuum to give the desired pyrrol-5-yl-(4-methylpiperazin-1-yl)methanone derivative (e.g., (4H-furo[3,2-b]pyrrol-5-yl)(4-methylpiperazin-1-yl)methanone).

### General Procedure 10.1: Synthesis of pyrrole N-substituted analogs

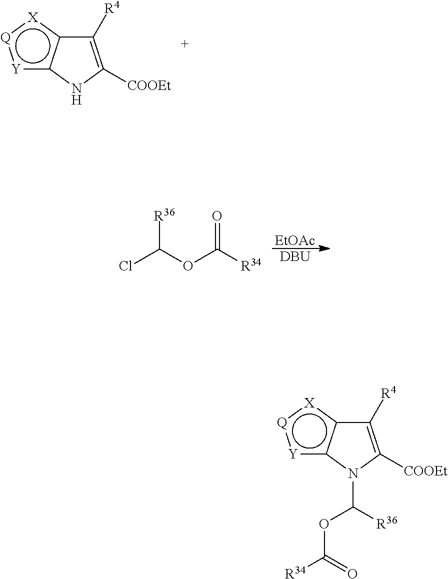

To a solution of a fused pyrrole carboxylic acid ethyl ester (e.g., ethyl 4H-furo[3,2-b]pyrrole-5-carboxylate) (0.57 mmol) and the 1-chloromethyl acyl ester (e.g., chloromethyl benzoate) (200 mg, 1.17 mmol) in EtOAc or THF (0.04-0.06 M in fused pyrrole carboxylic acid ethyl ester) was added DBU (150 mg) dropwise. In some examples, a catalytic amount of NaI was added. The reaction mixture was stirred at rt overnight. It was then treated with H₂O and extracted with EtOAc. The separated organic layer was dried over MgSO₄, filtered, and concentrated to give the crude product. The crude product was purified by column chromatography to give the desired product (e.g., ethyl 4-(benzoyloxymethyl)-4H-furo[3,2-b]pyrrole-5-carboxylate).

### General Procedure 10.2: Synthesis of pyrrole N-substituted analogs

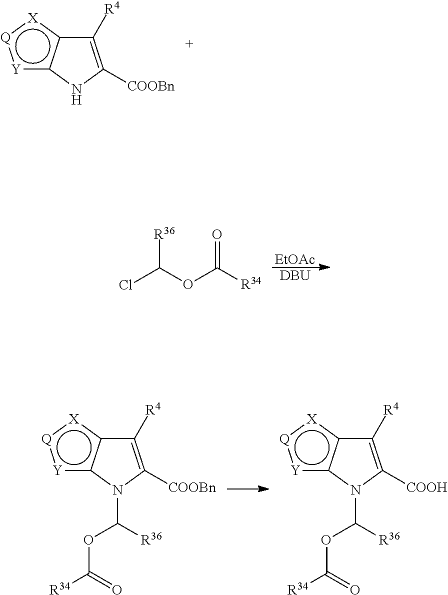

### 10.2.A) Synthesis of pyrrole N-substituted analogs as benzyl esters

The title compounds (e.g., benzyl 4-(benzoyloxymethyl)-4H-furo[3,2-b]pyrrole-5-carboxylate) were synthesized from the fused pyrrole carboxylic acid benzyl esters (e.g., benzyl 4H-furo[3,2-b]pyrrole-5-carboxylate) and the 1-chloromethyl acyl ester (e.g., chloromethyl benzoate) according to General Procedure 10.1.

### 10.2.B) Synthesis of pyrrole N-substituted analogs as carboxylic acids

The fused pyrrole carboxylic acid benzyl ester (e.g., benzyl 4H-furo[3,2-b]pyrrole-5-carboxylate) was dissolved in EtOAc. To the mixture was added one drop of 35% HCl solution and a catalytic amount of Pd/C. The reaction flask was evacuated and then back filled with hydrogen gas three times. The solution was stirred for 4 h under atmospheric pressure hydrogen. The reaction mixture was filtered through a pad of Celite and washed with methanol. The combined filtrate was concentrated and purified by preparative TLC or column chromatography to provide the desired pyrrole N-substituted analogs as carboxylic acid (e.g., 4-(benzoyloxymethyl)-4H-furo[3,2-b]pyrrole-5-carboxylic acid).

### General Procedure 10.3: Synthesis of pyrrole N-substituted analogs

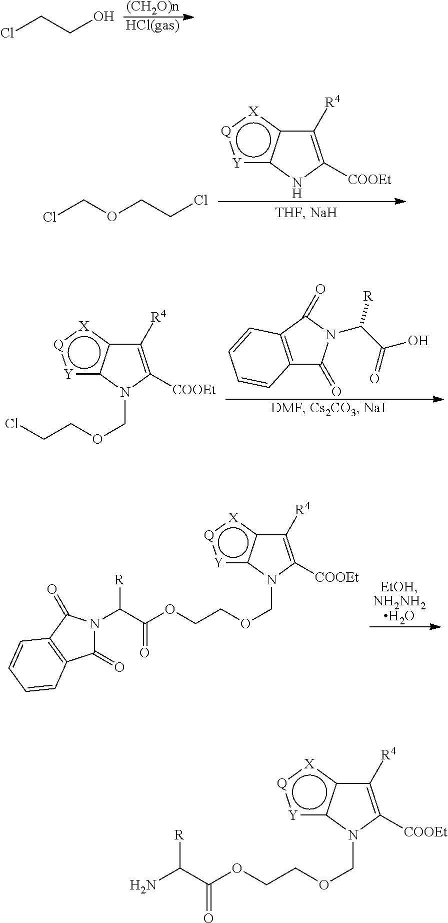

### 10.3.A) Synthesis of 1-chloro-2-(chloromethoxy)ethane

A suspension of 2-chloroethanol (0.65 g, 10 mmol) and paraformaldehyde (0.7 g) in dry methylene chloride (18 mL) was cooled to 0° C. Dry hydrogen chloride gas was bubbled through the stirred suspension for 3 h until saturated. The mixture was allowed to stand in a refrigerator overnight and then dried over anhydrous CaCl₂. The reaction mixture was filtered and the solvent was removed under reduced pressure to give the title compound as an oily residue, which was used directly in the next step.

### 10.3.B) Synthesis of (2-chloroethoxy)methyl-substituted fused pyrrole carboxylic acid ethyl esters

To a suspension of the fused pyrrole carboxylic acid ethyl ester (e.g., ethyl 4H-furo[3,2-b]pyrrole-5-carboxylate) (2.23 mmol) and NaH (0.31 g, 7.75 mmol) in THF was added 1-chloro-2-(chloromethoxy)ethane (1.0 g, 7.81 mmol) in THF. The reaction mixture was stirred at rt for 2 h, quenched with a saturated NH₄Cl solution, and extracted with EtOAc three times. The combined organic layer was dried over Na₂SO₄ and concentrated in vacuo. The residue was purified by column chromatography to afford the desired compound (e.g., ethyl 4-((2-chloroethoxy)methyl)-4H-furo[3,2-b]pyrrole-5-carboxylate).

### 10.3.C) Synthesis of pyrrole N-substituted analog intermediates

A solution of the (2-chloroethoxy)methyl-substituted fused pyrrole carboxylic acid ethyl ester (e.g., ethyl 4-((2-chloroethoxy)methyl)-4H-furo[3,2-b]pyrrole-5-carboxylate) (3.2 mmol), the protected amino acid (e.g., (R)-2-(1,3-dioxoisoindolin-2-yl)-3-methylbutanoic acid) (2.4 mmol.), Cs₂CO₃ (0.789 g, 2.4 mmol), and a catalytic amount of sodium iodide in DMF (20 mL) was heated at 110° C. overnight. After cooling to rt, the insoluble solid was removed by filtration. The filtrate was poured into water and extracted with ethyl acetate (3 times). The combined organic layer was dried over Na₂SO₄ and concentrated in vacuo. The residue oil was purified by column chromatography to afford the desired product (e.g., (R)-ethyl 4-((2-(2-(1,3-dioxoisoindolin-2-yl)-3-methylbutanoyloxy)ethoxy)methyl)-4H-furo[3,2-b]pyrrole-5-carboxylate).

### 10.3.D) Synthesis of (2-chloroethoxy)methyl-substituted fused pyrrole carboxylic acid ethyl esters

NH₂NH₂.H₂O (0.5 g) was added to a solution of the starting material (e.g., (R)-ethyl 4-((2-(2-(1,3-dioxoisoindolin-2-yl)-3-methylbutanoyloxy)ethoxy)methyl)-4H-furo[3,2-b]pyrrole-5-carboxylate) (0.42 mmol) in EtOH (20 mL). The reaction mixture was heated to 60° C. for 1 h. After cooling to rt, the solid was removed by filtration and the filtrate was concentrated under reduced pressure. The residual oil was purified by column chromatography to afford the desired product (e.g., (R)-ethyl 4-((2-(2-amino-3-methylbutanoyloxy)ethoxy)methyl)-4H-furo[3,2-b]pyrrole-5-carboxylate).

### General Procedure 11: Plasma stability—Conversion of Prodrugs to the Parent Acid

Plasma (mouse, rat, dog, monkey, or human) stocks were warmed to rt, then 1 mL was removed and added to 5 μL of a 1.0 mM DMSO solution of the test compound. 60 μL aliquots were removed at pre-determined time points (e.g., 0, 5, 15, 30, 60, 120 min), added to a 96 deep well plate pre-loaded with 300 μL of CH₃CN, vortexed for 10 seconds, and then immediately analyzed for the amount of parent acid at each time point as determined using a SCIEX API2000 LC/MS/MS.

### General Procedure 12: Pharmacokinetic-Pharmacodynamic Evaluation of Compounds in Rats

Rats (Harlan male Sprague) (N=3) were dosed orally with an amount of test compound, suspended in 45% (w/v) hydroxy-β-cyclodextrin vehicle, dose normalized to 10 mg/kg of the corresponding carboxylic acid. Additional male cannulated rats (N=3) were dosed orally with the parent carboxylic acid, suspended in 45% (w/v) hydroxy-β-cyclodextrin vehicle, at 10 mg/kg. Animals were sacrificed at pre-determined time points (e.g., 0.5, 2 and/or 6 hours post administration) with an N=3 at each time point. At sacrifice, trunk blood was collected into tubes containing potassium EDTA, which were then centrifuged to permit isolation of plasma. The cerebellum was dissected from each animal. Plasma and cerebellum samples were stored at −80° C. until samples were analyzed using a SCIEX API2000 or SCIEX API5000 LC/MS/MS. Samples were analyzed for the amount of corresponding parent acid, D-serine, and/or the amount of test compound.

### General Procedure 13: Pharmacokinetic-Pharmacodynamic Evaluation of Compounds in Cannulated Rats

Male cannulated rats (N=4) (Charles River CD) were dosed orally with an amount of test compound, suspended in 45% (w/v) hydroxy-β-cyclodextrin vehicle, dose normalized to 5 mg/kg of the corresponding carboxylic acid. Additional male cannulated rats (N=4) were dosed both orally and by i.v. administration with the parent carboxylic acid, suspended in 45% (w/v) hydroxy-β-cyclodextrin vehicle, at 5 mg/kg. Plasma samples were removed from a cannula at pre-determined time points (e.g., 0.0833, 0.5, 1, 3, 6, and 24 hours post administration), and were collected into tubes containing potassium EDTA, which were then centrifuged to permit isolation of plasma. Plasma samples were stored at −80° C. until samples were analyzed using a SCIEX API2000 or SCIEX API5000 LC/MS/MS. Samples were analyzed for the amount of corresponding parent acid, D-serine, and/or the amount of test compound.

### General Procedure 14: Hydrolysis Rate of Test Compounds in pH 7.4 and 2.0 Aqueous Solution

10 mM DMSO solutions of test compounds were diluted with a 0.05 M sodium phosphate buffer (pH 7.4) or water adjusted with phosphoric acid to pH 2.0, with a final concentration of 0.013 mg/mL. Samples were analyzed over either 2 or 6 hours, by HPLC.

The amount of test compound and the corresponding acid were determined. The rate of conversion of the test compound was calculated based on formation of the corresponding acid and loss of parent.

### Example 1

### Synthesis of fused pyrrole[1,2-c]oxazol-1(3H)-one Derivatives

### 1.1 Synthesis of 4-ethyl-1,5-dioxa-3b-aza-cyclopenta[a]pentalen-6-one (1)

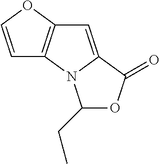

The title compound was synthesized from 4H-furo[3,2-b]pyrrole-5-carboxylic acid according to General Procedure 1. LC-MS: m/z=192 (M+1).

### 1.2 Synthesis of 4,4-dimethyl-1,5-dioxa-3b-aza-cyclopenta[a]pentalen-6-one (2)

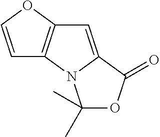

The title compound was synthesized from 4H-furo[3,2-b]pyrrole-5-carboxylic acid according to General Procedure 1. LC-MS: m/z=192 (M+1).

### 1.3 Synthesis of 4-methyl-1,5-dioxa-3b-aza-cyclopenta[a]pentalen-6-one (3)

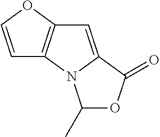

The title compound was synthesized from 4H-furo[3,2-b]pyrrole-5-carboxylic acid and acetaldehyde according to General Procedure 1. LC-MS: m/z=178 (M+1).

### Example 2

### Synthesis of Alkyl Ester and Aryl Ester Derivatives

### 2.1 Synthesis of propyl 4H-furo[3,2-b]pyrrole-5-carboxylate (4)

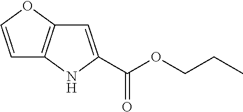

The title compound was synthesized from 4H-furo[3,2-b]pyrrole-5-carboxylic acid according to General Procedure 2.1. LC-MS: m/z=194 (M+1).

### 2.2 Synthesis of butyl 4H-furo[3,2-b]pyrrole-5-carboxylate (5)

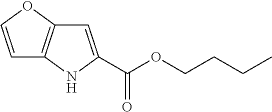

The title compound was synthesized from 4H-furo[3,2-b]pyrrole-5-carboxylic acid according to General Procedure 2.1. LC-MS: m/z=208 (M+1).

### 2.3 Synthesis of heptyl 4H-furo[3,2-b]pyrrole-5-carboxylate (6)

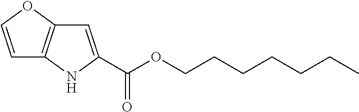

The title compound was synthesized from 4H-furo[3,2-b]pyrrole-5-carboxylic acid according to General Procedure 2.1. LC-MS: m/z=250 (M+1); ¹H NMR (CDCl₃, 500 MHz) δ7.52 (s, 1H), 6.80 (s, 1H), 6.46 (s, 1H), 4.32-4.28 (t, 2H), 1.92-1.72 (m, 2H), 1.57-1.29 (m, 8H), 0.98-0.88 (t, 3H).

### 2.4 Synthesis of isobutyl 4H-furo[3,2-b]pyrrole-5-carboxylate (7)

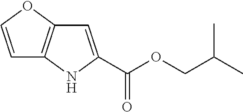

The title compound was synthesized from 4H-furo[3,2-b]pyrrole-5-carboxylic acid according to General Procedure 2.1. LC-MS: m/z=208 (M+1).

### 2.5 Synthesis of 4-methylpentyl 4H-furo[3,2-b]pyrrole-5-carboxylate (8)

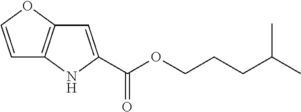

The title compound was synthesized from 4H-furo[3,2-b]pyrrole-5-carboxylic acid according to General Procedure 2.1. LC-MS: m/z=236 (M+1).

### 2.6 Synthesis of benzyl 4H-furo[3,2-b]pyrrole-5-carboxylate (9)

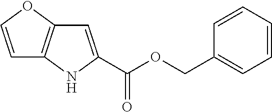

The title compound was synthesized from 4H-furo[3,2-b]pyrrole-5-carboxylic acid according to General Procedure 2.1. LC-MS: m/z=242 (M+1).

### 2.7 Synthesis of (tetrahydrofuran-2-yl)methyl 4H-furo[3,2-b]pyrrole-5-carboxylate (10)

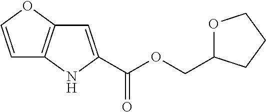

The title compound was synthesized from 4H-furo[3,2-b]pyrrole-5-carboxylic acid (90 g, 0.596 mol) and 2-(bromomethyl)tetrahydrofuran (98.3 g, 0.596 mol) according to General Procedure 2.2. The crude product was crystallized (EtOAc:n-hexane=1:4) to produce the desired product (50.8 g). LC-MS: MS m/z: 236 (M+1).

### 2.8 Synthesis of pyridin-4-ylmethyl 4H-furo[3,2-b]pyrrole-5-carboxylate (11)

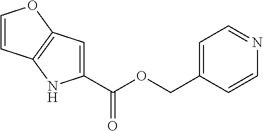

The title compound was synthesized from 4H-furo[3,2-b]pyrrole-5-carboxylic acid (100 mg, 0.66 mmol) and pyridin-4-ylmethanol according to General Procedure 2.1. LC-MS: m/z=236 (M+1). The crude product was purified by prepared TLC to give the desired product (26.8 mg). LC-MS: MS m/z: 243 (M+1).

### 2.9 Synthesis of 2-methoxyphenyl 4H-furo[3,2-b]pyrrole-5-carboxylate (12)

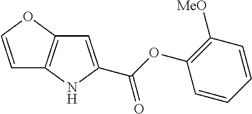

The title compound was synthesized from 4H-furo[3,2-b]pyrrole-5-carbonyl chloride (General Procedure 2.3.B) (170 mg, 1 mmol) and sodium 2-methoxyphenolate (General Procedure 2.3.A) (146 mg, 1 mmol) according to General Procedure 2.3.C. The crude product was purified by column chromatography to give the desired product (73.1 mg). LC-MS: MS m/z: 258 (M+1).

### 2.10 Synthesis of isopentyl 4H-furo[3,2-b]pyrrole-5-carboxylate (13)

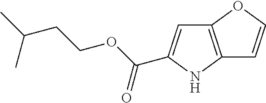

The title compound was synthesized from 4H-furo[3,2-b]pyrrole-5-carboxylic acid (200 mg, 1.32 mmol) and 3-methylbutan-1-ol (233 mg) according to General Procedure 2.4. The crude product was purified by column chromatography to give the desired product (80 mg). LC-MS: MS m/z: 222 (M+1).

### 2.11 Synthesis of isopropyl 4H-furo[3,2-b]pyrrole-5-carboxylate (14)

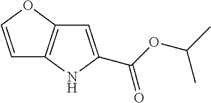

The title compound was synthesized from 4H-furo[3,2-b]pyrrole-5-carboxylic acid and propan-2-ol according to General Procedure 2.4. LC-MS: MS m/z: 194 (M+1).

### 2.12 Synthesis of pentyl 4H-furo[3,2-b]pyrrole-5-carboxylate (15)

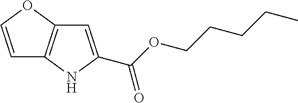

The title compound was synthesized from 4H-furo[3,2-b]pyrrole-5-carboxylic acid and pentan-1-ol according to General Procedure 2.4. LC-MS: MS m/z: 222 (M+1).

### 2.13 Synthesis of tert-butyl 4H-furo[3,2-b]pyrrole-5-carboxylate (16)

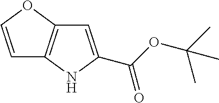

The title compound was synthesized from 4H-furo[3,2-b]pyrrole-5-carboxylic acid and 2-methylpropan-2-ol according to General Procedure 2.4. LC-MS: MS m/z: 208 (M+1).

### 2.14 Synthesis of cyclohexylmethyl 4H-furo[3,2-b]pyrrole-5-carboxylate (17)

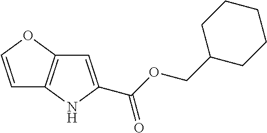

The title compound was synthesized from 4H-furo[3,2-b]pyrrole-5-carboxylic acid and cyclohexylmethanol according to General Procedure 2.4. LC-MS: MS m/z: 248 (M+1).

### Example 3

### Synthesis of Amino Acid Derived Ester Derivatives

### 3. Synthesis of 3-(4H-furo[3,2-b]pyrrole-carbonyloxy)-2-aminopropanoic acid trifluoroacetic acid salt (18)

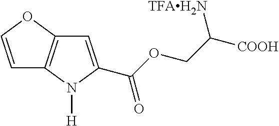

The title compound was synthesized from 4H-furo[3,2-b]pyrrole-5-carboxylic acid according to General Procedure 3. LC-MS: m/z=239 (M+1)

### Example 4

### Synthesis of Glycolamide Ester Derivatives

### 4.1. Synthesis of 2-(azetidin-1-yl)-2-oxoethyl 4H-furo[3,2-b]pyrrole-5-carboxylate (19)

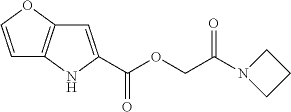

### 4.1.1 Synthesis of 1-(azetidin-1-yl)-2-chloroethanone

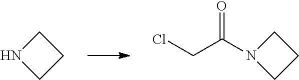

1-(azetidin-1-yl)-2-chloroethanone was synthesized from azetidine (120 mg, 2.105 mmol) and chloroacetyl chloride (250 mg, 2.212 mmol) according to General Procedure 4.A to afford the title compound as a light yellow liquid (200 mg).

### 4.1.2 Synthesis of 2-(azetidin-1-yl)-2-oxoethyl 4H-furo[3,2-b]pyrrole-5-carboxylate (19)

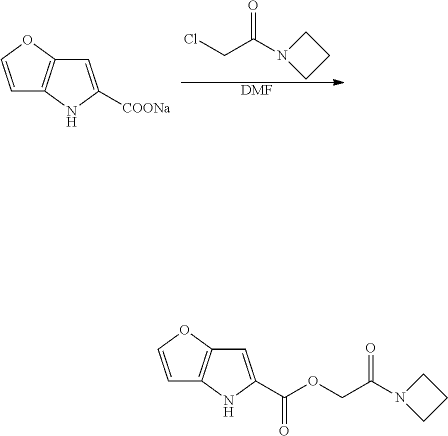

The title compound was synthesized from sodium 4H-furo[3,2-b]pyrrole-5-carboxylate and 1-(azetidin-1-yl)-2-chloroethanone according to General Procedure 4.C. LC-MS: m/z=249 (M+1).

### 4.2 Synthesis of 2-(dimethylamino)-2-oxoethyl 4H-furo[3,2-b]pyrrole-5-carboxylate (20)

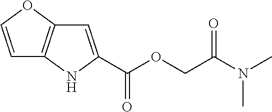

The title compound was synthesized from sodium 4H-furo[3,2-b]pyrrole-5-carboxylate and 2-chloro-N,N-dimethylacetamide according to General Procedure 4.C. LC-MS: m/z=237 (M+1).

### 4.3 Synthesis of 2-morpholino-2-oxoethyl 4H-furo[3,2-b]pyrrole-5-carboxylate (21)

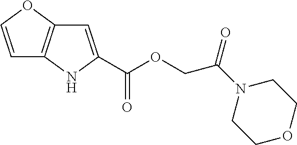

The title compound was synthesized from sodium 4H-furo[3,2-b]pyrrole-5-carboxylate and 2-chloro-1-morpholinoethanone according to General Procedure 4.C. LC-MS: m/z=265 (M+1) ; ¹H NMR (DMSO-d₆, 500 MHz) δ 11.68 (s, 1H), 7.81-7.80 (s, 1H), 6.79 (s, 1H), 6.62 (s, 1H), 4.97 (d, 2H), 3.61-3.58 (d, 4H), 3.44 (s, 1H).

### 4.4 Synthesis of 2-(dimethylamino)-2-oxoethyl 4H-furo[3,2-b]pyrrole-5-carboxylate (22)

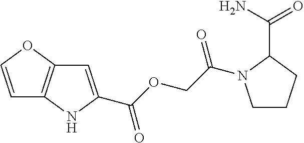

### 4.4.1 Synthesis of 1-(2-chloroacetyl)pyrrolidine-2-carboxylic acid

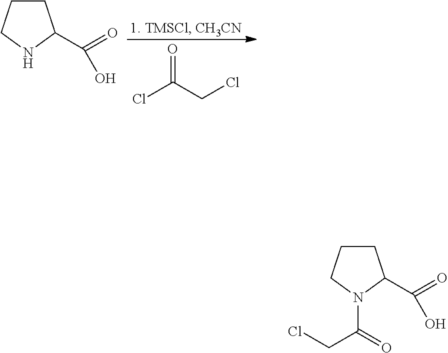

The title compound was synthesized from L-proline (1.15 g, 10 mmol, in dry CH₃CN (5 mL)) and chloroacetyl chloride (0.8 mL, 10 mmol) according to General Procedure 4.B. The crude product (yellow oil) was used directly in the next step without further purification.

### 4.4.2 Synthesis of 1-(2-chloroacetyl)pyrrolidine-2-carboxamide

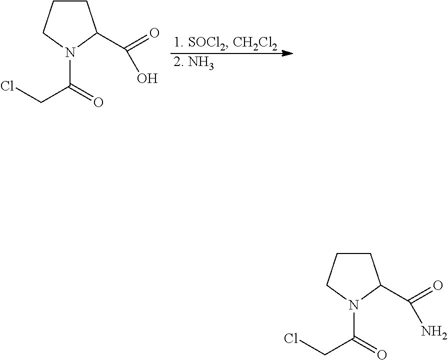

The above crude 1-(2-chloroacetyl)pyrrolidine-2-carboxylic acid (0.828 g, 4.3 mmol) was suspended in DCM and cooled to 0° C. SOCl₂ (4 mL, 54.8 mmol) was added dropwise over 10 min, and the resulting solution was warmed to rt while stirring. After 1 h at rt, the LC/MS indicated that the reaction was complete. NH₃ gas was then bubbled into the reaction solution for 2 h. The mixture was concentrated under reduced pressure to yield the title compound as a yellow solid (0.35 g). LC-MS: m/z=191 (M+1).

### 4.4.3 Synthesis of 2-(2-carbamoylpyrrolidin-1-yl)-2-oxoethyl 4H-furo[3,2-b]pyrrole-5-carboxylate (22)

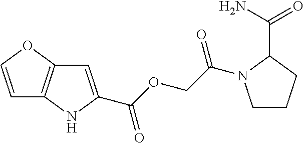

The title compound was synthesized from sodium 4H-furo[3,2-b]pyrrole-5-carboxylate according to General Procedure 4.C. LC-MS: m/z=306 (M+1).

### 4.5 Synthesis of 2-(diethylamino)-2-oxoethyl 4H-furo[3,2-b]pyrrole-5-carboxylate (23)

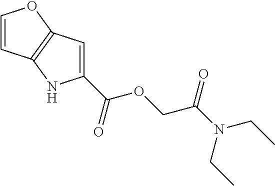

The title compound was synthesized from sodium 4H-furo[3,2-b]pyrrole-5-carboxylate (169.9 mg, 0.98 mmol) and 2-chloro-N,N-diethylacetamide (148.6 mg, 0.99 mmol according to General Procedure 4.C. LC-MS: MS m/z: 267 (M+1).

### 4.6 Synthesis of 2-oxo-2-(pyrrolidin-1-yl)ethyl 4H-furo[3,2-b]pyrrole-5-carboxylate (24)

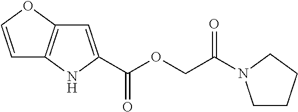

The title compound was synthesized from sodium 4H-furo[3,2-b]pyrrole-5-carboxylate according to General Procedure 4.C. LC-MS: MS m/z: 263 (M+1).

### 4.7 Synthesis of 2-oxo-2-(piperidin-1-yl)ethyl 4H-furo[3,2-b]pyrrole-5-carboxylate (25)

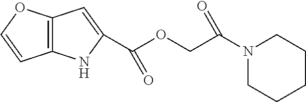

The title compound was synthesized from sodium 4H-furo[3,2-b]pyrrole-5-carboxylate according to General Procedure 4.C. LC-MS: MS m/z: 277 (M+1).

### 4.8 Synthesis of 2-(4-methylpiperazin-1-yl)-2-oxoethyl 4H-furo[3,2-b]pyrrole-5-carboxylate (26)

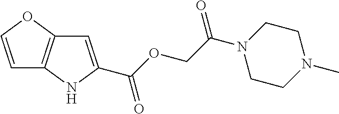

The title compound was synthesized from sodium 4H-furo[3,2-b]pyrrole-5-carboxylate according to General Procedure 4.C. LC-MS: MS m/z: 292 (M+1).

### Example 5

### Synthesis of Morpholino- and Imidazolino Ester Derivatives

### 5.1 Synthesis of 2-(1H-imidazol-1-yl)ethyl 4H-furo[3,2-b]pyrrole-5-carboxylate (27)

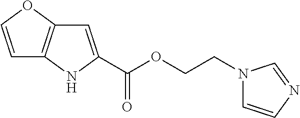

### 5.1.1 Synthesis of 2-chloroethylimidazole

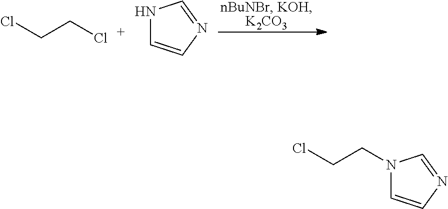

To a stirred mixture of 1,2-dichloroethane (80 mL), tetrabutylammonium bromide (0.21 g, 0.64 mmol), KOH (11.2 g, 200 mmol), K₂CO₃ (8.84 g, 64 mmol) was added imidazole (2.04 g, 30 mmol). The resulting reaction mixture was stirred at 45-50° C. for 5 h. After cooling, the insoluble was filtered off. The organic solution was washed with water (2×25 mL) and dried over anhydrous Na₂SO₄. After filtration, the filtrate was concentrated to give the desired product as an oil (3.09 g), which was used directly for the next step without further purification. LC-MS: m/z=131 (M+1).

### 5.1.2 Synthesis of 2-(1H-imidazol-1-yl)ethyl 4H-furo[3,2-b]pyrrole-5-carboxylate (27)

The title compound was synthesized from sodium 4H-furo[3,2-b]pyrrole-5-carboxylate and 2-chloroethylimidazole according to General Procedure 5. LC-MS: m/z=246 (M+1).

### 5.2 Synthesis of (1H-imidazol-1-yl)methyl 4H-furo[3,2-b]pyrrole-5-carboxylate (28)

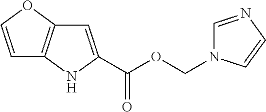

### 5.2.1 Synthesis of 1-hydromethylimidazole

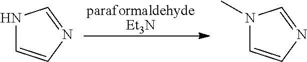

A mixture of imidazole (3 g, 44 mmol), paraformaldehyde (1.46 g, 49 mmol), and 2-3 drops of triethylamine were stirred at 80° C. until the solid completely melted to form a viscous oil. The oil mixture was then cooled to rt and allowed to solidify to give the titled product (3.5 g) as a white solid. LC-MS: m/z=99 (M+1).

### 5.2.2 Synthesis of chloromethylimidazole

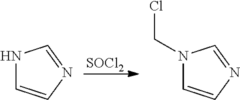

To a solution of 1-hydromethylimidazole (1 g, 10.2 mmol) in anhydrous 1,4-dixanone (20 mL) was added SOCl₂(3.64 g, 30.6 mmol) at rt. After 2 h, the mixture was evaporated to dryness. The resulting crude product (2.5 g) was directly used for the next step without further purification. LC-MS: m/z=117 (M+1); ¹H NMR (CDCl₃, 500 MHz) δ 7.88 (br s, 1H), 7.82 (br s, 1H), 6.95 (s, 1H), 6.79 (s, 1H), 6.79 (br s, 1H), 6.15 (s, 2H).

### 5.2.3 Synthesis of (1H-imidazol-1-yl)methyl 4H-furo[3,2-b]pyrrole-5-carboxylate (28)

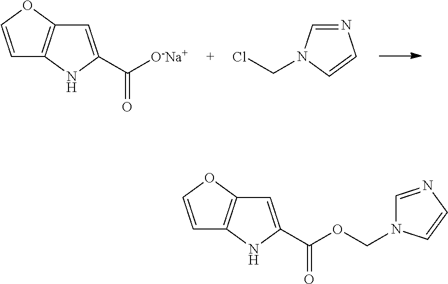

The title compound was synthesized from sodium 4H-furo[3,2-b]pyrrole-5-carboxylate and 1-chloromethylimidazole according to General Procedure 5. LC-MS: m/z=232 (M+1).

### 5.3 Synthesis of 2-(2-methyl-1H-imidazol-1-yl)ethyl 4H-furo[3,2-b]pyrrole-5-carboxylate (29)

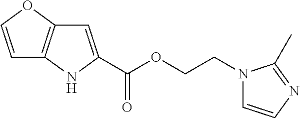

### 5.3.1 Synthesis of 2-chloroethylimidazole

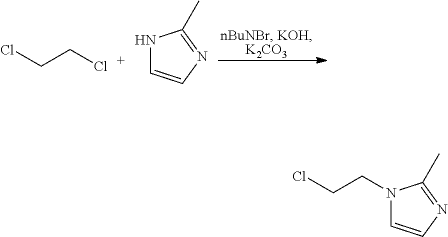

A mixture of 2-methyl-1H-imidazole (1 g, 12.35 mmol), Bu₄NBr (0.1 g), KOH (4.6 g), and K₂CO₃ (3.6 g) in 1,2-dichloroethane (40 mL) was stirred at 45° C. for 6.5 h. After cooling to rt, the mixture was filtered. The filtrate was washed with brine, dried over Na₂SO₄, and concentrated to give the crude product. The crude product was used for the next step without further purification. LC-MS: MS m/z: 145 (M+1).

### 5.3.2 Synthesis of 2-(2-methyl-1H-imidazol-1-yl)ethyl 4H-furo[3,2-b]pyrrole-5-carboxylate (29)

The title compound was synthesized from sodium 4H-furo[3,2-b]pyrrole-5-carboxylate and 2-chloroethylimidazole according to General Procedure 5. The crude product was crystallized from EtOAc and Et₂O to give the desired product as white solid. LC-MS: MS m/z: 260 (M+1).

### Example 6

### Synthesis of 2-morpholinoethyl 4H-furo[3,2-b]pyrrole-5-carboxylate Analogs

### 6.1 Synthesis of 2-morpholinoethyl 4H-furo[3,2-b]pyrrole-5-carboxylate (30)

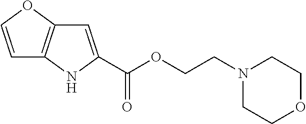

The title compound was synthesized from sodium 4H-furo[3,2-b]pyrrole-5-carboxylate and N-(2-chloroethyl)morpholine hydrochloride according to General Procedure 6. LC-MS: m/z=265(M+1).

### 6.2 Synthesis of 2-(diethylamino)ethyl 4H-furo[3,2-b]pyrrole-5-carboxylate (31)

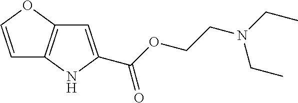

The title compound was synthesized from sodium 4H-furo[3,2-b]pyrrole-5-carboxylate (200 mg, 1.16 mmol) and 2-chloro-N,N-diethylethanamine hydrochloride (300 mg, 1.74 mmol) according to General Procedure 6. LC-MS: m/z=251(M+1).

### 6.3 Synthesis of 2-(dimethylamino)ethyl 4H-furo[3,2-b]pyrrole-5-carboxylate (32)

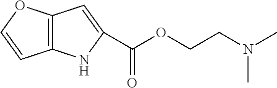

The title compound was synthesized from sodium 4H-furo[3,2-b]pyrrole-5-carboxylate and 2-chloro-N,N-dimethylethanamine according to General Procedure 6. LC-MS: m/z=223(M+1).

### Example 7

### Synthesis of 1-acetoxyethyl 4H-furo[3,2-b]pyrrole-5-carboxylate Analogs

### 7.1 Synthesis of 1-acetoxyethyl 4H-furo[3,2-b]pyrrole-5-carboxylate (33)

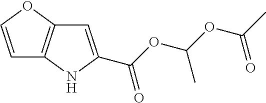

### 7.1.1 Synthesis of 1-chloroethyl acetate

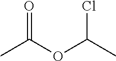

The title compound was synthesized from acetyl chloride according to General Procedure 7.A.

### 7.1.2 Synthesis of 1-acetoxyethyl 4H-furo[3,2-b]pyrrole-5-carboxylate (33)

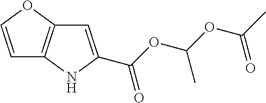

The title compound was synthesized from 4H-furo[3,2-b]pyrrole-5-carboxylic acid and 1-chloroethyl acetate according to General Procedure 7.D. LC-MS: m/z=238 (M+1).

### 7.2 Synthesis of 1-(propionyloxy)ethyl 4H-furo[3,2-b]pyrrole-5-carboxylate (34)

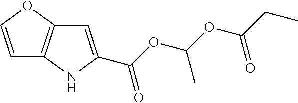

### 7.2.1 Synthesis of 1-chloroethyl propionate

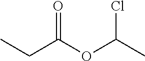

The title compound was synthesized from propionyl chloride according to General Procedure 7.A.

### 7.2.2 Synthesis of 1-(propionyloxy)ethyl 4H-furo[3,2-b]pyrrole-5-carboxylate (34)

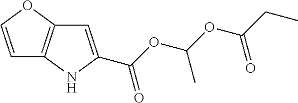

The title compound was synthesized from 4H-furo[3,2-b]pyrrole-5-carboxylic acid and 1-chloroethyl propionate according to General Procedure 7.D. LC-MS: m/z=252 (M+1); ¹H NMR (CDCl₃, 500 MHz) δ 7.54 (s, 1H), 7.14-7.10 (m, 1H), 6.84 (s, 1H), 6.47 (s, 1H), 2.39-2.34 (m, 2H), 1.60-1.55 (d, 3H), 1.16-1.13 (m, 3H).

### 7.3 Synthesis of 1-(isobutyryloxy)ethyl 4H-furo[3,2-b]pyrrole-5-carboxylate (35)

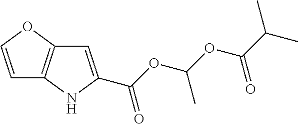

### 7.3.1 Synthesis of 1-chloroethyl isobutyrate

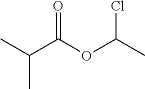

The title compound was synthesized from isobutyryl chloride according to General Procedure 7.A.

### 7.3.2 Synthesis of 1-(isobutyryloxy)ethyl 4H-furo[3,2-b]pyrrole-5-carboxylate (35)

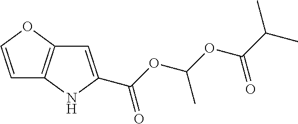

The title compound was synthesized from 4H-furo[3,2-b]pyrrole-5-carboxylic acid and 1-chloroethyl isobutyrate according to General Procedure 7.D. LC-MS: m/z=266 (M+1).

### 7.4 Synthesis of 1-(isopropoxycarbonyloxy)ethyl 4H-furo[3,2-b]pyrrole-5-carboxylate (36)

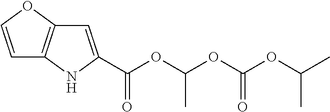

### 7.4.1 Synthesis of 1-chloroethyl isopropyl carbonate

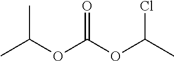

The title compound was synthesized from 1-chloroethyl carbonochloridate according to General Procedure 7.C.

### 7.4.2 Synthesis of 1-(isopropoxycarbonyloxy)ethyl 4H-furo[3,2-b]pyrrole-5-carboxylate (36)

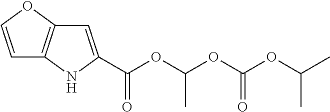

The title compound was synthesized from 4H-furo[3,2-b]pyrrole-5-carboxylic acid and 1-chloroethyl isopropyl carbonate according to General Procedure 7.D. LC-MS: m/z=282 (M+1).

### 7.5 Synthesis of 1-(tert-butoxycarbonyloxy)ethyl 4H-furo[3,2-b]pyrrole-5-carboxylate (37)

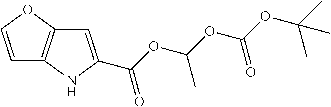

### 7.5.1 Synthesis of tert-butyl 1-chloroethyl carbonate

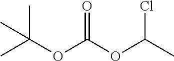

The title compound was synthesized from 1-chloroethyl carbonochloridate according to General Procedure 7.C.

### 7.5.2 Synthesis of 1-(tert-butoxycarbonyloxy)ethyl 4H-furo[3,2-b]pyrrole-5-carboxylate (37)

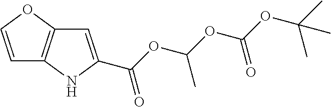

The title compound was synthesized from 4H-furo[3,2-b]pyrrole-5-carboxylic acid and tert-butyl 1-chloroethyl carbonate according to General Procedure 7.D. LC-MS: m/z=296 (M+1).

### 7.6 Synthesis of acetoxymethyl 4H-furo[3,2-b]pyrrole-5-carboxylate (38)

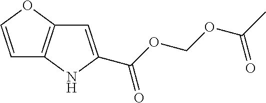

### 7.6.1 Synthesis of chloromethyl acetate

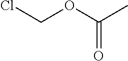

The title compound was synthesized from acetyl chloride according to General Procedure 7.B.

### 7.6.2 Synthesis of acetoxymethyl 4H-furo[3,2-b]pyrrole-5-carboxylate (38)

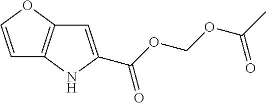

The title compound was synthesized from 4H-furo[3,2-b]pyrrole-5-carboxylic acid and chloromethyl acetate according to General Procedure 7.D. LC-MS: m/z=224 (M+1).

### 7.7 Synthesis of 1-(cyclohexyloxycarbonyloxy)ethyl 4H-furo[3,2-b]pyrrole-5-carboxylate (39)

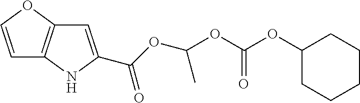

### 7.7.1 Synthesis of 1-chloroethyl cyclohexyl carbonate

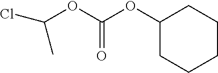

The title compound was synthesized from 1-chloroethyl carbonochloridate according to General Procedure 7.C.

### 7.7.2 Synthesis of 1-(cyclohexyloxycarbonyloxy)ethyl 4H-furo[3,2-b]pyrrole-5-carboxylate (39)

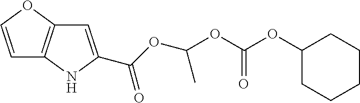

The title compound was synthesized from 4H-furo[3,2-b]pyrrole-5-carboxylic acid and 1-chloroethyl cyclohexyl carbonate according to General Procedure 7.D. LC-MS: m/z=322 (M+1).

### 7.8 Synthesis of 1-acetoxy-2-methylpropyl 4H-furo[3,2-b]pyrrole-5-carboxylate (40)

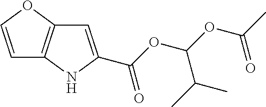

### 7.8.1 Synthesis of 1-chloro-2-methylpropyl acetate

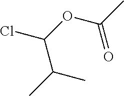

The title compound was synthesized from acetyl chloride according to General Procedure 7.A.

### 7.8.2 Synthesis of 1-acetoxy-2-methylpropyl 4H-furo[3,2-b]pyrrole-5-carboxylate (40)

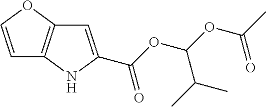

The title compound was synthesized from 4H-furo[3,2-b]pyrrole-5-carboxylic acid and 1-chloro-2-methylpropyl acetate according to General Procedure 7.D. LC-MS: m/z=266 (M+1).

### 7.9 Synthesis of methylene bis(4H-furo[3,2-b]pyrrole-5-carboxylate) (41)

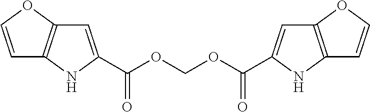

### 7.9.1 Synthesis of chloromethyl 4H-furo[3,2-b]pyrrole-5-carboxylate (76)

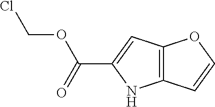

To a solution of sodium 4H-furo[3,2-b]pyrrole-5-carboxylate (346 mg, 1 mmol), Na₂CO₃ (848 mg, 4 mmol), water (15 mL), and (n-Bu)₄NHSO₄ (0.6 mg) in DCM (30 mL) at 0° C. was added chloromethyl sulfochloridate (0.28 ml, 1.53 mmol). After stirred at 0° C. for 0.5 h, the reaction mixture was stirred at rt for 18 h. The mixture was then extracted with DCM, dried over Na₂SO₄. The filtrate was concentrated and purified by prepared TLC to give the title compound. LC-MS: MS m/z: 200 (M+1).

### 7.9.2 Synthesis of 1-acetoxy-2-methylpropyl 4H-furo[3,2-b]pyrrole-5-carboxylate (41)

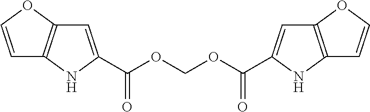

The title compound was synthesized from 4H-furo[3,2-b]pyrrole-5-carboxylic acid (53.8 mg, 0.356 mmol), chloromethyl 4H-furo[3,2-b]pyrrole-5-carboxylate (78 mg, 0.392 mmol), NaI (5 mg), Et₃N (72 mg) in 1,4-dioxane (10 mL) according to General Procedure 7.D. LC-MS: m/z=315 (M+1).

### 7.10 Synthesis of propionyloxymethyl 4H-furo[3,2-b]pyrrole-5-carboxylate (42)

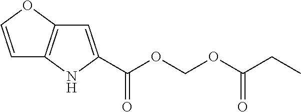

### 7.10.1 Synthesis of chloromethyl propionate

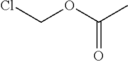

The title compound was synthesized from propionyl chloride (4 mL, 2.88 mmol) and paraformaldehyde according to General Procedure 7.B. The crude product was used directly in the next step without further purification.

### 7.10.2 Synthesis of propionyloxymethyl 4H-furo[3,2-b]pyrrole-5-carboxylate (42)

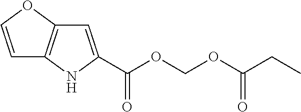

The title compound was synthesized from 4H-furo[3,2-b]pyrrole-5-carboxylic acid (300 mg, 2 mmol), chloromethyl propionate (294 mg, 2.4 mmol), catalytic NaI, Et₃N (303 mg, 3 mmol) in 1,4-dioxane (25 mL) according to General Procedure 7D. LC-MS: m/z=238 (M+1).

### 7.11 Synthesis of butyryloxymethyl 4H-furo[3,2-b]pyrrole-5-carboxylate (43)

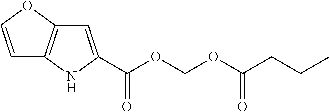

### 7.11.1 Synthesis of chloromethyl butyrate

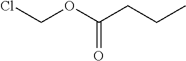

The title compound was synthesized from butyryl chloride and paraformaldehyde according to General Procedure 7.B. The crude product was used directly in the next step without further purification.

### 7.11.2 Synthesis of butyryloxymethyl 4H-furo[3,2-b]pyrrole-5-carboxylate (43)

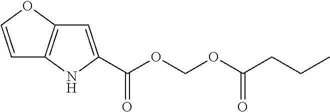

The title compound was synthesized from 4H-furo[3,2-b]pyrrole-5-carboxylic acid and chloromethyl butyrate according to General Procedure 7.D. LC-MS: m/z=252 (M+1).

### 7.12 Synthesis of 1-(butyryloxy)ethyl 4H-furo[3,2-b]pyrrole-5-carboxylate (44)

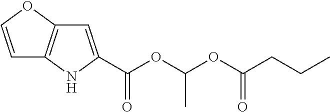

### 7.12.1 Synthesis of 1-chloroethyl butyrate

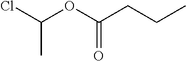

The title compound was synthesized from butyryl chloride and acetaldehyde according to General Procedure 7.A. The crude product was used directly in the next step without further purification.

### 7.12.2 Synthesis of 1-(butyryloxy)ethyl 4H-furo[3,2-b]pyrrole-5-carboxylate (44)

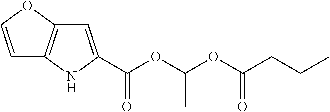

The title compound was synthesized from 4H-furo[3,2-b]pyrrole-5-carboxylic acid and 1-chloroethyl butyrate according to General Procedure 7.D. LC-MS: m/z=266 (M+1).

### 7.13 Synthesis of 1-acetoxypropyl 4H-furo[3,2-b]pyrrole-5-carboxylate (45)

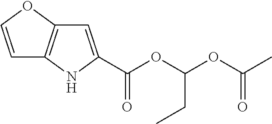

### 7.13.1 Synthesis of 1-chloropropyl acetate

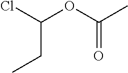

The title compound was synthesized from acetyl chloride and propionaldehyde according to General Procedure 7.A. The crude product was used directly in the next step without further purification.

### 7.13.2 Synthesis of 1-acetoxypropyl 4H-furo[3,2-b]pyrrole-5-carboxylate (45)

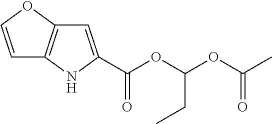

The title compound was synthesized from 4H-furo[3,2-b]pyrrole-5-carboxylic acid and 1-chloropropyl acetate according to General Procedure 7.D. LC-MS: m/z=252 (M+1).

### 7.14 Synthesis of 1-(isobutyryloxy)-2-methylpropyl 4H-furo[3,2-b]pyrrole-5-carboxylate (46)

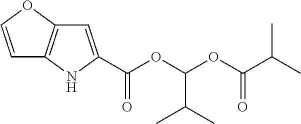

### 7.14.1 Synthesis of 1-chloro-2-methylpropyl isobutyrate

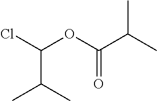

The title compound was synthesized from isobutyryl chloride and isobutyraldehyde according to General Procedure 7.A. The crude product was used directly in the next step without further purification.

### 7.14.2 Synthesis of 1-(isobutyryloxy)-2-methylpropyl 4H-furo[3,2-b]pyrrole-5-carboxylate (46)

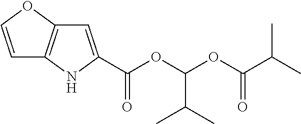

The title compound was synthesized from 4H-furo[3,2-b]pyrrole-5-carboxylic acid and 1-chloro-2-methylpropyl isobutyrate according to General Procedure 7.D. LC-MS: m/z=294 (M+1).

### 7.15 Synthesis of isobutyryloxymethyl 4H-furo[3,2-b]pyrrole-5-carboxylate (47)

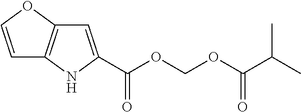

### 7.15.1 Synthesis of chloromethyl isobutyrate

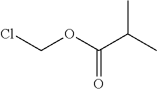

The title compound was synthesized from isobutyryl chloride and paraformaldehyde according to General Procedure 7.B. The crude product was used directly in the next step without further purification.

### 7.15.2 Synthesis of 1-(isobutyryloxy)-2-methylpropyl 4H-furo[3,2-b]pyrrole-5-carboxylate (47)

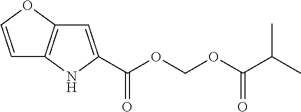

The title compound was synthesized from 4H-furo[3,2-b]pyrrole-5-carboxylic acid and chloromethyl isobutyrate according to General Procedure 7.D. LC-MS: m/z=252 (M+1).

### 7.16 Synthesis of pivaloyloxymethyl 4H-furo[3,2-b]pyrrole-5-carboxylate (48)

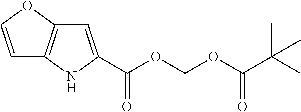

### 7.16.1 Synthesis of chloromethyl pivalate

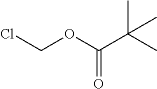

The title compound was synthesized from pivaloyl chloride and paraformaldehyde according to General Procedure 7.B. The crude product was used directly in the next step without further purification.

### 7.16.2 Synthesis of pivaloyloxymethyl 4H-furo[3,2-b]pyrrole-5-carboxylate (48)

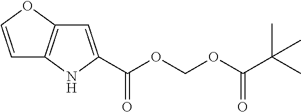

The title compound was synthesized from 4H-furo[3,2-b]pyrrole-5-carboxylic acid and chloromethyl pivalate according to General Procedure 7.D. LC-MS: m/z=266 (M+1).

### 7.17 Synthesis of 2-methyl-1-(propionyloxy)propyl 4H-furo[3,2-b]pyrrole-5-carboxylate (49)

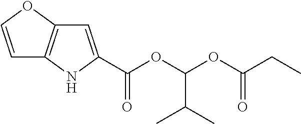

### 7.17.1 Synthesis of 1-chloro-2-methylpropyl propionate

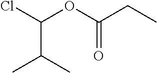

The title compound was synthesized from propionyl chloride and isobutyraldehyde according to General Procedure 7.A. The crude product was used directly in the next step without further purification.

### 7.17.2 Synthesis of 2-methyl-1-(propionyloxy)propyl 4H-furo[3,2-b]pyrrole-5-carboxylate (49)

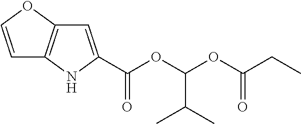

The title compound was synthesized from 4H-furo[3,2-b]pyrrole-5-carboxylic acid and 1-chloro-2-methylpropyl propionate according to General Procedure 7.D. LC-MS: m/z=280 (M+1).

### 7.18 Synthesis of 1-(pivaloyloxy)ethyl 4H-furo[3,2-b]pyrrole-5-carboxylate (50)

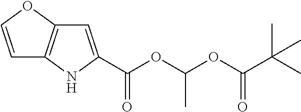

### 7.18.1 Synthesis of 1-chloroethyl pivalate

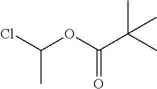

The title compound was synthesized from pivaloyl chloride and acetaldehyde according to General Procedure 7.A. The crude product was used directly in the next step without further purification.

### 7.18.2 Synthesis of 1-(pivaloyloxy)ethyl 4H-furo[3,2-b]pyrrole-5-carboxylate (50)

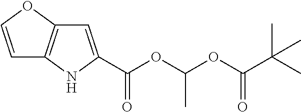

The title compound was synthesized from 4H-furo[3,2-b]pyrrole-5-carboxylic acid and 1-chloroethyl pivalate according to General Procedure 7.D. LC-MS: m/z=280 (M+1).

### Example 8

### Synthesis of Ethylene-Linked Diester Derivatives

### 8.1 Synthesis of 2-(2-morpholinoacetoxy)ethyl 4H-furo[3,2-b]pyrrole-5-carboxylate (51)

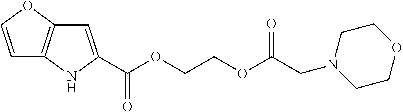

### 8.1.1 Synthesis of 2-hydroxyethyl 4H-furo[3,2-b]pyrrole-5-carboxylate

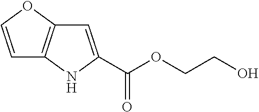

The title compound was synthesized from sodium 4H-furo[3,2-b]pyrrole-5-carboxylate (1.0 g, 5.8 mmol) and 2-chloroethanol (0.51 g, 6.3 mmol) according to General Procedure 8.A. The crude product was triturated with a solution of EtOAc and hexane (1/5 ratio) to give the desired product. LC-MS: MS m/z: 196 (M+1).

### 8.1.2 Synthesis of 2-(4-chlorobutanoyloxy)ethyl 4H-furo[3,2-b]pyrrole-5-carboxylate

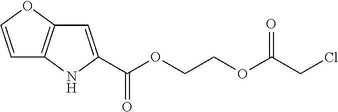

The title compound was synthesized from 2-hydroxyethyl 4H-furo[3,2-b]pyrrole-5-carboxylate and 2-chloroacetic acid according to General Procedure 8.B.

### 8.1.3 Synthesis of 2-(2-morpholinoacetoxy)ethyl 4H-furo[3,2-b]pyrrole-5-carboxylate (51)

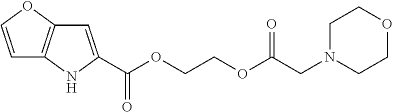

The title compound was synthesized from 2-(4-chlorobutanoyloxy)ethyl 4H-furo[3,2-b]pyrrole-5-carboxylate and morpholine according to General Procedure 8.C. LC-MS: m/z=323 (M+1).

### 8.2 Synthesis of 2-(4-morpholinobutanoyloxy)ethyl 4H-furo[3,2-b]pyrrole-5-carboxylate (52)

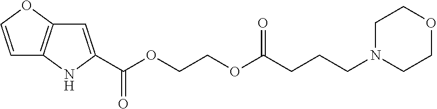

### 8.2.1 Synthesis of 2-(4-chlorobutanoyloxy)ethyl 4H-furo[3,2-b]pyrrole-5-carboxylate

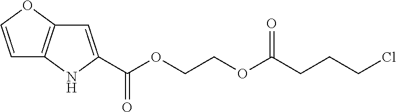

The title compound was synthesized from 2-hydroxyethyl 4H-furo[3,2-b]pyrrole-5-carboxylate (0.3 g, 1.54 mmol) and 4-chlorobutanoic acid (1.3 g, 10 mmol) according to General Procedure 8.B. The crude product was purified by column chromatography to give 0.147 g of the desired product.

### 8.2.2 Synthesis of 2-(2-morpholinoacetoxy)ethyl 4H-furo[3,2-b]pyrrole-5-carboxylate (52)

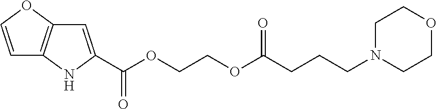

The title compound was synthesized from 2-(4-chlorobutanoyloxy)ethyl 4H-furo[3,2-b]pyrrole-5-carboxylate and morpholine according to General Procedure 8.C. LC-MS: m/z=351 (M+1).

### 8.3 Synthesis of 2-(4-(4-methylpiperazin-1-yl)butanoyloxy)ethyl 4H-furo[3,2-b]pyrrole-5-carboxylate (53)

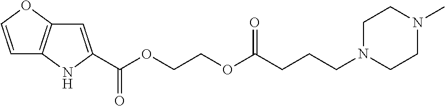

The title compound was synthesized from 2-(4-chlorobutanoyloxy)ethyl 4H-furo[3,2-b]pyrrole-5-carboxylate (0.418 g, 1.4 mmol) and 1-methylpiperazine (0.162 g, 1.6 mmol) according to General Procedure 8.C. The crude product was purified by column chromatography to give the desired product. LC-MS: MS m/z: 364 (M+1).

### 8.4 Synthesis of 2-(2-(4-methylpiperazin-1-yl)acetoxy)ethyl 4H-furo[3,2-b]pyrrole-5-carboxylate (54)

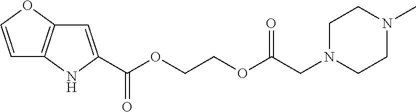

The title compound was synthesized from 2-(4-chlorobutanoyloxy)ethyl 4H-furo[3,2-b]pyrrole-5-carboxylate (0.418 g, 1. 4 mmol) and 1-methylpiperazine according to General

Procedure 8.C. The crude product was purified by column chromatography to give the desired product. LC-MS: MS m/z: 336 (M+1).

### 8.5 Synthesis of ethane-1,2-diyl bis(4H-furo[3,2-b]pyrrole-5-carboxylate) (55)

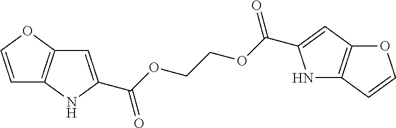

A solution of sodium 4H-furo[3,2-b]pyrrole-5-carboxylate (150 mg, 0.87 mmol, 2.1 equiv), 1, 2-dibromoethane (77.6 mg, 0.4129 mmol, 1 equiv) in DMF (20 ml) was stirred at 100° C. overnight. After cooling to rt, the mixture was diluted with H₂O and extracted with EtOAc (3 times). The combined organic layer was washed with brine, and dried over Na₂SO₄. The filtrate was concentrated and purified by column chromatography to give the desired product. LC-MS: MS m/z: 329 (M+1).

### Example 9

### Pyrrole-5-carboxamide Derivatives

### 9.1 Synthesis of 4H-furo[3,2-b]pyrrole-5-carboxamide (56)

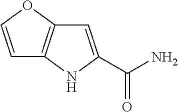

The title compound was synthesized from 4H-furo[3,2-b]pyrrole-5-carboxylic acid and ammonia according to General Procedure 9. LC-MS: m/z=151 (M+1).

### 9.2 Synthesis of N-(2-amino-2-oxoethyl)-4H-furo[3,2-b]pyrrole-5-carboxamide (57)

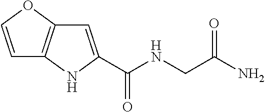

The title compound was synthesized from 4H-furo[3,2-b]pyrrole-5-carboxylic acid and ethyl 2-aminoacetate hydrochloride according to General Procedure 9.2. LC-MS: m/z=208 (M+1).

### 9.3 Synthesis of (4H-furo[3,2-b]pyrrol-5-yl)(4-methylpiperazin-1-yl)methanone (58)

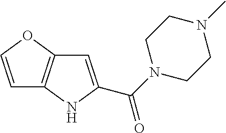

The title compound was synthesized from 4H-furo[3,2-b]pyrrole-5-carboxylic acid and 1-methylpiperazine according to General Procedure 9.3. LC-MS: m/z=234 (M+1).

### Example 10

### Synthesis of Pyrrole N-Substituted Analogs

### 10.1 Synthesis of ethyl 4-(benzoyloxymethyl)-4H-furo[3,2-b]pyrrole-5-carboxylate (59)

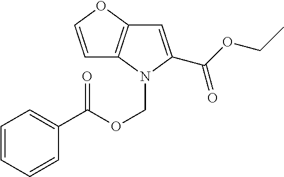

The title compound was synthesized from ethyl 4H-furo[3,2-b]pyrrole-5-carboxylate (100 mg, 0.57 mmol) and chloromethyl benzoate (200 mg, 1.17 mmol) according to General Procedure 10.1. The crude product was purified by column chromatography to give the desired product. LC-MS: MS m/z: 314 (M+1).

### 10.2 Synthesis of 4-(benzoyloxymethyl)-4H-furo[3,2-b]pyrrole-5-carboxylic acid (60)

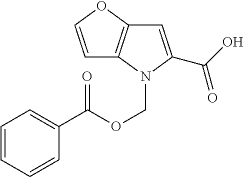

The title compound was synthesized in two steps from benzyl 4H-furo[3,2-b]pyrrole-5-carboxylate and chloromethyl benzoate according to General Procedure 10.2. LC-MS: MS m/z: 286 (M+1).

### 10.3 Synthesis of ethyl 4-((2-aminoacetoxy)methyl)-4H-furo[3,2-b]pyrrole-5-carboxylate (61)

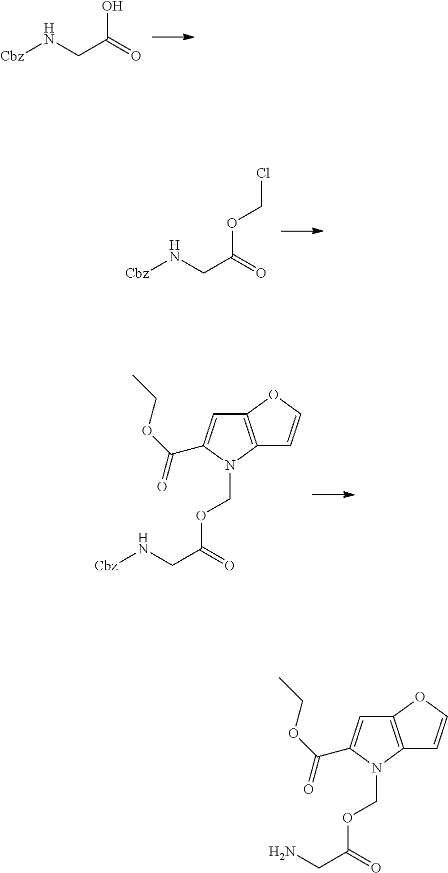

### 10.3.1 Synthesis of chloromethyl 2-(benzyloxycarbonylamino)acetate

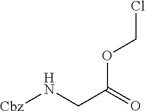

The title compound was synthesized from 2-(benzyloxycarbonylamino)acetic acid according to General Procedure 7.A. LC-MS: MS m/z: 258 (M+1).

### 10.3.2 Synthesis of ethyl 4-((2-(benzyloxycarbonylamino)acetoxy)methyl)-4H-furo[3,2-b]pyrrole-5-carboxylate

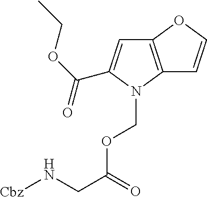

The title compound was synthesized from ethyl 4H-furo[3,2-b]pyrrole-5-carboxylate and chloromethyl 2-(benzyloxycarbonylamino)acetate according to General Procedure 10.1. LC-MS: MS m/z: 401 (M+1).

### 10.3.3 Synthesis of ethyl 4-((2-aminoacetoxy)methyl)-4H-furo[3,2-b]pyrrole-5-carboxylate (61)

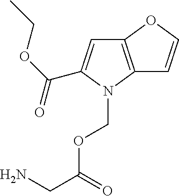

Ethyl 4-((2-(benzyloxycarbonyl)acetoxy)methyl)-4H-furo[3,2-b]pyrrole-5-carboxylate (200 mg) was dissolved in EtOAc. To the mixture was added one drop of 35% HCl solution and a catalytic amount of Pd/C. The reaction vessel was evacuated and then back filled with hydrogen gas three times. The solution was stirred for 4 h under the atmospheric pressure hydrogen. The reaction mixture was filtered through a pad of Celite and washed with methanol. The combined filtrate was concentrated and purified by preparative TLC to give the desired product. LC-MS: MS m/z: 267 (M+1).

### 10.4 Synthesis of ethyl 4-((2-aminopropanoyloxy)methyl)-4H-furo[3,2-b]pyrrole-5-carboxylate (62)

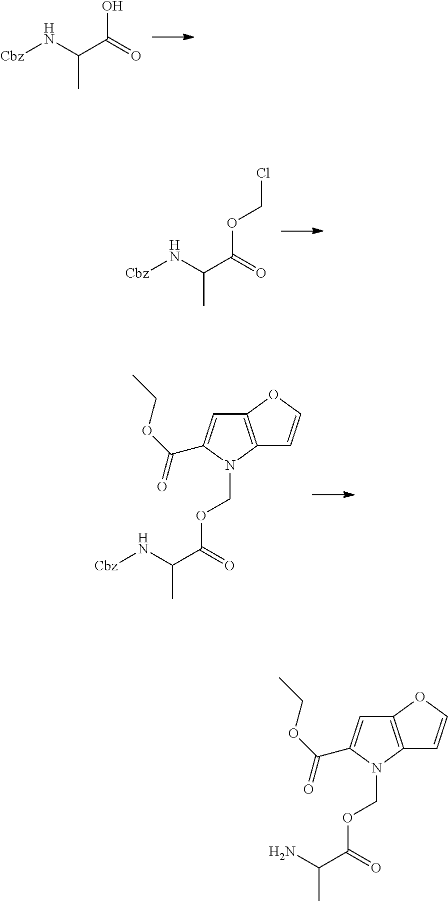

### 10.4.1 Synthesis of chloromethyl 2-(benzyloxycarbonylamino)propanoate

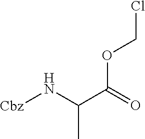

The title compound was synthesized from 2-(benzyloxycarbonylamino)propanoic acid according to General Procedure 7.A. LC-MS: MS m/z: 272 (M+1).

### 10.4.2 Synthesis of ethyl 4-((2-(benzyloxycarbonylamino)propanoyloxy)methyl)-4H-furo[3,2-b]pyrrole-5-carboxylate

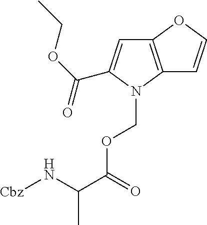

The title compound was synthesized from ethyl 4H-furo[3,2-b]pyrrole-5-carboxylate and chloromethyl 2-(benzyloxycarbonylamino)propanoate according to General Procedure 10.1. LC-MS: MS m/z: 415 (M+1).

### 10.4.3 Synthesis of ethyl 4-((2-aminopropanoyloxy)methyl)-4H-furo[3,2-b]pyrrole-5-carboxylate (62)

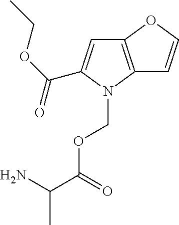

The title compound was synthesized from ethyl 4-((2-(benzyloxycarbonylamino)propanoyloxy)methyl)-4H-furo[3,2-b]pyrrole-5-carboxylate according to the procedure described in Example 10.3.3. LC-MS: MS m/z: 281 (M+1).

### 10.5 Synthesis of ethyl 4-((2-amino-3-hydroxypropanoyloxy)methyl)-4H-furo[3,2-b]pyrrole-5-carboxylate (63)

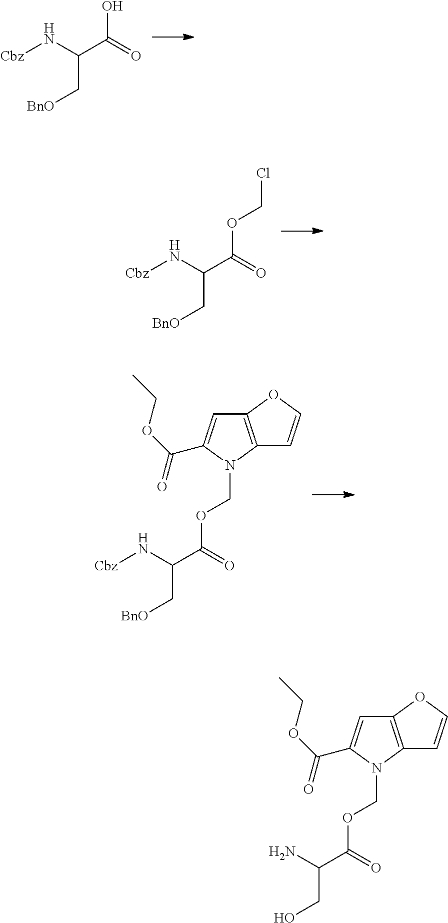

### 10.5.1 Synthesis of chloromethyl 3-(benzyloxy)-2-(benzyloxycarbonylamino)propanoate

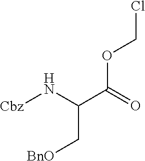

The title compound was synthesized from 3-(benzyloxy)-2-(benzyloxycarbonylamino)propanoic acid according to General Procedure 7.A. LC-MS: MS m/z: 378 (M+1).

### 10.5.2 Synthesis of ethyl 4-((3-(benzyloxy)-2-(benzyloxycarbonylamino)propanoyloxy)methyl)-4H-furo[3,2-b]pyrrole-5-carboxylate

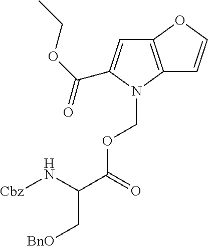

The title compound was synthesized from ethyl 4H-furo[3,2-b]pyrrole-5-carboxylate and chloromethyl 3-(benzyloxy)-2-(benzyloxycarbonylamino)propanoate according to General Procedure 10.1. LC-MS: MS m/z: 521 (M+1).

### 10.5.3 Synthesis of ethyl 4-((2-amino-3-hydroxypropanoyloxy)methyl)-4H-furo[3,2-b]pyrrole-5-carboxylate (63)

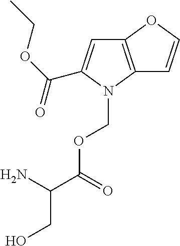

The title compound was synthesized from ethyl 4-((3-(benzyloxy)-2-(benzyloxycarbonylamino)propanoyloxy)methyl)-4H-furo[3,2-b]pyrrole-5-carboxylate according to example 10.3.3. LC-MS: MS m/z: 297 (M+1).

### 10.6 Synthesis of ethyl 4-(acetoxymethyl)-4H-furo[3,2-b]pyrrole-5-carboxylate (64)

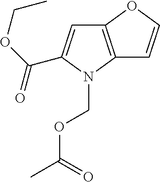

To a solution of ethyl 4H-furo[3,2-b]pyrrole-5-carboxylate (179 mg, 1 mmol), a catalytic amount of NaI, and chloromethyl acetate (325 mg, 3 mmol) in 2-methyl-tetrahydrofuran (25 mL) was added DBU (228 mg, 1.5 mmol). The reaction mixture was stirred at rt for two days. The solid was removed by filtration and the filtrate was concentrated in vacuo. The crude product was purified by column chromatography to give the desired product. LC/MS: MS m/z: 252 (M+1).

### 10.7 Synthesis of ethyl 4-(isobutyryloxymethyl)-4H-furo[3,2-b]pyrrole-5-carboxylate (65)

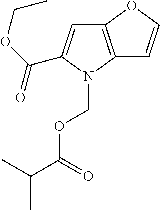

The title compound was synthesized from ethyl 4H-furo[3,2-b]pyrrole-5-carboxylate and chloromethyl isobutyrate according to General Procedure 10.1. LC-MS: MS m/z: 280 (M+1).

### 10.8 Synthesis of 4-(acetoxymethyl)-4H-furo[3,2-b]pyrrole-5-carboxylic acid (66)

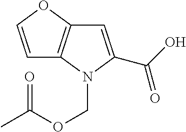

The title compound was synthesized in two steps from benzyl 4H-furo[3,2-b]pyrrole-5-carboxylate and chloromethyl acetate according to General Procedure 10.2. The crude product was purified by column chromatography to afford the desired product. LC/MS: MS m/z: 224 (M+1).

### 10.9 Synthesis of 4-(isobutyryloxymethyl)-4H-furo[3,2-b]pyrrole-5-carboxylic acid (67)

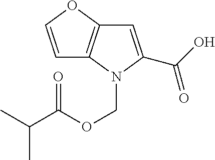

The title compound was synthesized in two steps from benzyl 4H-furo[3,2-b]pyrrole-5-carboxylate and chloromethyl isobutyrate according to General Procedure 10.2. The crude product was purified by column chromatography to afford the desired product. LC/MS: MS m/z: 252 (M+1).

### 10.10 Synthesis of (R)-ethyl 4-((2-(2-amino-3-methylbutanoyloxy)ethoxy)methyl)-4H-furo[3,2-b]pyrrole-5-carboxylate (68)

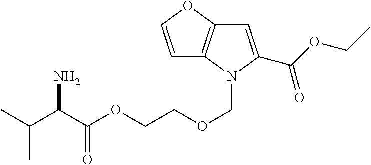

### 10.10.1 Synthesis of ethyl 4-((2-chloroethoxy)methyl)-4H-furo[3,2-b]pyrrole-5-carboxylate

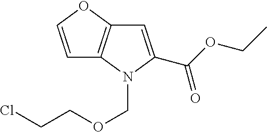

The title compound was synthesized from ethyl 4H-furo[3,2-b]pyrrole-5-carboxylate (0.4 g, 2.23 mmol) and 1-chloro-2-(chloromethoxy)ethane (General Experimental 10.3.A) (1.0 g, 7.81 mmol) according to General Procedure 10.3.B. The crude product was purified by column chromatography to afford the title compound. LC/MS: MS m/z: 272 (M+1).

### 10.10.2 Synthesis of (R)-ethyl 4-((2-(2-(1,3-dioxoisoindolin-2-yl)-3-methylbutanoyloxy)ethoxy)methyl)-4H-furo[3,2-b]pyrrole-5-carboxylate

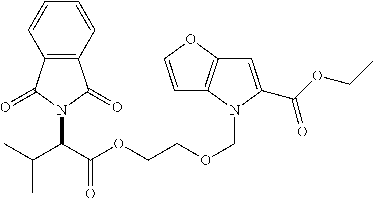

The title compound was synthesized from ethyl 4-((2-chloroethoxy)methyl)-4H-furo[3,2-b]pyrrole-5-carboxylate and (R)-2-(1,3-dioxoisoindolin-2-yl)-3-methylbutanoic acid (0.658 g, 2.4 mmol) according to General Procedure 10.3.C. The residue oil was purified by column chromatography to afford the desired product. LC/MS: MS m/z: 483 (M+1).

### 10.10.3 Synthesis of (R)-ethyl 4-((2-(2-amino-3-methylbutanoyloxy)ethoxy)methyl)-4H-furo[3,2-b]pyrrole-5-carboxylate (68)

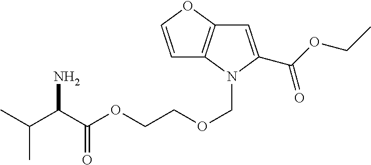

The title compound was synthesized from (R)-ethyl 4-((2-(2-(1,3-dioxoisoindolin-2-yl)-3-methylbutanoyloxy)ethoxy)methyl)-4H-furo[3,2-b]pyrrole-5-carboxylate and NH₂NH₂.H₂O (0.5 g) according to General Procedure 10.3.D.

### 10.11 Synthesis of (R)-ethyl 4-((2-(2-(2-aminoacetamido)-3-methylbutanoyloxy)ethoxy)methyl)-4H-furo[3,2-b]pyrrole-5-carboxylate (69)

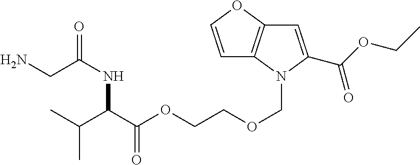

The title compound was synthesized in two steps from ethyl 4-((2-chloroethoxy)methyl)-4H-furo[3,2-b]pyrrole-5-carboxylate and (R)-2-(2-(1,3-dioxoisoindolin-2-yl)acetamido)-3-methylbutanoic acid according to General Procedures 10.3.0 and 10.3.D. LC/MS: MS m/z: 410 (M+1).

### 10.12 Synthesis of (R)-4-((2-(2-amino-3-methylbutanoyloxy)ethoxy)methyl)-4H-furo[3,2-b]pyrrole-5-carboxylic acid (70)

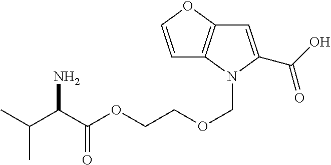

### 10.12.1 Synthesis of benzyl 4-((2-chloroethoxy)methyl)-4H-furo[3,2-b]pyrrole-5-carboxylate

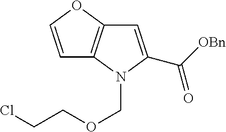

The title compound was synthesized from benzyl 4H-furo[3,2-b]pyrrole-5-carboxylate (0.4 g, 2.23 mmol) and 1-chloro-2-(chloromethoxy)ethane (General Experimental 10.3.A) (1.0 g, 7.81 mmol) according to General Procedure 10.3.B. The crude product was purified by column chromatography to afford the title compound. LC/MS: MS m/z: 334 (M+1).

### 10.12.2 Synthesis of (R)-benzyl 4-((2-(2-(1,3-dioxoisoindolin-2-yl)-3-methylbutanoyloxy)ethoxy)methyl)-4H-furo[3,2-b]pyrrole-5-carboxylate

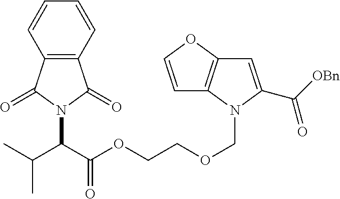

The title compound was synthesized from benzyl 4-((2-chloroethoxy)methyl)-4H-furo[3,2-b]pyrrole-5-carboxylate and (R)-2-(1,3-dioxoisoindolin-2-yl)-3-methylbutanoic acid (0.658 g, 2.4 mmol) according to General Procedure 10.3.C. The residue oil was purified by column chromatography to afford the desired product. LC/MS: MS m/z: 545 (M+1).

### 10.12.3 Synthesis of (R)-benzyl 4-((2-(2-amino-3-methylbutanoyloxy)ethoxy)methyl)-4H-furo[3,2-b]pyrrole-5-carboxylate

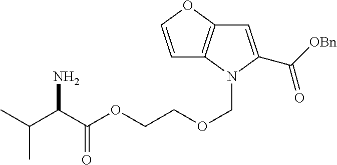

The title compound was synthesized from (R)-benzyl 4-((2-(2-(1,3-dioxoisoindolin-2-yl)-3-methylbutanoyloxy)ethoxy)methyl)-4H-furo[3,2-b]pyrrole-5-carboxylate and NH₂NH₂.H₂O according to General Procedure 10.3.D.

### 10.12.4 Synthesis of (R)-4-((2-(2-amino-3-methylbutanoyloxy)ethoxy)methyl)-4H-furo[3,2-b]pyrrole-5-carboxylic acid (70)

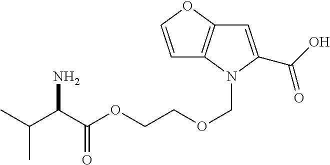

The (R)-benzyl 4-((2-(2-amino-3-methylbutanoyloxy)ethoxy)methyl)-4H-furo[3,2-b]pyrrole-5-carboxylate (116 mg) was dissolved in EtOH (20 mL). A catalytic amount of Pd/C was added and the reaction was placed under a hydrogen atmosphere using a balloon. After stirred at rt overnight, the reaction mixture was filtered through a pad of Celite. The filtrate was concentrated and purified by column chromatography to afford the pure product. LC/MS: MS m/z: 325 (M+1).

### 10.13 Synthesis of acetoxymethyl4-(acetoxymethyl)-4H-furo[3,2-b]pyrrole-5-carboxylate (71)

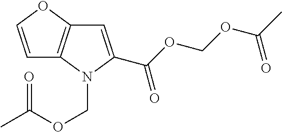

The title compound was synthesized from acetoxymethyl 4H-furo[3,2-b]pyrrole-5-carboxylate (Example 7.6) according to General Procedure 10.1. The crude product was purified by column chromatography to afford the desired product. LC-MS: MS m/z: 296 (M+1).

### Example 11

### Synthesis of Cyclic Carbonate Analogs

### 11.1 Synthesis of (5-methyl-2-oxo-1,3-dioxol-4-yl)methyl 4H-furo[3,2-b]pyrrole-5-carboxylate (72)

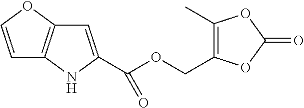

A solution of 4H-furo[3,2-b]pyrrole-5-carboxylic acid sodium salt (173 mg, 1 mmol.) and 4-(chloromethyl)-5-methyl-1,3-dioxol-2-one (148 mg, 1 mmol) in DMF (10 mL) was heated to 80° C. for 5 h. After cooling to rt, the mixture was diluted with H₂O (20 mL), and then the insoluble was removed by filtration. The filter cake was triturated with n-hexane to form (5-methyl-2-oxo-1,3-dioxol-4-yl)methyl 4H-furo[3,2-b]pyrrole-5-carboxylate. LC-MS: m/z=264 (M+1).

### 11.2 Synthesis of 2-oxo-1,3-dioxolan-4-yl 4H-furo[3,2-b]pyrrole-5-carboxylate (73)

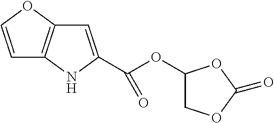

A catalytic amount of sodium iodide was added to a stirred solution of 4-chloro-1,3-dioxolan-2-one (333.9 mg, 1.41 mmol), triethylamine (290.3 mg, 2.87 mmol) and 4H-furo[3,2-b]pyrrole-5-carboxylic acid sodium salt (212.8 mg, 2.73 mmol) in dry dioxane (5 mL) at rt. The reaction mixture was stirred at 90° C. for 24 h. The reaction mixture was concentrated in vacuo. The crude product was isolated by silica gel column chromatographic purification. LC-MS : m/z=238 (M+1).

### Example 12

### Synthesis of Benzofuran Ester Analogs

### 12.1 Synthesis of 3-oxo-1,3-dihydroisobenzofuran-1-yl 4H-furo[3,2-b]pyrrole-5-carboxylate (74)

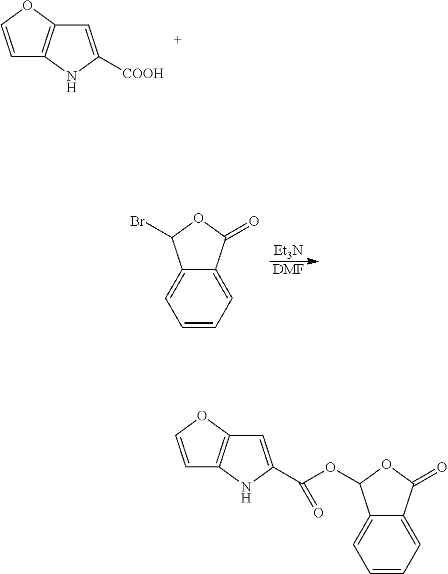

A solution of 4H-furo[3,2-b]pyrrole-5-carboxylic acid (200 mg, 1.33 mmol), 3-bromoisobenzofuran-1(3H)-one (284 mg, 1.33 mmol), and Et₃N (269 mg, 2.66 mmol.) in DMF (10 mL) was stirred at 60° C. for 24 h. After cooling to rt, the reaction mixture was diluted with H₂O and extracted with EtOAc (150 mL×3). The organic layer was washed with H₂O (3 times) and dried over MgSO₄. The filtrate was concentrated and purified by column chromatography to afford the product as solid (60 mg). LC-MS: MS m/z: 284 (M+1).

### Example 13

### Synthesis of Fluoromethylesters

### 13.1 Synthesis of S-fluoromethyl 4H-furo[3,2-b]pyrrole-5-carbothioate (75)

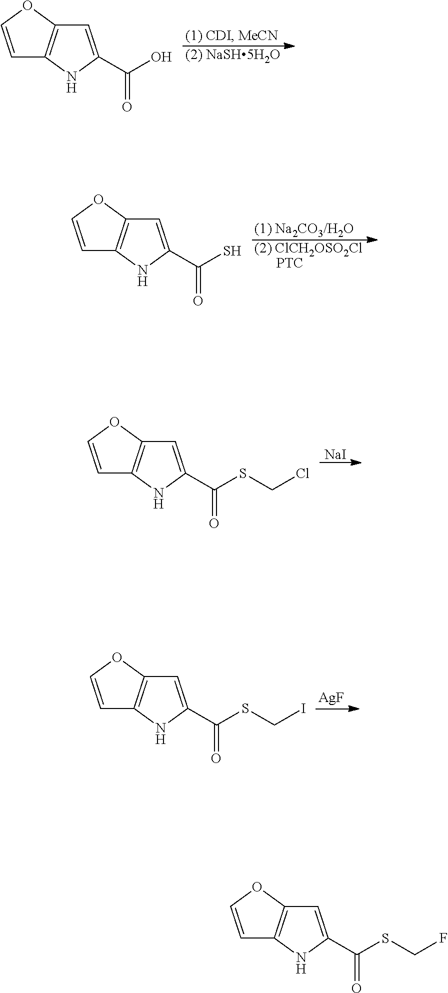

### 13.1.1 Synthesis of 4H-furo[3,2-b]pyrrole-5-carbothioic S-acid

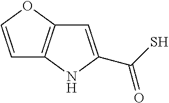

A solution of 4H-furo[3,2-b]pyrrole-5-carboxylic acid (5 g, 0.033 mol), CDI (10 g, 0.062 mol) in MeCN (100 mL) was stirred at rt overnight. DMF (50 mL) and NaSH (27.9 g, 0.191 mol) were added to the reaction mixture and stirred at rt overnight. The reaction mixture was poured into 2M HCl and washed with EtOAc (3×500 mL). The pH of the combined aqueous layer was adjusted with HCl (36%) to 2-3, extracted with EtOAc (3×500 mL) and dried over MgSO₄, filtered, the filtration was concentrated and purified by column chromatography affording the product as solid (0.7 g). LC-MS: MS m/z: 168 (M+1).

### 13.1.2 Synthesis of S-chloromethyl 4H-furo[3,2-b]pyrrole-5-carbothioate

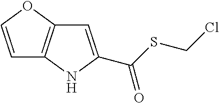

The title compound was synthesized from 4H-furo[3,2-b]pyrrole-5-carbothioic S-acid and chloromethyl sulfochloridate as described in Example 7.9.

### 13.1.3 Synthesis of S-iodomethyl 4H-furo[3,2-b]pyrrole-5-carbothioate

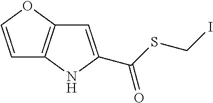

To a solution of S-chloromethyl 4H-furo[3,2-b]pyrrole-5-carbothioate (338 mg, 1.57 mmol) in acetone (40 mL) at rt was added NaI (2.8 g, 18.6 mmol) and the resulting mixture was heated to reflux for 30 h. After cooled to rt, the reaction mixture was diluted with H₂O and extracted with EtOAc (3×150 mL). The organic layer was washed with 5% Na₂CO₃ solution (3 times), brine, dried over MgSO₄, filtered, and concentrated to give a yellow solid (45 mg). LC-MS: MS m/z: 308 (M+1).

### 13.1.4 Synthesis of S-fluoromethyl 4H-furo[3,2-b]pyrrole-5-carbothioate (75)

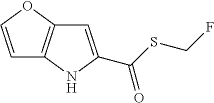

A solution of (S)-iodomethyl-(4H)-furo[3,2-b]pyrrole-5-carbothioate (45 mg, 0.147 mmol) and AgF (75 mg, 0.6 mmol) in MeCN (5 mL) was stirred at rt for 6 h. The reaction mixture was diluted with H₂O and extracted with EtOAc (3×150 mL). The organic layer was washed with 5% Na₂CO₃ solution (3 times), brine, dried over Mg50₄, and filtered. The filtrate was concentrated and purified by column chromatography to afford the product as solid (15 mg). LC-MS: MS m/z: 200 (M+1).

### Example 14.1

### Stability in Rat, Dog, and Human Plasma

### Methods

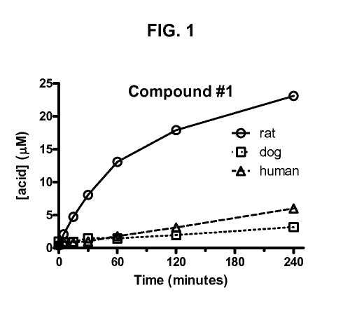

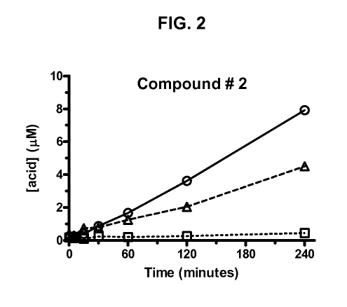

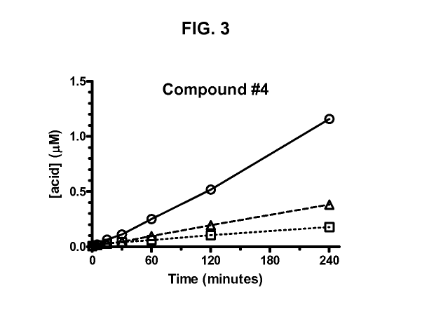

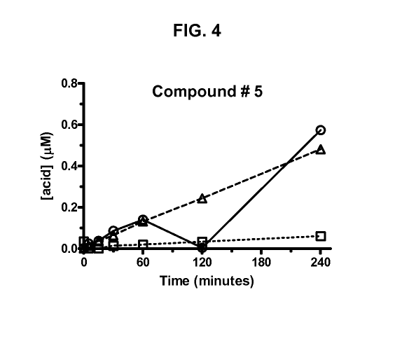

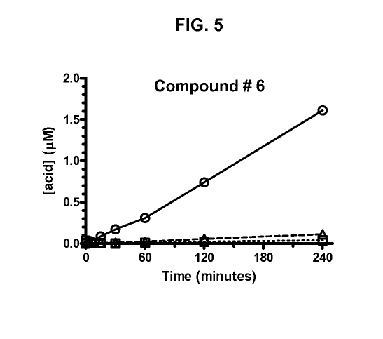

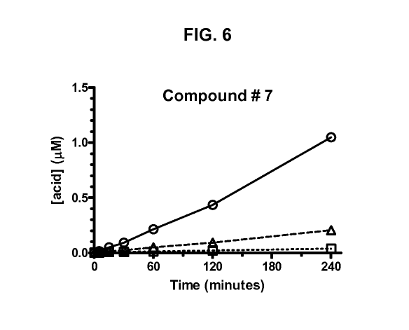

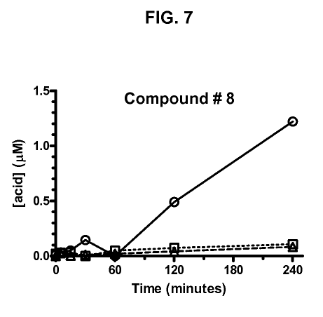

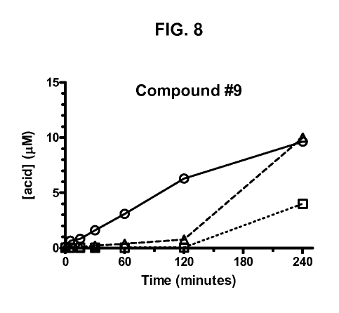

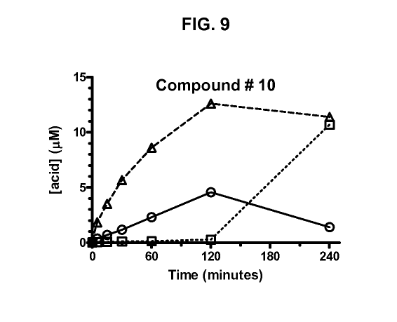

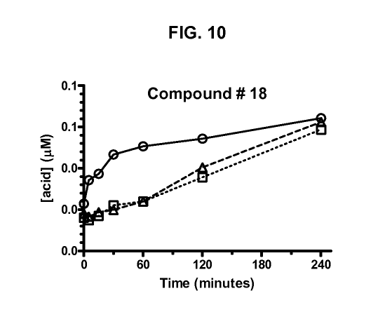

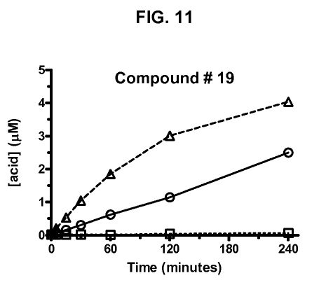

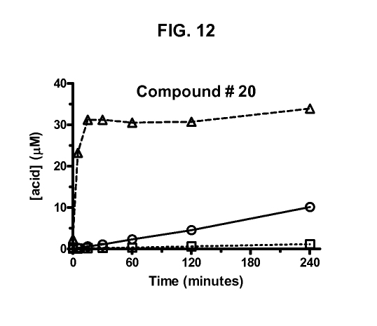

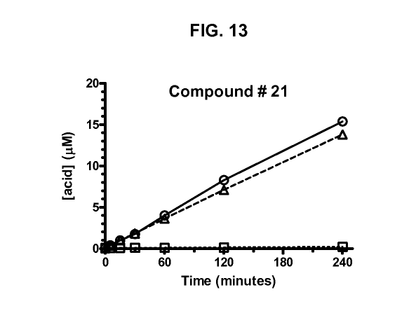

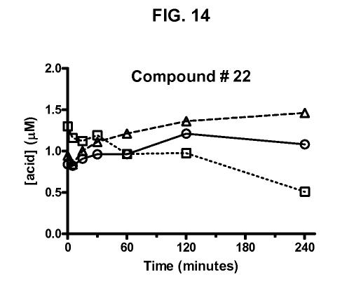

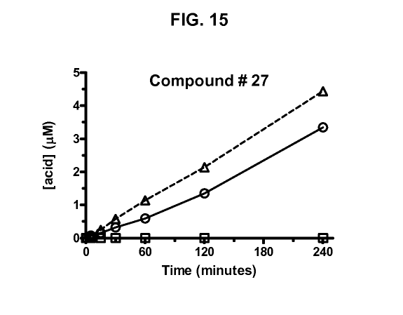

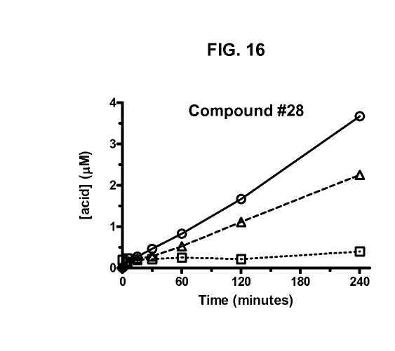

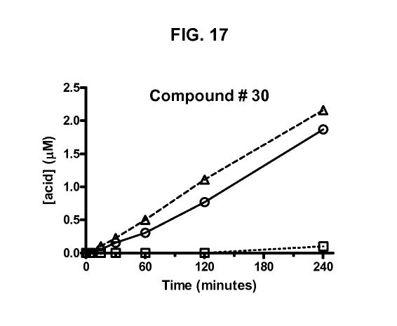

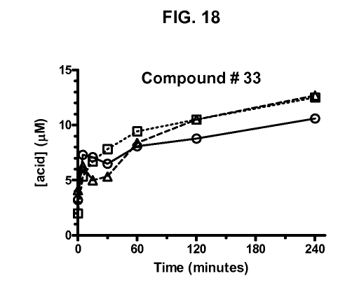

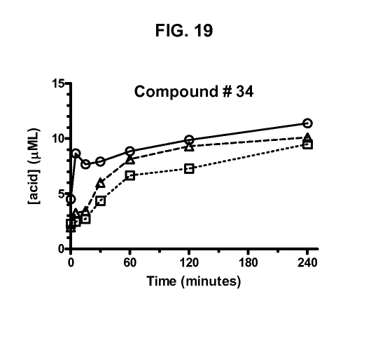

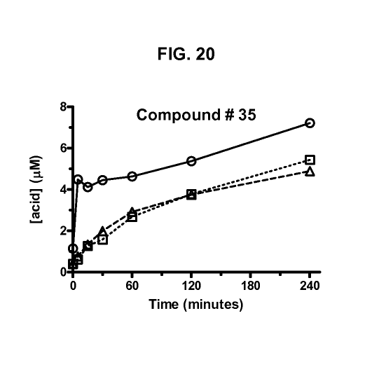

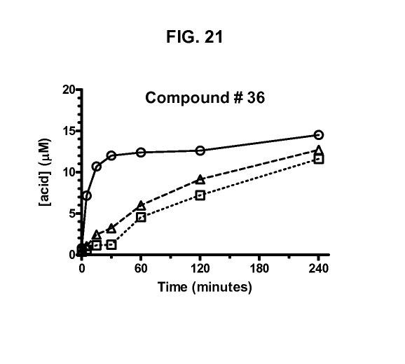

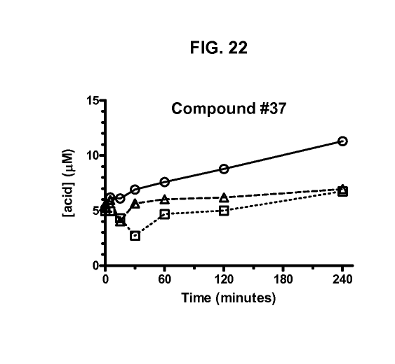

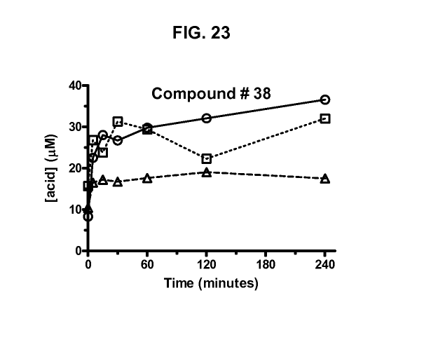

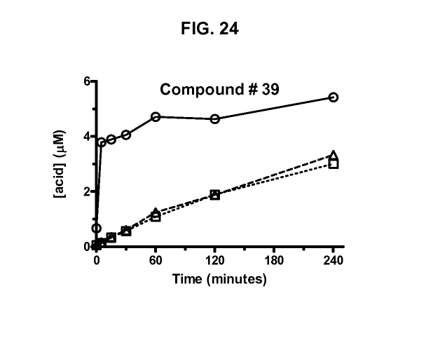

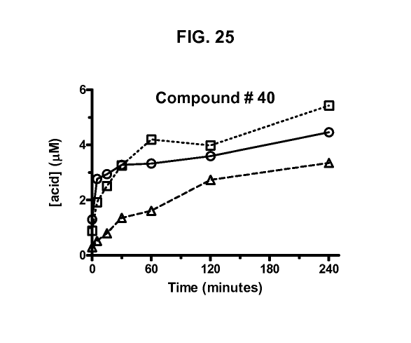

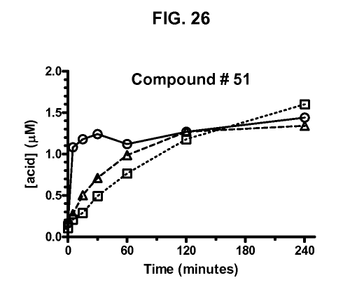

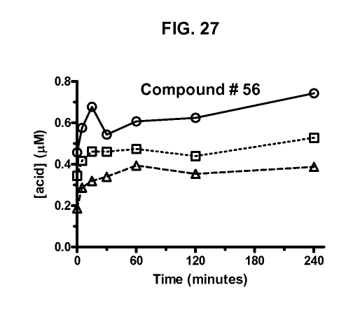

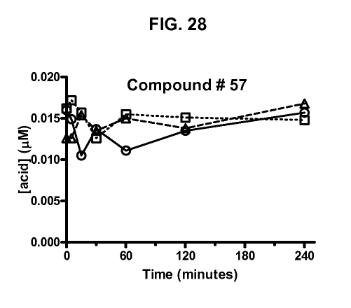

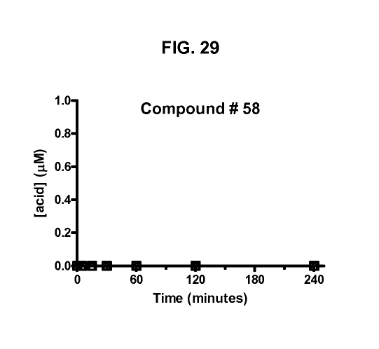

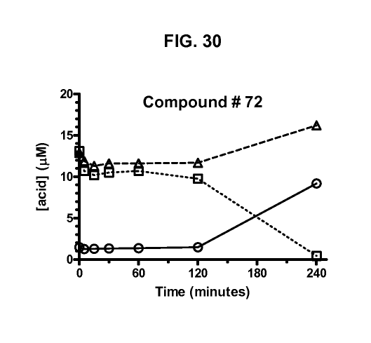

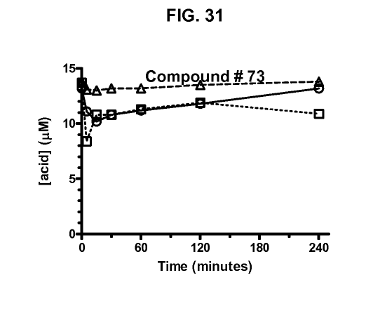

Conversion of compounds to 4H-furo[3,2-b]pyrrole-5-carboxylic acid (“acid”) in rat, dog, and human plasma (n=1 for each data point) was evaluated according to General Procedure 11. In the graphs of this example (see FIGS. 1-31), “[acid]” refers to the concentration of 4H-furo[3,2-b]pyrrole-5-carboxylic acid.

### Example 14.2

### Stability in Human Plasma

### Methods

Conversion of compounds to 4H-furo[3,2-b]pyrrole-5-carboxylic acid (“acid”) in human plasma were evaluated according to General Procedure 11. n=3 for each data point In the table of this example, “[acid]” refers to the concentration of 4H-furo[3,2-b]pyrrole-5-carboxylic acid. The “hydrolysis rate” is obtained from the slope of a line fit by regression analysis and is in units of μM acid evolved per minute. The “Time Zero” column refers to the concentration of acid (μM) measured in the first time point, obtained immediately following addition of the compound.

TABLE 5
Conversion of the test compound to the corresponding acid in human plasma
(n = 3).

| Compound | | Hydrolysis | |
|---|---|---|---|
| number | Chemical Name | Rate | Time Zero |
| 2 | 11,11-dimethyl-5,10-dioxa-1- | 0.0024 | 0.0 |
| | azatricyclo[6.3.0.0^{2,6}]undeca-2(6),3,7-trien-9-one | | |
| 3 | 11-methyl-5,10-dioxa-1- | 0.0045 | 0.2 |
| | azatricyclo[6.3.0.0^{2,6}]undeca-2(6),3,7-trien-9-one | | |
| 5 | butyl 4H-furo[3,2-b]pyrrole-5-carboxylate | 0.0015 | 0.0 |
| 10 | (tetrahydrofuran-2-yl)methyl 4H-furo[3,2-b]pyrrole- | 0.0417 | 0.0 |
| | 5-carboxylate | | |
| 11 | pyridin-4-ylmethyl 4H-furo[3,2-b]pyrrole-5- | 0.005 | 0.2 |
| | carboxylate | | |
| 12 | 2-methoxyphenyl 4H-furo[3,2-b]pyrrole-5- | 0.0211 | 0.1 |
| | carboxylate | | |
| 13 | isopentyl 4H-furo[3,2-b]pyrrole-5-carboxylate | 0.0074 | 0.0 |
| 14 | isopropyl 4H-furo[3,2-b]pyrrole-5-carboxylate | 0.0002 | 0.0 |
| 15 | pentyl 4H-furo[3,2-b]pyrrole-5-carboxylate | 0.0008 | 0.0 |
| 16 | tert-butyl 4H-furo[3,2-b]pyrrole-5-carboxylate | 0.0001 | 0.0 |
| 19 | 2-(azetidin-1-yl)-2-oxoethyl 4H-furo[3,2-b]pyrrole- | 0.0186 | 0.0 |
| | 5-carboxylate | | |
| 20 | 2-(dimethylamino)-2-oxoethyl 4H-furo[3,2- | 0.3708 | 0.1 |
| | b]pyrrole-5-carboxylate | | |
| 21 | 2-morpholino-2-oxoethyl 4H-furo[3,2-b]pyrrole-5- | 0.009 | 0.0 |
| | carboxylate | | |
| 23 | 2-(diethylamino)-2-oxoethyl 4H-furo[3,2-b]pyrrole- | 0.8662 | 0.7 |
| | 5-carboxylate | | |
| 24 | 2-oxo-2-(pyrrolidin-1-yl)ethyl 4H-furo[3,2- | 0.0088 | 0.0 |
| | b]pyrrole-5-carboxylate | | |
| 25 | 2-oxo-2-(piperidin-1-yl)ethyl 4H-furo[3,2-b]pyrrole- | 0.0078 | 0.0 |
| | 5-carboxylate | | |
| 26 | 2-(4-methylpiperazin-1-yl)-2-oxoethyl 4H-furo[3,2- | 0.003 | 0.0 |
| | b]pyrrole-5-carboxylate | | |
| 27 | 2-(1H-imidazol-1-yl)ethyl 4H-furo[3,2-b]pyrrole-5- | 0.0033 | 0.1 |
| | carboxylate | | |
| 28 | (1H-imidazol-1-yl)methyl 4H-furo[3,2-b]pyrrole-5- | 0.0019 | 0.1 |
| | carboxylate | | |
| 29 | 2-(2-methyl-1H-imidazol-1-yl)ethyl 4H-furo[3,2- | 0.0117 | 0.0 |
| | b]pyrrole-5-carboxylate | | |
| 30 | 2-morpholinoethyl 4H-furo[3,2-b]pyrrole-5- | 0.0019 | 0.0 |
| | carboxylate | | |
| 31 | 2-(diethylamino)ethyl 4H-furo[3,2-b]pyrrole-5- | 0.4414 | 0.2 |
| | carboxylate | | |
| 32 | 2-(dimethylamino)ethyl 4H-furo[3,2-b]pyrrole-5- | 0.018 | 2.2 |
| | carboxylate | | |
| 33 | 1-acetoxyethyl 4H-furo[3,2-b]pyrrole-5-carboxylate | 0.0077 | 2.8 |
| 34 | 1-(propionyloxy)ethyl 4H-furo[3,2-b]pyrrole-5- | 0.0402 | 1.3 |
| | carboxylate | | |
| 35 | 1-(isobutyryloxy)ethyl 4H-furo[3,2-b]pyrrole-5- | 0.021 | 0.4 |
| | carboxylate | | |
| 36 | 1-(isopropoxycarbonyloxy)ethyl 4H-furo[3,2- | 0.0257 | 0.2 |
| | b]pyrrole-5-carboxylate | | |
| 37 | 1-(tert-butoxycarbonyloxy)ethyl 4H-furo[3,2- | −0.004 | 7.1 |
| | b]pyrrole-5-carboxylate | | |
| 38 | acetoxymethyl 4H-furo[3,2-b]pyrrole-5-carboxylate | 0.0109 | 5.5 |
| 39 | 1-(cyclohexyloxycarbonyloxy)ethyl 4H-furo[3,2- | 0.0062 | 0.1 |
| | b]pyrrole-5-carboxylate | | |
| 40 | 1-acetoxy-2-methylpropyl 4H-furo[3,2-b]pyrrole-5- | 0.019 | 1.0 |
| | carboxylate | | |
| 41 | methylene bis(4H-furo[3,2-b]pyrrole-5- | 0.0141 | 0.5 |
| | carboxylate) | | |
| 43 | butyryloxymethyl 4H-furo[3,2-b]pyrrole-5- | 0.0096 | |
| | carboxylate | | |
| 44 | 1-(butyryloxy)ethyl 4H-furo[3,2-b]pyrrole-5- | 0.0472 | 1.5 |
| | carboxylate | | |
| 45 | 1-acetoxypropyl 4H-furo[3,2-b]pyrrole-5- | 0.0258 | 2.1 |
| | carboxylate | | |
| 50 | 1-(pivaloyloxy)ethyl 4H-furo[3,2-b]pyrrole-5- | 0.0206 | 0.1 |
| | carboxylate | | |
| 55 | ethane-1,2-diyl bis(4H-furo[3,2-b]pyrrole-5- | 0.0035 | 0.2 |
| | carboxylate) | | |
| 72 | (5-methyl-2-oxo-1,3-dioxol-4-yl)methyl 4H- | −0.012 | 6.2 |
| | furo[3,2-b]pyrrole-5-carboxylate | | |
| 73 | 2-oxo-1,3-dioxolan-4-yl 4H-furo[3,2-b]pyrrole-5- | −0.007 | 6.7 |
| | carboxylate | | |
| 74 | 3-oxo-1,3-dihydroisobenzofuran-1-yl 4H-furo[3,2- | 0.0016 | 5.3 |
| | b]pyrrole-5-carboxylate | | |
| 76 | chloromethyl 4H-furo[3,2-b]pyrrole-5-carboxylate | 0.0018 | 3.5 |

### Example 15

### Pharmacokinetic-Pharmacodynamic Evaluation of Compounds in Rats

Conversion of test compounds to 4H-furo[3,2-b]pyrrole-5-carboxylic acid (“acid”; 77) in rats was evaluated according to General Procedure 12. 4H-furo[3,2-b]pyrrole-5-carboxylic acid and D-serine levels in plasma and cerebellum were measured. Animals were sacrificed at 6 hours post administration. In Table 6, “[D-serine] cerebellum” refers to D-serine levels, in nmol/g, in the rat cerebellum. In Table 6, “[acid] cerebellum” refers to the concentration, in μM, of the 4H-furo[3,2-b]pyrrole-5-carboxylic acid in the cerebellum. In Table 6, “[acid] plasma” refers to the concentration, in μM, of the 4H-furo[3,2-b]pyrrole-5-carboxylic acid in the plasma. The row labeled “acid”, denotes the data obtained from dosing 4H-furo[3,2-b]pyrrole-5-carboxylic acid. The row labeled “vehicle” denotes the data obtained from dosing vehicle, and the remaining row in the table denote the data obtained from dosing the test compounds.

TABLE 6
PD effect following oral dosing.

| | | [D-serine] | [acid] | |
|---|---|---|---|---|
| Compound # | Compound Name | cerebellum | cerebellum | [acid] plasma |
| | Vehicle | | | |
| 77 | 4H-furo[3,2-b]pyrrole-5-carboxylic | | | |
| | acid | | | |
| 2 | 11,11-dimethyl-5,10-dioxa-1- | 17 ± 1.0(3) | 0.37 ± 0.040(3) | 1.30 ± 0.552(3) |
| | azatricyclo[6.3.0.0^{2,6}]undeca- | | | |
| | 2(6),3,7-trien-9-one | | | |
| 3 | 11-methyl-5,10-dioxa-1- | 17 ± 1.0(3) | 0.47 ± 0.076(3) | 2.44 ± 0.977(3) |
| | azatricyclo[6.3.0.0^{2,6}]undeca- | | | |
| | 2(6),3,7-trien-9-one | | | |
| 4 | propyl 4H-furo[3,2-b]pyrrole-5- | 20 ± 1.3(3) | 0.58 ± 0.163(3) | 4.44 ± 2.068(3) |
| | carboxylate | | | |
| 5 | butyl 4H-furo[3,2-b]pyrrole-5- | 18 ± 1.2(3) | 0.48 ± 0.039(3) | 2.84 ± 0.331(3) |
| | carboxylate | | | |
| 6 | heptyl 4H-furo[3,2-b]pyrrole-5- | 18 ± 0.7(3) | 0.56 ± 0.039(3) | 2.99 ± 0.437(3) |
| | carboxylate | | | |
| 7 | isobutyl 4H-furo[3,2-b]pyrrole-5- | 19 ± 1.5(3) | 0.56 ± 0.025(3) | 3.76 ± 0.598(3) |
| | carboxylate | | | |
| 8 | 4-methylpentyl 4H-furo[3,2- | 20 ± 1.5(3) | 0.54 ± 0.127(3) | 3.16 ± 0.648(3) |
| | b]pyrrole-5-carboxylate | | | |
| 9 | benzyl 4H-furo[3,2-b]pyrrole-5- | 21 ± 0.4(3) | 0.62 ± 0.095(3) | 3.58 ± 0.995(3) |
| | carboxylate | | | |
| 10 | (tetrahydrofuran-2-yl)methyl 4H- | 19 ± 2.0(6) | 0.64 ± 0.207(6) | 5.20 ± 3.067(6) |
| | furo[3,2-b]pyrrole-5-carboxylate | | | |
| 19 | 2-(azetidin-1-yl)-2-oxoethyl 4H- | 17 ± 0.7(3) | 0.38 ± 0.043(3) | 1.24 ± 0.390(3) |
| | furo[3,2-b]pyrrole-5-carboxylate | | | |
| 20 | 2-(dimethylamino)-2-oxoethyl 4H- | 19 ± 0.8(6) | 0.43 ± 0.062(6) | 3.14 ± 2.082(6) |
| | furo[3,2-b]pyrrole-5-carboxylate | | | |
| 21 | 2-morpholino-2-oxoethyl 4H- | 19 ± 1.0(6) | 0.69 ± 0.332(6) | 7.73 ± 7.758(6) |
| | furo[3,2-b]pyrrole-5-carboxylate | | | |
| 23 | 2-(diethylamino)-2-oxoethyl 4H- | 18 ± 2.3(3) | 0.59 ± 0.192(3) | 3.35 ± 1.846(3) |
| | furo[3,2-b]pyrrole-5-carboxylate | | | |
| 27 | 2-(1H-imidazol-1-yl)ethyl 4H- | 17 ± 0.6(3) | 0.49 ± 0.068(3) | 1.80 ± 0.811(3) |
| | furo[3,2-b]pyrrole-5-carboxylate | | | |
| 28 | (1H-imidazol-1-yl)methyl 4H- | 19 ± 1.9(3) | 0.53 ± 0.081(3) | 2.46 ± 0.615(3) |
| | furo[3,2-b]pyrrole-5-carboxylate | | | |
| 30 | 2-morpholinoethyl 4H-furo[3,2- | 14 ± 1.2(3) | 0.34 ± 0.049(3) | 1.69 ± 0.294(3) |
| | b]pyrrole-5-carboxylate | | | |
| 32 | 2-(dimethylamino)ethyl 4H- | 9 ± 0.7(3) | 0.24 ± 0.003(3) | 0.52 ± 0.114(3) |
| | furo[3,2-b]pyrrole-5-carboxylate | | | |
| 33 | 1-acetoxyethyl 4H-furo[3,2- | 19 ± 1.4(3) | 0.54 ± 0.065(3) | 2.77 ± 0.603(3) |
| | b]pyrrole-5-carboxylate | | | |
| 34 | 1-(propionyloxy)ethyl 4H-furo[3,2- | 20 ± 1.7(3) | 0.49 ± 0.084(3) | 1.08 ± 0.452(3) |
| | b]pyrrole-5-carboxylate | | | |
| 35 | 1-(isobutyryloxy)ethyl 4H-furo[3,2- | 16 ± 0.9(3) | 0.42 ± 0.071(3) | 1.62 ± 0.530(3) |
| | b]pyrrole-5-carboxylate | | | |
| 36 | 1-(isopropoxycarbonyloxy)ethyl | 20 ± 2.9(6) | 0.57 ± 0.088(6) | 2.59 ± 0.880(6) |
| | 4H-furo[3,2-b]pyrrole-5- | | | |
| | carboxylate | | | |
| 37 | 1-(tert-butoxycarbonyloxy)ethyl | 18 ± 1.1(3) | 0.42 ± 0.063(3) | 2.18 ± 0.958(3) |
| | 4H-furo[3,2-b]pyrrole-5- | | | |
| | carboxylate | | | |
| 38 | acetoxymethyl 4H-furo[3,2- | 19 ± 4.4(4) | 0.52 ± 0.119(4) | 2.29 ± 0.753(4) |
| | b]pyrrole-5-carboxylate | | | |
| 39 | 1-(cyclohexyloxycarbonyloxy)ethyl | 17 ± 2.6(3) | 0.57 ± 0.237(3) | 3.67 ± 2.581(3) |
| | 4H-furo[3,2-b]pyrrole-5- | | | |
| | carboxylate | | | |
| 40 | 1-acetoxy-2-methylpropyl 4H- | 22 ± 2.7(3) | 0.49 ± 0.025(3) | 1.70 ± 0.414(3) |
| | furo[3,2-b]pyrrole-5-carboxylate | | | |
| 41 | methylene bis(4H-furo[3,2- | 14 ± 0.2(3) | 0.61 ± 0.136(3) | 3.57 ± 1.257(3) |
| | b]pyrrole-5-carboxylate) | | | |
| 42 | propionyloxymethyl 4H-furo[3,2- | 20 ± 1.5(3) | 0.58 ± 0.111(3) | 10.57 ± 3.484(3) |
| | b]pyrrole-5-carboxylate | | | |
| 44 | 1-(butyryloxy)ethyl 4H-furo[3,2- | 21 ± 1.3(3) | 0.62 ± 0.124(3) | 8.87 ± 4.474(3) |
| | b]pyrrole-5-carboxylate | | | |
| 45 | 1-acetoxypropyl 4H-furo[3,2- | 22 ± 1.9(3) | 0.55 ± 0.095(3) | 9.55 ± 2.449(3) |
| | b]pyrrole-5-carboxylate | | | |
| 47 | isobutyryloxymethyl 4H-furo[3,2- | 22 ± 3.0(3) | 0.56 ± 0.070(3) | 11.56 ± 3.196(3) |
| | b]pyrrole-5-carboxylate | | | |
| 55 | ethane-1,2-diyl bis(4H-furo[3,2- | 13 ± 1.1(3) | 0.40 ± 0.042(3) | 1.34 ± 0.349(3) |
| | b]pyrrole-5-carboxylate) | | | |
| 56 | 4H-furo[3,2-b]pyrrole-5- | 7 ± 0.7(2) | 0.15 ± 0.050(2) | 0.16 ± 0.047(2) |
| | carboxamide | | | |
| 57 | N-(2-amino-2-oxoethyl)-4H- | 5 ± 0.5(3) | 0.06 ± 0.007(3) | 0.12 ± 0.012(3) |
| | furo[3,2-b]pyrrole-5-carboxamide | | | |
| 58 | (4H-furo[3,2-b]pyrrol-5-yl)(4- | 5 ± 0.3(3) | 0.04 ± 0.012(3) | 0.00 ± 0.000(3) |
| | methylpiperazin-1-yl)methanone | | | |
| 59 | ethyl 4-(benzoyloxymethyl)-4H- | 11 ± 1.9(3) | 0.38 ± 0.116(3) | 4.16 ± 1.658(3) |
| | furo[3,2-b]pyrrole-5-carboxylate | | | |
| 60 | 4-(benzoyloxymethyl)-4H-furo[3,2- | 13 ± 1.1(3) | 0.39 ± 0.091(3) | 4.55 ± 1.755(3) |
| | b]pyrrole-5-carboxylic acid | | | |
| 64 | ethyl 4-(acetoxymethyl)-4H- | 14 ± 3.6(3) | 0.40 ± 0.111(3) | 4.72 ± 1.309(3) |
| | furo[3,2-b]pyrrole-5-carboxylate | | | |
| 65 | ethyl 4-(isobutyryloxymethyl)-4H- | 11 ± 2.6(3) | 0.41 ± 0.074(3) | 5.70 ± 2.095(3) |
| | furo[3,2-b]pyrrole-5-carboxylate | | | |
| 66 | 4-(acetoxymethyl)-4H-furo[3,2- | 12 ± 0.7(3) | 0.41 ± 0.070(3) | 3.87 ± 2.191(3) |
| | b]pyrrole-5-carboxylic acid | | | |
| 67 | 4-(isobutyryloxymethyl)-4H- | 14 ± 0.9(3) | 0.47 ± 0.112(3) | 6.02 ± 2.763(3) |
| | furo[3,2-b]pyrrole-5-carboxylic | | | |
| | acid | | | |
| 68 | (S)-ethyl 4-((2-(2-amino-3- | 5 ± 0.4(3) | 0.00 ± 0.000(3) | 0.00 ± 0.000(3) |
| | methylbutanoyloxy)ethoxy)methyl)- | | | |
| | 4H-furo[3,2-b]pyrrole-5- | | | |
| | carboxylate | | | |
| 71 | acetoxymethyl 4-(acetoxymethyl)- | 14 ± 0.9(3) | 0.34 ± 0.056(3) | 3.95 ± 1.806(3) |
| | 4H-furo[3,2-b]pyrrole-5- | | | |
| | carboxylate | | | |
| 72 | (5-methyl-2-oxo-1,3-dioxol-4- | 21 ± 3.5(9) | 0.74 ± 0.198(9) | 8.62 ± 5.823(9) |
| | yl)methyl 4H-furo[3,2-b]pyrrole-5- | | | |
| | carboxylate | | | |
| 73 | 2-oxo-1,3-dioxolan-4-yl 4H- | 24 ± 2.0(2) | 0.89 ± 0.037(2) | 3.94 ± 0.262(2) |
| | furo[3,2-b]pyrrole-5-carboxylate | | | |
| 74 | 3-oxo-1,3-dihydroisobenzofuran-1- | 13 ± 1.2(3) | 0.60 ± 0.117(3) | 3.63 ± 0.552(3) |
| | yl 4H-furo[3,2-b]pyrrole-5- | | | |
| | carboxylate | | | |
| 76 | chloromethyl 4H-furo[3,2- | 12 ± 1.5(3) | 0.45 ± 0.016(3) | 1.80 ± 0.643(3) |
| | b]pyrrole-5-carboxylate | | | |

[D-serine] in the cerebellum and plasma, and [acid] in the plasma 6 hours after PO dosing at 10 mg/kg in rats.

### Example 16

### Pharmacokinetic-Pharmacodynamic Evaluation of Compounds in Cannulated Male Rats

4H-Furo[3,2-b]pyrrole-5-carboxylic acid (“acid”) and test compounds were evaluated in cannulated rats according to General Procedure 13. For all compounds, levels of 4H-furo[3,2-b]pyrrole-5-carboxylic acid (“acid”) and D-serine in plasma were measured for each time point. Standard PK parameters were calculated for all compounds using WinNonlin software. For test compounds, reported oral bioavailability is for 4H-furo[3,2-b]pyrrole-5-carboxylic acid obtained after oral dosing of test compounds, in comparison to i.v. dosing of 4H-furo[3,2-b]pyrrole-5-carboxylic acid.

TABLE 7
PK parameters following oral dosing

| | | | | % F |
|---|---|---|---|---|
| Compound # | Compound name | Cmax ng/mL | Tmax hr | % |
| acid | 4H-furo[3,2-b]pyrrole-5-carboxylic acid | 1190 | 0.3 | 51 |
| 3 | 11-methyl-5,10-dioxa-1- | 2115 | 0.2 | 56 |
| | azatricyclo[6.3.0.0^{2,6}]undeca-2(6),3,7- | | | |
| | trien-9-one | | | |
| 10 | (tetrahydrofuran-2-yl)methyl 4H-furo[3,2- | 1270 | 0.6 | 62 |
| | b]pyrrole-5-carboxylate | | | |
| 12 | 2-methoxyphenyl 4H-furo[3,2-b]pyrrole- | 530 | 0.9 | 31 |
| | 5-carboxylate | | | |
| 19 | 2-(azetidin-1-yl)-2-oxoethyl 4H-furo[3,2- | 720 | 0.5 | 24 |
| | b]pyrrole-5-carboxylate | | | |
| 20 | 2-(dimethylamino)-2-oxoethyl 4H- | 1300 | 0.8 | 57 |
| | furo[3,2-b]pyrrole-5-carboxylate | | | |
| 21 | 2-morpholino-2-oxoethyl 4H-furo[3,2- | 1100 | 1.0 | 73 |
| | b]pyrrole-5-carboxylate | | | |
| 22 | 2-(dimethylamino)-2-oxoethyl 4H- | 21 | 3 | 2 |
| | furo[3,2-b]pyrrole-5-carboxylate | | | |
| 23 | 2-(diethylamino)-2-oxoethyl 4H-furo[3,2- | 1631 | 0.4 | 34 |
| | b]pyrrole-5-carboxylate | | | |
| 28 | (1H-imidazol-1-yl)methyl 4H-furo[3,2- | 1300 | 0.6 | 50 |
| | b]pyrrole-5-carboxylate | | | |
| 29 | 2-(2-methyl-1H-imidazol-1-yl)ethyl 4H- | 619 | 1 | 27 |
| | furo[3,2-b]pyrrole-5-carboxylate | | | |
| 30 | 2-morpholinoethyl 4H-furo[3,2-b]pyrrole- | 500 | 0.5 | 16 |
| | 5-carboxylate | | | |
| 31 | 2-(diethylamino)ethyl 4H-furo[3,2- | 496 | 1 | 22 |
| | b]pyrrole-5-carboxylate | | | |
| 35 | 1-(isobutyryloxy)ethyl 4H-furo[3,2- | 1100 | 0.5 | 52 |
| | b]pyrrole-5-carboxylate | | | |
| 36 | 1-(isopropoxycarbonyloxy)ethyl 4H- | 672 | 0.8 | 34 |
| | furo[3,2-b]pyrrole-5-carboxylate | | | |
| 37 | 1-(tert-butoxycarbonyloxy)ethyl 4H- | 1100 | 0.5 | 43 |
| | furo[3,2-b]pyrrole-5-carboxylate | | | |
| 38 | acetoxymethyl 4H-furo[3,2-b]pyrrole-5- | 1253 | 0.5 | 47 |
| | carboxylate | | | |
| 39 | 1-(cyclohexyloxycarbonyloxy)ethyl 4H- | 1600 | 0.6 | 64 |
| | furo[3,2-b]pyrrole-5-carboxylate | | | |
| 51 | 2-(2-morpholinoacetoxy)ethyl 4H- | 270 | 0.7 | 12 |
| | furo[3,2-b]pyrrole-5-carboxylate | | | |
| 55 | ethane-1,2-diyl bis(4H-furo[3,2- | 81 | 1 | 5 |
| | b]pyrrole-5-carboxylate) | | | |
| 72 | (5-methyl-2-oxo-1,3-dioxol-4-yl)methyl | 1300 | 0.6 | 80 |
| | 4H-furo[3,2-b]pyrrole-5-carboxylate | | | |

### Example 17

### Rate of Hydrolysis of Test Compounds to 4H-furo[3,2-b]pyrrole-5-carboxylic acid in pH 7.4 and 2.0 Aqueous Solutions

The rate of hydrolysis of test compounds to 4H-furo[3,2-b]pyrrole-5-carboxylic acid was evaluated in pH 7.4 aqueous solution over 6 hours, and in pH 2.0 aqueous solution over 2 hours, according to General Procedure 14.

TABLE 8
Hydrolysis Rates at ph 7.4 and pH 2.0

| | | Hydrolysis rate at | Hydrolysis rate at |
|---|---|---|---|
| Compound # | Compound name | pH 7.4 | pH 2.0 |
| 10 | (tetrahydrofuran-2-yl)methyl 4H- | No hydrolysis | No hydrolysis |
| | furo[3,2-b]pyrrole-5-carboxylate | observed over 6 | observed over 2 |
| | | hours | hours |
| 19 | 2-(azetidin-1-yl)-2-oxoethyl 4H- | No hydrolysis | No hydrolysis |
| | furo[3,2-b]pyrrole-5-carboxylate | observed over 6 | observed over 2 |
| | | hours | hours |
| 20 | 2-(dimethylamino)-2-oxoethyl 4H- | No hydrolysis | No hydrolysis |
| | furo[3,2-b]pyrrole-5-carboxylate | observed over 6 | observed over 2 |
| | | hours | hours |
| 21 | 2-morpholino-2-oxoethyl 4H-furo[3,2- | No hydrolysis | No hydrolysis |
| | b]pyrrole-5-carboxylate | observed over 6 | observed over 2 |
| | | hours | hours |
| 30 | 2-morpholinoethyl 4H-furo[3,2- | No hydrolysis | No hydrolysis |
| | b]pyrrole-5-carboxylate | observed over 6 | observed over 2 |
| | | hours | hours |
| 35 | 1-(isobutyryloxy)ethyl 4H-furo[3,2- | 3.46%/hr | |
| | b]pyrrole-5-carboxylate | | |
| 39 | 1-(cyclohexyloxycarbonyloxy)ethyl 4H- | 0.21%/hr | |
| | furo[3,2-b]pyrrole-5-carboxylate | | |
| 72 | (5-methyl-2-oxo-1,3-dioxol-4-yl)methyl | 0.20%/hr | No hydrolysis |
| | 4H-furo[3,2-b]pyrrole-5-carboxylate | | observed over 2 |
| | | | hours |

### REFERENCE ITEMS

Further, procedures that may be useful to one of ordinary skill in the art in synthesizing various embodiments of the present inventions can be found in Reference items 1 to 28 listed below, each of which is incorporated herein by reference in its entirety.
1.(a) J. Org. Chem. 2004, 69, 3620-3627; (b) Org. Letters 2001, 3, 1395-1397.
2.(a) Eur. Pat. Appl. Nov. 19, 2003, 1362864 A1; (b) WO 2001079208 A1; (c) Eur. J. Pharm. Sci. 2005, 24, 441-449; (d) Helv. Chim. Acta 2002, 85, 788-798; (e) Heterocycles 1996, 43, 2361-2364; (f) WO 9517381 A1.
3.(a) Synthesis 2003, 18, 2795-2798; (b) J. Org. Chem. 2004, 69, 9313-9315; (c) J. Org. Chem. 2003, 68, 5720-5723.
4.(a) Eur. Pat. Appl. Nov. 19, 2003, 1362864 A1; (b) WO 2001079208 Al; (c) Eur. J. Pharm. Sci. 2005, 24, 441-449; (d) Helv. Chim. Acta 2002, 85, 788-798; (e) Heterocycles 1996, 43, 2361-2364; (f) WO 9517381 A1.
5. (a) Shangqiu Shifan Xueyuan Xuebao 2004, 20, 90-94; (b) Bioorg. Med. Chem. Lett. 2005, 15, 2239-2242; (c) J. Pharm. Pharmacol. 1995, 47, 861; (d) Eur. J. Med. Chem. 1985, 20, 467;. (e) Eur. J. Med. Chem. 1985, 20, 459; (f) Pharm. Res. 1991, 8, 832; (g) Int. J. Pharm. 1990, 63, 129; (h) J. Pharmaceut. Sci. 1998, 87, 880-885.
6. (a) J. Med. Chem. 2000, 43, 19-21; (b) J. Med. Chem. 1992, 35, 687-694; (c) J. Med. Chem. 1986, 29, 448-453; (d) Drug Metabolism and Disposition 2005, 33(12), 1911.

7. WO 2005046575A2.
8. (a) J. Med. Chem. 1985, 28, 106; (b) J. Pharm. Pharmacol. 1991, 43, 815; (c) Pharm. Res. 1990, 7, 5222; (d) Adv. Drug Delivery Rev. 1994, 14, 227.
9. (a) J. Med. Chem. 1987, 30, 451-454; (b) Huaxue Shiji, 2006, 28, 371-372; (c) Bioorg. Med. Chem. Lett. 2006, 14, 4820-4833; (d) Pharm. Res. 1989, 6, 683; (e) Pharm. Res. 1992, 9, 1058; (f) Pharm. Res. 1996, 13, 284; (g) J. Pharm. Res. 1988, 77, 285.
10. (a) J. Med. Chem. 1995, 38, 3488-3501; (b) J. Pharm. Sci. 1994, 83, 644. (c) Pharm. Res. 1992, 9, 492-496; (d) J. Heterocyclic Chem. 1990, 27, 215-219.
11. J. Med. Chem. 2000, 43, 1489-1494.
12. (a) J. Med. Chem. 1989, 32, 493-503; (b) J. Med. Chem. 1992, 35, 687-694; (c) Eur. J. Pharmaceut. Sci. 2005, 24, 433-440; (d) J. Med. Chem. 2005, 48, 1042-1054; (e) Tetrahedron Lett. 2007, 48, 4609-4611; (f) J. Med. Chem. 1990, 33, 291; (g) J. Med. Chem. 1990, 33, 344; (h) J. Pharm. Sci. 2006, 95, 1177; (i) Current Drug Metabolism 2003, 4, 461; (j) Syn. Commun. 1998, 28, 4345-4354.
13. (a) Biorg. Med. Chem. Lett. 2006, 14 (12), 4193; (b) J. Org. Chem. 1995, 60 (4) 946; (c) J. Pharm. Sci. 2006, 95, 1177; (d) Current Drug Metabolism 2003, 4, 461; (e) Syn. Commun. 1998, 28, 4345-4354.
14. Tetrahedron Lett. 2007, 48, 4609-4611.
15. (a) Pharmaceut. Res. 2003, 20, 205-210; (b) Cancer Chemotherapy and Pharmacology 1997, 39(6), 486; (c) J. Pharm. Exp. Thera. 1995, 274(2), 877; (d) PCT Int. Appl. 2001, 128 pp. WO 2001002427. (e) PCT Int. Appl. 2004, 66 pp. WO 2004039787. (f) Org. Lett. 2004, 6, 4089-4092. (g) J. Pharm. Sci. 2005, 94, 2198. (h) Cancer Chemotherapy and Pharmacology 1997, 39(6), 486; (i) Bioorg. Med. Chem. Let. 1995, 5(3), 247.
16. (a) Bioorg. Med. Chem. Let. 1995, 5(3), 247-252; (b) Bioorg. Med. Chem. Let. 1993, 3, 1761.
17. (a) WO 2006021000A2; (b) J. Med. Chem. 2007, 50, 901-914.
18. (a) J. Med. Chem. 2004, 47, 3892-2896; (b) Chem. Pharm. Bull. 2006, 54(8), 1067; (c) Bioorg. Med. Chem. Lett. 2005, 15(6), 1609; (d) Current Opinion in Neurology 2003, 16(2), 203; (e) Peptide Science 2002, Volume Date 2001, 38th 249.
19. Bioorg. Med. Chem. Lett. 2007, 17, 2908-2912.
20. (a) Bioorg. Med. Chem. Lett. 2007, 17, 683-687; (b) Bioorg. Med. Chem. Lett. 2006, 14, 2569-2580.
21. Bioorg. Med. Chem. 2004, 12, 5213-5224.
22. (a) Faming Zhuanli Shenqing Gongkai Shuomingshu (2005), CN 1709871 A 20051221; (b) Eur. Pat. Appl. (2004), EP 1420028 A2; (c) Journal of the American Chemical Society (2003), 125(1), 163-168.
23. (a) Current Med. Chem. 2005, 12, 2095-2162; (b) US 2006/0229286 A1; (c) WO0982477A1; (d) J. Chem. Soc., Perkins 1, 1986, 12, 2181-2186; (e) US 2003/0087803 A1.
24. (a) Tetrahedron Lett. 2002, 43, 577-579; (b) Org. Biomol. Chem. 2006, 4, 2869-2873; (c) Tetrahedron Lett. 2002, 43, 577-579.
25. (a) Synthetic Commun. 1994, 24, 767-772; (b) Bioorg. Med. Chem. Lett. 2007, 17, 465-470.
26. (a) J. Indian Chemical Society 2005, 82, 158-160; (b) Jpn. Kokai Tokkyo Koho, 54059269, 12 May 1979; (c) Bioorg. Med. Chem. Lett. 2002, 10, 415-424; (d) J. Am. Chem. Soc. 2005, 127, 5970-5978; (e) Org. Biomol. Chem. 2004, 2, 2874-2883; (f) Tetrahedron 2003, 59, 1265-1275; (g) Faming Zhuanli Shenqing Gongkai Shuomingshu, 1962656, 16 May 2007; (h) Tetrahedron Lett. 2007, 48, 4609-4611.
27. J. Med. Chem. 2005, 48, 5305-5320.
28. J. Heterocyclic Chem. 1982, 19, 1025-1028.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

All statements made herein with regard to compounds of formulae “A, A^{x}, B, C and/or Q” or “A-Q (including Formula A^{x})” are meant to equally apply to all compounds of the present invention.

What is claimed is:

1. A compound having the formula: 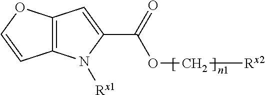
wherein
R^{x1} is a member selected from H and 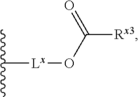
wherein
L^{x} is a member selected from substituted or unsubstituted alkyl and substituted or unsubstituted heteroalkyl; and
R^{x3} is a member selected from substituted or unsubstituted alkyl and substituted or unsubstituted aryl;
n1 is selected from the integers from 0 to 10; and
R^{x2} is a member selected from H, unsubstituted alkyl, substituted or unsubstituted heteroaryl, substituted or unsubstituted aryl, substituted or unsubstituted cycloalkyl, 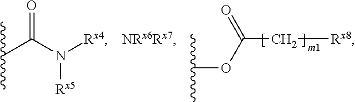
halogen and 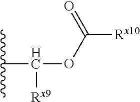
wherein
R^{x4} and R^{x5} are independently selected substituted or unsubstituted C₂-C₁₀ alkyl; and
R^{x4} and R^{x5}, together with the nitrogen atom to which they are attached, are optionally joined to form a substituted or unsubstituted 3- to 8-membered heterocycloalkyl ring,
wherein
said heterocycloalkyl ring comprises up to one additional non-oxygen heteroatom; and
said heterocycloalkyl ring is other than unsubstituted azetidine or substituted pyrrolidine;
R^{x6} and R^{x7} are independently selected substituted or unsubstituted C₁-C₁₀ alkyl
m1 is selected from the integers from 0 to 3;
R^{x8} is a member selected from substituted or unsubstituted piperazinyl and 4H-furo[3,2-b]pyrrol-5-yl;
R^{x9} is a member selected from H, methyl, ethyl, and iso-propyl; and
R^{x10} is a member selected from unsubstituted C₁-C₄ alkyl and 4H-furo[3,2-b]pyrrol-5-yl,
wherein
when R^{x1} and R^{x9} are H, R^{x10} is other than methyl;
when R^{x1} is H and R^{x9} is methyl, R^{x10} is other than methyl, ethyl or iso-propyl; and
when R^{x1} is H and R^{x9} is iso-propyl, R^{x10} is other than methyl;
with the proviso that:
when R^{x1} is H, R^{x2} is other than H or unsubstituted alkyl;
when R^{x1} is H and n1 is 1, R^{x2} is other than phenyl or imidazol-1-yl;
when R^{x1} is H and n1 is 2, R^{x2} is other than imidazol-1-yl;
when n1 is other than 0, R^{x2} is other than 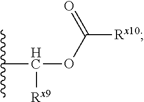
and
when R^{x2} is H, n1 is 0 and R^{x1} is

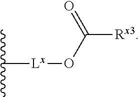

2. The compound according to claim 1, wherein
R^{x1} is 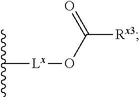
and
R^{x2} is a member selected from H, methyl, ethyl and —CH₂O(CO)CH₃.

3. A pharmaceutical composition comprising a compound according to claim 1, or a pharmaceutically acceptable salt thereof, and a pharmaceutically acceptable carrier.

4. A method for treating or preventing a condition which is a member selected from a neurological disorder, pain, ataxia, convulsion, loss of memory, loss of cognition and a combination thereof, said method comprising administering to a subject in need thereof a therapeutically effective amount of a compound according to claim 1.

5. The method according to claim 4, wherein said condition is pain, wherein said pain is neuropathic pain.

6. The method according to claim 4, wherein said condition is selected from loss of memory, loss of cognition and a combination thereof

7. The method according to claim 6, wherein said condition is associated with Alzheimer's disease.

8. The method according to claim 6, wherein said condition is associated with schizophrenia.

9. The method according to claim 4, wherein said compound is administered as a pharmaceutical composition comprising a pharmaceutically acceptable carrier and a dosage of from about 1 mg to about 7000 mg of said compound.

10. A compound having a formula selected from:

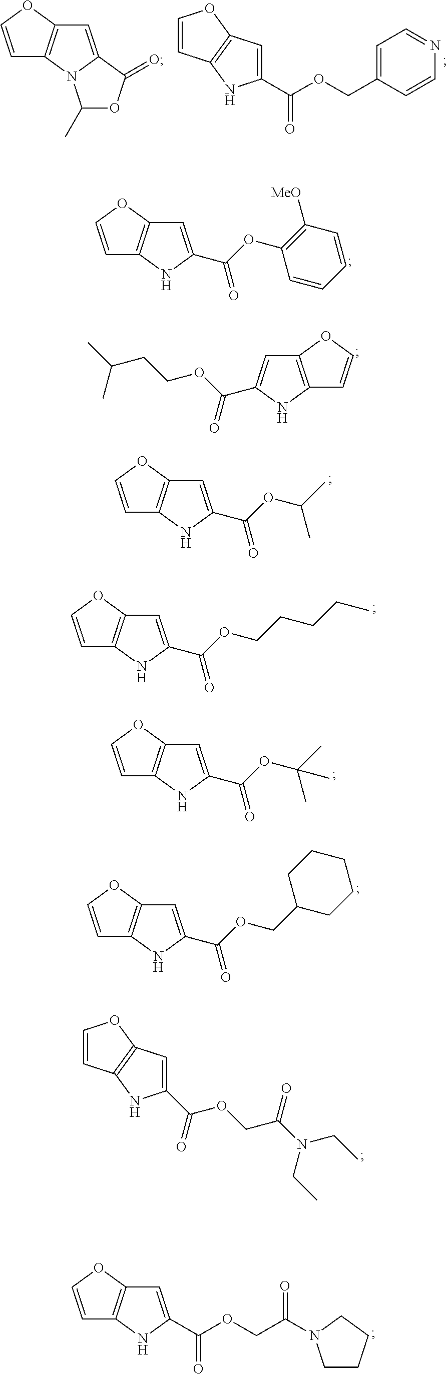

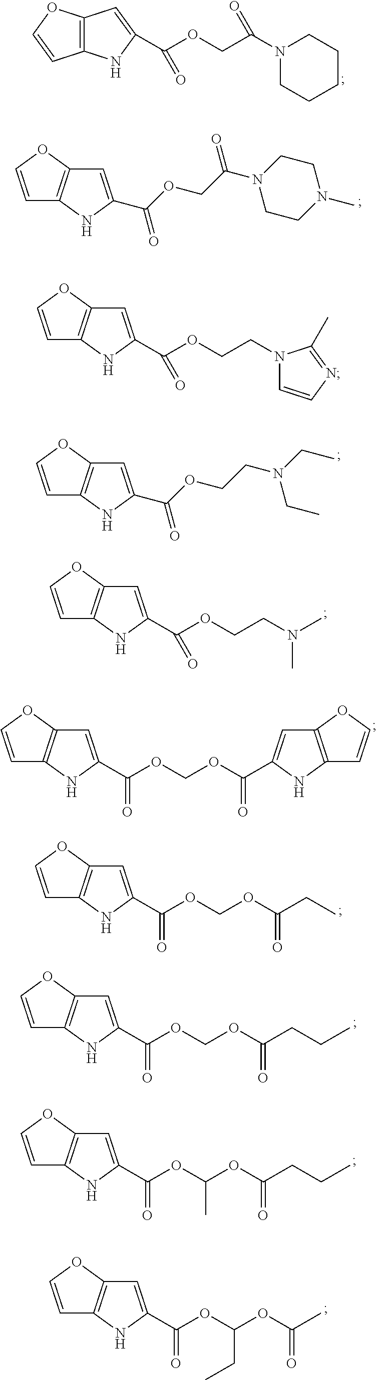

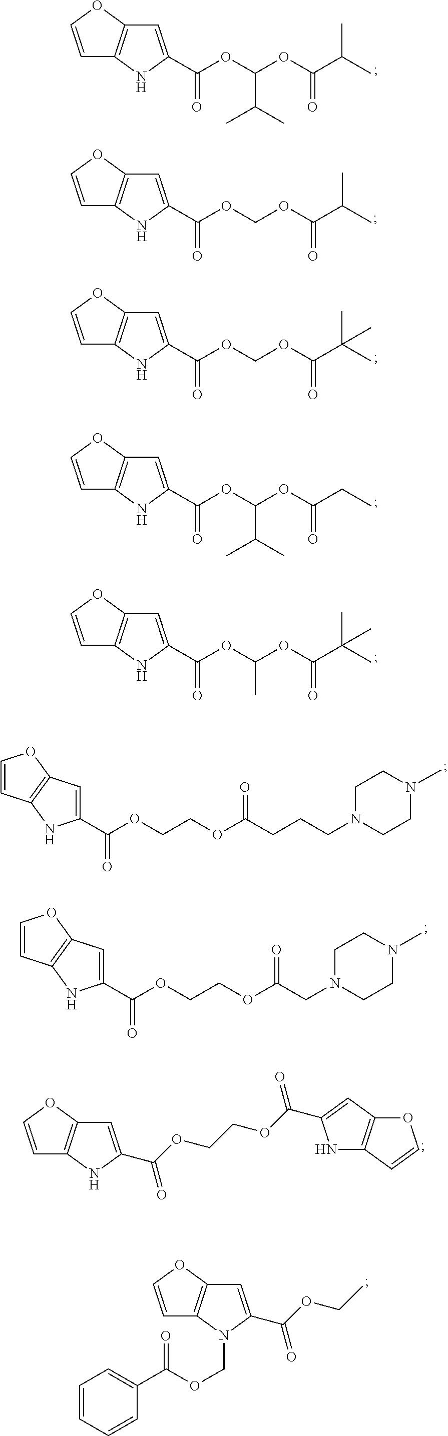

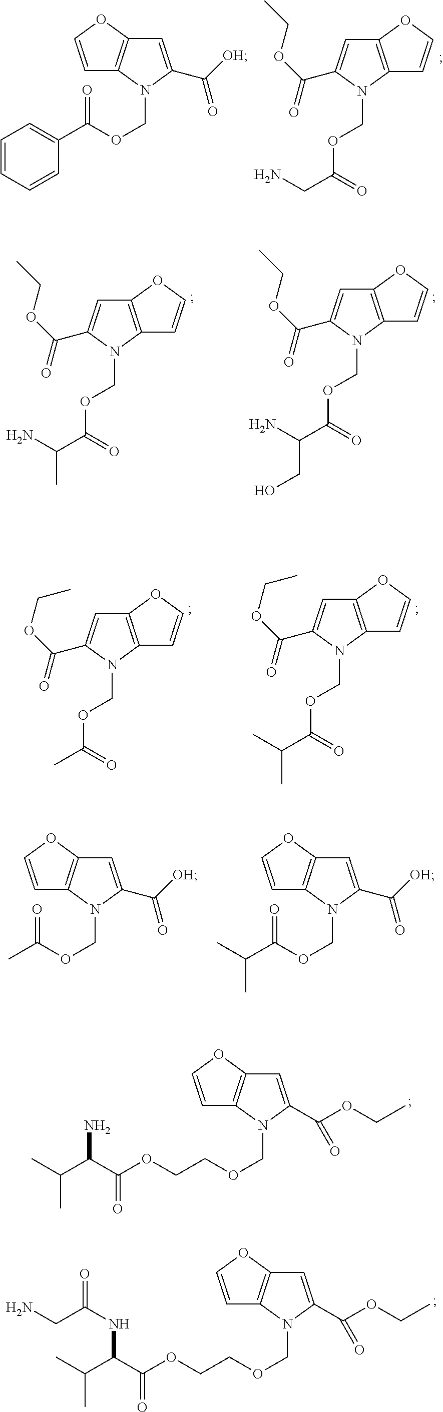

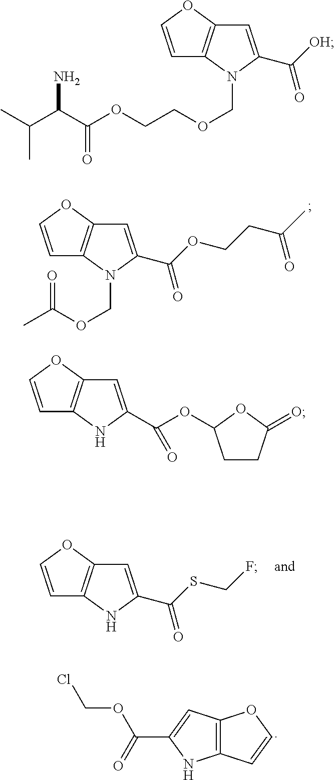

11. A pharmaceutical composition comprising a compound according to claim 10, or a pharmaceutically acceptable salt thereof, and a pharmaceutically acceptable carrier.

12. A method for treating or preventing a condition which is a member selected from a neurological disorder, pain, ataxia, convulsion, loss of memory, loss of cognition and a combination thereof, said method comprising administering to a subject in need thereof a therapeutically effective amount of a compound according to claim 10.

13. The method according to claim 12, wherein said condition is pain, wherein said pain is neuropathic pain.

14. The method according to claim 12, wherein said condition is selected from loss of memory, loss of cognition and a combination thereof

15. The method according to claim 14, wherein said condition is associated with Alzheimer's disease.

16. The method according to claim 14, wherein said condition is associated with schizophrenia.

17. The method according to claim 12, wherein said compound is administered as a pharmaceutical composition comprising a pharmaceutically acceptable carrier and a dosage of from about 1 mg to about 7000 mg of said compound.
